(19) United States
(12) Patent Application Publication
(10) Pub. No.: US 2025/0250636 A1
(43) Pub. Date: Aug. 7, 2025

(54) ULTRA-SENSITIVE LIQUID BIOPSY THROUGH DEEP LEARNING EMPOWERED WHOLE GENOME SEQUENCING OF PLASMA

(71) Applicants: Cornell University, lthaca, NY (US); Memorial Sloan-Kettering Cancer Center, New York, NY (US); New York Genome Center, Inc., New York, NY (US)

(72) Inventors: Dan Landau, New York, NY (US); Adam Widman, New York, NY (US); Cole Khamnei, New York, NY (US); Jacob Bass, New York, NY (US)

(21) Appl. No.: 18/682,736

(22) PCT Filed: Aug. 10, 2022
(86) PCT No.: PCT/US22/39945

§ 371 Date: Feb. 9, 2024

Related U.S. Application Data
(60) Provisional application No. 63/296,356, filed on Jan. 4, 2022.
(60) Provisional application No. 63/231,542, filed on Aug. 10, 2021.

(51) Int. Cl. C12Q 1/6886; C12Q 1/6874; G16B 30/00; G16B 40/20; G16H 10/40

(52) U.S. Cl. CPC C12Q 1/6886; C12Q 1/6874; G16B 30/00; G16B 40/20; G16H 10/40; C12Q 2600/156; C12Q 2600/158

(57) ABSTRACT

Systems, methods, and computer program products are provided for classifying sequence fragments and labelling sequence fragments that represent tumor markers. A plurality of reference sequences are read. A plurality of sequence fragments obtained from a biological sample of a patient are read. A first read and a second read are selected from the plurality of sequence fragments. A regional probability based on a plurality of regional features from the patient is received from a first trained classifier. A tensor is generated comprising a corresponding reference sequence, the first read, the second read, a first position, a second position, and an alt position. A local probability based on the tensor is received from a second trained classifier comprising a convolutional neural network. A label associated with a tumor marker is determined when the regional probability is above a first predetermined threshold and the local probability is above a second predetermined threshold.

### CROSS-REFERENCE TO RELATED APPLICATIONS

This application claims the benefit of U.S. Provisional Application Nos. 63/231,542, filed Aug. 10, 2021 and 63/296,356, filed Jan. 4, 2022, which are hereby incorporated by reference in their entirety.

### TECHNICAL FELD

Embodiments of the disclosure generally relate to the field of medical diagnostics. In particular, embodiments of the disclosure relate to compositions, methods, and systems for circulating tumor DNA detection and cancer diagnosis.

### BACKGROUND

The tremendous burden imposed by cancers such as solid tumors of lung, breast, prostate, liver, and brain, on human health is well-documented in medical literature. Most subjects are diagnosed with high tumor burden disease, which is associated with dismal outcome. Recently, computed tomography (CT) was found to improve early detection of non-small cell lung cancer and was adopted for screening high-risk populations by the US Preventative Services Task Force. Nevertheless, this approach is limited by high false positive rate, leading to costly and potentially harmful follow-up evaluation.

One approach used in cancer diagnosis is the analysis of tumor samples for genetic cues or markers. The cancer genome acquires somatic mutations which drive its proliferative capacity (Lawrence et al, Nature, 505(7484):495-501, 2014). Mutations in the cancer genome also provide critical information regarding the evolutionary history and mutational processes active in each cancer (Martincorena et al, Cell, 171(5):1029-1041.e21, 2017; Alexandrov et al, Nature, 500(7463): 415∧-21, 2013). Cancer mutation calling in patient tumor biopsies has become a pivotal step in prognostication and therapeutic nomination. Identifying cancer mutations through noninvasive liquid biopsy, such as the detection of circulating tumor DNA (ctDNA) among cell-free DNA (cfDNA), has been suggested as a transformative platform for early-stage cancer screening, detection of minimal residual disease (MRD) after surgery, and therapeutic monitoring.

Statistical methods for analyzing genomic markers such as somatic mutations in DNA, e.g., single-nucleotide variants (SNVs), require multiple independent observations (supporting reads) of the somatic variant at any genomic location to distinguish true mutations from sequencing errors. One technique used in differentiating true mutations from sequencing errors is consensus mutation calling, which is useful as long as the tumor or plasma sample contains sufficient tumor or ctDNA content and is sequenced to an adequate depth to allow for multiple observations of candidate mutations. When tumor content in the sample is low, for example due to the dilution of ctDNA among healthy cfDNA in a liquid biopsy plasma sample, each somatic variant is no longer supported by multiple reads, precluding the use of these mutation callers. MUTECT for example is the current state-of-the-art low-allele frequency somatic mutation caller. At its core, MUTECT subjects a SNV to two Bayesian classifiers, one assumes that the SNV results from random noise and the other that the site contains a true variant. It then filters a SNV based on a log-likelihood ratio from the two models. This is fundamentally different from the sparsity of ctDNA in the liquid biopsy setting. In a benchmarking setting when the mutation allele frequency drops to .05 and the tumor sample sequencing depth goes down to 10×, MUTECT's sensitivity decreases to below 0.1 (Cibulskis et al, Nature Biotechnology, 31(3), 213, 2013). While MUTECT is currently the state-of-the art somatic mutation caller in low-frequency settings, it is still unable to identify somatic mutations in tumor fractions like those observed in liquid biopsy of low disease burden cancer.

A fundamental limitation of MUTECT and other mutation callers is the below-acceptable level of clinical sensitivity when input material is limited (such as in the low burden cancer disease setting). A typical plasma sample contains only a few thousand of cfDNA genome equivalents. Thus, ultra-deep sequencing (e.g., 100,000×) may be rendered ineffective by the limited number of physical cfDNA fragments that cover each site that are present in the sample (e.g., 5,000 genomic equivalents in 5 mL of plasma). Even with ultra-deep sequencing and advanced molecular error suppression, the limited input material imposes a detection limit on tumor fraction (TF) frequencies lower than 0.1-1%.

This limitation was exemplified by Abbosh et al. (Nature, 545 (7655):446-451, 2017), which applied advanced sequencing methods, including technically-challenging lung adenocarcinoma patient-specific targeted deep sequencing, to identify about 18 mutations at a median sequencing depth of 42,000×. However, ctDNA was detected in only 19% of subjects with early stage disease, even with the inclusion of more advanced stage III tumors in the study group. Moreover, all of these positively-identified patients had lesions detectable by CT scanning. These data demonstrate that in the early-stage disease context, even ultra-deep sequencing currently underperforms the sensitivity and precision achievable with radiographic imaging.

Cell-free DNA (cfDNA) released from dying cells enables surveys of the somatic genome and epigenome dynamically over time for clinical purposes. The ability to obtain a biopsy through a simple blood draw allows for dynamic genomic measurement in a non-invasive manner. It can overcome spatial limitations, such as inaccessibility of lung tissue.

Circulating tumor DNA (ctDNA), not to be confused with cell-free DNA (cfDNA), can be found and measured in the blood of cancer patients. ctDNA has been shown to correlate with tumor burden and change in response to treatment or surgery (Diehl et al, Nature medicine, 14(9):985-990, 2008). ctDNA can be detected even in early stage non-small cell lung cancer (NSCLC) and therefore has the potential to transform NSCLC diagnosis and treatment (Sozzi et al., Journal of Clinical Oncology, 21 (21), 3902-3908, 2003; Tie et al, Science translational medicine, 8 (346): 346ra92-346ra92, 2016; Bettegowda et al, Science translational medicine, 6 (224): 224ra24-224ra24, 2014; Wang et al., Clinical Cancer Research, 16 (4): 1324-1330, 2010).

One of the major areas of future promise for cfDNA-based cancer studies is in the detection of minimal residual disease (MRD) after surgery or systemic therapy to guide clinical interventions. For example, detection of postoperative residual disease after surgical resection can inform recurrence risk and help clinicians and patients assess the need for potentially toxic adjuvant therapies. However, in the context of low burden disease following surgery, e.g., MRD, ctDNA is sparse and therefore tumor fraction (TF) is low, often considerably below 1:1000. To enable mutation detection of low TF cfDNA, the prevailing paradigm has been to increase the depth of sequencing of a limited set of gene targets (e.g., common cancer drivers and/or deep-targeted sequencing of patient-specific/tumor-informed bespoke (e.g., sequenced to a depth of about 10,000 to 100,000 reads/base). Additionally, molecular and analytic approaches have been integrated with ultra-deep sequencing to reduce sequencing error and improve sensitivity of detection at low tumor fraction (TF).

While these state-of-the-art methods provide detection with high accuracy in some instances, they are hindered by a fundamental limitation that reduces detection sensitivity-limited input material. Typical plasma samples contain only 1-10 ng/ml of cfDNA. The low amount of cfDNA translates into only a few thousand genome equivalents. Thus, the prevailing technique relying on ultra-deep targeted sequencing (e.g., 100,000C) may be rendered ineffective by the limited number of physical fragments that cover each site that are present in the sample (e.g., 5,000 cfDNA genomic equivalents in a 5 mL plasma sample). Even with ultra-deep sequencing and advanced molecular error suppression, the limited input material imposes a detection limit on tumor fraction (TF) frequencies lower than 0.1%, as is commonly seen in low tumor burden settings such as MRD. As such, although detection of cancer with low tumor burden is clinically beneficial to patients and clinicians, existing methods that rely on the identification of somatic mutations face significant challenges due to the low frequency of ctDNA among much more abundant cfDNA. For example, MRD identified via bespoke panels in urothelial carcinoma is strongly prognostic of disease recurrence, though up to 40% of ctDNA-negative patients experienced relapse¹⁹. Similar ‘false negatives’ were seen in breast⁵ and colorectal cancer²²⁻²⁴, suggesting that further improvement in sensitivity is needed.

Accordingly, there is a need for improved methods and systems for identifying low abundance disease markers, such as ctDNA. Additionally, there is a need for systems and methods that utilize these markers in the early diagnosis of tumors, thereby arming clinicians with better options for disease management and/or therapeutic intervention and also greatly improving outcome of disease (e.g., improved survival and/or quality of life).

### BRIEF SUMMARY

In various embodiments, a method is provided for detecting circulating tumor DNA where a plurality of reference sequences is read. A plurality of sequence fragments obtained from a biological sample of a patient is read. A first read and a second read is selected from the plurality of sequence fragments. The first read includes a first portion of a corresponding reference sequence in the plurality of reference sequences and a first position. The second read includes a second portion of the corresponding reference sequence and a second position, and at least one of the first read and the second read includes an alt position. A regional probability is received from a first trained classifier based on a plurality of regional features of the patient. A tensor including the corresponding reference sequence, the first read, the second read, the first position, the second position, and the alt position is generated. The tensor is provided to a second trained classifier including a convolutional neural network, and received therefrom is a local probability based on the tensor. A label associated with a tumor marker is determined when the regional probability is above a first predetermined threshold and the local probability is above a second predetermined threshold.

In various embodiments, a system is provided for detecting circulating tumor DNA including a reference sequence database, a sequence fragment database, a regional feature database, and a computing node comprising a computer readable storage medium having program instructions embodied therewith. The program instructions are executable by a processor of the computing node to cause the processor to perform a method where a plurality of reference sequences is read. A plurality of sequence fragments obtained from a biological sample of a patient is read. A first read and a second read is selected from the plurality of sequence fragments. The first read includes a first portion of a corresponding reference sequence in the plurality of reference sequences and a first position. The second read includes a second portion of the corresponding reference sequence and a second position, and at least one of the first read and the second read includes an alt position. A regional probability is received from a first trained classifier based on a plurality of regional features of the patient. A tensor including the corresponding reference sequence, the first read, the second read, the first position, the second position, and the alt position is generated. The tensor is provided to a second trained classifier including a convolutional neural network, and received therefrom is a local probability based on the tensor. A label associated with a tumor marker is determined when the regional probability is above a first predetermined threshold and the local probability is above a second predetermined threshold.

In various embodiments, a computer program product is provided for detecting circulating tumor DNA comprising a computer readable storage medium having program instructions embodied therewith. The program instructions are executable by a processor of the computing node to cause the processor to perform a method where a plurality of reference sequences is read. A plurality of sequence fragments obtained from a biological sample of a patient is read. A first read and a second read is selected from the plurality of sequence fragments. The first read includes a first portion of a corresponding reference sequence in the plurality of reference sequences and a first position. The second read includes a second portion of the corresponding reference sequence and a second position, and at least one of the first read and the second read includes an alt position. A regional probability is received from a first trained classifier based on a plurality of regional features of the patient. A tensor including the corresponding reference sequence, the first read, the second read, the first position, the second position, and the alt position is generated. The tensor is provided to a second trained classifier including a convolutional neural network, and received therefrom is a local probability based on the tensor. A label associated with a tumor marker is determined when the regional probability is above a first predetermined threshold and the local probability is above a second predetermined threshold.

### BRIEF DESCRIPTION OF THE DRAWINGS

FIG. 1 illustrates a schematic view of a paired-end read according to embodiments of the present disclosure.

FIGS. 2A-2B illustrate an exemplary tensor according to embodiments of the present disclosure.

FIG. 3 illustrates an exemplary multilevel perceptron (MLP) according to embodiments of the present disclosure.

FIG. 4A illustrates an exemplary workflow for classifying ctDNA according to embodiments of the present disclosure. FIG. 4B illustrates an exemplary workflow for classifying ctDNA according to embodiments of the present disclosure.

FIG. 5A illustrates an exemplary parallel workflow for classifying ctDNA according to embodiments of the present disclosure. FIG. 5B illustrates an exemplary sequential workflow for classifying ctDNA according to embodiments of the present disclosure.

FIG. 6 illustrates a table of data on ctDNA classification according to embodiments of the present disclosure.

FIG. 7A illustrates an exemplary ROC curve according to embodiments of the present disclosure. FIG. 7B illustrates an exemplary signal-to-noise enrichment graph according to embodiments of the present disclosure.

FIG. 8A illustrates signal-to-noise enrichment across various processing methods.

FIG. 8B illustrates a mixing study that demonstrates the minimum mix fraction of ctDNA needed to identify melanoma ctDNA among a subset of healthy control patients. FIG. 8C illustrates a graph of sensitivity vs specificity. FIG. 8D illustrates performance of mrdetect-dl vs. standard assays.

FIG. 9 shows application of disease-specific deep learning classifier to distinguish ctDNA SNV fragments from cfDNA artifacts. A) Illustration of whole genome sequencing (WGS)-based ctDNA single nucleotide variant (SNV) detection in plasma with MRD-EDGE. Healthy cfDNA and ctDNA are admixed in the plasma pool. Both cfDNA and ctDNA are subjected to WGS, and SNVs are identified against the reference genome and subjected to quality pre-filters designed to reduce artifact from sequencing error and germline variants. A complex feature space designed to distinguish ctDNA signal from cfDNA noise serves as input to a deep learning neural network, where fragments containing SNVs are classified as ctDNA or cfDNA with sequencing artifacts. B) Heatmap of selected post-filter model features and the single variable area under the receiver operating curve (svAUC) between individual features and label (ctDNA or cfDNA) in LUAD, CRC, and melanoma. In this comparison, ctDNA SNV fragments and cfDNA SNV artifacts are drawn from within the same plasma sample to remove potential inter-sample biases when establishing predictive capacity of individual features. For categorical features, AUC was assessed on a held-out validation set of fragments after a linear classifier was trained to predict positive or negative label based on one-hot encoded categorical features. Features are annotated with whether they are used in MRDetect or MRD-EDGE. C) Selected feature density plots for post-filter ctDNA and cfDNA SNV artifacts: trinucleotide context, replication timing³⁷, PCAWG⁸¹ tumor SNV mutation density, read edit distance, and fragment length. D) (top) Illustration of the fragment tensor, an 18×240 matrix encoding of the reference sequence, R1 and R2 read pairs (including padding where reads do not overlap the reference sequence), R1 read length and R2 read length, and the position of the SNV in the fragment (‘Alt position’). The fragment architecture allows for integration of fragment-specific features such as trinucleotide context, fragment length, and edit distance, among others. The fragment tensor is passed as input to a convolutional neural network. (bottom) Illustration of the relationship between regional features and local ctDNA SNV mutation density at the chromosome level. Disease-specific inaccessible⁸² and quiescent⁸³ genomic regions, as well as late replicating regions³⁷, are associated with somatic mutagenesis as represented by increased density of tumor-confirmed ctDNA SNVs. Regional features (Appendix 2) are encoded as tabular values and passed as input to a multilayer perceptron. An ensemble classifier takes input from both the fragment and regional models to determine the likelihood that each fragment is ctDNA or cfDNA SNV artifact. E) In silico studies of cfDNA from the metastatic cutaneous melanoma sample MEL-01 mixed into cfDNA from a healthy plasma sample (‘C-16’) at mixing fractions TF=10⁻⁷, 10⁻⁴ at 16×depth, performed in 20 technical replicates with independent sampling seeds. Tumor-informed MRD-EDGE enables sensitive TF detection as measured by Z score against unmixed control plasma (TF=0, n=20 randomly chosen replicates) as low as TF=5×10⁻⁷ (AUC 0.70). Box plots represent median, lower and upper quartiles; whiskers correspond to 1.5×IQR. An AUC heatmap benchmarks detection sensitivity vs. TF=0 at different mixed TFs. IQR, interquartile range.

FIG. 10 depicts machine learning-based error suppression and additional features to enhance plasma WGS-based copy number variation (CNV) detection sensitivity. A) (left) Illustration depicting use of copy number denoising for inference of plasma read depth. (top, left) Patient-specific CNV segments are selected through the comparison of tumor and germline WGS. In plasma, these CNV segments may be obscured within noisy raw read depth profiles (middle, left). Machine-learning guided denoising through use of a panel of normal samples (PON) drawn from healthy control plasma samples removes recurrent background noise to produce denoised plasma read depth profiles (bottom, left). Plasma samples used in the PON are subsequently excluded from downstream CNV analysis. (middle) Loss of heterozygosity (LOH) results in replacement of heterozygous single nucleotide polymorphisms (SNPs) with homozygous variants and can be measured via changes in the B-allele frequency of SNPs in cfDNA. (right) Increased or decreased fragment length heterogeneity is expected in regions of tumor amplifications or deletions, respectively, due to varying contribution of ctDNA (shorter fragment size) to the plasma cfDNA pool. Fragment length heterogeneity is measured through Shannon's entropy of fragment insert sizes. Fragment entropy signal is aggregated based on matched tumor amplifications (positive signal) or deletions (negative signal). B-E) In silico mixing studies of admixed high and low TF samples from the melanoma patient AD-12. Pretreatment plasma (TF=17%) was mixed into posttreatment plasma (TF undetectable following a major response to immunotherapy) in 50 replicates. Admixtures model tumor fractions of 10⁻⁶, 10⁻³. Box plots represent median, lower and upper quartiles; whiskers correspond to 1.5×IQR. An AUC heatmap demonstrates detection performance vs. TF=0 at the different mixedadmixed TFs vs. negative controls (TF=0, n=25 replicates used to generate the noise distribution and n=25 used to benchmark performance) as measured by Z score. B) (top) Copy number denoising with the read depth classifier demonstrates detection sensitivity above TF=0 as low as 1*10⁻⁵ (AUC 0.72). (bottom) Normalized error at different mixed TFs between MRD-EDGE read-depth classifier and MRDetect. Error is measured as

$$\frac{T {F}_{estimated} - T {F}_{mixed}}{T {F}_{mixed}} .$$

C-D) SNP BAF (C) and fragment length entropy (D) classifiers demonstrate Z score detection sensitivity at 5*10⁻⁵ (AUC 0.82 and 0.81, respectively). E) Empiric measurement of the MRD-EDGE lower limit of detection for the combined feature set as a function of the CNV load and admixture modeled TF. Sensitive detection (AUC 0.74) is observed at TF=5*10⁻⁵ at 200 Mb. IQR, interquartile range. AUC, area under the receiver operating curve.

FIG. 11 illustrates detection of postoperative colorectal ctDNA and tracking neoadjuvant response to immune checkpoint inhibition and radiation in non-small cell lung cancer. A) ROC analysis on preoperative colorectal SNV mutational compendia for MRD-EDGE (blue) and MRDetect (red). Preoperative plasma samples (n=19) were used as the true label, and the panel of control plasma samples against all patient mutational compendia (n=646; 19 mutational compendia assessed across 34 control samples from Control Cohort A) was used as the false label. B) ROC analysis on preoperative colorectal CNV mutational compendia for MRD-EDGE (blue) and MRDetect (red) methods. Preoperative plasma samples (n=18, 1 sample excluded due to insufficient aneuploidy) were used as the true label, and the panel of control plasma samples against all patient mutational compendia (n=180; 18 mutational compendia assessed across 10 control samples from Control Cohort A) was used as the false label. Twenty-four samples from Control Cohort A were included in the read-depth classifier panel of normal samples (PON, FIG. 10A) and were held out from the CNV ROC analysis. C) Kaplan-Meier disease-free survival analysis was done over all patients with detected (n=9) and non-detected (n=10) postoperative ctDNA. Postoperative ctDNA detection shows association with shorter recurrence-free survival (two-sided log-rank test). D) Illustration of the neoadjuvant non-small cell lung cancer (NSCLC) clinical treatment protocol⁵⁰. Plasma TF is tracked throughout the preoperative period to evaluate for response to SBRT and ICI therapy and after surgery to detect the presence of MRD. The detection threshold for MRD reflects 90% specificity in an independent cohort of preoperative patients with early-stage LUAD evaluated previousuly²⁸ (FIG. 18C). E) Serial tumor burden monitoring on neoadjuvant immunotherapy with MRD-EDGE in 2 NSCLC patients on ICI therapy (no SBRT). Tumor burden estimates are measured as the Z score of the patient-specific mutational compendia against healthy control plasma. In both patients, unchanged plasma TF Z score demonstrates lack of response to ICI prior to surgery. (top) Upon surgical resection, there is no evidence of MRD and no recurrence at 29 months (patient Neo-02). (bottom) Upon surgical resection, plasma TF is above the detection threshold indicative of MRD, and disease recurrence is seen at 12 months postoperatively (patient Neo-03). F) demonstration of plasma TF decrease following radiation in a patient who was randomized to receive SBRT. ctDNA remains detectable following SBRT, and tumor burden increases postoperatively indicating MRD. The patient had disease recurrence at 18 months. ROC, Receiver operating curve. MRD, minimal residual disease. SBRT, stereotactic body radiation therapy. ICI, immune checkpoint inhibition.

FIG. 12 depicts MRD-EDGE tumor-informed detection of ctDNA from screen-detected adenomas and pT1 lesions. A) Detection status of the cohort of Stage IV colorectal (CRC, n=5), screen-detected pT1 lesions (n=10) and screen-detected adenoma plasma samples (n=19) according to MRD-EDGE SNV and CNV classifiers. Samples with a Z score in excess of the detection threshold as prespecified in the early-stage CRC cohort (FIG. 11A-B) are highlighted. B) ROC analysis for MRD-EDGE SNV (top) and CNV (bottom) classifiers in screen-detected adenomas (left) and pT1 lesions (right). Preoperative plasma samples were used as the true label, and the panel of control plasma samples (Control Cohort B) against all patient mutational compendia were used as the false label. For SNVs, 4 of 15 control samples were used in SNV model training and thus excluded from this analysis, yielding 11 control samples as a comparator. For CNVs, 5 of 15 control samples were used in a panel of normal samples (PON) for our read depth classifier (FIG. 10A) and thus excluded from this analysis, yielding 10 control samples as a comparator. C) Plasma TF inference using genome-wide SNV integration for Stage IV CRC (n=5), early-stage preoperative CRC (n=19), SNV detected pT1 lesions (n=3), and SNV detected adenomas (n=46) shows decreasing estimated TF by CRC stage. Lines indicate median estimated TF. D) (left) histology image of the pT1 lesion Aar-14 (top) demonstrates invasion of the submusoca by dysplastic cancer cells, while an image of the adenoma Aar-17 (bottom) demonstrates the presence of dysplasia and absence of submucosal invasion. (right) barplots demonstrate number of plasma samples with detected ctDNA in patients with pT1 lesions (top) and adenomas (bottom). Detections are shaded by dark blue (MRD-EDGE SNV detections), light blue (MRD-EDGE CNV detections), light purple (SNV and CNV detections), and white (non-detected). ROC, receiver operating curve.

FIG. 13 depicts MRD-EDGE detection of ctDNA from colorectal pT1 carcinomas and adenomas. A) MRD-EDGE SNV Z score discrimination between signal detected in patient plasma (blue dots, n=33 patients) and healthy control plasma from Control Cohort B (white boxes, n=11). Four additional samples from Control Cohort B were used in model training and were therefore excluded from downstream SNV analysis. Signal is measured on patient plasma and the control plasma samples using the same patient-specific SNV compendium. The SNV ctDNA detection threshold (dashed horizontal line) was prespecified, reflecting 90% specificity defined in an independent cohort of preoperative patients with early-stage CRC (FIG. 11A). B) Cross patient SNV evaluation. SNV Z-score discrimination is calculated as in (A) using cross-patient evaluation instead of healthy control plasma. Cross-patient signal is calculated via application of the patient-specific mutational compendium to all other patient plasma (white boxes, n=32). The ctDNA detection threshold (dashed horizontal line) was prespecified, reflecting 90% specificity defined in an independent cohort of preoperative patients with early-stage CRC (FIG. 3a). C) Z-score discrimination between MRD-EDGE CNV on patient plasma (blue, n=19 patients) compared to signal detected in neutral regions (as a negative control, red), and cross-patient cohort (n=18, white). Z-score was calculated using the noise parameters estimated by the control plasma cohort. Samples not evaluated due to insufficient aneuploidy (n=9) and samples from Stage IV patients (n=5) were excluded from analysis, the latter due to a sparsity of neutral regions in these advanced cancer samples. The CNV ctDNA detection threshold (dashed horizontal line) was prespecified, reflecting 90% specificity defined in an independent cohort of preoperative patients with early-stage CRC (FIG. 11B).

FIG. 14 depicts determination of MRD-EDGE de novo mutation calling classification threshold. A) Fragment-level signal to noise enrichment, defined as the fraction of remaining ctDNA fragments (signal) over remaining cfDNA SNV artifacts (noise), for different MRD-EDGE classification thresholds in the melanoma held-out validation set derived from tumor-confirmed ctDNA SNVs from the melanoma patient MEL-01 and post-quality filtered cfDNA artifacts from healthy control plasma (Appendix 2). The MRD-EDGE SNV deep learning classifier uses a sigmoid activation function that outputs the likelihood between 0 and 1 that a candidate SNV fragment is a mutated ctDNA fragment or cfDNA harboring a sequencing error, and the classification threshold is used as a decision boundary for these two classes. Signal to noise enrichment increases at higher classification thresholds, as expected. B) As increased specificity will ultimately eliminate most of the signal, to choose an optimal threshold for classification, we compared sensitivity vs. TF=0 in an in silico study of cfDNA from the metastatic melanoma sample MEL-01 mixed in n=20 replicates against cfDNA from a healthy plasma sample (TF=0) at 5*10⁻⁵ at 16×coverage depth. We found optimal performance at a classifier threshold of 0.995 as measured by AUC of mixed replicates against TF=0. This threshold was subsequently applied in de novo mutation calling analyses. C) (left) ctDNA detection rates for pretreatment cutaneous melanoma samples from the adaptive dosing cohort (n=26, orange, detection rate was capped at 0.0005) compared to acral melanoma samples (n=3, blue, pre- and posttreatment timepoints from 1 patient with acral melanoma) sequenced within the same batch and flow cell. (right) ctDNA detection rates for healthy control plasma (n=30, gray). ctDNA is not detected from acral melanoma plasma, demonstrating absence of batch effect and the specificity of MRD-EDGE for the UV signatures associated specifically with cutaneous melanoma.

FIG. 15 depicts MRD-EDGE SNV feature selection, model architecture and performance. A) Feature density plots for post-quality filtered ctDNA and cfDNA SNV artifacts used in the LUAD model. In this comparison, ctDNA SNV fragments are identified from consensus mutation calls in high burden LUAD plasma samples (Appendix 2) and cfDNA SNV artifacts are drawn from within the same plasma sample to remove potential inter-sample biases when establishing predictive ability of individual features. B) SNV classification performance for different machine learning models. F1 score was assessed on tumor-confirmed melanoma ctDNA SNV fragments vs. cfDNA artifacts from healthy controls. Random subsamplings were drawn from the held-out melanoma validation set (Appendix 2), which was split into tenths for this analysis. We compared performance between MRD-EDGE and its separate components (left), as well as to other ML architectures (right) C) Fragment-level ROC analysis for MRD-EDGE SNV classifier for different cancer types. Performance is assessed on post-quality filtered fragments (˜90% of low-quality cfDNA artifacts are excluded by quality filters) in held-out validation sets (Appendix 2) for melanoma, LUAD, and CRC. D) Signal to noise enrichment analysis for MRDetect and for each step of the MRD-EDGE tumor-informed pipeline. Final pipeline enrichment is 118-fold for MRD-EDGE vs. 8.3-fold for the MRDetect in the same datasets.

FIG. 16 depicts MRD-EDGE CNV detection in neutral regions and non-small cell lung cancer. A-E) In silico mixing studies in which high TF plasma samples were admixed into low TF samples from the melanoma patient AD-12 and the NSCLC patient Neo-03. For melanoma, pretreatment plasma was mixed into posttreatment plasma as described in FIG. 2b. For NSCLC, preoperative plasma was mixed into postoperative plasma in 20 technical replicates (each subsampling seed represents a technical replicate). Admixtures model tumor fractions of Oct. 6, 2010-3 (see Methods for detailed description of in silico admixture process). Box plots represent median, lower and upper quartiles; whiskers correspond to 1.5×IQR. An AUC heatmap demonstrates detection performance vs. TF=0 at different mixed TFs as measured by a sample Z score compared to TF=0 distribution for each replicate. The read depth (A), fragment entropy (B), and SNP BAF (C) classifiers demonstrate similar performance in preoperative NSCLC admixtures compared to melanoma admixtures (FIG. 2B-D). d-e, Z scores for the read-depth classifier in neutral regions (no copy number gain or loss in the matched tumor WGS data) for melanoma (D) and NSCLC (E) demonstrates the expected absence of ctDNA detection at different TF admixtures, consistent with no expected read depth changes in copy neutral regions. F) Assessment of preoperative plasma, postoperative plasma, and PBMC BAF in SNPs before (left) and after (right) SNP quality filters in CRC (patient CRC-16). Filters include minimum coverage and outlier exclusion criteria (Methods). BAF signal is calculated as the mean window-level (1 Mb) deviation from the 0.5 SNP reference in LOH events identified on matched tumor WGS (Methods), and these values are summed across genome-wide LOH events to calculate sample level signal. To demonstrate the relationship between signal and phased SNPs, the major allele in plasma is randomly permuted to be in phase or out of phase at the percentage specified along the x axis. Following quality filtering, signal can be appropriately inferred and demonstrates the expected relationship between preoperative plasma (highest signal), postoperative MRD (intermediate signal), and PBMC BAF (minimal signal).

FIG. 17 depicts CNV load across tumor types. CNV load in WGS samples across cancer types from the TCGA cohort measured as a function of the size of genome altered by CNV (in log 10 Mb). Dashed lines represent the percentage of samples that have CNV load of over 200 Mb, the lower limit of detection for the MRD-EDGE CNV classifier. Cancer types include LUSC: Lung squamous cell carcinoma (n=50), HNSC: Head and Neck squamous cell carcinoma (n=50), CESC: Cervical squamous cell carcinoma and endocervical adenocarcinoma (n=18), OV: Ovarian serous cystadenocarcinoma (n=50), KICH: Kidney Chromophobe (n=50), COAD: Colon adenocarcinoma (n=53), THCA: Thyroid carcinoma (n=50), LUAD: Lung adenocarcinoma (n=152), ESCA: Esophageal carcinoma (n=19).

FIG. 18 depicts clinical performance of MRD-EDGE in perioperative CRC and LUAD tumor burden monitoring. A) Cross-patient ROC analysis on preoperative colorectal SNV mutational compendia for MRD-EDGE demonstrates similar performance to control (non-cancer) plasma ROC analysis (FIG. 11A). Preoperative plasma samples (n=19) were used as the true label, and SNVs identified from the patient-specific mutational compendia in other preoperative CRC patients (n=342; 19 mutational compendia assessed across 18 cross-patient samples) was used as the false label. B) Cross-patient ROC analysis on preoperative colorectal CNV mutational compendia for MRD-EDGE. Preoperative plasma samples (n=18) were used as the true label, and cross patient plasma was used as the false label (n=306; 18 mutational compendia assessed across 17 cross-patient samples) was used as the false label. One sample was excluded due to insufficient aneuploidy. C) ROC analysis on preoperative LUAD SNV mutational compendia for MRD-EDGE (blue) and MRDetect SNV+CNV mutational compendia (published previously²⁸, red). Preoperative plasma samples (n=36) were used as the true label, and the panel of control plasma samples against all patient mutational compendia (n=1,224; 36 mutational compendia assessed across 34 control samples from Control Cohort A) was used as the false label. D) Kaplan-Meier disease-free survival analysis was done over all LUAD patients with detected (n=12) and non-detected (n=10) postoperative ctDNA. Postoperative ctDNA detection shows association with shorter recurrence-free survival (two-sided log-rank test). E) Cross-patient ROC analysis on LUAD colorectal SNV mutational compendia for MRD-EDGE demonstrates similar performance to control (non-cancer) plasma ROC analysis. Preoperative plasma samples (n=36) were used as the true label, and SNVs identified from the patient-specific mutational compendia in other preoperative LUAD patients (n=1,260; 36 mutational compendia assessed across 35 cross-patient samples) were used as the false label.

FIG. 19 depicts accurate monitoring of ctDNA in melanoma with sensitivity comparable to plasma WGS using MRD-EDGE detects, without matched tumor-informed methods. A) In silico studies of cfDNA from the metastatic melanoma sample MEL-01 (pretreatment TF of 3.5%) mixed in n=20 replicates against cfDNA from a healthy plasma sample (TF=0) at mix fractions 10⁻⁶−10⁻² at 16×coverage depth. MRD-EDGE enables sensitive TF detection as measured by Z score against healthy controls at TF=5*10⁻⁵ (AUC 0.77) without matched tumor tissue to guide SNV identification. Box plots represent median, bottom and upper quartiles; whiskers correspond to 1.5×IQR. An AUC heatmap measures detection vs. TF=0 at different mixed TFs. B) Signal to noise enrichment analysis for MRDetect SVM and for each step of the MRD-EDGE de novo mutation calling pipeline. Final pipeline enrichment is 2,518-fold for MRD-EDGE vs. 8.3-fold for the MRDetect SVM in the same plasma samples. MRD-EDGE provides for a cumulative 301-fold enrichment over MRDetect. C) Study schematic for adaptive dosing melanoma cohort (n=26 patients with advanced melanoma). All patients began treatment with combination ipilimumab (3 mg/kg) and nivolumab (1 mg/kg). Plasma was collected at pretreatment timepoint at week 0, at second dose of combination ICI at Week 3, and at Week 6. Beginning at Week 6 patients received either combination ICI or ICI monotherapy based on imaging response: patients with stable or shrinking disease on Week 6 CT received nivolumab monotherapy and those with tumor growth received additional combination therapy. Further CT imaging was performed at Week 12. D) ROC analysis for the detection of pretreatment melanoma using MRD-EDGE for healthy individuals (n=30, false label) and patients with melanoma (n=25, true label). One pretreatment melanoma plasma sample with high TF used in model training was withheld from this analysis. Detection rate cutoff was selected as the first operational point with specificity of 90% or greater. E) Fourteen of 26 patients from the adaptive dosing cohort underwent sequencing with a tumor-informed targeted panel⁸ (‘tumor-informed panel’). Vertical bars demonstrate pretreatment detection sensitivity for MRD-EDGE, the tumor-informed panel, a de novo panel based on the de novo calling thresholds⁸ used for the tumor-informed panel, and ichorCNA. Error bars represent 95% binomial confidence interval for empiric sensitivity within 14 trials. F) Serial tumor burden monitoring on ICI with MRD-EDGE, tumor-informed panel, and de novo panel for 3 patients with melanoma. Tumor burden estimates are measured as a detection rate normalized to the pretreatment sample (normalized detection rate, nDR) for MRD-EDGE and as variant allele fraction (VAF) normalized to the pretreatment VAF (normalized VAF, nVAF) in the tumor-informed panel and de novo panel. MRDetect accurately captures trends in TF, while the de novo panel faces sensitivity barriers in low TF settings where plasma VAF<0.005. Blue highlights surrounding sample name indicate samples with 14 or more SNVs covered in the tumor-informed panel. G) Forty-three pre- and posttreatment samples from the adaptive dosing melanoma cohort underwent sequencing with MRD-EDGE and the tumor-informed panel. (top) Heatmap demonstrating detection overlap (measured as the agreement between platforms of detected ctDNA and undetectable ctDNA) between MRD-EDGE and the tumor-informed panel shows high concordance (88%) between the two platforms. (bottom) Lower detection overlap (60%) is present between MRD-EDGE and the de novo targeted panel due to sensitivity floors in the de novo panel. H) Barplot of Cohen's Kappa agreement metric for Week 6 ctDNA trend (increase or decrease) compared to pretreatment baseline between 3 mutation callers and the tumor-informed panel: MRD-EDGE, de novo panel, and iChorCNA. MRD-EDGE demonstrates most agreement with the tumor-informed panel (Cohen's Kappa 0.75). ROC, Receiver operating curve. IQR, interquartile range. IQR, interquartile range. CT, computed tomography.

FIG. 20 depicts serial monitoring of clinical response to immunotherapy with MRD-EDGE. A) Study schematics of two advanced melanoma cohorts. (left) conventional immunotherapy cohort received nivolumab monotherapy or combination ICI. Plasma was collected at pretreatment timepoint and weeks 3, 6, and 12. Cross sectional imaging to evaluate response to treatment was performed at 12 weeks. (right) adaptive dosing cohort received combination immunotherapy as described in FIG. 19C. B) Serial plasma TF monitoring with MRD-EDGE corresponds to changes seen on imaging. TF estimates are measured as a detection rate normalized to the pretreatment sample (normalized detection rate, nDR) for MRD-EDGE. (top) ctDNA nDR grossly increases over time in a patient with disease refractory to ICI. The patient had progressive disease at Week 6 and Week 12 CT assessment. (bottom) ctDNA nDR decreased at Week 3 in a patient with a partial response to therapy. CT imaging demonstrates tumor shrinkage at Week 6 and Week 12. C) Kaplan-Meier progression-free and overall survival analysis for Week 3 ctDNA trend in patients with decreased (n=27) or increased (n=7) nDR as measured by MRD-EDGE. Patients with undetectable pretreatment ctDNA (n=3) were excluded from the analysis. Increased nDR at Week 3 shows association with shorter progression-free and overall survival (two-sided log-rank test). D) (top, left) pretreatment CT imaging of a patient with decreased ctDNA in response to ICI at Week 3 on both MRD-EDGE (nDR, blue) and a tumor-informed panel (normalized variant allele frequency, nVAF, red). Following the administration of methylprednisone at Week 3, estimated TF on both ctDNA detection platforms increased. At Week 6, progressive disease is seen on CT imaging (top right). E) Early steroids for irAEs within the combination ICI dosing period (prior to Week 8) further stratify Week 3 survival analyses. Kaplan-Meier progression-free and overall survival analysis was performed on patients with primary refractory disease (‘primary refractory’, blue, n=7), defined as rising nDR seen at Week 3 following first dose of treatment, decreasing ctDNA who did not receive steroids (“no steroids”, red, n=18), and patients who received steroids for immune-related adverse events within the combination ICI dosing period (‘steroids’, green, n=9). P value reflects multivariate logrank test. ICI, immune checkpoint inhibition. CT, computed tomography.

FIG. 21 depicts a computing node according to embodiments of the present disclosure.

FIG. 22 depicts trends in plasma TF using MRD-EDGE, a tumor-informed panel, and a de novo panel. Serial tumor burden monitoring on ICI with MRD-EDGE, tumor-informed panel, and de novo panel for 11 patients with melanoma (see FIG. 19f for remaining 3 patients with matched WGS and panel data). Tumor burden estimates are measured as a detection rate normalized to the pretreatment sample (normalized detection rate, nDR) for MRD-EDGE and as variant allele fraction (VAF) normalized to the pretreatment VAF (normalized VAF, nVAF) in the tumor-informed panel and de novo panel. Outcome is reported as RECIST response on Week 12 CT imaging including partial response (‘PR’), stable disease (‘SD’), or progressive disease (‘PD’). Blue highlights surrounding sample names indicate samples with 14 or more mutations covered in the tumor-informed panel.

FIG. 23 depicts monitoring response to immunotherapy with MRD-EDGE. A) Forest plot demonstrating relationship between ctDNA TF trend (increase or decrease) and progression-free survival (PFS) and overall survival (OS) at serial posttreatment timepoints. MRD-EDGE TF estimates are measured as a detection rate normalized to the pretreatment sample (normalized detection rate, nDR). Each posttreatment timepoint is prognostic of PFS outcomes. B) (left) Kaplan-Meier overall survival analysis for Week 6 RECIST response (n=10 partial response, ‘PR’, n=8 stable disease, ‘SD’, n=6 progressive disease, ‘PD’) in the adaptive dosing melanoma cohort (n=26 patients) where CT imaging was available at Week 6 shows no significant relationship with OS (multivariate logrank test). C) Kaplan-Meier OS analysis for Week 6 ctDNA trend in adaptive dosing melanoma patients with decreased (n=17) or increased (n=5) nDR compared to pretreatment timepoint as measured by MRD-EDGE. Patients with undetectable pretreatment ctDNA (n=2) were excluded from the analysis as were 2 patients where Week 6 plasma was not available for analysis. Increased nDR at Week 6 shows association with shorter overall survival (two-sided log-rank test). TF, tumor fraction; CT, computed tomography.

FIG. 24 depicts the accurate monitoring of ctDNA in small cell lung cancer plasma WGS using MRD-EDGE, without matched tumor. Top panel; ROC analysis for the detection of pretreatment melanoma using MRD-EDGE for healthy individuals (n=30, false label) and patients with small cell lung cancer melanoma (n=17, true label). No samples involved in model training were used in this analysis. Detection rate cutoff was selected as the first operational point with specificity of 90% or greater. Bottom panel; serial tumor burden monitoring on immune checkpoint inhibition with MRD-EDGE for 3 patients with small cell lung cancer. Tumor burden estimates are measured as a detection rate normalized to the pretreatment sample (normalized detection rate, nDR).

### DETAILED DESCRIPTION

The ability to monitor malignant tumor burden below the limit of radiographic detection remains a major unmet need of modern healthcare systems. Liquid biopsy for circulating tumor DNA (ctDNA) offers promise, however, deep targeted sequencing methods—the conventional approach in the field—face a sensitivity plateau in low volume cancer due to the sparsity of ctDNA signal. Whole genome sequencing (WGS) of plasma overcomes this sensitivity barrier by expanding the number of informative sites to the thousands of somatic single nucleotide variants observed across the genome in solid tumors. Systems and methods for determining the presence of ctDNA is described in U.S. Patent Application Publication No. 2021-0002728 and U.S. Patent Application Publication No. 2021-0043275, each of which is hereby incorporated by reference herein in its entirety.

In various embodiments, WGS of plasma allows for ultra-sensitive inference of ctDNA signal in low volume cancers. However, the fundamental challenge in this approach is to distinguish the tens to hundreds of true ctDNA SNVs in low volume disease from the sequencing errors found in WGS (e.g., sometimes numbering in the millions). One method, MRDetect, uses advanced error suppression with support vector machines, but only provided a ctDNA signal-to-noise enrichment of 10-20×, and therefore required a matched tumor SNV compendium to reach a sensitivity of 10⁻⁵ (the value needed to detect postoperative residual disease after surgery in early stage lung cancer). Matched tumor tissue may not be available in low volume cancer settings and may add considerable expense given the need to sequence tumor/normal pairs.

In various embodiments, to expand applicability and overcome MRDetect's need for matched tumor tissue, the disclosed systems, methods, and computer program products provide a tumor-agnostic (de novo) classifier that uses advanced machine learning to increase error suppression and amplify ctDNA signal, allowing for ultra-sensitive ctDNA inference in low volume cancer settings using plasma WGS alone. In various embodiments, the system includes a novel machine learning ensemble model including a ctDNA fragment level neural network, such as a convolutional neural network (CNN) taking, as input, a sequential tensor. In various embodiments, the machine learning ensemble model includes a regional-level multilayer perceptron (MLP) taking, as input, one or more regional features. In various embodiments, the MLP and CNN operate sequentially, with the MLP being applied first and the CNN being applied second (or vice versa). In various embodiments, the MLP and CNN operate in parallel, both executing at approximately the same time with the respective inputs.

In various embodiments, the machine learning ensemble model uses a unique feature space in liquid biopsy including fragmentomics, nucleosomics, regional, and/or other epigenetic context to predict whether candidate cell-free DNA single nucleotide fragments are ctDNA or artifact from sequencing error. In various embodiments, the machine learning ensemble model may be trained on tumor-confirmed ctDNA fragments (e.g., for melanoma). In various embodiments, after training on tumor-confirmed melanoma ctDNA fragments, the disclosed machine learning ensemble model may generate a ctDNA signal-to-noise enrichment of about 1,000×(whereas MRDetect only generates a signal to noise enrichment of 10-20×) in held-out validation plasma samples from melanoma patients with advanced disease. In various embodiments, this transformative improvement allows for ultra-sensitive liquid biopsy monitoring without need for matched tumor tissue and has numerous clinical applications in modern solid tumor oncology.

In various embodiments, disclosed are novel machine learning architectures that enable ultra-sensitive liquid biopsy for circulating tumor DNA through whole genome sequencing of plasma without need for matched tumor tissue. In various embodiments, the disclosure provides 1) a novel machine learning architecture for the encoding of cell-free DNA fragments and accompanying site-level/regional features and 2) a software workflow that takes a list of cell-free DNA single nucleotide variants (SNVs) as input and outputs a circulating tumor DNA tumor burden estimate based on prediction from a trained circulating tumor DNA SNV classifier.

In various embodiments, the disclosed methods determine cell-free DNA mutations using novel deep learning architectures for advanced error suppression. In various embodiments, the deep learning architectures use fragmentomics and regional features to inform ctDNA predictions. In various embodiments, classifiers may be trained to be cancer specific (e.g., a melanoma-specific classifier, lung cancer-specific classifier, colorectal cancer-specific classifier, etc.)

In various embodiments, the machine learning platform includes a novel fragment-level (2-paired reads) machine learning architecture, use of fragmentomics, use of regional features such as replication timing, DNase hypersensitivity, RNA transcription (among other features described in more detail below), use of nucleosomics (nucleosome positioning), use of an ensemble machine learning model architecture to include simple and sequential features, and use of unique melanoma, NSCLC, and colorectal training sets for validation of the ensemble model.

In various embodiments, the disclosed fragment CNN classifier and regional MLP ensemble model may be implemented with non-paired read fragments. In various embodiments, the non-paired reads may be determined from a flow based sequencing technology that puts a single fragment on one read.

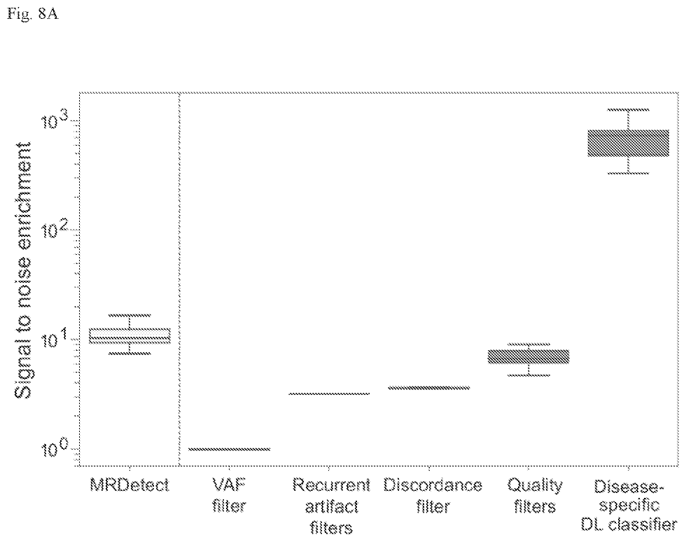

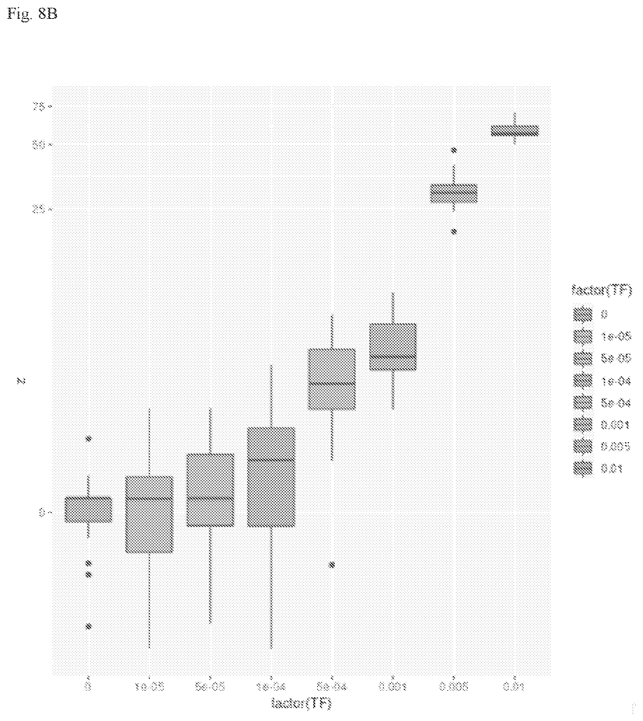

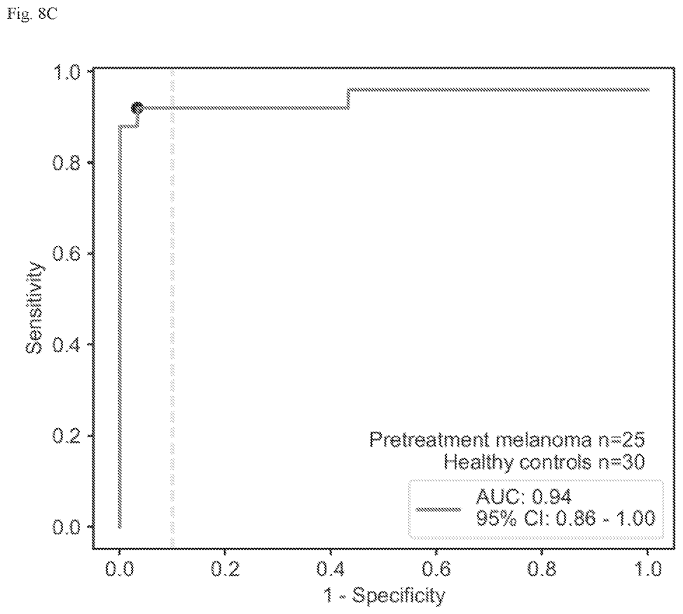

In various embodiments, disclosed methods have clinical utility in that it provides high ROC and F1 scores for ctDNA vs. noise during training and validation, and improved signal to noise enrichment of about 1000×(whereas MRDetect is only 10-20×), as shown in FIG. 8A, allows for de novo (rather than tumor-informed in MRDetect) ultra-sensitive cell free DNA mutation calling. In various embodiments, the disclosed machine learning ensemble model allows for accurate ctDNA tumor burden inference using standard WGS alone. In various embodiments, the disclosed machine learning ensemble model has demonstrated clinical utility in therapeutic disease monitoring, and accurately captures the nadir of response to immunotherapy in metastatic melanoma samples.

In various embodiments, the multilayer perceptron takes one or more regional features as input to assess whether a given locus is prone to cancer mutagenesis. In various embodiments, the MLP may be combined in an ensemble model with the CNN to jointly inform predictions of ctDNA. In various embodiments, the MLP may include regional features such as nucleosome position, chromatin state, and chromatin accessibility. In various embodiments, the MLP may include fragment level and genomic features. In various embodiments, each of the two classifiers (MLP and/or fragment CNN) can function independently of one another.

In various embodiments, the disclosed machine learning ensemble models may be used in the following non-limiting examples: ultra-sensitive therapeutic monitoring of response to systemic therapy in advanced melanoma, small cell lung cancer, and non-small cell lung cancer (NSCLC), detection of postoperative minimal residual disease following surgical resection of early stage cancer (which can nominate patients for additional therapy), early noninvasive detection of relapse following complete response to immunotherapy (which can allow patients to switch treatments while disease burden is low), early detection of cancer without prior diagnosis, noninvasive lung cancer screening, noninvasive colon cancer screening, etc. In various embodiments, the disclosed machine learning ensemble models may be used in other types of cancer screening.

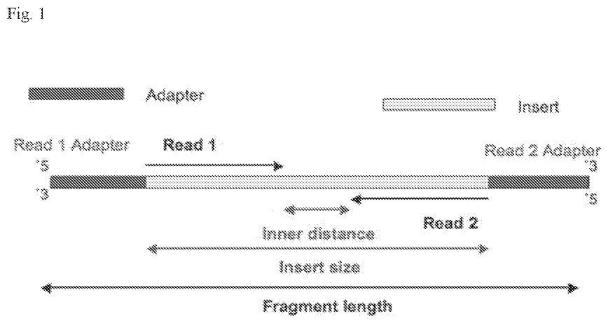

In various embodiments, the disclosed machine learning ensemble models evaluate reads at the fragment level. In various embodiments, the reads are paired reads, as shown in FIG. 1. In various embodiments, a custom preprocessing pipeline may trim adaptors from fragments and remove duplicates.

In various embodiments, one or more fragment filters may be applied prior to classification. In various embodiments, the fragment filters may replace another classifier, such as the support vector machine (SVM) used in MRDetect based on sequencing quality metrics. In various embodiments, the one or more fragment filters may remove candidate fragments that are highly likely to be recurrent local sequencing artifact (variant blacklist) or candidate fragments that are likely to be noise as indicated by quality metrics.

In various embodiments, the fragment filters may include an artifact blacklist. In various embodiments, the artificial blacklist may include a custom plasma WGS blacklist for n=3 or more appearances in the WGS plasma database to remove recurrent/predictable artifact from sequencing (HiSeq and Novaseq). In various embodiments, this form of artifact may be biased to the local sequencing machine(s) (e.g., Illumina machines) rather than variants identified in large public databases.

In various embodiments, the fragment filters may be based on quality metric filters. In various embodiments, the fragment filters may exclude fragments that do not meet specific quality criteria. In various embodiments, the fragment filter may filter discordant reads. For example, a discordant read may include one or more fragments with a variant that is not present on both R1 and R2 and, thus, may be excluded. In various embodiments, the discordant reads may be highly enriched for sequencing error. In various embodiments, the fragment filter may filter for variant base quality. For example, if the variant base quality is less than 25 (e.g., on an Illumina Phred scale), the fragment may be excluded. In various embodiments, the fragment filters may include a filter for depth of read. For example, for a depth less than 10, the fragment may be excluded. In various embodiments, the fragment filters may include a filter for mapping quality. For example, if the mapping quality is less than 10, a fragment may be excluded. In various embodiments, the fragment filters may include a filter for a predetermined number of low quality bases. In various embodiments, where base quality is less than 20, a base may be considered low quality. For example, if a fragment (e.g., R1 read) has less than or equal to 24 low-quality bases, the fragment may be excluded. In various embodiments, base quality may be a feature included in the regional MLP model.

In various embodiments, the number of low quality bases may be determined using methods as described in Ma, X. et al. “Analysis of error profiles in deep next-generation sequencing data.” Genome Biol 20, 50 (2019). (accessible online at https://doi.org/10.1186/s13059-019-1659-6), which is hereby incorporated by reference in its entirety.

In various embodiments, the fragment filters may include a filter for fragment length. For example, a fragment having a fragment length of less than 240 base pairs (bp) may be included and fragments having a fragment length of greater than or equal to 240 bp may be excluded. In various embodiments, a higher fragment base pair lengths may be enriched for contamination from genomic DNA. In various embodiments, the fragment filters may include a filter for variant allele fraction. For example, fragments having a variant allele fraction of less than 0.2 may be included and fragments having a variant allele fraction of greater than or equal to 0.2 may be excluded. This example of a filter may be used to reduce germline single nucleotide polymorphism (SNP) contamination (germline SNPs may have peaks at 0.5 and 1). In various embodiments, fragment filters may remove approximately 70% of candidate fragments prior to deep learning classification. In various embodiments, a signal to noise enrichment plot may be transmitted for each step of prefiltering and deep learning classification pipeline.

FIG. 1 illustrates a schematic view of a paired-end read. Paired-end sequencing allows users to sequence both ends of a fragment and generate high-quality, alignable sequence data. In various embodiments, paired-end sequencing facilitates detection of genomic rearrangements and repetitive sequence elements, as well as gene fusions and novel transcripts. In various embodiments, in addition to producing twice the number of reads for the same time and effort in library preparation, sequences aligned as read pairs enable more accurate read alignment and the ability to detect insertion-deletion (indel) variants, which is not possible with single-read data.

“Read 1”, often called the “forward read”, extends from the “Read 1 Adapter” in the 5′-3′ direction towards “Read 2” along the forward DNA strand.

“Read 2”, often called the “reverse read”, extends from the “Read 2 Adapter” in the 5′-3′ direction towards “Read 1” along the reverse DNA strand.

In various embodiments, there may be an arbitrary DNA sequence inserted between “Read 1” and “Read 2,” which may be called an “Inner sequence.” In various embodiments, the length of this sequence is measured as the “Inner distance.” In various embodiments, by definition, the “Insert” is the concatenation of “Read 1”, the “Inner distance” sequence and “Read 2.” In various embodiments, the length of the “Insert” is the “Insert size.” In various embodiments, a single “Fragment” may include the “Read 1 Adapter,” “Read 1,” “Inner sequence,” “Read 2,” and “Read 2 Adapter.” In various embodiments, the length of this “Fragment” is a “Fragment length.” In various embodiments, the length of each read (e.g., read 1 and read 2) is a “Fragment length.”

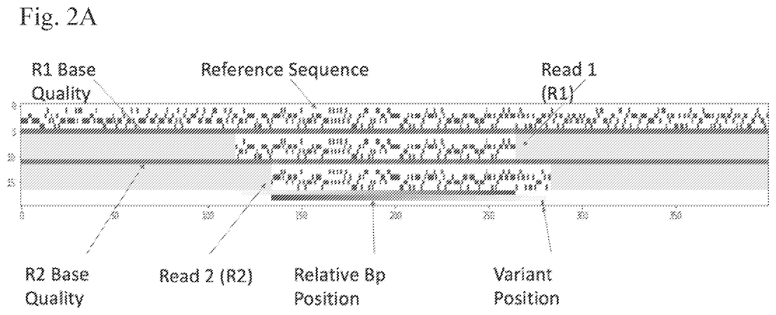

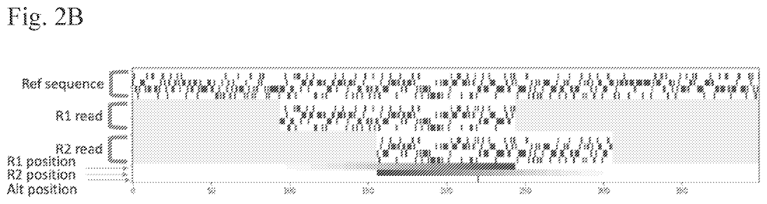

FIGS. 2A-2B illustrate an exemplary tensor. FIGS. 2A-2B illustrate a novel representation of cfDNA fragments (paired R1 and R2 sequencing reads). In various embodiments, the representation may be a 18×400 tensor in which the rows are features and the columns are base pairs along a fragment sequence. In various embodiments, the representation may be a 19×400 tensor in which the rows are features (using one additional feature than the 18×400 tensor) and the columns are base pairs along a fragment sequence. In various embodiments, the representation may be a 18×240 tensor in which the rows are features and the columns are base pairs along a fragment sequence. In various embodiments, the representation may be a 14×240 tensor to represent unpaired reads. In various embodiments, fragments may include a mean of 170 bp. In various embodiments, fragments may range in length from 40 to 240 bp to filter longer fragments that are likely to be contaminants from germline DNA. In various embodiments, fragments may be are centered within the 400 base pair length of the tensor or within the 240 base pair length of the tensor. One of skill in the art will recognize that any suitable dimensions may be used for the tensor.

In various embodiments, the fragment may be centered within the fragment length (e.g., 240) such that the start position for R1 is [(window_size−fragment_length)/2] and the end position for R2=window size−(window_size−fragment length)/2.

In various embodiments, the reads may be derived from the same fragment at the time of sequencing. In various embodiments, the reads may share a common unique read ID which may be paired computationally at the time of alignment by an aligners (e.g., BWA_mem).

In various embodiments, a pileup may be performed of all alts against the reference sequence. In various embodiments, fragments (e.g., all) that are present at the alt position are identified and whether or not each fragment has the alt of interest is determined. In various embodiments, multiple fragments may include the same alt position. In various embodiments, all of these fragments having the same alt position may be presented to the pipeline (e.g., quality filters, blacklist, deep learning classifier, etc.) for consideration as potential ctDNA.

In various embodiments, the tensors illustrated in FIGS. 2A-2B may include a reference sequence in consecutive rows 0 to 4. In various embodiments, the reference sequence may be the specific base at the reference genome (e.g., GRCh38). In various embodiments, each row in the reference sequence may be encoded to represent one of the 4 nucleotides (G,C,T,A) and N for undefined.

In various embodiments, the tensor may include a R1 read sequence in consecutive rows 5 to 9. In various embodiments, similar to the reference sequence, each row may encode for a respective nucleotide along R1 (G, C, T, A, N). In various embodiments, the tensor may include a R2 read sequence in consecutive rows 10 to 14. In various embodiments, similar to the reference sequence, each row may encode for a respective nucleotide along R2 (G, C, T, A, N).

In various embodiments, the tensor may include a R1_pir in a single row. In various embodiments, the R1_pir may tracks the length of R1 from 0 at first nucleotide of fragment to a length Len_R1 at the last nucleotide of the fragment. In various embodiments, the tensor may include a R2_pir in a single row. In various embodiments, the R2_pir may tracks the length of R2 from 0 at first nucleotide of fragment to a length Len_R2 at the last nucleotide of the fragment.

In various embodiments, the tensor may include an alt position in a single row. In various embodiments, the alt position is a position in the fragment that is the alt being evaluated by the classifier. In various embodiments, this row may be all 0s with a 1 at the position of the single nucleotide variant. In various embodiments, the tensor may include a corresponding lymphocyte nucleosome track in a single row (e.g., in the 19×400 tensor). In various embodiments, the unique tensor structure is coded to account for all possible CIGAR outputs, including insertions, deletions, mismatches, clips, and soft masks.

In various embodiments, fragments may be analyzed at the ‘alt’ level. In various embodiments, if there are multiple mismatches against the reference sequence per fragment, each may be independently analyzed by the fragment classifier. In various embodiments, the classifiers may only analyze single nucleotide variants. In various embodiments, insertions and/or deletions may be filtered from the analysis.

In various embodiments, the fragment tensor may provide access to key genomic features including mutation type, trinucleotide context, and leading or lagging strand as well as quality metrics such as the position of the alt within the fragment (ends of reads are enriched for sequencing error), edit distance (how many alts against the reference sequence are present), and/or alignment score of the fragment against the reference sequence. In various embodiments, the fragment tensor may provide access to fragment length (ctDNA fragments are often shorter than cfDNA fragments, a key feature for our models). In various embodiments, the fragment tensor may provide access to latent features around the reference sequence and/or other ‘hidden’ features uncovered from deep learning.

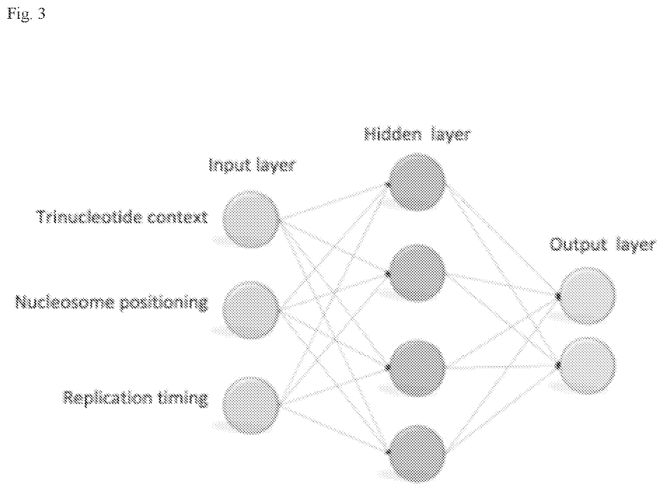

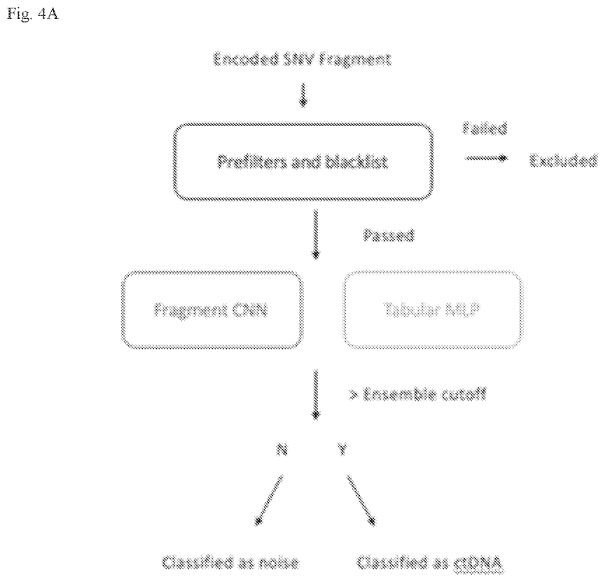

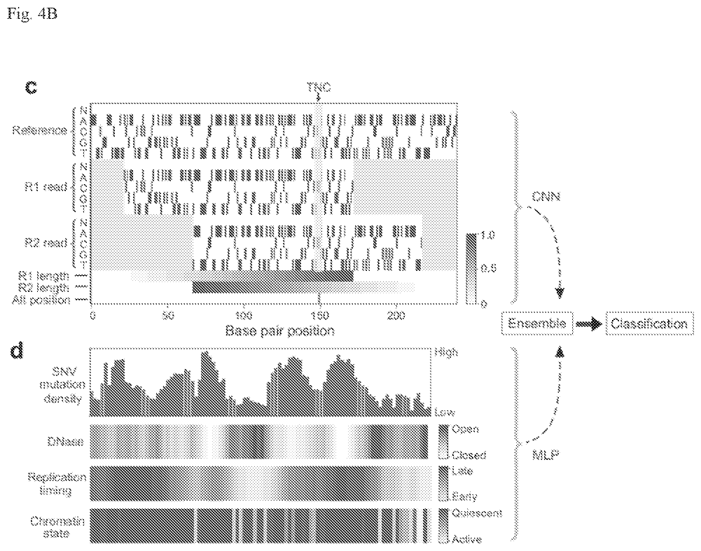

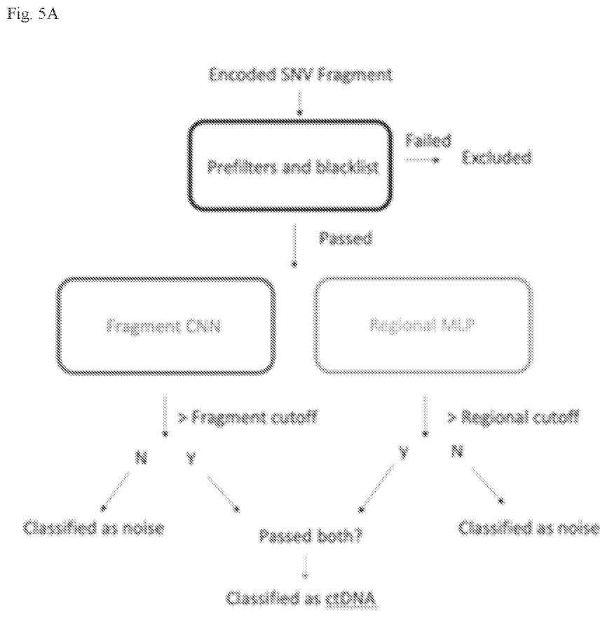

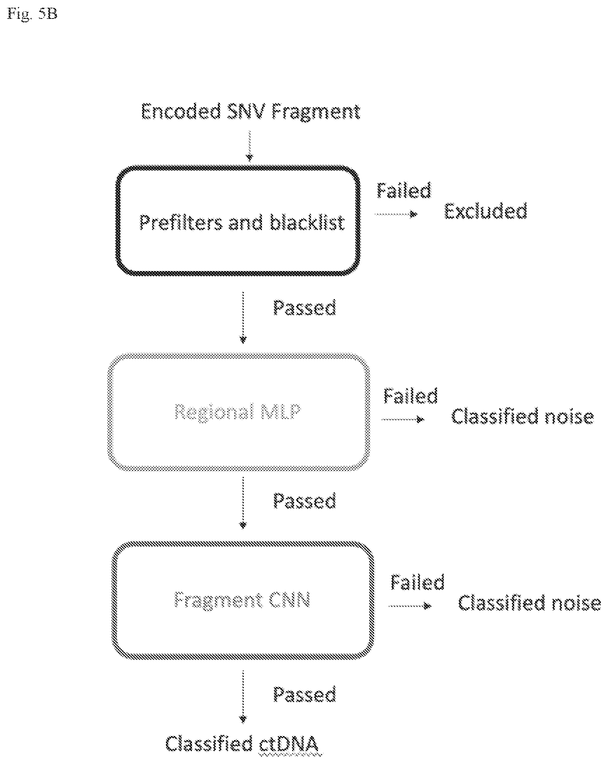

FIG. 3 illustrates an exemplary multilevel perceptron (MLP). In various embodiments, the MLP model may be a regional model. In various embodiments, the regional model may classify site-level features.

In various embodiments, while prefilters may account for variant base quality and other sequencing quality metrics, the fragment classifier (CNN) may account for fragment level features and the regional model (MLP) may account for the local chromosomal environment surrounding the fragment (e.g., local genetic and regional context). In various embodiments, the MLP may be used to determine whether the chromatin are accessible or inaccessible, whether the chromatin are late replicating or early replicating, among other things. In various embodiments, chromosomal context may be a key feature of somatic mutagenesis and closely tied to mutation density.

In various embodiments, all of the regional features may be centered around the alt at the time of encoding. For example, the regional classifier may determine what the chromosomal accessibility is in the 50,000 base pair interval on either side of the alt position.

In various embodiments, the regional MLP may include a local tumor-type specific ATAC density (e.g., amount of ATAC peaks per 100,000 bp as measured by a peak calling algorithm, drawn from a public database). In various embodiments, the regional MLP may include a local primary cell DNAse hypersensitivity (e.g., amount of DNase peaks per 100,000 bp, drawn from ENCODE). In various embodiments, the regional MLP may include a local histone chip-seq density (e.g., measured in RPKM over 100,000 bp intervals, optimized by comparing all possible histone chip-seq bams from the ENCODE database against ctDNA and noise with the highest correlation value between bam and label ultimately chosen for each histone methyltransferase). In various embodiments, the regional MLP may include a local cancer type specific mutational density from PCAWG, a public WGS dataset (e.g., how many mutations are present in a large tumor WGS dataset in a 20,000 bp interval around the SNV). In various embodiments, the regional MLP may include a local chromatin state (e.g., how active or quiescent are the local chromatin, as measured by chrom_hmm algorithm). In various embodiments, the regional MLP may include a Hi C compartmentalization—are the chromatin in the A (open) or B (closed) compartment. In various embodiments, the regional MLP may include replication timing (e.g., whether the area replicated early or late during the cell replication cycle). In various embodiments, the regional MLP may include a transcription direction (e.g., whether the area was transcribed in a right or left direction). In various embodiments, the regional MLP may include an indication of forward or reverse DNA transcription (e.g., indicating whether transcription moves forward or backward). In various embodiments, the regional MLP may include a distance to bound transcription factors (e.g., a base pair distance to the nearest bound transcription factor; for example, if there are fewer true SNVs around bound transcription factors). In various embodiments, the regional MLP may include the local RNA expression (e.g., a measure of bulk RNA seq RPKM of the primary tissue). In various embodiments, the regional MLP may include a measure (e.g., number) of low quality bases on the candidate fragment, as described above.

FIG. 4A illustrates an exemplary workflow for classifying ctDNA. In various embodiments, an encoded SNV fragment may be filtered by one or more fragment filters as described above. In various embodiments, the resulting filtered SNV fragments may be passed to a fragment CNN and a regional MLP that each output a probability. If the probability for each classifier is above the respective predetermined thresholds, the SNV fragment is classified as ctDNA. If the probability for either classifier is below the respective predetermined threshold, the SNV fragment is classified as noise.

In various embodiments, by training both a CNN and MLP jointly, the machine learning ensemble architecture combines a CNN's ability to learn sequence-related info and the MLP's ability to learn regionally-related info. In various embodiments, for both the CNN and MLP, the final layer that was responsible for outputting a prediction may be removed; instead the learned representation in latent space may be taken from their respective prior layers and concatenated together. In various embodiments, this new combined vector is passed through multiple fully-connected layers that then output the predicted probability that the given fragment is ctDNA.

FIG. 4B illustrates an exemplary workflow for classifying ctDNA. In particular, FIG. 4B illustrates an exemplary tensor provided to the CNN for fragment-level classification. FIG. 4B also illustrates exemplary regional features provided to the regional MLP. In this example, SNV mutation density (ranging from high to low), DNase (ranging from open to closed), Replication timing (ranging from late to early), and Chromatin state (ranging from quiescent to active) are provided as features. In various embodiments, any of the regional features may have binary values. In various embodiments, any of the regional features may have a range of values.

FIG. 5A illustrates an exemplary parallel workflow for classifying ctDNA. In various embodiments, the classifiers may generate a consensus on a SNV fragment in parallel.

FIG. 5B illustrates an exemplary sequential workflow for classifying ctDNA. In various embodiments, a regional MLP may be applied to appropriate SNV fragments and the SNV fragments that pass through the MLP (e.g., have a probability above a predetermined threshold) to the fragment CNN classifier. After classification at the fragment CNN, the SNV fragments having a probability above a predetermined threshold may be classified as a ctDNA (e.g., labelled with a ctDNA label from a plurality of ctDNA labels).

In various embodiments, the regional MLP may receive as input a tabular feature representation. In various embodiments, the regional MLP may include five fully-connected layers with ReLU activation functions of decreasing size. In various embodiments, each layer of the MLP may be preceded by a batch normalization layer. In various embodiments, each layer in the MLP may be followed by a dropout layer (with the exception of dropout following the final layer). In various embodiments, the final layer of the regional MLP may include a sigmoid activation, which represents the predicted probability that the given input fragment is ctDNA.

In various embodiments, the predetermined threshold for the MLP to pass a SNV fragment is 0.995. In various embodiments, the predetermined threshold for the MLP to pass a SNV fragment is 0.99. In various embodiments, the predetermined threshold for the MLP to pass a SNV fragment is 0.98. In various embodiments, the predetermined threshold for the MLP to pass a SNV fragment is 0.95. In various embodiments, the predetermined threshold for the MLP to pass a SNV fragment is 0.90. In various embodiments, such as in the tumor-informed setting, the predetermined threshold for the MLP to pass a SNV fragment is 0.85. In various embodiments, such as in the tumor-informed setting, the predetermined threshold for the MLP to pass a SNV fragment is 0.80. In various embodiments, such as in the tumor-informed setting, the predetermined threshold for the MLP to pass a SNV fragment is 0.75. In various embodiments, such as in the tumor-informed setting, the predetermined threshold for the MLP to pass a SNV fragment is 0.70. In various embodiments, such as in the tumor-informed setting, the predetermined threshold for the MLP to pass a SNV fragment is 0.60. In various embodiments, such as in the tumor-informed setting, the predetermined threshold for the MLP to pass a SNV fragment is 0.50.

In various embodiments, the fragment representation (i.e., tensor) that is input to the CNN may be two-dimensional, as described above. In various embodiments, the fragment CNN includes four one-dimensional convolution layers. In various embodiments, the convolution layers may perform convolution over the base pair width dimension. In various embodiments, each convolution layer may be followed by a max pooling operation. In various embodiments, the convolution and max pooling layers may be followed by three fully-connected layers (with ReLU activation). In various embodiments, the fully-connected layers may be followed by a subsequent dropout layer. In various embodiments, the, the final layer in the fragment CNN may be a single sigmoid-activated fully-connected layer (e.g., similar to the MLP).

In various embodiments, each classifier may include a final layer that is a sigmoid activation function configured to output a probability between 0 and 1 that a fragment is noise (e.g., 0) or ctDNA (e.g., 1). In various embodiments, each classifier may evaluate the respective input (e.g., fragment tensor for CNN, regional features of the fragment for MLP) for the specific disease type it is trained for (e.g., melanoma, NSCLC, colorectal, etc.). For example, a score of 1 in a melanoma classifier may indicate that the model is highly confident that the fragment is melanoma ctDNA rather than post-filter noise. In various embodiments, each classifier may evaluate the same fragment for multiple cancer types (e.g., lung, melanoma, colon, etc.). In various embodiments, where a classifier evaluates a fragment for multiple cancer types, the label with the highest probability among the different cancer types may be selected. In various embodiments, the probability may be biased towards pre-test likelihood (e.g., if evaluating for ctDNA in a lifelong smoker, the results may be more biased for lung cancer than melanoma, for example).

In various embodiments, the predetermined threshold for the CNN to pass a SNV fragment is 0.995. In various embodiments, the predetermined threshold for the CNN to pass a SNV fragment is 0.99. In various embodiments, the predetermined threshold for the CNN to pass a SNV fragment is 0.98. In various embodiments, the predetermined threshold for the CNN to pass a SNV fragment is 0.95. In various embodiments, the predetermined threshold for the CNN to pass a SNV fragment is 0.90. In various embodiments, such as in the tumor-informed setting, the predetermined threshold for the CNN to pass a SNV fragment is 0.85. In various embodiments, such as in the tumor-informed setting, the predetermined threshold for the CNN to pass a SNV fragment is 0.80. In various embodiments, such as in the tumor-informed setting, the predetermined threshold for the CNN to pass a SNV fragment is 0.75. In various embodiments, such as in the tumor-informed setting, the predetermined threshold for the CNN to pass a SNV fragment is 0.70. In various embodiments, such as in the tumor-informed setting, the predetermined threshold for the CNN to pass a SNV fragment is 0.60. In various embodiments, such as in the tumor-informed setting, the predetermined threshold for the CNN to pass a SNV fragment is 0.50.

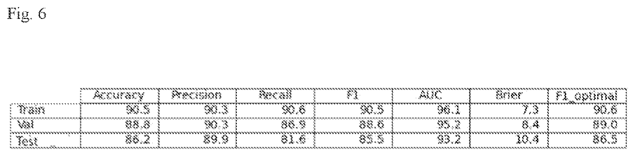

FIG. 6 illustrates a table of data on ctDNA classification. In various embodiments, specificity and recall may vary depending on the cancer type being evaluated. FIG. 6 shows results for analysis of a melanoma. The disclosed machine learning ensemble model was trained, validated, and tested on sets consisting of positive (ctDNA) and negative labels (post-filter ‘noise’-SNVs from pileups that screen alts against the reference sequence in our WGS samples). True SNV mutations were identified among 20-40 million+noise variants in pileups. In various embodiments, the training, validation, and/or test sets may be balanced between positive and negative label. As shown in FIG. 6, more noise is present in the test set label than in training or validation sets. In a tumor informed setting, the general accuracy of the model may be used since the alt was found in the tumor and must be therefore be either a true somatic SNV, artifactual noise, or a germline SNV. The likelihood that a variant is a true somatic SNV is much higher than in the tumor agnostic (de novo) setting.

In various embodiments, in a tumor agnostic setting (de novo mutation calling), there may be a skew towards specificity because the signal to noise ratio may be 1:10,000 according to tumor informed data in metastatic melanoma. In various embodiments, the results may be skewed towards specificity in ROC (see validation ROC) to minimize false positives, and the performance of the model is less about accuracy and more about the highest recall at a given specificity. In various embodiments, this is done through using a high classifier prediction cutoff, often in excess of 0.99, with a target FPR rate of 0.01 to 0.001 depending on the model. In various embodiments, the prediction cutoff may be informed by mixing studies that demonstrate the minimum mix fraction of ctDNA needed to identify melanoma ctDNA among a subset of healthy control patients, an example of which is shown in FIG. 8B.

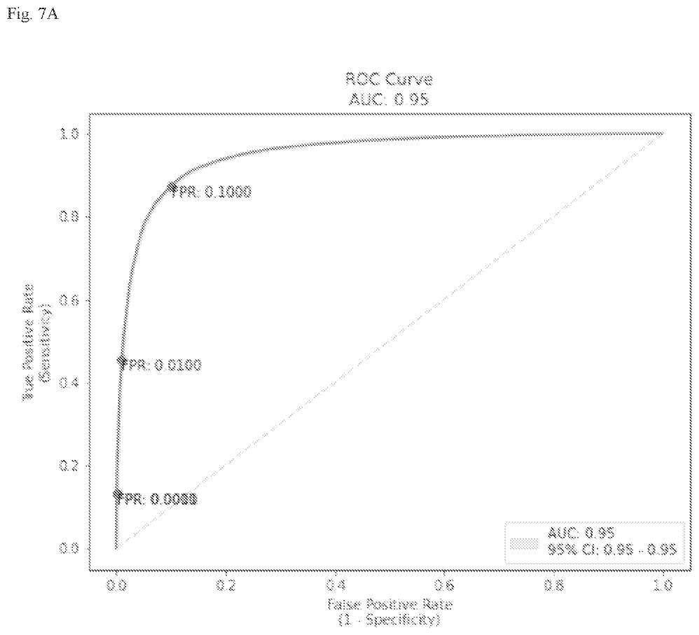

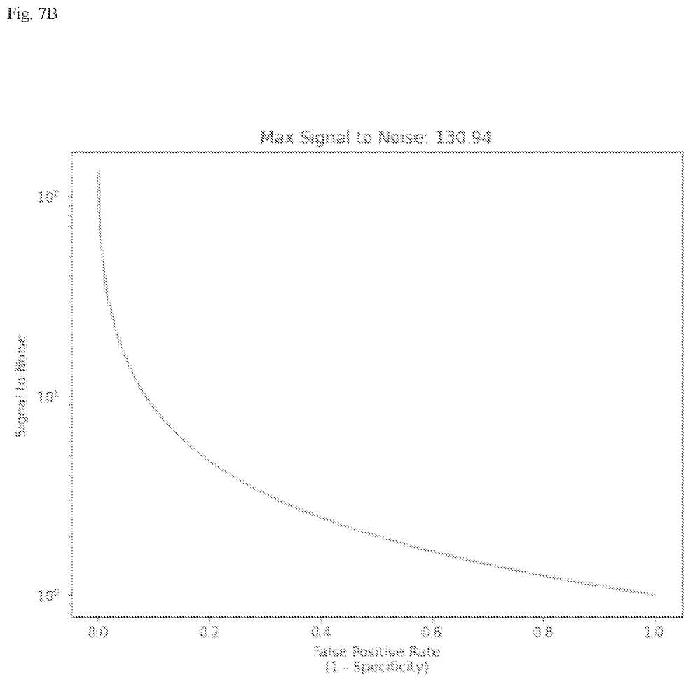

FIG. 7A illustrates a ROC curve. In various embodiments, a detection rate in clinical samples may be quantified (post filter variants classified as ctDNA/total post filter variants evaluated). In various embodiments, a detection rate threshold can be set against healthy controls to mark the presence or absence of ctDNA in plasma at high specificity, an example of which is shown in FIG. 8C. In various embodiments, accuracy of the classifier at the sample level may be evaluated by comparing to standard assays (example shown in FIG. 8D illustrating performance vs. standard assays for mrdetect-dl) and by aligning to actual clinical outcomes in the retrospective patient population (e.g., determining whether detection rate going up or down and did the patient respond to treatment on imaging). In various embodiments, metrics such as durability of response and progression free survival may be used to ensure tumor burden estimates match true treatment response and resistance.

FIG. 7B illustrates an exemplary signal-to-noise enrichment graph. In particular, the signal-to-noise (y-axis) is on a logarithmic scale from 10∧0 to 10∧2 and the false positive rate (x-axis) is on a linear scale from 0.0 to 1.0. As shown, the signal-to-noise decreases as the false positive rate increases. In various embodiments, the signal-to-noise may have an inverse relationship with the false positive rate.

It has been have previously demonstrated²⁴⁻²⁷ that sensitivity barriers in deep targeted panels arise from the limited number of ctDNA fragments recovered at targeted loci. Even with ideal error suppression and ultra-deep sequencing, a somatic mutation cannot be observed if it is not sampled in the limited plasma volume collected in routine testing, which imposes a hard barrier on effective coverage depth. Sensitivity is therefore tied to the limited number of genome equivalents (GE) in a plasma sample (typically 1,000 s per mL²⁸), and when TF is below harvested GEs, MRD detection is diminished. Targeted approaches have sought to overcome this limitation by increasing the number of panel-covered mutations to dozens^{3,8,19-21} or even 100s²⁴ or enriching for biological features of ctDNA such as altered fragment size^{7,29}.

An alternative approach was previously proposed in which breadth of sequencing could supplant depth of sequencing via integration of thousands of single nucleotide variants (SNVs) and copy number variants (CNVs) across the cancer genome²⁷. Whole genome sequencing (WGS) of plasma and matched tumor was implemented for enhanced MRD signal recovery in colorectal cancer (CRC) and lung adenocarcinoma (LUAD). The accompanying denoising approach MRDetect enabled the detection of plasma TFs as low as 1*10⁻⁵ and identified postoperative MRD linked to early disease recurrence²⁷, supporting WGS as a viable strategy for MRD detection.

WGS allows for increased signal recovery at the expense of increased sequencing noise, yet denoising tools such as high sequencing depth and molecular tags leveraged by deep targeted panels are not typically deployed in the WGS setting. In previous MRDetect work, a support vector machine learning approach was designed to identify patterns specific to WGS sequencing error and suppress low quality SNV artifacts. Herein it is contemplated that learning patterns specific to ctDNA mutagenesis can offer signal enrichment in addition to sequencing error suppression. MRD-EDGE (Enhanced ctDNA Genomewide signal Enrichment) was developed, which integrates complementary signal from SNVs and CNVs to increase ctDNA signal enrichment in plasma WGS. For SNVs, MRD-EDGE uses deep learning to integrate the myriad local and regional properties of somatic mutations to identify ctDNA mutations among sequencing error. For CNVs, MRD-EDGE uses machine learning-based denoising and an expanded feature space including fragmentomics and allelic frequency of germline single nucleotide polymorphisms (SNPs) to enable ultrasensitive ctDNA detection at lower degrees of aneuploidy than MRDetect. The increased performance of MRD-EDGE enabled ultrasensitive MRD and tumor burden monitoring in tumor-informed settings, as well as the detection of ctDNA shedding from precancerous colorectal adenomas. Further, the signal to noise enrichment from MRD-EDGE enabled de novo (non-tumor-informed) detection of melanoma ctDNA SNVs at sensitivity on par with tumor-informed targeted panels. Demonstrated herein is the clinical utility of this de novo approach by using plasma ctDNA response to immune checkpoint inhibition (ICI) to predict long-term treatment outcomes.

Provided herein is MRD-EDGE, a composite machine learning-guided WGS ctDNA single nucleotide variant (SNV) and copy number variant (CNV) detection platform designed to increase signal enrichment. MRD-EDGE uses deep learning and a ctDNA-specific feature space to increase SNV signal to noise enrichment in WGS by 300×compared to our previous noise suppression platform MRDetect. MRD-EDGE also reduces the degree of aneuploidy needed for ultrasensitive CNV detection through WGS from 1 Gb to 200 Mb, thereby expanding its applicability to a wider range of solid tumors. This improved performance was harnessed to track changes in tumor burden in response to neoadjuvant immunotherapy in small cell lung cancer and non-small cell lung cancer and demonstrate ctDNA shedding in precancerous colorectal adenomas. Finally, the radical signal to noise enrichment in MRD-EDGE enables de novo mutation calling in melanoma without matched tumor, yielding clinically informative TF monitoring for patients on immune checkpoint inhibition.

Provided herein are methods of identifying plasma allelic imbalance in a sample from a patient indicative of ctDNA tumor fraction. In some embodiments, said methods comprise receiving a plurality of normal sequences from the patient, comprising a first plurality of single-nucleotide polymorphisms (SNPs). In some such embodiments, the method comprises receiving a plurality of tumor sequences comprising a second plurality of SNPs. In some embodiments, the method comprises receiving a plurality of sequence fragments obtained from a plasma sample of the patient, the plasma sample comprising cell-free DNA, and the plurality of sequence fragments comprising a plurality of plasma SNPs.

In various embodiments, the plasma SNPs are evaluated against the first and second plurality of SNPs to identify major alleles. Evaluating the plasma SNPs may comprise:

determining a plurality of tumor SNPs based on the first and second plurality of SNPs, grouping the tumor SNPs and the plasma SNPs into non-overlapping genomic windows, thereby enriching for a local signal, applying at least one quality filter to the tumor SNPs and/or plasma SNPs at the individual SNP level, discarding those of the genomic windows having less than a predetermined number of tumor SNPs, determining a BAF value for each of the tumor SNPs, identifying major alleles based on those of the BAF values that exceed a predetermined threshold. In some such embodiments, an aggregate allelic imbalance score is generated from each of the plurality of genomic windows based on the BAF scores of the major alleles and an expected balance value.

In some embodiments, the SNPs are germline SNPs. In some such embodiments, the first plurality of SNPs are determined from a peripheral blood mononuclear cells (PBMC) fraction of a sample and the plasma sample comprises a plasma fraction of the sample.

In some embodiments, the samples disclosed herein comprise bodily fluid such as blood, plasma, serum, saliva, synovial fluid, lymph, urine, or cerebrospinal fluid. In preferred embodiments the sample is a blood sample.

In various embodiments, determining the plurality of tumor SNPs comprises filtering to regions of imbalance.

In some embodiments, the regions of imbalance are determined based on loss of heterozygosity (LOH).

In the some embodiments of the invention, the non-overlapping genomic windows are 1 Mb.

The invention provided herein may further comprise applying one or more quality filters to the first and/or second plurality of SNPs. In some such embodiments, the quality filters comprise minimal coverage thresholds. As a non-limiting example, the minimal coverage threshold is a read depth greater than or equal to 20 reads. In some embodiments, the quality filters comprise outlier criteria for plasma BAF defined as 0.3<plasma BAF<0.7 and 0.4<PBMC BAF<0.6. In preferred embodiments, the quality filters comprise an outlier criterion for PBMC BAF defined as 0.4<PBMC BAF<0.6.

In some embodiments, the predetermined threshold is regional-specific.

In some aspects of the invention, provided herein are methods of diagnosis comprising performing the methods disclosed herein, and comparing the aggregate allelic imbalance score to a predetermined threshold to determine the presence of a cancer in the patient.

In some embodiments, determining the BAF value comprises normalizing the BAF value for each of the sample SNPs according to a number of window-level sample SNPs and a number of genome-wide SNPs to generate a window-level BAF value, subtracting window-level PBMC BAF values from window-level plasma BAF values to produce a window-level BAF score that reflects the BAF signal from the contribution of circulating tumor DNA (ctDNA) in cancer plasma in excess of BAF signal from cancer plasma variants alone, and aggregating window-level BAF scores to produce a mean per-window sample-level BAF score. The BAF score from cancer plasma can be compared to BAF scores from healthy control plasma, or to neutral regions in other cancer plasma, to determine a score indicative of ctDNA tumor fraction. In some embodiments this score is a sample level Z score for the cancer sample of interest compared to a control or cross patient noise distribution.

In accordance with the various embodiments, provided herein are methods comprising: determining an aggregate allelic imbalance; receiving a read-depth comprising a regional probability of variant sequence; receiving fragment entropy comprising heterogeneity of fragment insert size for circulating free DNA (cfDNA) fragments; and combining the aggregate allelic imbalance score, the read-depth, and the fragment entropy as independent inputs at the sample level to assess plasma tumor fraction (TF).

In some embodiments, the heterogeneity of fragment insert size is determined within consecutive non-overlapping 100 kb genomic windows having an insert size between 100-240 bp.

In various embodiments, said combining comprises determining Z-scores using Stouffer's method

$$\frac{Z = {\sum }_{i = 1}^{k} {Z}_{i}}{\sqrt{k}} .$$

Without being bound by theory, fragment entropy may be determined from changes in the cfDNA fragmentome indicative of increased or decreased ctDNA contribution. For a tumor sequence this may comprise, tagging a plurality of windows according to tumor aneuploidy; determining in matching windows in plasma a distribution of window-level fragment sizes; measuring the distribution of these fragment sizes through Shannon's entropy in different size ranges or measuring outright fragment length; normalizing tagged windows to the entropy of other all windows within a sample, tagging each window with a chromatin state annotation (e.g., active or quiescent chromatin), using a trained classifier to adjust the fragment entropy contribution according to underlying chromatin state (e.g., transcription start site, enhancer, quiescent chromatin), producing a per tagged window fragment size score, aggregating this score at a sample level. The fragment size score from cancer plasma may be compared to fragment size scores from healthy control plasma, or to neutral regions in other cancer plasma, to determine a score indicative of ctDNA tumor fraction. In some embodiments this score is a sample level Z score for the cancer sample of interest compared to a control or cross patient noise distribution. Thus, in some aspects of the invention, disclosed herein are methods of determining fragment size entropy comprising: for a tumor sequence, tagging a plurality of windows according to tumor aneuploidy; determining the chromatin state for each of the plurality of genomic windows; providing the tags and the chromatic state to a trained classifier and receiving therefrom fragment size entropy. In some embodiments, the fragment entropy is determined according to the methods provided herein. In some such embodiments, the method may further comprise: determining a circulating tumor DNA (ctDNA) contribution to the cfDNA pool based on the fragment entropy in one or more of the plurality of genomic windows.

In accordance with the various embodiments, a system comprising: a computing node comprising a computer readable storage medium having program instructions embodied therewith, the program instructions executable by a processor of the computing node to cause the processor to perform a method is provided.

Also provided herein is a non-transitory computer readable storage medium having program instructions embodied therewith, the program instructions executable to perform a method in accordance with the embodiments disclosed herein.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### Examples

The invention now being generally described, it will be more readily understood by reference to the following examples, which are included merely for purposes of illustration of certain aspects and embodiments of the present invention, and are not intended to limit the invention.

### Example 1: Methods

Human subjects and sample processing. This study was approved by the local ethics committee and by the institutional review board (IRB) and was conducted in accordance with the Declaration of Helsinki protocol. Blood samples were collected in blood collection tubes from patient and healthy adult volunteers enrolled in clinical research protocols at NewYork-Presbyterian/Weill Cornell Medical Center, Memorial Sloan Kettering Cancer Center, Massachusetts General Hospital, the Royal Marsden NHS Foundation Trust in the United Kingdom, or Aarhus University Hospital, Bispebjerg Hospital, Randers Hospital, Herning Hospital, Hvidovre Hospital, and Viborg Hospital in Denmark. Melanoma tumor, normal and plasma samples from the Royal Marsden NHS Foundation Trust were obtained under an ethically approved protocol (Melanoma TRACERx, Research Ethics Committee Reference 11/LO/0003). Tumor tissues were collected from resected lung, melanoma, colorectal cancer, and adenoma specimens. The diagnosis of cutaneous melanoma, NSCLC, CRC, and adenoma was established according to World Health Organization criteria and confirmed in all cases by an independent pathology review. Informed consent on IRB-approved protocols for genomic sequencing of patients' samples was obtained before the initiation of sequencing studies.

Germline and tumor DNA processing. Tumor tissue and matched germline DNA from peripheral blood mononuclear cells (PBMCs) or adjacent normal tissue were collected and stored at −80° C. until they were processed for extraction. Genomic DNA was extracted from tumor tissue using the QIAamp DNA Mini Kit (Qiagen). Genomic DNA was extracted from PBMCs using the QIAamp DNA Blood Kit (Qiagen). Libraries were prepared using the TruSeq DNA PCR-Free Library Preparation Kit (Illumina) with 1 μg of DNA input after the recommended protocol⁸⁴, with minor modifications as described below. Intact genomic DNA was concentration normalized and sheared using the Covaris LE220 sonicator to a target size of 450 bp. After cleanup and end repair, an additional double-sided bead-based size selection was added to produce sequencing libraries with highly consistent insert sizes. This was followed by A-tailing, ligation of Illumina DNA Adapter Plate adapters and two post-ligation bead-based library cleanups. These stringent cleanups resulted in a narrow library size distribution and the removal of remaining unligated adapters. Final libraries were run on a Fragment Analyzer (Agilent) to assess their size distribution and quantified by qPCR with adapter-specific primers (Kapa Biosystems). Libraries were pooled together based on expected final coverage and sequenced across multiple flow cell lanes to reduce the effect of lane-to-lane variations in yield. WGS was performed on the HiSeq X or NovaSeq v1.0 (Illumina) at 2×150-bp read length, using SBS v3 (Appendix 1).

Plasma DNA processing. At the same day of blood collection, blood collection tubes (Streck or K2-EDTA, Appendix 1) were centrifuged at 2,000 r.p.m. for 10 min to separate plasma. cfDNA was then extracted from human blood plasma by using the Mag-Bind cfDNA Kit (Omega Bio-Tek). The protocol was optimized and modified to optimize yield²⁸. Elution time was increased to 20 min on a thermomixer at 1,600 r.p.m. at room temperature and eluted in 35-μl elution buffer. The concentration of the samples was quantified by a Qubit Fluorometer (Thermo Fisher), and samples were run on a fragment analyzer by using the High Sensitivity NGS Fragment Analysis Kit (Agilent) to define the size of cfDNA extracted and genomic DNA contamination. For plasma samples that were found to have significant genomic DNA contamination (fragment size>240 base pairs for more than 20% of fragments at library preparation) we performed a 0.4× cleanup using SPRIselect magnetic beads (Beckman Coulter) on the extracted cfDNA.

A subset of plasma samples was sequenced at Aarhus University in Denmark (Appendix 1). For these samples, blood samples were collected in K2-EDTA 10 ml tubes (Becton Dickinson). Within two hours of blood collection, blood collection tubes were centrifuged at 2,000 r.p.m. for 10 min to separate plasma. Isolated plasma was centrifuged again at 2,000 r.p.m. for 10 min. cfDNA was then extracted from human blood plasma using the QIAmp Circulating Nucleic Acids kit (Qiagen), eluted in 60-μl elution buffer (10 mM Tris-Cl, pH 8.5). The concentration of the samples was quantified by droplet digital PCR (ddPCR; Bio-Rad Laboratories), using assays specific to two highly conserved regions on Chr3 and Chr7, as previously described⁸⁵. In addition, all samples were screened for contamination of genomic DNA from leucocytes using a ddPCR assay targeting the VDJ rearranged IGH locus specific for B cells, as previously described⁸⁵. No samples were contaminated by genomic DNA from leucocytes.

Plasma cfDNA library preparation and sequencing. Samples sequenced at the New York genome Center were processed using KAPA Hyper Library Preparation. Cohorts included in Zviran et al. were processed as previously described²⁸. Samples with a mass above 5 ng were prepared for next-generation sequencing on Illumina's HiSeq X or NovaSeq by using a modified manufacturer's protocol. The protocol was scaled down to half reaction by using 25 μl of extracted cfDNA. IDT for Illumina TruSeq Unique Dual Indexes⁸⁴ was used by diluting 1:15 with EB (elution buffer), and ligation reaction was adjusted to 30 min. Additional 0.8× SPRIselect magnetic beads (Beckman Coulter) cleanup was included after post-ligation cleanup to remove excess adapters and adapter dimers. cfDNA from 1 ml of plasma was used for all of the plasma samples in this study. For samples with low concentration, an additional 1 ml of plasma was extracted, and the DNA aliquot with the highest mass was used for library preparation. The number of PCR cycles was dependent on initial cfDNA total mass. For samples with more than 5 ng of total cfDNA, 5-7 PCR cycles were performed. For samples with less than 5 ng of total cfDNA, 7-10 PCR cycles were performed. (Appendix 1). Quality metrics were performed on the libraries by Qubit Fluorometer, High Sensitivity DNA Analysis Kit and KAPA SYBR FAST qPCR Kit (Roche). WGS was performed on the HiSeq X (HCS HD 3.5.0.7; RTA v2.7.7) at 2×150-bp read length or NovaSeq v1.0 at 2×150-bp read length (Appendix 1) to a target depth of 30×.

Plasma samples sequenced at Aarhus University also used KAPA Hyper Library Preparation. cfDNA from 2 mL plasma (see Appendix 1 for DNA mass) was used as input for library preparation using a modified manufacturer's protocol. xGen UDI-UMI Adapters were used and the ligation reaction was adjusted to 30 min. Agencourt AMPure XP beads (Beckman Coulter) were used for both cleanup step with a bead: DNA ratio of 1.2× and 1.0× for the post-ligation and post-PCR cleanup, respectively. The number of PCR cycles was 7 for all cfDNA samples. Qubit Fluorometer and TapeStation D1000 were used for library quality control. WGS was performed on sequenced on NovaSeq v1.5 at 2×150-bp read length to a target depth of 30×.

Preprocessing, quality control analysis and sample identification and concordance. WGS reads for primary tumor, matched germline and plasma samples were demultiplexed using Illumina's bcl2fastq (v2.17.1.14) to generate FASTQ files. The primary tumor and matched germline WGS were submitted to the New York Genome Center somatic preprocessing pipeline, which includes alignment to the GRCh38 reference (1000 Genomes version) with BWA-MEM (v0.7.15)⁸⁶. For plasma cfDNA, a modified alignment pipeline was used to accommodate adapter trimming after observing increased adapter contaminated reads in cfDNA samples as compared to tumor samples, due to the fact that cfDNA has shorter fragment size, which can lead to R1 and R2 overhang. Skewer⁸⁷ was used for adapter trimming (default settings) and subsequently aligned samples using BWA-MEM (default settings) to the GRCh38 reference (1000 Genomes version). For all samples, duplicate marking and sorting was done using NovoSort MarkDuplicates (v3.08.02), a multi-threaded bam sort/merge tool by Novocraft Technologies; www.novocraft.com), followed by indel realignment (done jointly for the tumor and matched germline) and base quality score recalibration using GATK (v4.1.8; https://software.broadinstitute.org/gatk), resulting in a final coordinate sorted bam file per sample. Alignment quality metrics were computed using Picard (v2.23.6; Quality ScoreDistribution, MeanQualityByCycle, CollectBaseDistributionByCycle, CollectAlignmentSummaryMetrics, CollectInsertSizeMetrics, CollectGcBiasMetrics) and GATK (average coverage, percentage of mapped and duplicate reads). To specifically assess for sample contamination, Conpair⁸⁸ was applied, which validated genetic concordance among the matched germline, tumor and plasma samples, as well as evaluated any inter-individual contamination in the samples. Samples that showed low concordance (<0.99) were excluded from further analysis. Specifically, three preoperative plasma samples from LUAD patients 37, 38 and 39 (described previously in MRDetect²⁸) and one set of serially monitored cutaneous melanoma samples from the melanoma patient MSK-55 were rejected from analysis due to low concordance score. As an additional quality metric, read depth skews were used in copy number neutral plasma regions where available (see Plasma read-depth denoising). Here, sample level Z scores were computed in CNV neutral regions (Appendix 1) using our read depth classifier and samples with a Z score value >10 were excluded. One adenoma plasma sample, Aar-35, was excluded under these criteria. An additional tumor sample, Aar-15, was excluded due to low tumor purity (<30% as assessed by Sequenza⁸⁹, Appendix 1), which precluded accurate SNV identification (number of somatic mutations <1,000, Appendix 1) in FFPE tumor tissue (see Tumor/Normal somatic mutation calling).

Tumor/Normal somatic mutation calling. The primary tumor and matched germline bam files were processed through the NYGC somatic variant calling pipeline⁹⁰. To achieve stringent somatic variant calling, high-confidence calls were enforced. Variants were further excluded that were present at any allelic fraction in the matched normal sample. It was noted that in the case of LUAD cohort, where tumor purity was lower (Appendix 1), and fewer overlapping reads between plasma and tumor mutations were available, and adjacent normal with potential tumor contamination was used rather than PBMC, the union of calls among mutation callers was used to broaden read availability. To further broaden read availability in this cohort, we did not enforce paired-read concordance (Appendix 3). To maintain consistency these standards were also applied to the neoadjuvant (Neo) lung cancer cohort. Small deletions and insertions (indels) were excluded. CNVs, including deletions, amplifications and copy-neutral LOH, were called using Sequenza (v3.0.0)⁸⁹. Only CNVs in autosomal regions (chr1-22) of the genome were considered, where the size of the CNV was greater than 1.5 Mb. Segments with Depth Ratio of 1 were characterized as neutral while those with Depth Ratio in excess of 1 (Depth Ratio >1.2) were selected as amplifications, and Depth Ratios less than 1 (Depth Ratio <0.8) were selected as deletions. LOH segments, including copy neutral LOH segments, were selected when Minor Copy-number was assigned 0 by Sequenza. To filter noise in FFPE tumors⁵⁸, we generated a FFPE tumor blacklist to remove any variant site present in 2 or more tumors in our Aarhus University cohort (n=35, Appendix 1). Only variants with a VAF greater than 0.2 were selected for analysis to exclude variants with minimal supporting reads in FFPE tissue.

Tumor-informed plasma cfDNA SNV identification. Detection of patient-specific compendia of SNVs was performed by searching the plasma WGS for all sites from the matched patient-tumor compendium with corresponding mutations in the same genomic site and the same substitution. To efficiently identify variants present in the sequencing data, a custom Python script (Python version 3.6.8) was used, which uses the pysam module to efficiently extract alignments harboring variants and extracted any read that both uniquely maps to a variant of interest and was in an aligned portion of the read (no clipping or soft masking at the position of the variant). In all plasma samples a subset of variants was removed through the use of a local recurrent artifact plasma ‘blacklist’ filter generated by aggregating pileup SNVs within our plasma WGS database (n=239 WGS plasma samples included in the analysis). Variants with a population allele frequency >4 or more appearances across patients within our plasma sample database were excluded. We generated a similar blacklist across all plasma sequenced at Aarhus University (n=50, Appendix 1) to account for local artifact bias⁹¹ and excluded any variants present in 2 or more plasma samples due to the smaller number of samples in this cohort. To further exclude potential germline variants, the gnomAD database (version 3.0) was used which contains genetic variants from >70,000 whole genomes⁹². The gnomAD version 3.0 variant call format (VCF) file that was available in hg38 coordinates from the gnomAD browser was downloaded. Single base changes were annotated that were identified with their population allele frequency and removed any candidate variants if the variant was present in gnomAD with an allele frequency >1/100. Finally, variants were excluded from simple repeat regions and centromeres from a problematic region blacklist⁹³.

Construction of ctDNA SNV training sets and feature space. All training sets were derived from plasma enriched for ctDNA SNV fragments (true label) from specific tumor types and cfDNA SNV fragments (false label) from healthy controls without known cancer processed in the same location and sequenced under the same settings. Appendix 2 lists samples used in training for LUAD, CRC, and melanoma. To identify informative features, quality filters were implemented to filter low-quality noise, germline SNPs, and genomic DNA (gDNA) contamination (see Appendix 3 for quality filters by model type). Broadly, filters focused on removing SNV fragments with low base quality (<25 on Phred scale), low depth (<10 supporting reads), and fragment size within 40 bp-240 bp to reduce gDNA contamination. Germline variants were excluded through filtering high VAF variants (VAF<0.2) except in cases where estimated iChorCNA TF was >0.2. The presence of candidate variants on overlapping paired reads was further enforced.

To maximize the accuracy of true (positive) labels, the following strategies were devised to limit noise contamination in our ctDNA (true label) SNV fragment sets. In all true label settings, training samples from patients with high burden metastatic disease (TF 9-24% as called by iChorCNA¹⁰, Appendix 2) were used. In samples where matched tumor tissue was obtained, ctDNA SNVs were nominated by intersecting tumor high confidence somatic calls from the NYGC Somatic Pipeline⁹⁰ with SNVs in plasma. When matched tumor tissue was not available, mutations were called directly in the plasma against normal germline sample using Mutect2⁹⁴, leveraging the high TF in these samples to identify consensus somatic mutations (Appendix 2). To further filter noise, when possible the intersection of ctDNA SNV fragments from two high TF timepoints from the same patient (Appendix 2) was used.

Candidate feature evaluation was performed on SNV fragments after applying quality prefiltering (Appendix 3) in both true and false labels. Features and corresponding single variant AUC scores are reported in Appendix 2. Several strategies were employed to create tissue-specific regional features that could inform the regional likelihood of somatic mutagenesis. Quantitative features were min/max normalized to values between 0 and 1. To evaluate local tumor mutational density, WGS SNV mutation calls from the PCAWG database⁸¹ were aggregated and the aggregate number of SNV mutations across all available tumor samples in a specific primary disease (e.g. melanoma) counted. Local transcription factor and histone CHiP-Seq marks as well as tissue specific bulk RNA expression values were calculated as reads per kilo base per million mapped reads (RPKM) and were drawn from primary tissue alignments in ENCODE⁹⁵. For each feature category (e.g. H3K4me3 ChIP-Seq marks), all alignments were assessed in ENCODE and selected alignments with the highest Pearson correlation between training set true and false label SNVs on Chromosome 1. In certain cases where strong (>0.15) positive and negative correlations were observed, alignments for both positive and negative correlations as separate model features. DNase peaks were downloaded as narrowpeak files from ENCODE^{95,96} and lifted to GRCh38. Disease-specific ATAC peak calls⁸⁰ were also downloaded from TCGA⁸². Plasma WGS sequencing error density was calculated by aggregating all SNV pileup variants from non-cancer control plasma sequenced at the New York Genome Center (Control Cohorts A and C, Appendix 4). For each of these features, quantitative values were calculated in a sliding interval window around candidate SNV fragments. The length of this window was optimized by comparing the correlation between feature and label between our training set true and false label SNVs on Chromosome 1 alone. Interval lengths are reported in Appendix 3. ChromHMM⁸³ chromatin annotation tracks were downloaded from ENCODE and lifted to GRCh38. HI-C compartment information was drawn from Hi-C SNIPER⁹⁷ bed files. Replication timing and mean expression values were drawn from prior work³⁷ and lifted to GRCh38. Other features, including distance to bound transcription factor⁹⁸ and SNV distance to nearest nucleosomal dyad in lymphocytes⁹⁹, were drawn from prior work and lifted to GRCh38. Appendix 3 lists features used in each model type.

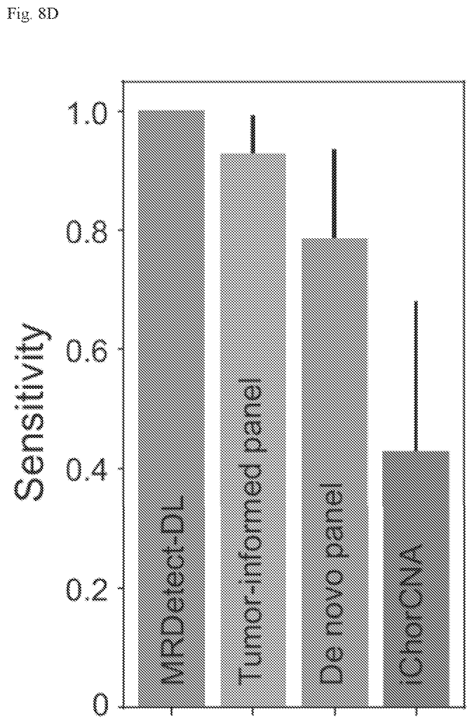

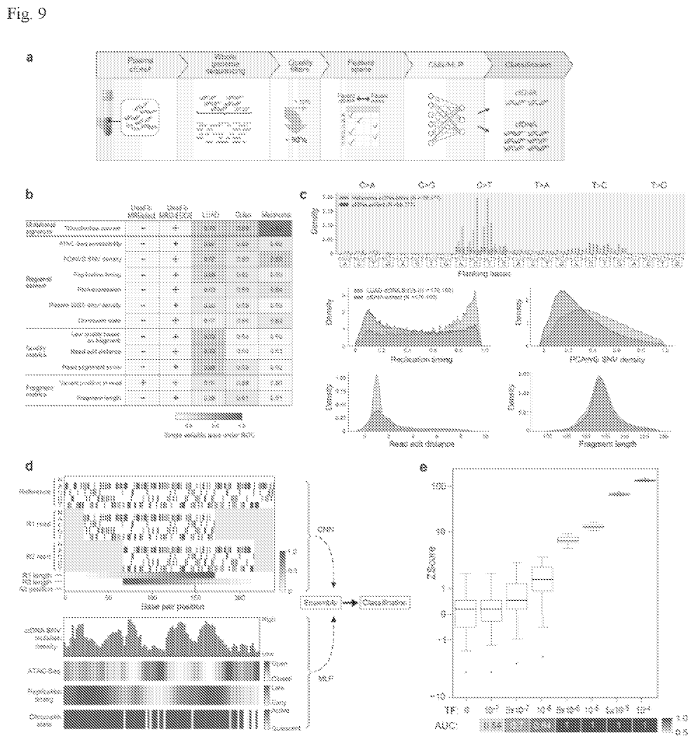

SNV deep learning model architecture and model training. To evaluate SNV fragments with the machine learning architecture, candidate SNV fragments were pulled from alignment files using pysam (v0.15.2) and salient features were encoded as input to the deep learning model architecture (FIG. 9D) with a custom python (v3.6.8) script. There are two main components of our deep learning SNV model architecture: a regional MLP, and a fragment CNN. The MLP takes a tabular feature representation as input and consists of five fully-connected layers with ReLU activation functions of decreasing size. Each layer is preceded by a batch normalization layer and followed by a dropout layer (with the exception of dropout following the final layer).

cfDNA fragments were represented as an 18×240 tensor (FIG. 9D). Within the rows of the tensor the one-hot encoded reference sequence was compared to the R1 and R2 sequence of a cfDNA fragment containing a variant (either true somatic mutation or sequencing artifact). The length and position of R1 and R2 was also encoded, and the position of the SNV to be classified as ctDNA or noise marked. The columns of the matrix mark individual nucleotides along the length of the fragment. The R1 and R2 regions are padded with neutral values (0.2 in each of the 5 possible nucleotides N, A, C, T, G) where the read does not overlap the reference sequence. This tensor serves as input to a CNN which consists of 4 one dimensional convolution layers (convolving over the base pair width dimension), each followed by a max pooling operation. This is then followed by three fully-connected layers (with ReLU activation) and a subsequent dropout layer, and ends with a single sigmoid-activated fully-connected layer (parallel to the MLP). Model architectures were built in Keras (v.2.3.0) with a Tensorflow base (1.14.0). The fragment tensor has potential access to features including fragment length, key genomic features including mutation type, trinucleotide context, and leading or lagging strand, and quality metrics such as PIR and edit distance (how many variants against the reference sequence are present in a fragment). The tensor structure is coded to account for all possible CIGAR outputs, including insertions, deletions, skips, and soft masks, by inserting ‘N’ (base undetermined) values in reads (deletions, soft skips, soft masks) or the reference sequence and as needed in the alternate read (insertions).

Finally, to integrate fragment and regional information, an ensemble classifier with sigmoid activation jointly evaluates the latent space outputs from both the fragment CNN and regional MLP to generate a score between 0 and 1, reflecting the model-based likelihood that a candidate variant containing cfDNA fragment harbors a true somatic mutation (1) vs. a sequencing artifact (0).

Deep learning classifiers (melanoma, CRC, LUAD) were trained using Keras with tensorflow background on fragments from disease specific training sets (LUAD, CRC, and melanoma, Appendix 2) chosen at the sample level. Validation sets were held out from training and drawn from separate patient samples. All performance metrics, including F1, AUC and accuracy within balanced sets, are reported for training sets and validation sets (Appendix 2).

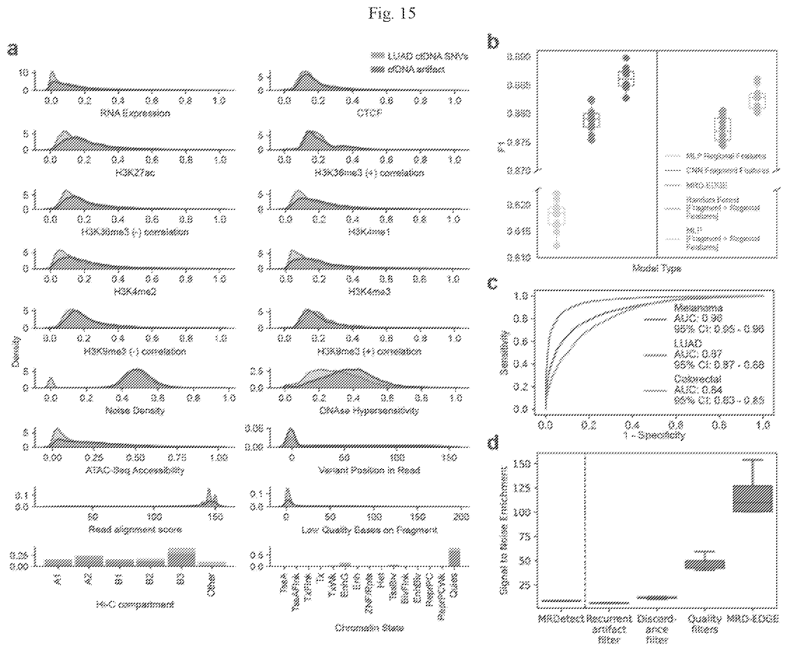

Comparison of MRD-EDGE SNV deep learning classifier performance to other machine learning models. The MRD-EDGE ensemble classifier (FIG. 9D) was compared to its individual components (fragment CNN and regional MLP) and other machine learning architectures (MLP and random forest model) by randomly subsampling without replacement in ten parts ctDNA and cfDNA SNV fragments from the held-out melanoma validation set (Appendix 2) and assessing F1 performance on each subsampling set (FIG. 15B). To assess fragment-level features in the Random Forest and MLP models, salient features were encoded as tabular values, including one-hot categorical encodings for trinucleotide context and mutation type of the candidate SNV as well as numerical representation of fragment-length, position of the variant within the read (PIR), read 1 length, and read 2 length. The MLP for Fragment+Regional Features has the same architecture as the Regional MLP (see SNV deep learning model architecture and model training). The Random Forest Fragment+Regional Features model was constructed using the Python (version 3.6.8) module sklearn sklearn. ensemble.RandomForestClassifier with default settings.

Generation of synthetic-plasma DNA admixtures. For MRD-EDGE SNV performance evaluations, in silico admixtures (range, 10⁻⁷-10⁻³) from MEL-01 plasma and plasma from a healthy control patient without known cancer (patient C-16) were generated. For MRD-EDGE CNV performance evaluations, given the challenges of applying LOH-based classification on samples with different germline SNPs, in silico dilutions were generated, with varying fractions (range, 10⁻⁶-10⁻³), of reads from a pretreatment high burden melanoma plasma sample (AD-12 pretreatment timepoint, TF 17% with 1.6 GB of total aneuploidy) into a posttreatment plasma sample from the same patient following a major response to immunotherapy (AD-12 Week 6 Timepoint, TF<5% without observable aneuploidy,). A pre- and postoperative plasma sample from a patient with NSCLC (Neo-03, TF 3.6% with aneuploidy matching tumor CNVs preoperatively, no aneuploidy postoperatively, Appendix 2) was similarly admixed. SAMtools (v1.1, view -s and merge commands) was used to downsample and admix high burden cancer plasma cfDNA reads into low burden (for CNV performance evaluation) or healthy control (for SNV performance evaluation) plasma cfDNA reads accounting for TF and tumor ploidy.

The downsampling ratio S to generate dilutions at various TFs was described previously²⁷ and is as follows:

$$S = \frac{T {F}_{required}}{{H}_{TF}} = {TF}_{required} * \frac{{H}_{TF} * {P}_{L} + ( 1 - {H}_{TF} ) * 2}{{H}_{TF} * {P}_{L}} \qquad Eq . 1$$

Where H_{TF} denotes ctDNA TF in the high burden cfDNA sample, P_{L} denotes ploidy in the tumor sample, High burden and control coverage is scaled followed by merging of reads:

$$high burden read ratio = S * \frac{{cov}_{req}}{{cov}_{H}} \qquad Eq . 2$$

$$control read ratio = ( 1 - S ) * \frac{{cov}_{req}}{{cov}_{C}}$$

Where cov_{req} is the required read depth coverage for the admixture sample and cov_{H}, cov_{C} are the read depth coverage of the high burden and control samples, respectively.

Plasma SNV-based ctDNA detection and quantification in the tumor-informed approach. As described previously²⁷, the relationship was modeled between coverage, mutation load (SNV/tumor), number of detected variants in cfDNA WGS, and the tumor fraction according to the following equation:

$$M = N ( 1 - {( 1 - TF )}^{cov} ) + μ * R \qquad Eq . 3$$

Where M denotes the number of SNVs detected in the plasma sample, N denotes the number of SNVs (mutation load) in the patient-specific mutational compendium, TF denotes the tumor fraction, cov denote the local coverage in sites with a tumor-specific SNV, u denoted the mean noise rate (number of_errors/number of reads evaluated) that corresponds to the patient-specific SNV compendium evaluated in control plasma WGS data (see below), and R denotes the total number of reads covering the patient-specific mutational compendium. This relationship allows the calculation of the plasma TF from the mutation detection rate, even in extremely low allele fraction where the mutation allele fraction itself is not informative (random sampling between 0 and 1 supporting read at best).

To address variation in sequencing artifact noise (μ) across patients with different mutational compendia, the patient-specific mutational compendium was applied to calculate the expected noise distribution across the cohort of control plasma samples. The process described herein is performed to detect the patient-specific SNVs in control plasma samples or other patients (cross-patient analysis). These detections represent the background noise model for which the mean and standard-deviation (μ,σ) of artifactual mutation detection rate was calculated. Confident ctDNA tumor detection can then be defined by converting the patient-specific detection rate (det rate=number of SNVs detected in cfDNA/number of reads checked=M/R) to a

$$Z - score = \frac{det\_rate - μ}{σ} ,$$

and define a threshold that will keep the specificity above 90%. Specificity and sensitivity performance values were further validated using receiver operating characteristic (ROC) curve using the Python (version 3.6.8) module sklearn sklearn.metrics.roc_curve.

Calculating the patient tumor fraction (TF) from point mutation detection was then carried out by the following equation (which is an inversion of Eq.3) as described previously²⁸.

$$T F = 1 - {( 1 - [ M - μ * R ] / N )}^{1 / c o v} \qquad Eq . 4$$

Where M denotes the number of SNVs detected in the plasma sample, N denotes the number of SNVs (mutation load) in the patient-specific mutational compendium, TF denotes the tumor fraction, cov denotes the local coverage in sites with a tumor-specific SNV, μ denotes the noise rate (number of errors/number of reads evaluated) that corresponds to the patient-specific SNV compendium, and R denotes the total number of reads covering the patient-specific mutational compendium.

Selection of control plasma samples for tumor-informed approaches. In the tumor-informed setting, patient-specific mutational compendia are applied to both matched plasma and control plasma. To exclude batch specific biases, control plasma samples obtained from the same collection site, sequencing platform and sequencing location as our cancer plasma samples were employed. For example, early-stage CRC plasma, sequenced at the New York Genome Center on Illumina HiSeq X, was compared to similarly sequenced healthy control plasma (Control Cohort A), while adenomas and pT1 lesions, sequenced with Illumina NovaSeq 1.5 at Aarhus University in Denmark, was compared to healthy control plasma sourced and sequenced from that institution (Control Cohort B). Control plasma samples used in model training or to construct a read-depth classifier PON were not used in downstream analyses (e.g., ROC analyses).

Plasma read-depth denoising. A read-depth denoising approach was recently introduced for reducing recurrent noise and bias for WGS-based tumor CNV detection⁴⁰. The read-depth pipeline separates foreground (CNV signal) from background (technical and biological bias) in read depth data by learning a low rank subspace across a panel of normal samples (PON) using robust Principal Component Analysis (rPCA) and applies this subspace to a tumor sample to infer CNV events. To optimize the approach for plasma, PONs were first created from healthy controls plasma generated with the same sequencing preparation (see Selection of control plasma for tumor-informed approaches, Appendix 3). Log transformed, zero centered read depths were then created across the PON for each sample within 1 Kb genomic windows. A window-based rPCA decomposition was performed on the PON to yield a subspace of biases that define “background” noise. Cancer plasma samples were subsequently projected on this background subspace to produce two vectors: a background bias projection and a residual corresponding to plasma CNV read-depth skews. Genomic windows were further filtered in plasma where read depth was ‘NA’ or was outside of 2.5 standard deviations away from the sample mean.

To generate sample read-depth scores for the read-depth classifier, window-level read depth values were median-normalized either to sample or chromosome based on mean plasma cohort autocorrelation (to sample <0.06<to chromosome, Appendix 1). This signal was then aggregated based on the direction of the CNV change in tumor (−1*deletion and +1*amplification) to produce a mean per-window read-depth score as described previously²⁸. This sample level read-depth score was compared to read-depth scores from held-out control plasma samples in matched genomic regions to generate a final sample-level Z score.

Plasma CNV-based TF estimation for use in read-depth skews. Estimated TFs for the read-depth classifier and MRDetect-CNV at different TF admixtures were calculated as:

$${TF}_{est} = \frac{{RDS}_{mixed} - μ}{{RDS}_{initial} - μ} * T {F}_{i nitial} \qquad Eq . 5$$

Where RDS_{mixed} is the aggregated median-normalized read depth signal for a specific mixing replicate, RDSᵢₙᵢₜᵢₐₗ is the aggregated median-normalized read depth signal for the initial high burden sample, μ (noise rate) is the average of aggregated median-normalized read depth signal across held-out plasma controls, and TFinitial is the tumor fraction of the initial high burden sample.

Evaluation of B-allele frequency in plasma. GATK (v3.5.0, software. broadinstitute.org/gatk) HaplotyeCaller was applied to identify genome-wide germline SNPs in PBMC WGS data. Major alleles were then identified in matched tumor tissue by selecting SNPs with BAF>0.6 in tumor regions with LOH (see Tumor/Normal somatic mutation calling). To enrich for local signal, SNPs were grouped into non-overlapping 1 Mb genomic windows. To ensure evaluation of only true SNPs and that signal was not biased by coverage or subtle clonal mosaicism in PBMCs, stringent quality filters were implemented, including minimal coverage thresholds (plasma and PBMC read depth ≥20 reads) and outlier criteria (0.3<plasma BAF<0.7, 0.4<PBMC BAF<0.6) at the individual SNP level. At the 1 Mb window level, bins with few SNPs (≤50 SNPs/bin) and outlier bins in which the mean plasma or PBMC BAF was outside of 2.5 standard deviations from mean window-level plasma and PBMC BAF from samples sequenced within the same sequencing platform (HiSeq X or NovaSeq) were further filtered. Because 1 Mb window-level mean BAF variance is a function of number of SNPs (higher BAF variance with fewer SNPs), window-level BAF values were converted to Z scores normalized for number of window-level SNPs in intervals of 50 SNPs for both plasma and PBMC BAFs, using the range of BAF values for all windows seen in that sequencing platform (HiSeq X or NovaSeq).

Short-read genome sequencing of plasma cannot place SNP variants in phase due to read length limits and the distance between successive SNPs¹⁰⁰. A technical obstacle of comparing phased variants in cancer plasma samples (identified only through LOH in tumor) to unphased variants in control plasma was faced. To remove the underlying contribution of phasing to aggregate BAF signal, window-level PBMC BAF values were subtracted, where deviations from 0.5 may be due to chance or subtle underlying clonal mosaicism, from window-level plasma BAF values to produce a window-level BAF score that reflects the BAF signal from the contribution of ctDNA in cancer plasma in excess of BAF signal from phased variants alone. In control plasma, where variants cannot be phased, the major allele was chosen randomly and individual SNPs aggregated to form window-level BAF noise distributions.

At the sample level, window-level BAF scores are aggregated to produce a mean per-window sample-level BAF score. Sample-level BAF scores in cancer plasma are compared to controls in matching genomic regions to produce a final sample-level Z score that reflects the BAF contribution of ctDNA in cancer plasma compared to matched noise.

Evaluation of tumor-informed fragment size entropy. Fragment length entropy was calculated to capture the heterogeneity of fragment insert size for cfDNA fragments within consecutive non-overlapping 100 kb genomic windows. Analyses was restricted to fragments with insert size between 100-240 bp. First, in each window the fraction of fragment sizes in each 5 bp interval from 100-240 bp was calculated. Shannon's entropy was then calculated on the set of these fractional inputs. At the sample level, window entropy values were converted from all 100 kb windows (neutral and CNV) to median-normalized robust Z scores. By normalizing to the distribution of entropy values in each sample, neutral regions serve as an internal control that accounts for the baseline fragment length heterogeneity within each sample inclusive of entropy noise from different sample preparations and pre-analytic biases. Following normalization, window-level Z scores were multiplied based on the direction of the CNV change using the underlying knowledge of tumor events. More fragment entropy was expected from the contribution of additional ctDNA fragments in tumor amplifications and thus multiplied these values by +1, versus less fragment entropy from the contribution of fewer ctDNA fragments in tumor deletions and therefore multiplied these values by −1. Regions surrounding transcription start sites (TSS) are known to harbor altered fragmentation profiles including an increase in short fragments^{14,44,101}, and this is particularly impactful for regions with deletions in matched tumors, where the shorter TSS fragment signal would confound the anticipated signal of less entropy due to lower contribution of short ctDNA fragments. Bins containing and flanking TSS sites identified in tissue specific ChromHMM⁸³ annotations (e.g., primary colon TSS for CRC samples) in deletions were therefore excluded. Outlier regions were further excluded where window-level Z score was greater than 5 median absolute deviations (MADs) from the sample median. It was noted that recurrent amplifications in chromosome 1p and 22q were uniformly present in control plasma samples in Control Cohort A (n=34 plasma samples) and Control Cohort C (n=30 plasma samples), and these regions were excluded from analysis as likely cfDNA WGS-specific artifacts.

At the sample level, signed window-level CNV Z scores (after multiplication by expected direction based on matched tumor amplification/deletions) were aggregated across windows to generate a sample-level fragment entropy score. Sample level fragment entropy scores in cancer plasma were compared to controls in matching genomic regions to produce a final sample-level Z score that reflects the contribution of ctDNA in cancer plasma compared to noise in non-cancerous control plasma.

Removing artifactual CNV events. To reduce CNV artifacts genomic bins overlapping centromere and telomere regions (as defined in genome.ucsc.edu/for GRCh38)+/−5 Mb around each region) were filtered out. Somatic CNV events originating from possible clonal hematopoiesis can also create biases in plasma cfDNA CNV analysis, as most cfDNA is derived from blood cells. To identify such events the genome-wide distribution of BAF in PBMC samples were evaluated, as assessed by ascatNgs (v4.2.1) and excluded any regions (variable segment sizes) where the mean BAF was above 0.6. Three patients had detectable somatic PBMC events as described previously²⁸: LUAD10 (amp Chr12: 60138-133841502), LUAD26 (CN-LOH Chr4: 50400000-191044164) and CRC03 (del Chr3: 234305-80851349; del Chr5: 75605307-180877637; del Chr7: 95649215-125071428; del Chr7: 144889607-159128563; del Chr10: 50003039-108417985; del Chr15: 36365636-63901029; del Chr17: 7602691-13317308; del Chr17: 17598183-20374289; del Chr18: 24227106-78017148).

Aggregation of CNV scores. The 3 CNV features (read-depth, fragment entropy, and BAF) independently inform the estimation of ctDNA signal. The features were therefore aggregated by combining Z scores using Stouffer's method

$$\frac{Z = {\sum }_{i = 1}^{k} {Z}_{i}}{\sqrt{k}} .$$

The MRD-EDGE CNV platform was not applied to our early-stage LUAD cohort due to low tumor purity (median 0.23, range 0.05-0.53, 12/39 samples with tumor purity ≤15%, Appendix 1) which prevented Sequenza from assigning tumor ploidy and total and minor copy number calls in over 30% of samples. Further, in the LUAD cohort, adjacent normal tissue was used rather than PBMC, and therefore the underlying PBMC tissue could not be assessed for clonal hematopoiesis events that could serve as a major confounder to our BAF analyses. To assess neoadjuvant (‘Neo’) NSCLC cohort, the same standards as were applied to the LUAD cohort was used to demonstrate generalizability of the SNV-only approach across sequencing platforms (Illumina HiSeq X in LUAD cohort and Illumina NovaSeq v1.0 in Neo cohort).

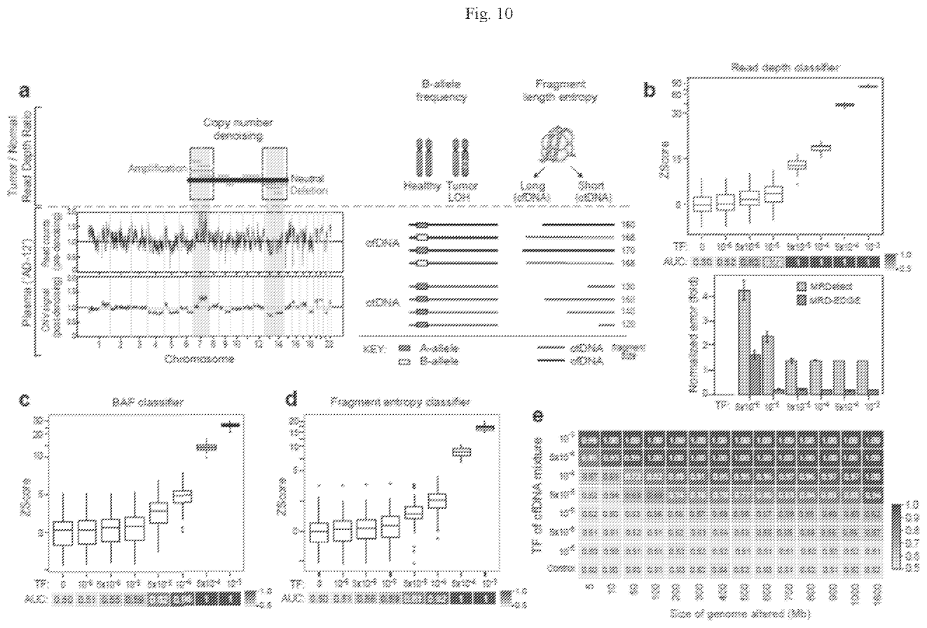

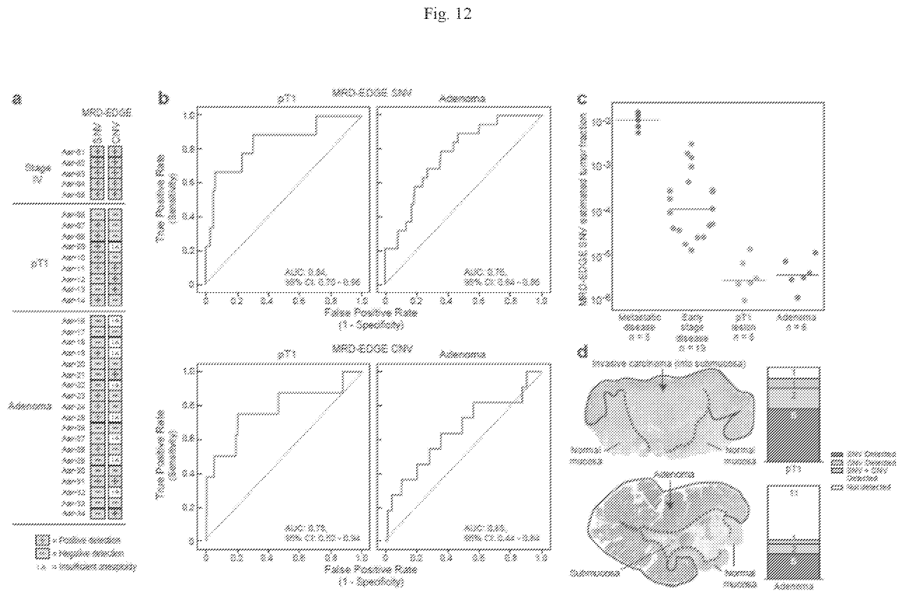

For the cohort of adenomas and pT1 lesions, MRD-EDGE SNV classifier was used to first estimate the TF of detected samples. The estimated TFs of detected lesions by SNV was median 2.88*10⁻⁶ (range 1.02*10⁻⁶-1.45*10⁻⁵) in pT1 lesions and 3.78*10⁻⁶ (range 1.17*10⁻⁶-1.21*10⁻⁵) in adenomas. (FIG. 12C) It was therefore reasoned that the LLOD demonstrated in benchmarking for the BAF and fragment entropy CNV features (5*10⁻⁵) would preclude use in these extremely low TF lesions (FIG. 2c-d), and indeed the BAF classifier and fragment entropy classifier in these cohorts failed to detect signal in these lesions (AUC 0.51 and 0.48, respectively). It was therefore decided to proceed solely with use of the read-depth classifier, which demonstrated sensitivity down to 5*10⁻⁶ in in silico admixtures (FIG. 10B).

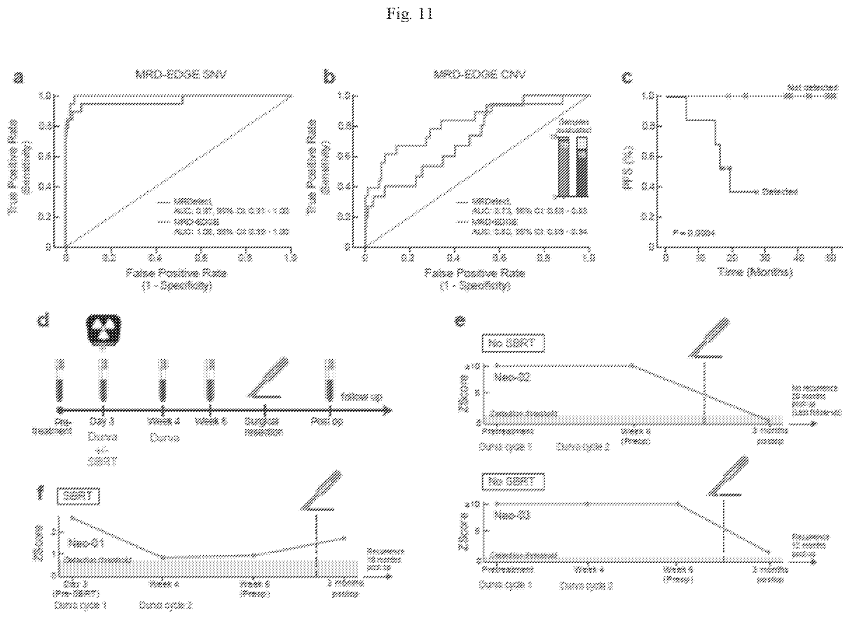

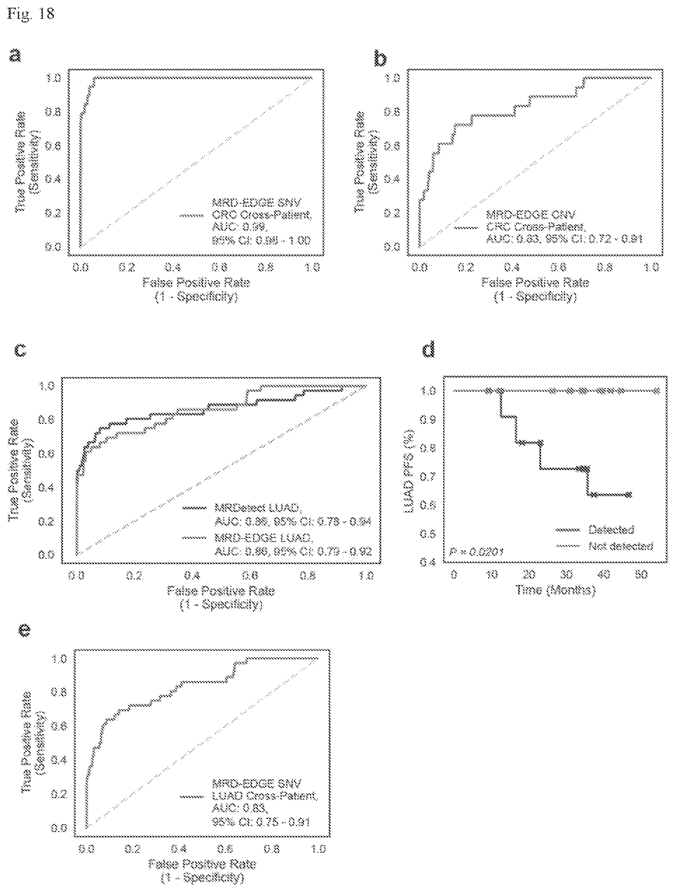

Integration of SNV and CNV scores. SNV and CNV classifiers provide orthogonal sources of information and were used to independently quantify ctDNA. MRD and pT1/adenoma detection was evaluated as a sample level Z score in excess of either the CNV or SNV Z score threshold as obtained through calculating the 90% specificity boundary compared to plasma from healthy controls in preoperative early-stage cancer samples. For example, in CRC, a positive detection was defined as a Z score threshold in excess of 90% specificity against healthy control plasma in the preoperative early-stage CRC cohort. These same pre-specified Z score thresholds were applied to identify postoperative MRD (FIG. 11C) and the pT1 and adenoma lesions (FIG. 12A). The same was done in lung cancer for the early stage LUAD and neoadjuvant therapy (‘Neo’) cohorts (FIG. 11D, FIG. 18C).

Quantification of mutational spectra for colorectal carcinomas and adenomas. Tumor somatic mutations (see Tumor/Normal mutation calling) were functionally annotated using GATK (v4.1.8) Funcotator (FUNCtional annOTATOR). Gene mutations were defined as missense mutations, nonsense mutations, nonstop mutations, frameshifts due to insertions and deletions (INDELs), and insertions and deletions causing nonframeshift coding mutations. Gene mutations were aggregated at the sample level and compared between CRC lesions of different stages.

Evaluating SNVs for de novo mutation calling. All variants against the hg38 reference genome were collected through samtools (v.3.1) mpileup with no exclusion filters. Only SNVs mapping to chromosomes 1-22 were included in the analysis. Indels were excluded. A custom python (v3.6.8) script was run to collect all fragments containing SNVs that matched pileup variants from the bam alignment. Fragments were then subjected to quality filters and the recurrent artifact blacklist and encoded as inputs to the model architecture (see SNV deep learning model architecture and model training). SNV detection rate, a function of the two unknown variables plasma TF and tumor mutational burden (TMB), was defined as the number of fragments classified as ctDNA over the number of post-filter fragments evaluated.

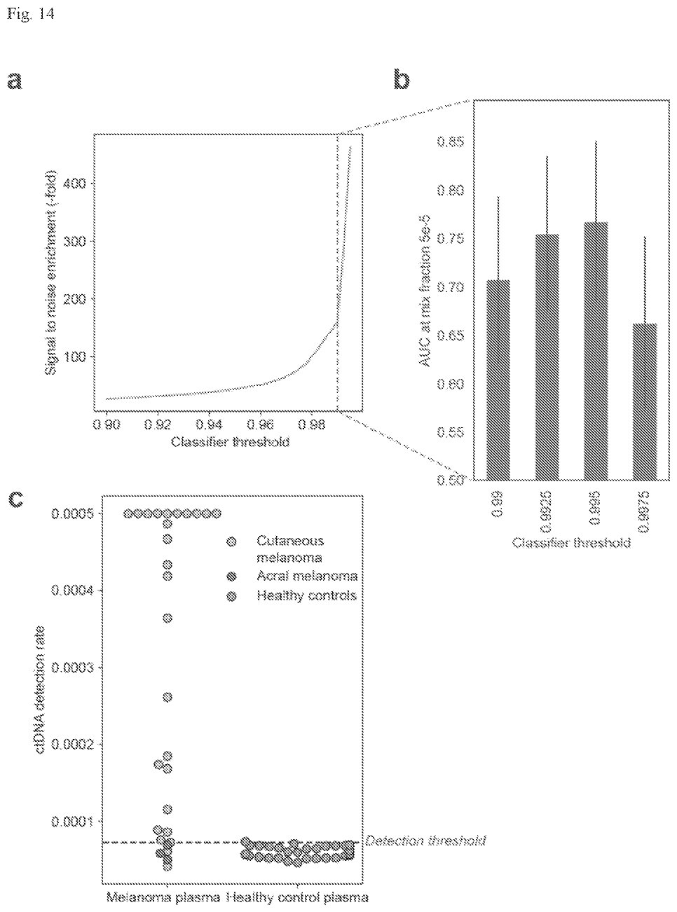

Determination of de novo mutation calling specificity threshold. In a tumor agnostic setting (de novo mutation calling), the datasets were more heavily imbalanced between signal and noise than in the tumor-informed setting, where knowledge of tumor SNVs is used to inform candidate variants. The specificity threshold was determined for de novo mutation calling within the MRD-EDGE SNV deep learning classifier by optimizing the trade-off at the fragment level between increasing signal enrichment at higher specificity thresholds (FIG. 14A) vs. decreasing signal availability from overly stringent filtering (FIG. 14B). Performance of the classifier was therefore evaluated at high specificity thresholds within in silico TF admixtures of MEL-01 and a healthy control plasma sample (C-16, Appendix 2). Detection sensitivity vs TF=0 in admixtures TF=5*10⁻⁵ was evaluated and AUC was found to be highest at a specificity threshold of 0.995 (FIG. 14B), with decreasing AUC at 0.9975 and 0.9925. This empirically chosen specificity threshold was used for evaluation of plasma TF in subsequent de novo mutation calling analyses. Notably, the cancer MEL-01 sample used in threshold determination was excluded from all downstream analysis.

ichorCNA. ichorCNA¹⁰ (version 2.0) was used as an orthogonal CNA-based method for cfDNA detection and the estimation of plasma TF in high burden plasma samples. The input setting was optimized for more sensitive detection in low-tumor-burden disease using the modified flags-altFracThreshold 0.001, -normal 99 along with a GRCh38 panel of normal (gatk.broadinstitute.org/). All other settings were set to default values.

Tumor-informed and de novo targeted panel. MSK-ACCESS⁸ was used as an orthogonal SNV-based method for evaluation of plasma TF in melanoma samples. MSK-ACCESS was run independently on a subset of pre- and posttreatment plasma samples for 14 patients with cutaneous melanoma with available material allowing concurrent analysis. Application of MSK-ACCESS panel and data analysis was performed by the MSK-ACCESS team. Results for the tumor-informed panel were informed by somatic mutations found in matched tumor samples through MSK-IMPACT¹⁰² and were reported as average adjusted VAF across evaluated genes. VAF was adjusted to account for copy number alterations at the locus of interest. Copy number alterations are inferred by applying FACETS¹⁰³ to Whole Exome or Whole Genome tumor tissue used in MSK-IMPACT analysis. The ACCESS team assumes that there are no changes to copy numbers of these segments between the IMPACT and ACCESS samples. Adjusted VAF is calculated as follows

$$VAF = \frac{{T}_{ALT} * T F}{{T}_{CN} * T F + {N}_{CN} * ( 1 - T F )} \qquad Eq . 6$$

Where VAF is the expected variant allele fraction, TF is tumor fraction, T_{ALT}=alternate copies in tumor, T_{CN}=total copies in tumor, N_{CN}=total copies in normal. Solving the equation for TF yields:

$$TF = \frac{{N}_{CN} * VAF}{{T}_{ALT} + ( {N}_{CN} - {T}_{CN} ) * VAF} \qquad Eq . 7$$

For ACCESS samples, this TF value is computed and named adjusted VAF (VAF_{adj}). For the de novo panel, only adjusted VAFs above 0.005 contributed to average VAF.

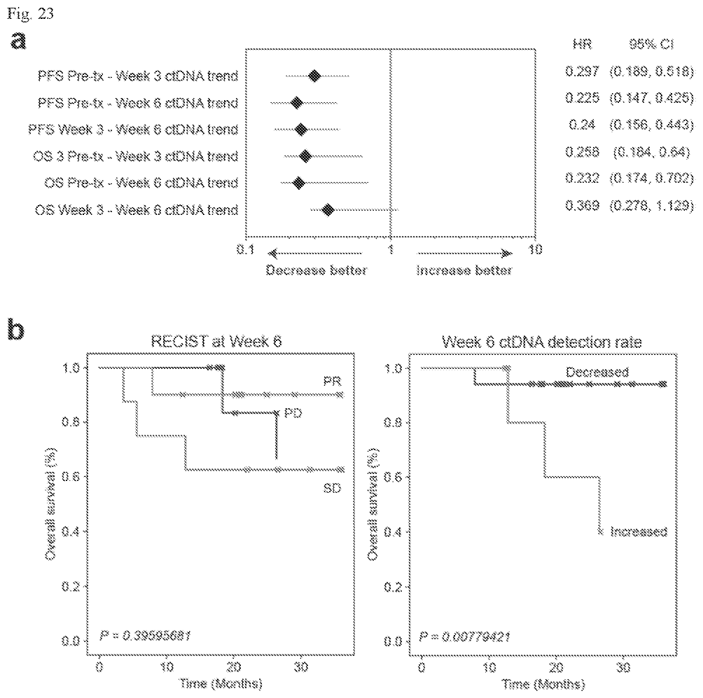

Statistical analysis. Statistical analysis was performed with Python 3.6.8 and R version 3.6.1. Continuous variables were compared using Student's t-test, the Wilcoxon rank-sum test or the nonparametric permutation test, as appropriate. All P values are two sided and considered significant at the 0.05 level, unless otherwise noted. Cox proportional hazards models were fit using lifelines¹⁰⁴ and forest plots (FIG. 23A) were plotted using EffectMeasurePlot from zEpid (0.9.0, zepid.readthedocs.io/).

### Example 2: Deep Learning Integrates Mutagenesis Features to Distinguish ctDNA SNVs from Sequencing Error

A prominent obstacle to WGS-based detection of ctDNA SNVs is distinguishing true tumor mutations from far more abundant sequencing error. In previous work²⁸, an error suppression framework was developed that operates at the individual fragment (rather than locus) level. This significant departure from traditional consensus mutation callers was driven by the expectation that in standard WGS coverage (e.g., 30X) of low TF samples (e.g., TF<1:1000), at best only a single supporting fragment will be detected for any given mutation. A support vector machine (SVM) classification framework was applied to exclude error associated with lower quality sequencing metrics including variant base quality (VBQ), mean read base quality (MRBQ), variant position in read (PIR), and paired-read mutation overlap. Focused solely on eliminating sequencing error, the classifier was trained on reads with germline SNPs (true labels) vs. reads with sequencing errors (false labels).

It was posited that signal to noise enrichment may emerge not only from characterizing features specific to sequencing errors (decreasing noise), but also from learning features indicative of true ctDNA mutations (increasing signal).

Learning features specific to ctDNA required a rethinking of the machine learning training paradigm, as germline SNPs can no longer serve as a source for true (positive) labels. Instead, cfDNA samples were leveraged with high TF (range 9-24%, Appendix 2) across three common cancer types with high mutational burden: melanoma, LUAD, and colorectal cancer. These high TF plasma samples (range n=2-4) provided an abundant (51,160 to 270,648, Appendix 2) source of fragments enriched with somatic mutations (true labels) from which to develop a ctDNA SNV feature space. The ctDNA SNVs were compared to cfDNA fragments containing sequencing errors drawn from controls (range n=4-5) without a known malignancy (Appendix 2 and Methods). To ensure that classification is optimized to detect more subtle differences between signal and noise, a set of quality filters was implemented to remove germline SNPs, recurrent plasma WGS artifacts, and variants with low base or mapping quality scores (Appendix 3 and Methods).

After obtaining a large, pre-filtered training corpus of ctDNA SNVs and cfDNA SNV artifacts, a broader feature space was next explored to help distinguish the two. First, single base substitutions (SBS) sequence patterns are closely associated with cancers driven by distinct mutational processes^{31,32} such as SBS4 signature (tobacco exposure) in LUAD or SBS6 (ultraviolet light) in melanoma. Second, ctDNA has been associated with shorter fragment size^{30,33,34}. Third, SNVs are overrepresented in distinct locations within the genome, including a predilection for quiescent chromatin and late replicating regions³⁵⁻³⁸, allowing for inference of the local (e.g., 20 Kb) mutation likelihood. This evaluation allowed for the identification of informative features with varying contribution across tumor types (FIG. 9B, FIG. 15A, Appendix 3).

To integrate this expanded feature set for optimal classification, it was reasoned that neural networks would best serve the size of the training sets (100,000 s of fragments) and the underlying feature complexity. A two-dimensional matrix tensor was developed to represent a cfDNA fragment (FIG. 9D, top and Methods) and therefore capture fragment-level features such as SBS, fragment length, and quality metrics like read edit distance and PIR. In parallel, a second model architecture was designed to capture regional context, whereby each SNV-containing fragment is scored based on salient regional features associated with mutation frequency (FIG. 9D, bottom). For example, a fragment can be annotated with the local density of melanoma tumor SNVs in a 20 Kb interval surrounding the candidate SNV (Methods, Appendix 3 for a full list of features by cancer type). The fragment and regional architectures were combined as inputs to an ensemble model featuring a convolutional neural network (fragment CNN) for the fragment architecture and a multilayer perceptron (regional MLP) for the regional architecture. This ensemble model uses a sigmoid activation function to output a score between 0 and 1 to indicate the likelihood that a candidate SNV is either cfDNA sequencing error or a ctDNA mutation. The ensemble model outperformed both the fragment and region models individually and other machine learning architectures in a melanoma validation plasma sample (‘MEL-01’) held out from training and paired with SNV artifacts from healthy control plasma (FIG. 15B, Appendix 2). The deep learning methods were applied to a more stringent classification task than in previous work, as the classifier was applied to heavily pre-filtered fragments in which the majority of low quality cfDNA sequencing errors were excluded (mean 92.8%, range 91.2%-93.6%). In this context, the classification method yielded area under the receiver operating curves (AUCs) at the fragment level of 0.95 (95%: 0.94-0.95) in melanoma, 0.87 (0.86-0.88) in LUAD, and 0.84 (0.83-0.84) in colorectal cancer in validation plasma samples held out from training (FIG. 15C, Appendix 2).

Benchmark of the platform's enrichment capacity in the tumor-informed setting was then sought, in which a patient-specific mutational compendia drawn from resected tumor tissue was used to nominate SNVs for classification. Tumor-confirmed ctDNA SNVs from MEL-01 admixed with SNV artifacts drawn from 6 healthy control plasma samples that were held out from model training (‘Melanoma held-out validation fragments’, Appendix 2) were used. First, signal to noise enrichment was measured for the pipeline as a whole and at individual stages (FIG. 15D). Given the higher likelihood of a true positive in the tumor-informed setting, a balanced classification threshold (0.5) on the final ensemble model was used to classify ctDNA signal from noise. In a matched analysis in which both platforms were applied to the same data, a higher signal to noise (S2N) enrichment for MRD-EDGE (mean 118 fold, range 100-153 fold) was found compared to MRDetect (mean 8.3 fold, range 8-9 fold), which translates to a mean additional 14 fold S2N enrichment, (range 12-18 fold).

The lower limit of detection (LLOD) for the tumor-informed MRD-EDGE classifier in in silico TF admixtures (TFs 10⁻⁴-10⁻⁷, n=20 in silico admixture replicates, Methods) was next evaluated using reads from MEL-01 mixed into control cfDNA from an individual (‘C-16’) with no known cancer (FIG. 9E). When compared to the noise distribution in randomly chosen TF=0 replicates, higher performance was found even in the parts per million range and below (AUC of 0.84 at TF 1*10⁻⁶ and 0.7 at 5*10⁻⁷ for MRD-EDGE, compared to 0.77 and 0.65 for MRDetect, respectively).

### Example 3: Advanced Denoising and an Enriched Feature Space Enable Enhanced CNV-Based ctDNA Detection

Aneuploidy is observed in the vast majority of solid tumors and is a prominent hallmark of the cancer genome³⁹. It has been shown that MRDetect-based CNV detection can monitor disease burden in cancers with a high degree of aneuploidy but low SNV mutation burden²⁸. MRDetect sought to identify plasma read depth skews corresponding to matched tumor-informed CNV profiles to measure MRD in CRC and LUAD. While the results demonstrated a 2 order of magnitude improvement in sensitivity compared to leading CNV-based ctDNA algorithms^{10,28}, it required substantial aneuploidy (>1 Gb altered genome) to detect TFs of 5*10⁻⁵.

It was reasoned that detection of subtle read depth skews related to low TF ctDNA may be hindered by biases that arise from sample-preparation (e.g., GC bias), alignment (e.g., variable mapping), and biological factors (e.g., replication timing). These biases can introduce distortions (‘waviness’) in read depth signal which interfere with CNV estimation in both tumors and plasma⁴⁰. To correct for such biases, a machine-learning guided CNV denoising platform was developed for use in plasma WGS. The plasma read depth classifier uses robust principal component analysis (rPCA) trained on a panel of normal samples (PON) to correct read depth distortions due to background artifacts related to assay, batch, and recurrent noise (Methods).

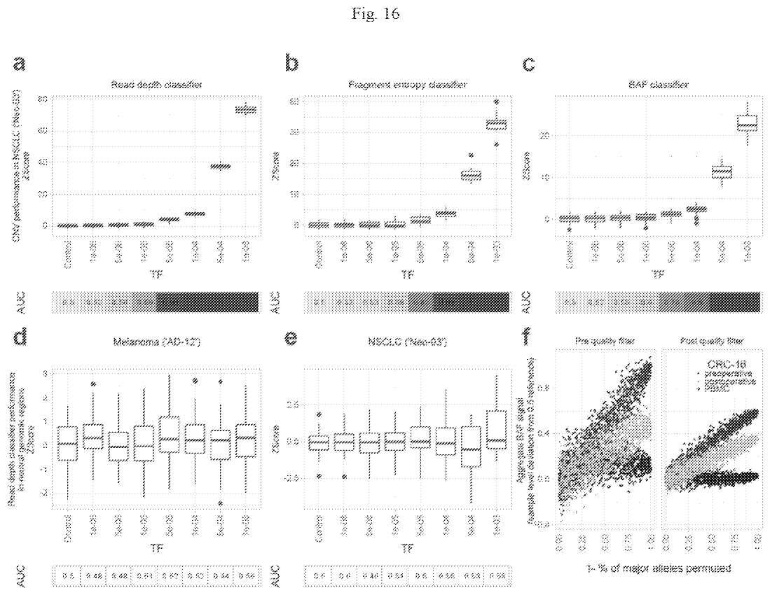

To evaluate the performance of ctDNA detection with the enhanced read-depth classifier, in silico reads from a pretreatment high burden melanoma plasma sample were admixed with a high degree of aneuploidy (′AD-12′, TF 17% with 1.6 GB of total aneuploidy, Appendix 2) into a posttreatment sample from the same patient following a major response to immunotherapy, varying the TF admixtures (range Oct. 3, 2010-6; n=50 technical admixing replicates with random independent seeds). Signal from read depth skews were identified at TF admixtures as low as 1*10-5 (FIG. 10B). Directional skew signal from copy neutral regions in the matched tumor served as a negative control (FIG. 16D).

In addition to enhanced denoising of read depth skews, it was reasoned that loss of heterozygosity (LOH) can serve as an important additional source of CNV signal. Copy neutral LOH cannot be captured by read depth skews but can be nonetheless measured through allelic imbalances in germline SNPs in plasma. Here, inference of the major allele in genomic regions affected by LOH was derived from tumor WGS^{41,42}, and perturbations of the B-allele frequency (BAF) in plasma were indicative of ctDNA contribution to the plasma cfDNA pool (FIG. 10A). To leverage LOH signal, plasma SNPs were aggregated in large genomic windows (1 Mbp) and assessed for window-wide allelic imbalance. To account for underlying biases and mosaicism within the cfDNA pool, BAF values were compared both to the expected contribution of 0.5 and to the underlying peripheral blood mononuclear cell (PBMC) BAF reference⁴³ (Methods), and quality filters were used to exclude aberrant signal due to low coverage and bias from PBMC (FIG. 16F). Benchmarking of BAF classifier in the same in silico admixtures yielded allelic imbalance signal in LOH regions in TF admixtures as low as 5*10⁻⁵ (FIG. 10C).

Finally, well-characterized abnormal ctDNA fragmentation patterns^{9,33,34,44,45} were leveraged as an additional source of aneuploidy signal. ctDNA is associated with shorter and more heterogenous fragment lengths than normal cfDNA^{9,44}. Fragment length entropy (measured as Shannon's entropy), a marker of heterogenous fragment lengths in cfDNA, in plasma WGS segments matched to amplifications and deletions in tumor was therefore measured. While existing approaches have sought to recognize altered fragmentation profiles inherently or compared to control (non-cancer) plasma^{9,46}, in the instant fragment entropy classifier, use of matched tumor tissue enables the cfDNA fragment pool in neutral plasma regions to act as an internal control. Fragment lengths in matched CNV segments can be assessed in comparison to copy-neutral segments rather than to an absolute baseline, removing confounding from baseline fragment length biases at the sample level. The entropy contributions was then measured from amplifications (greater plasma cfDNA entropy due to a larger contribution of ctDNA fragments) and deletions (less plasma cfDNA fragment entropy) to harness signal. In in silico admixtures, the fragment entropy classifier identified signal in TFs as low as 5*10⁻⁵ (FIG. 10D, Methods). To demonstrate sensitivity across cancer types, CNV features in TF admixtures derived from pre- and postoperative plasma from a patient with early-stage non-small cell lung cancer (NSCLC) was also benchmarked and similar performance was found (FIG. 16A-C).

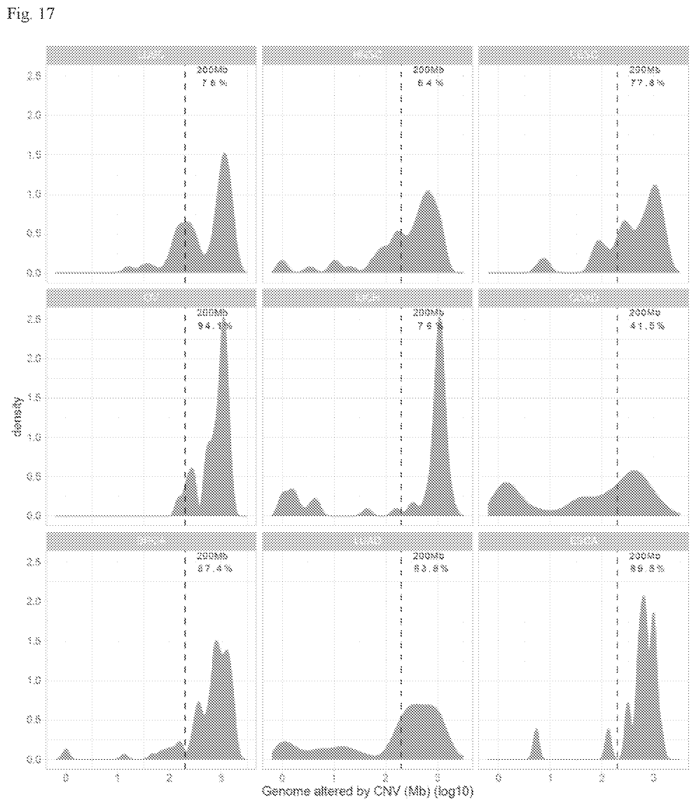

The three CNV classifiers-read depth, BAF, and fragment entropy-gather independent and complementary sources of CNV signal. MRD-EDGE combines signal from these classifiers as independent inputs at the sample level to comprehensively assess for plasma TF (Methods). Because the aneuploidy signal in plasma WGS is a function of both the proportion of the cancer genome affected by aneuploidy and the TF, classifier performance was evaluated by downsampling both the TF (as above in FIG. 10B-D) and the cumulative size of CNV segments to characterize a LLOD matrix (FIG. 10E). Classifier performance, as expected, improved with increased aneuploidy. However, while MRDetect required 1 Gb of aneuploidy²⁸ for a LLOD of 5*10⁻⁵, MRD-EDGE achieved an LLOD of 5*10⁻⁵ (AUC 0.74) with only 200 Mb of aneuploidy, which would extend applicability to many more solid tumors (FIG. 17).

### Example 4: MRD-EDGE Yields High Performance in Tumor-Informed Detection of Early-Stage Colorectal Cancer and Postoperative MRD

To evaluate MRD-EDGE in the tumor-informed early-stage cancer setting, the platform was tested on the previously reported²⁸ clinical cohort of plasma samples from patients with CRC (n=19, including 6 with microsatellite instability), compared with exposure matched controls without known cancer (n=34, ‘Control Cohort A’) and from the same sequencing platform (Illumina HiSeq X). Here, SNVs and CNVs from resected tumors form a patient-specific mutational compendia, which was then used to assess for ctDNA in pre- and postoperative plasma and to form noise (sequencing error) distributions in healthy control plasma. Z scores of patient plasma signal were derived from control plasma noise distributions and used assess for ctDNA detection in both the MRD-EDGE SNV and CNV platforms independently. The Z score detection threshold was set at 90% specificity against control plasma in the receiver operating curve (ROC) analysis, and a positive ctDNA detection was defined as patient plasma SNV or CNV Z score above this threshold.

In the early-stage CRC cohort, area under the curve (AUC) for preoperative ctDNA SNV detection with MRD-EDGE was 1.00 (95% CI: 0.99 to 1.00) and sensitivity was 100% at 90% specificity (compared with MRDetect AUC 0.97, 95% CI: 0.91-1.00, 95% sensitivity at 90% specificity, FIG. 11A). A cross-patient analysis, where the patient-specific mutational compendia was compared between matched and unmatched plasma, showed similar performance (FIG. 18A). It was noted that MRD-EDGE CRC SNV classifier was trained on high burden plasma sequenced with a different sequencing platform and at a different facility than the one used for the early-stage CRC samples (Illumina NovaSeq v1.5, Aarhus University, Denmark vs. Illumina HiSeq X, New York Genome Center, Appendix 1), demonstrating generalizability across platforms. MRD-EDGE for CNVs was applied independently to this preoperative cohort and demonstrated improved performance (AUC=0.82, 95% CI 0.71-0.91, 61% sensitivity at 90% specificity) compared to MRDetect (AUC=0.73 95% CI: 0.59-0.83, sensitivity=40% at 90% specificity, FIG. 11B). Moreover, the ability to evaluate copy neutral LOH in MRD-EDGE allowed application of CNV-based detection to 18/19 samples in this CRC cohort compared to 15/19 samples with MRDetect.

MRD was defined as a postoperative plasma Z score in excess of the same 90% detection threshold previously defined in preoperative plasma samples. MRD-EDGE detected postoperative MRD in 8/19 samples on plasma drawn a median of 43 days after surgery, four of which had confirmed disease recurrence. Postoperative MRD was found to be associated with shorter disease-free survival (FIG. 11C) over a median follow-up of 49 months (range, 18-76). Recurrence was not observed in any of the 11 patients in whom ctDNA was not detected. Of the 4 patients with postoperative detection who did not show evidence of recurrence, 1 received adjuvant therapy that may have eliminated residual disease, which has been demonstrated in other liquid biopsy settings²³. One patient had short overall survival at 18 months (unrelated death), below the median time to recurrence in CRC46, and the remaining 2 patients had microsatellite unstable tumors that have been shown to be associated with prolonged time to relapse and occasional spontaneous regression^{48,49}.

### Example 5: Tracking of Plasma Tumor Burden Throughout Neoadjuvant Therapy with MRD-EDGE

The MRD-EDGE SNV classifier was then applied to the challenging case of tracking plasma tumor burden in response to neoadjuvant immunotherapy. Tracking tumor burden in this setting could help optimize care during the crucial period between early-stage lung cancer detection and definitive surgery, with clinical implications such as extent of surgery planning for responders or moving to early surgery for non-responders. Plasma was evaluated from three patients with early-stage NSCLC on a neoadjuvant immunotherapy protocol⁵⁰ that randomized patients with early NSCLC to treatment with the ICI agent durvalumab with or without stereotactic body radiation therapy (SBRT) followed by surgical resection. Plasma was collected prior to the first ICI treatment or following day 3 SBRT (if applicable), at cycle 2 of ICI, prior to surgical resection, and after surgery (FIG. 11D).

To determine an appropriate specificity threshold for use in neoadjuvant lung cancer monitoring, we applied MRD-EDGE to a cohort of early-stage LUAD patients evaluated previously²⁸. MRD-EDGE maintained performance in this cohort compared to MRDetect (FIG. 18C-D) and allowed us to identify a Z score detection threshold in a larger, orthogonal cohort. Preoperative ctDNA was detected in each of these three neoadjuvant treatment patients using the detection threshold pre-specified from the early-stage LUAD cohort. One patient, Neo-01 (LUAD histology), had a marked decrease in plasma TF following SBRT, but ultimately plasma TF rose prior to surgery demonstrating a lack of response to ICI (FIG. 11F). This patient had detectable ctDNA postoperatively and was found to have disease recurrence at 18 months following surgery. Two patients who did not receive SBRT showed minimally changed tumor burden throughout ICI treatment and no evidence of pathological response at the time of surgery. The first, Neo-02 (non-specific histology), had undetectable ctDNA postoperatively and remains free of disease at 29 months. The second, Neo-03 (squamous histology), was found to have postoperative MRD and recurred at 12 months after surgery (FIG. 11E). These data highlight the potential of serial ctDNA monitoring during multi-pronged therapeutic regimens to define response to treatment and create opportunities for real-time therapeutic optimization.

### Example 6: MRD-EDGE Detects ctDNA Shedding in Precancerous Adenomas and Minimally Invasive pT1 Carcinomas

Whether noninvasive (precancerous) lesions shed ctDNA remains unresolved. The issue carries important implications for emerging early detection efforts where the presence of ctDNA from precancerous lesions may be advantageous in some settings, or alternatively diminish the precision of liquid biopsy screening tests. While MRD-EDGE requires a tumor prior and therefore cannot be used for screening, it was reasoned that the exquisite sensitivity of the approach provided herein could nonetheless address whether ctDNA is shed from adenomas and polyp cancers (pTlpN0), where ctDNA detection through existing methods such as droplet digital PCR and targeted sequencing has been limited^{51,52}.

Pre-resection plasma from 28 patients with malignant and premalignant lesions detected through screening at the Danish National Colorectal Screening Program was evaluated. Nine patients had pT1 lesions (defined as invasion of the submucosa but not the muscular layer, the earliest form of clinically relevant CRC⁵⁴), and 19 patients had screen-detected precancerous adenomas (including one adenoma with microsatellite instability). As a positive control, plasma from 5 patients with metastatic CRC were also evaluated. These samples were compared to healthy control plasma that was sequenced at the same location was used and with the same platform as the adenoma and pT1 lesion plasma (‘Control Cohort B’, Appendix 1 and Methods).

Consistent with prior reportsdecreased aneuploidy was found in adenomas (median 235 Mb of genomewide aneuploidy) compared to the early-stage CRC samples (median 594 Mb aneuploidy, P=0.02).

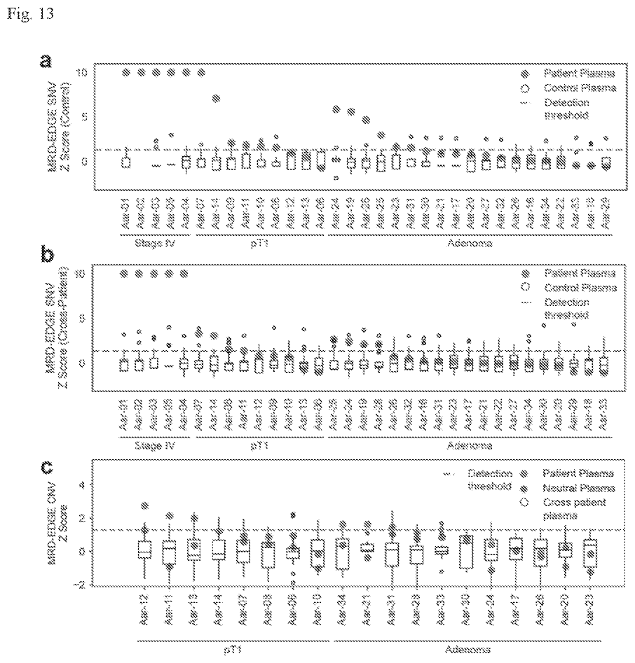

Performance of MRD-EDGE in this cohort was then assessed. To ensure generalizability of detection, the prespecified Z score threshold values from the preoperative early stage CRC cohort were applied (FIG. 11A-B). These thresholds yielded similar specificity for adenoma and pT1 detections for both SNVs and CNVs (89% and 93%, respectively) in this separate cohort of control plasma samples sequenced with Illumina NovaSeq v1.5 rather than Illumina HiSeq X (Appendix 1). MRD-EDGE detected ctDNA shedding in 8/9 (89%) pT1 lesions and 8/19 (42%) precancerous adenomas (FIG. 12A). Detection AUCs were higher for pT1 lesions than adenomas for both the SNV and CNV platforms, demonstrating decreased ctDNA signal in adenomas as expected (FIG. 12B). As in the early-stage CRC cohort, performance was analyzed in a cross-patient analysis (FIG. 13B-C) and similar detection ability was found. Notably, patient-specific mutational compendium in this setting was drawn from formalin-fixed paraffin-embedded (FFPE) tissue samples, which are prone to more SNV artifacts⁵⁸ than fresh frozen tissue samples used in our CRC and LUAD cohorts, further supporting the generalizability of classifiers among diverse tissue preparations. Using SNV-based TF estimations (Methods), lower TFs in detected lesions (median 2.88*10⁻⁶, range 1.02*10⁻⁶-1.45*10⁻⁵ in pT1 lesions and 3.78*10⁻⁶, range 1.17*10⁻⁶-1.21*10⁻⁵ in adenomas) than early-stage and metastatic CRC samples (FIG. 12C). Detections for pT1 and adenoma lesions were significantly above the expected false positive rate of 10% (binomial P=2.1*10⁻⁵ and 2.1*10⁻², respectively).

These data demonstrate that even without a significant invasive component, dysplastic tissue may shed ctDNA. The contribution of precancerous lesions or even benign clonal outgrowths to the cfDNA pool may thus form an important consideration as advanced non-tumor informed methods are deployed clinically, both for detection of adenomas and for early cancer detection efforts.

### Example 7: MRD-EDGE Enables ctDNA Monitoring in Melanoma Plasma WGS without Matched Tumor

Across solid tumors, tumor tissue may be scarce due to considerations ranging from scant biopsy material (e.g., stage II melanoma), lack of primary biopsies at tertiary care centers, or restrictions on access to primary tissue. For example, in prior bespoke panel studies the requirement for matched tissue led to the exclusion of a substantive proportion of eligible patients due to low tumor DNA purity or quality^{20,59}. Further, in several cancers, non-surgical treatment modalities like radiation are given with curative intent, again limiting opportunities for tumor-informed approaches. This introduces the need for tumor-agnostic (de novo) mutation calling platforms for clinical surveillance. The provided improved signal to noise enrichment in the tumor-informed setting (FIG. 15D) led to consideration of de novo mutation calling using the MRD-EDGE platform. In this setting, there is no a priori knowledge of high likelihood mutated loci, and ctDNA signal is therefore far more challenging to distinguish from sequencing error.

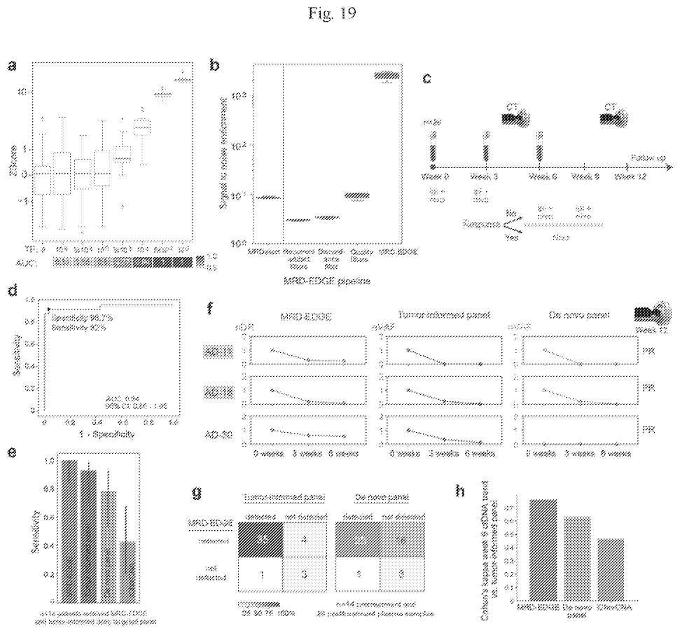

De novo mutation calling with MRD-EDGE requires the evaluation of all plasma fragments that harbor SNVs, which range from 1*107-1*108 per plasma sample in the WGS cohorts (Methods, Appendix 1). As these SNVs harbor far greater cfDNA sequencing noise compared to ctDNA signal, It was reasoned that higher specificity thresholds would need to be applied to the output of the deep learning classifier. To determine an appropriate de novo specificity threshold for the MRD-EDGE deep learning SNV classifier (FIG. 9D) the same in silico admixtures as in the tumor-informed setting (validation melanoma sample MEL-01 admixed with a held-out healthy control plasma sample, FIG. 9E). The signal to noise enrichment was compared with detection AUC at different specificity thresholds imposed on the MRD-EDGE ensemble model output (FIGS. 14A and 14B, Methods) to find an optimal threshold for classification of ultrasensitive TFs (TF 5*10⁻⁵). As expected, the empirically chosen threshold in the de novo classification context (0.995) was higher than the balanced threshold (0.5) used in the tumor-informed setting. At this threshold, AUC for ultrasensitive detection (5*10⁻⁵) was 0.77 (FIG. 19A). Signal to noise enrichment for MRD-EDGE was 2,518 fold (range 1,817-3,058 fold) compared to the MRDetect SVM (mean 8.3 fold, range 8-9 fold) in a matched analysis performed with the same samples used in the tumor-informed setting (FIG. 15D). This equates to 301-fold (range 211-357 fold, FIG. 19B) higher enrichment for MRD-EDGE compared to MRDetect.

After benchmarking fragment-level performance for de novo mutation calling with MRD-EDGE, performance was evaluated at the sample level in a cohort of patients with advanced cutaneous melanoma treated with combination ICI on The Adaptively Dosed Immunotherapy Trial⁶⁰ (′adaptive dosing cohort′, n=26 patients, 2-4 timepoints per patient, FIG. 19C). In this cohort, plasma was sampled at baseline (pretreatment) and prior to the second (Week 3) and third (Week 6) infusion of the ICI agents nivolumab and ipilimumab. The protocol aimed to spare excess combination ICI treatment by identifying responders through early imaging at Week 6 and transitioning these patients to monotherapy with nivolumab.

ctDNA detection rates were compared in the melanoma cohort to a cohort of controls (n=30 patients without known cancer, ‘Control Cohort C’) sequenced under similar conditions (Illumina NovaSeq v1.0 for melanoma and control groups) to avoid inter-platform bias. MRD-EDGE identified ctDNA in pretreatment plasma from cutaneous melanoma samples (n=25 after holding out one melanoma plasma sample with high TF used in neural network training), yielding an AUC of 0.94 (95% CI: 0.86-1.0, FIG. 19D). In keeping with the tumor-informed analyses, the first detection threshold was chosen at a specificity of 90% or greater (sensitivity of 92%, specificity of 96.7%). As a negative control, pre- and posttreatment plasma samples from a patient with acral melanoma (n=3 total plasma samples) within the same sequencing batch were included. As expected, no ctDNA detection was observed in these samples (FIG. 14C), confirming that the classifier is specific for the distinct mutational signatures of cutaneous melanoma.

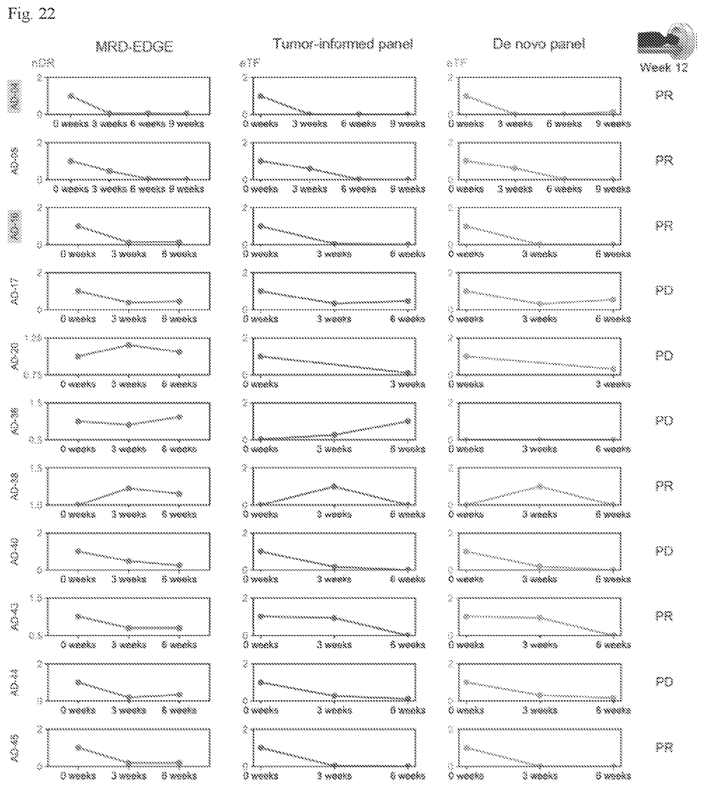

To benchmark MRD-EDGE ctDNA detection in pretreatment plasma against alternative methods, results were compared to a state-of-the-art targeted panel⁸ with tumor-informed mutation calling covering 129 common cancer genes (‘tumor-informed panel’) in a subset of 14 patients. Tumor-informed detection was based on an average of 9.4 panel-covered SNVs per sample (range 2-29, Appendix 4). Four patients had 14 or more SNVs (highlighted in FIG. 19F, FIG. 22), a range comparable to leading bespoke panels^{19,20,59}. In parallel, results were also compared to the same targeted panel with de novo mutation calling (‘de novo panel’) and to iChorCNA¹⁰, an established WGS CNV TF estimator. In cutaneous melanoma pretreatment plasma samples profiled across methods, sensitivity for MRD-EDGE ctDNA detection was 100% (binomial 95% CI 83.8%-100%), compared to 93% (71.2%-99.2%) for the tumor-informed panel, 79% (53.1%-93.6%) for the de novo panel and 43% for iChorCNA (20.2%-68.0%) (FIG. 19E).

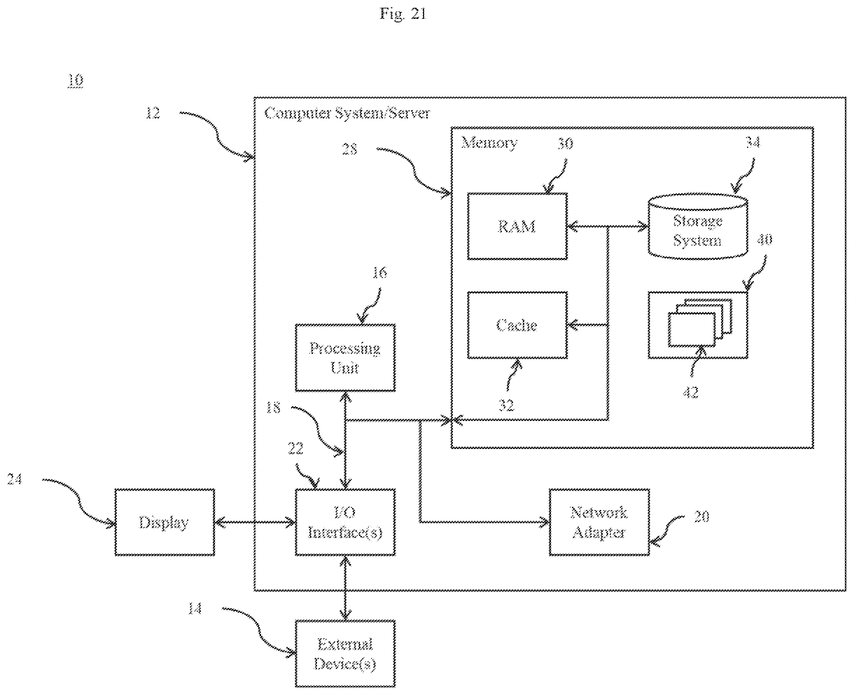

MRD-EDGE's ability to monitor changes in ctDNA TF following ICI treatment compared to alternative methods was next assessed. Given the unknown variable of tumor mutational burden in these samples and the influence of mutation load on detection rate, MRD-EDGE trends in TF were measured as a detection rate normalized to pretreatment TF (‘normalized detection rate’, nDR). For comparison in targeted panels, VAF was normalized to the pretreatment timepoint (‘normalized VAF’, nVAF). Side-by side comparisons demonstrate broadly similar trends in tumor burden following ICI treatment. (FIG. 19F, FIG. 21).

A sample detected by the tumor-informed panel was considered if estimated VAF across all surveyed genes was greater than zero, while detection in the de novo panel was measured as variant allele frequency (VAF)>0.005 per published methods⁸. Among samples evaluated across platforms (n=43 total, 14 pretreatment and 29 posttreatment samples), detection consistency (measured as the agreement between platforms of detected ctDNA and undetectable ctDNA) was highest between MRD-EDGE and the tumor-informed panel at 38 of 43 samples (88%, FIG. 19G, left). MRD-EDGE detected the lowest VAF detected by the tumor-informed panel, estimated at 1*10⁻⁴, validating the in silico benchmarking of detection sensitivity in clinical practice. Detection consistency was lower at 26 of 43 samples (60%) between MRD-EDGE and the de novo panel, likely due to the sensitivity floor of 0.005 in the latter method (FIG. 19G, right). To benchmark MRD-EDGE's utility in clinical surveillance, changes in ctDNA TF was compared at Week 6 following ICI treatment. Changes in nDR or nVAF showed higher agreement between MRD-EDGE and the tumor-informed panel, compared to the agreement with the de novo panel and iChorCNA (FIG. 19H). In summary, MRD-EDGE enables ultrasensitive melanoma ctDNA detection and TF monitoring on par with an established tumor-informed.

### Example 8: MRD-EDGE Accurately Monitors ctDNA in Small Cell Lung Cancer Plasma WGS without Matched Tumor

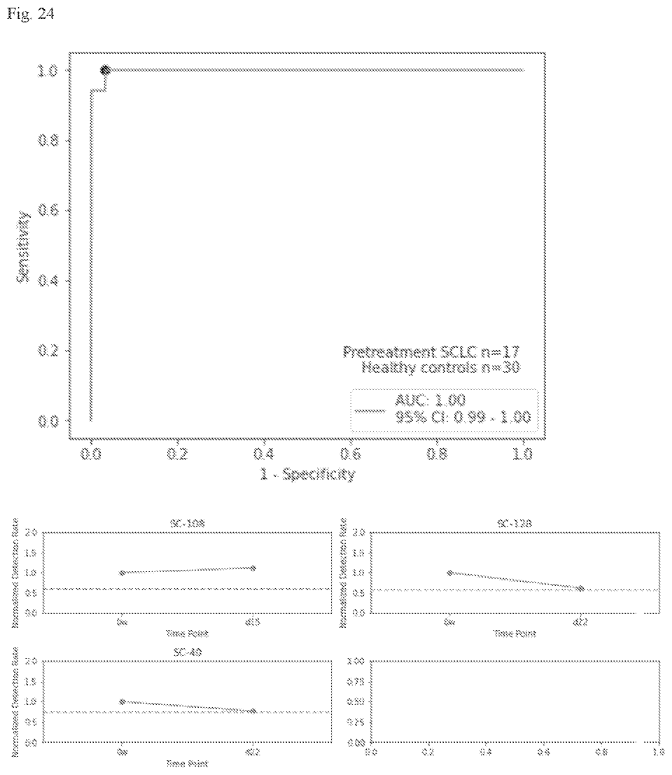

Serial tumor burden monitoring on immune checkpoint inhibition with MRD-EDGE was performed for 3 patients with small cell lung cancer. Tumor burden estimates were measured as a detection rate normalized to the pretreatment sample (normalized detection rate, nDR). According to FIG. 24, bottom panel, patient SC-108 did not respond to therapy at 6 week computed tomography imaging, and on day 15 and nDR rises above pretreatment level indicating tumor growth. Patients SC-40 and SC-128 showed a partial response to ICI on computed tomography imaging at 6 weeks, and posttreatment timepoint (days 22 and 15, respectively) shows a decline in nDR indicative of treatment response.

### Example 9: MRD-EDGE Sensitively Tracks Response to Immunotherapy in Metastatic Melanoma

In advanced melanoma, radiographic response may not be apparent for months after ICI initiation due to pseudo-progression or residual fibrous tissue^{61,62}, limiting the sensitivity of imaging to detect meaningful changes in tumor burden. Further, the absence of biomarkers that predict which patients will respond to therapy can lead to excess or futile treatment in unselected populations⁶³. Liquid biopsy can improve ICI care by providing faster readouts of response, orthogonal measurement of TF trends, and longitudinal noninvasive TF surveillance. Several panel approaches have demonstrated that changes in plasma TF as measured through increasing or decreasing ctDNA TF can complement imaging to predict response to ICI therapy^{20,21,59,64,65}.

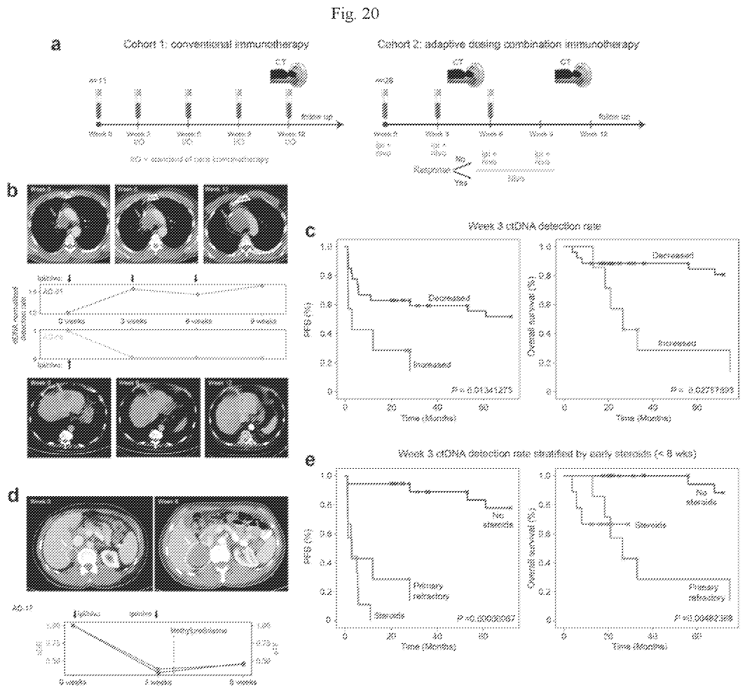

To explore the clinical utility of de novo (i.e., non tumor-informed) MRD-EDGE in ICI-treated patients with metastatic melanoma was sought. The adaptive dosing melanoma⁶⁰ cohort described above (n=26 patients, FIG. 20A right panel) was expanded to include additional patients treated with standard of care immunotherapy (′conventional immunotherapy′, n=11 patients, FIG. 20A left panel, Appendix 4). As further demonstration of applicability across platforms, the adaptive dosing cohort was sequenced on Illumina NovaSeq v1.0 while the standard of care immunotherapy cohort was sequenced on Illumina HiSeq X (Appendix 3). No tumor or matched normal tissue was used in this de novo plasma WGS analysis.

Trends in MRD-EDGE nDR tracked radiographic imaging results. For example, in a patient who progressed on treatment, progressive disease was seen on computed tomography (CT) at Week 6 and Week 12 while nDR concomitantly increased (FIG. 20B, top). Similarly, radiographic imaging demonstrated ongoing tumor shrinkage in a patient who responded to treatment, matched by a rapid and persistent decrease in nDR that occurred by Week 3 (FIG. 20B, bottom).

MRD-EDGE's ability to prognosticate clinical outcomes was next evaluated at serial plasma timepoints (122 pre- and posttreatment plasma samples from n=37 patients, Appendix 4). Patients with undetectable pretreatment ctDNA (n=3) were excluded from further clinical analyses. Change in ctDNA nDR, as measured by increased or decreased plasma TF following treatment, was found to be predictive of both PFS (P=0.01) and OS (P=0.03, FIG. 6d) as early as Week 3 after the first ICI infusion. This prognostic role for plasma TF changes after first ICI infusion and prior to any conventional imaging has also been noted in response to single-agent ICI in NSCLC²¹, and demonstrated a role for liquid biopsy TF surveillance in the earliest days of ICI treatment. Significant PFS and OS relationships for change in ctDNA nDR at Week 6 (FIG. 23A) was also found. In contrast, CT imaging was available for the adaptive dosing cohort at Week 6, and here no significant relationship was found between RECIST response and OS (P=0.15, FIG. 23B).

Notably, the first OS event in the Week 3 and Week 6 ctDNA survival analysis occurred in a patient with decreasing nDR at Week 3 and Week 6 who enrolled on protocol following prior treatment of brain metastases. CT imaging (partial response) and ctDNA trends for both MRD-EDGE and the tumor-informed panel identified an extracranial response to therapy. This patient, however, had intracranial progression at 5 months and was taken off protocol. Such findings are consistent with the melanoma ctDNA literature, where ctDNA trends are known to reflect extracranial rather than intracranial tumor burden⁶⁶, and suggest that ctDNA monitoring should be used with caution in patients at high risk of intracranial progression.

Despite significant PFS and OS relationships for ctDNA trends at Week 3, several instances were noted in which decreasing Week 3 nDR was not indicative of durable ICI response. It was reasoned that the high toxicity rate from combination ICI, where nearly 40% of patients will stop treatment early because of immune-related adverse events (irAEs)⁶⁷, may have confounded classification at Week 3. Clinically, severe irAEs are often treated with corticosteroids, and early steroid use (within 8 weeks of ICI treatment) is associated with shorter PFS and OS in melanoma⁶⁸. Melanoma patients were therefore stratified into 3 groups, patients with primary refractory disease (initial increase in ctDNA nDR, n=7), and patients with an initial ctDNA response either treated or untreated with early steroids (n=9 and n=18, respectively). This classification proved strongly predictive of both PFS (P=1.3*10⁻⁷) and OS (P=1.7*10⁻⁴, FIG. 19F), and suggests that early treatment responses, measured via ctDNA may be inhibited by steroids. In summary, with no need for matched tumor and a standard WGS workflow, MRD-EDGE offers the potential for real-time serial monitoring of plasma ctDNA in conjunction with imaging to assess immunotherapy response.

### Example 10: Discussion

The use of noninvasive liquid biopsy to detect MRD and track response to therapy heralds the next frontier in precision oncology. It was previously observed that the sensitivity of deep targeted sequencing approaches may be limited in the context of low plasma TF (e.g., MRD or the nadir of response to immunotherapy), and used WGS of plasma to expand the number of informative sites and therefore increase sensitivity in this setting. As disclosed herein, a machine learning-based classifier MRD-EDGE was designed to integrate an expanded feature set for SNVs and CNVs to substantially enhance ctDNA signal enrichment.

Broadly, MRD-EDGE can leverage both prior knowledge of tumor-specific mutational compendia and a biologically-informed feature space to enrich ctDNA signal. This MRD-EDGE SNV deep learning strategy differs markedly from other deep learning variant callers^{69,70} through the use of disease-specific biology to inform somatic mutation identification. The focus on classifying fragments rather than loci, as disclosed herein, allows one to overcome the inability to apply consensus mutation calling, the cornerstone of most variant calling strategies, in extremely low TF settings. Moreover, fragment-based classification enabled an increase in the size of training corpuses to hundreds of thousands of observations, which is critical to comprehensive pattern recognition with neural networks⁷¹. The deep learning SNV architecture in MRD-EDGE provides a flexible platform for integrating disease-specific molecular features, outperforms other machine learning approaches, and demonstrates generalizability across cancer types and sequencing preparations.

For CNVs, machine-learning guided signal denoising enables accurate inference of plasma read-depth skews, while fragmentomics and BAF provide orthogonal metrics for CNV assessment. The use of tumor-specific copy number profiles combined with powerful denoising enables increased sensitivity compared to established read-depth approaches^{10,11}. The use of neutral segments as a sample level internal control offers an additional specificity advantage compared to tumor-agnostic fragment-based methods^{9,23}. The lower degree of aneuploidy needed for ultrasensitive detection (FIG. 10E) and ability to capture signal from copy-neutral LOH will enable application to a diverse set of solid tumors even in the absence of high somatic SNV burden (FIG. 17).

It is expected that the simplified WGS workflow, which obviates the need for custom panel generation and molecular barcodes, and ability to work with limited input material (1 mL of plasma), will enhance MRD-EDGE translational impact in diverse clinical settings, especially given the rapid decline in raw sequencing costs. MRD-EDGE enabled the detection of postoperative CRC and LUAD MRD, as well as tracking of plasma TF dynamics in response to neoadjuvant ICI. The data provided herein highlight the potential for real-time therapeutic optimization in the neoadjuvant setting, which could potentially inform early surgery or treatment change for non-responders, in order to maximize curative opportunities.

The distinct sensitivity of MRD-EDGE allowed examination of the detection of ctDNA shedding from precancerous colorectal adenomas. While this tumor-informed approach cannot be used for screening, the detection of ctDNA in a substantial proportion of cases argues that ctDNA may be present without invasive disease. This carries important implications for ongoing efforts to develop liquid biopsy approaches for cancer screening^{9,13,72,73}. Considering the value of precancerous lesion detection in CRC screening⁷⁴, these data demonstrate that ctDNA-guided detection of premalignant lesions is a viable goal, provided that tools with sufficient sensitivity can be developed for this setting. On the other hand, the demonstration of ctDNA shedding without an invasive component suggests that clonal mosaicisms in normal tissues may impact cancer screening efforts in a manner similar to the observation of confounding clonal hematopoiesis mutations in targeted sequencing^{73,75-77}. This may be particularly important for hotspot mutations given the pervasive nature of clonal outgrowths⁷⁸⁻⁸⁰ and the potential of the plasma to aggregate signal across potentially thousands of separate clones. Similarly, it is unknown to what degree normal solid tissue clonal outgrowths differ from malignant counterparts in fragment length or methylation profiles, which may impact non-mutational ctDNA screening methods.

The enhanced signal to noise enrichment of MRD-EDGE was further leveraged to perform de novo (non-tumor informed) SNV mutation detection in advanced melanoma. The emerging role of early ctDNA trends in monitoring ICI response, seen here and elsewhere^{20,21,59}, is reflected in the recent Center for Medicare & Medicaid Services approval of tumor-informed bespoke assays to prognosticate response to immunotherapy after 6 weeks. In the phase 2 trial²⁰ that led to this approval, the requirement for a matched tumor sample for bespoke panel design led to the exclusion of one-third of patients due to low tumor DNA purity or quality. In contrast, MRD-EDGE required only plasma, and produced performance on par with a comparable tumor-informed panel. MRD-EDGE allowed for early and accurate assessment of response to ICI, a challenging clinical setting for prognostication^{63,64}. Future large-scale interventional studies will be critical to demonstrate the value of rapid and quantitative estimation of ICI response to inform real-time clinical decision making.

Collectively, the present data support the use of plasma WGS as a complimentary strategy to the prevailing paradigm of ctDNA mutation detection via deep targeted panel sequencing. This approach can complement targeted panels as well as other liquid biopsy tools such as methylation-based assays to create a comprehensive liquid biopsy toolkit that tailors sequencing approach to clinical application. For example, it is envision that improved cancer screening through early detection efforts will allow the diagnosis of cancers at less advanced stages^{9,12,13,73}. Low tumor-burden disease treated with surgical and/or non-surgical means will benefit from ultra-sensitive TF monitoring via MRD-EDGE. In the event of high burden disease relapse, deep targeted panels^{5,6,8,19,21}, better suited to provide mutational profiling through exhaustive coverage depth, can nominate gene targets for systemic targeted therapy. While the value of therapy-optimization based on MRD-EGDE monitoring requires investigation in large clinical cohorts, the present findings highlight the potential of ctDNA as a quantitative tumor burden biomarker that provides real-time feedback in response to therapy and early insight into relapsed disease.

### Computer Implemented Methods

Referring now to FIG. 21, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed computing environments where tasks are performed by remote processing devices that are linked through a communications network. In a distributed computing environment, program modules may be located in both local and remote computer system storage media including memory storage devices.

As shown in FIG. 7, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In various embodiments, a learning system is provided. In some embodiments, a feature vector is provided to a learning system. Based on the input features, the learning system generates one or more outputs. In some embodiments, the output of the learning system is a feature vector. In some embodiments, the learning system comprises a SVM.

In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### REFERENCES

1. Murtaza M, Dawson S-J, Tsui D W Y, et al. Non-invasive analysis of acquired resistance to cancer therapy by sequencing of plasma DNA. Nature. 2013; 497 (7447): 108-112.
2. Diehl F, Schmidt K, Choti M A, et al. Circulating mutant DNA to assess tumor dynamics. Nat Med. 2008; 14 (9): 985-990.
3. Newman A M, Lovejoy A F, Klass D M, et al. Integrated digital error suppression for improved detection of circulating tumor DNA. Nat Biotechnol. 2016; 34 (5): 547-555.
4. Newman A M, Bratman S V, To J, et al. An ultrasensitive method for quantitating circulating tumor DNA with broad patient coverage. Nat Med. 2014; 20 (5): 548-554.
5. Phallen J, Sausen M, Adleff V, et al. Direct detection of early-stage cancers using circulating tumor DNA. Sci Transl Med. 2017;9 (403). doi: 10.1126/scitranslmed.aan2415
6. Cohen J D, Li L, Wang Y, et al. Detection and localization of surgically resectable cancers with a multi-analyte blood test. Science. 2018; 359 (6378): 926-930.
7. Wan J C M, Heider K, Gale D, et al. ctDNA monitoring using patient-specific sequencing and integration of variant reads. Sci Transl Med. 2020; 12 (548). doi: 10.1126/scitranslmed.aaz8084
8. Rose Brannon A, Jayakumaran G, Diosdado M, et al. Enhanced specificity of clinical high-sensitivity tumor mutation profiling in cell-free DNA via paired normal sequencing using MSK-ACCESS. Nat Commun. 2021; 12 (1): 3770.
9. Cristiano S, Leal A, Phallen J, et al. Genome-wide cell-free DNA fragmentation in patients with cancer. Nature. 2019; 570 (7761): 385-389.
10. Adalsteinsson V A, Ha G, Freeman S S, et al. Scalable whole-exome sequencing of cell-free DNA reveals high concordance with metastatic tumors. Nat Commun. 2017;8 (1): 1324.
11. Lakatos E, Hockings H, Mossner M, Huang W, Lockley M, Graham T A. LiquidCNA: Tracking subclonal evolution from longitudinal liquid biopsies using somatic copy number alterations. iScience. 2021; 24 (8): 102889.
12. Shen S Y, Singhania R, Fehringer G, et al. Sensitive tumour detection and classification using plasma cell-free DNA methylomes. Nature. 2018; 563 (7732): 579-583.
13. Liu M C, Oxnard G R, Klein E A, Swanton C, Seiden M V, CCGA Consortium.

Sensitive and specific multi-cancer detection and localization using methylation signatures in cell-free DNA. Ann Oncol. 2020; 31 (6): 745-759.
14. Ulz P, Perakis S, Zhou Q, et al. Inference of transcription factor binding from cell-free DNA enables tumor subtype prediction and early detection. Nat Commun. 2019; 10 (1): 4666.
15. Sun K, Jiang P, Wong AIC, et al. Size-tagged preferred ends in maternal plasma DNA shed light on the production mechanism and show utility in noninvasive prenatal testing. Proc Natl Acad Sci USA. 2018; 115 (22):E5106-E5114.
16. Jiang P, Sun K, Peng W, et al. Plasma DNA End-Motif Profiling as a Fragmentomic Marker in Cancer, Pregnancy, and Transplantation. Cancer Discov. 2020; 10 (5): 664-673.
17. Wang S, An T, Wang J, et al. Potential clinical significance of a plasma-based KRAS mutation analysis in patients with advanced non-small cell lung cancer. Clin Cancer Res. 2010; 16 (4): 1324-1330.
18. Kobayashi S, Boggon T J, Dayaram T, et al. EGFR mutation and resistance of non-small-cell lung cancer to gefitinib. N Engl J Med. 2005; 352 (8): 786-792.
19. Powles T, Assaf Z J, Davarpanah N, et al. ctDNA guiding adjuvant immunotherapy in urothelial carcinoma. Nature. Published online Jun. 16, 2021. doi: 10.1038/s41586-021-03642-9
20. Bratman S V, Yang SYC, Iafolla MAJ, et al. Personalized circulating tumor DNA analysis as a predictive biomarker in solid tumor patients treated with pembrolizumab. Nature Cancer. 2020; 1 (9): 873-881.
21. Nabet B Y, Esfahani M S, Moding E J, et al. Noninvasive Early Identification of Therapeutic Benefit from Immune Checkpoint Inhibition. Cell. 2020; 183 (2): 363-376.e13.
22. Tie J, Wang Y, Tomasetti C, et al. Circulating tumor DNA analysis detects minimal residual disease and predicts recurrence in patients with stage II colon cancer. Sci Transl Med. 2016; 8 (346): 346ra92.
23. Reinert T, Henriksen T V, Christensen E, et al. Analysis of plasma cell-free DNA by ultradeep sequencing in patients with stages I to III colorectal cancer. JAMA Oncol. 2019; 5 (8): 1124-1131.
24. Henriksen T V, Tarazona N, Frydendahl A, et al. Circulating tumor DNA in stage III colorectal cancer, beyond minimal residual disease detection, towards assessment of adjuvant therapy efficacy and clinical behavior of recurrences. Clin Cancer Res. Published online Oct. 8, 2021. doi: 10.1158/1078-0432.CCR-21-2404
25. Kurtz D M, Soo J, Co Ting Keh L, et al. Enhanced detection of minimal residual disease by targeted sequencing of phased variants in circulating tumor DNA. Nat Biotechnol. Published online Jul. 22, 2021. doi: 10.1038/s41587-021-00981-w
26. Haque I S, Elemento O. Challenges in Using ctDNA to Achieve Early Detection of Cancer. bioRxiv. Published online Dec. 21, 2017:237578. doi: 10.1101/237578
27. Avanzini S, Kurtz D M, Chabon J J, et al. A mathematical model of ctDNA shedding predicts tumor detection size. bioRxiv. Published online Apr. 23, 2020:2020.02.12.946228. doi: 10.1101/2020.02.12.946228
28. Zviran A, Schulman R C, Shah M, et al. Genome-wide cell-free DNA mutational integration enables ultra-sensitive cancer monitoring. Nat Med. 2020; 26 (7): 1114-1124.
29. Devonshire A S, Whale A S, Gutteridge A, et al. Towards standardisation of cell-free DNA measurement in plasma: controls for extraction efficiency, fragment size bias and quantification. Anal Bioanal Chem. 2014; 406 (26): 6499-6512.
30. Mouliere F, Chandrananda D, Piskorz A M, et al. Enhanced detection of circulating tumor DNA by fragment size analysis. Sci Transl Med. 2018; 10 (466). doi: 10.1126/scitranslmed.aat4921
31. Alexandrov L B, Nik-Zainal S, Wedge D C, et al. Signatures of mutational processes in human cancer. Nature. 2013; 500 (7463): 415-421.
32. Alexandrov L B, Ju Y S, Haase K, et al. Mutational signatures associated with tobacco smoking in human cancer. Science. 2016; 354 (6312): 618-622.
33. Underhill H R, Kitzman J O, Hellwig S, et al. Fragment Length of Circulating Tumor DNA. PLOS Genet. 2016; 12 (7): e1006162.
34. Guo J, Ma K, Bao H, et al. Quantitative characterization of tumor cell-free DNA shortening. BMC Genomics. 2020; 21 (1): 473.
35. Gonzalez-Perez A, Sabarinathan R, Lopez-Bigas N. Local determinants of the mutational landscape of the human genome. Cell. 2019; 177 (1): 101-114.
36. Woo Y H, Li W-H. DNA replication timing and selection shape the landscape of nucleotide variation in cancer genomes. Nat Commun. 2012; 3 (1): 1004.
37. Haradhvala N J, Polak P, Stojanov P, et al. Mutational strand asymmetries in cancer genomes reveal mechanisms of DNA damage and repair. Cell. 2016; 164 (3): 538-549.
38. Donley N, Thayer M J. DNA replication timing, genome stability and cancer: late and/or delayed DNA replication timing is associated with increased genomic instability. Semin Cancer Biol. 2013; 23 (2): 80-89.
39. Taylor A M, Shih J, Ha G, et al. Genomic and Functional Approaches to Understanding Cancer Aneuploidy. Cancer Cell. 2018; 33 (4): 676-689.e3.
40. Deshpande A, Walradt T, Hu Y, Koren A, Imielinski M. Robust foreground detection in somatic copy number data. Cold Spring Harbor Laboratory. Published online Nov. 20, 2019:847681. doi: 10.1101/847681
41. Raine K M, Van Loo P, Wedge D C, et al. AscatNgs: Identifying somatically acquired copy-number alterations from whole-genome sequencing data. Curr Protoc Bioinformatics. 2016; 56:15.9.1-15.9.17.
42. Carter S L, Cibulskis K, Helman E, et al. Absolute quantification of somatic DNA alterations in human cancer. Nat Biotechnol. 2012; 30 (5): 413-421.
43. Sadeh R, Sharkia I, Fialkoff G, et al. ChIP-seq of plasma cell-free nucleosomes identifies gene expression programs of the cells of origin. Nat Biotechnol. 2021; 39 (5): 586-598.
44. Snyder M W, Kircher M, Hill A J, Daza R M, Shendure J. Cell-free DNA comprises an in vivo nucleosome footprint that informs its tissues-of-origin. Cell. 2016; 164 (1-2): 57-68.
45. Jiang P, Sun K, Tong Y K, et al. Preferred end coordinates and somatic variants as signatures of circulating tumor DNA associated with hepatocellular carcinoma. Proc Natl Acad Sci USA. 2018; 115 (46): E10925-E10933.
46. Renaud G, Nørgaard M, Lindberg J, et al. Discovering fragment length signatures of circulating tumor DNA using Non-negative Matrix Factorization. bioRxiv. Published online Jun. 10, 2021:2021.06.09.447533. doi: 10.1101/2021.06.09.447533
47. Guraya S Y. Pattern, Stage, and Time of Recurrent Colorectal Cancer After Curative Surgery. Clin Colorectal Cancer. 2019; 18 (2): e223-e228.
48. Karakuchi N, Shimomura M, Toyota K, et al. Spontaneous regression of transverse colon cancer with high-frequency microsatellite instability: a case report and literature review. World J Surg Oncol. 2019; 17 (1): 19.
49. Kim C G, Ahn J B, Jung M, et al. Effects of microsatellite instability on recurrence patterns and outcomes in colorectal cancers. Br J Cancer. 2016; 115 (1): 25-33.
50. Altorki N K, McGraw T E, Borczuk A C, et al. Neoadjuvant durvalumab with or without stereotactic body radiotherapy in patients with early-stage non-small-cell lung cancer: a single-centre, randomised phase 2 trial. Lancet Oncol. 2021; 22 (6): 824-835.
51. Myint NNM, Verma A M, Fernandez-Garcia D, et al. Circulating tumor DNA in patients with colorectal adenomas: assessment of detectability and genetic heterogeneity. Cell Death Dis. 2018; 9 (9): 894.
52. Junca A, Tachon G, Evrard C, et al. Detection of Colorectal Cancer and Advanced Adenoma by Liquid Biopsy (Decalib Study): The ddPCR Challenge. Cancers. 2020; 12 (6). doi: 10.3390/cancers12061482
53. Rasmussen L, Wilhelmsen M, Christensen I J, et al. Protocol Outlines for Parts 1 and 2 of the Prospective Endoscopy III Study for the Early Detection of Colorectal Cancer: Validation of a Concept Based on Blood Biomarkers. JMIR Res Protoc. 2016; 5 (3): e182.
54. Risio M. The Natural History of pTI Colorectal Cancer. Front Oncol. 2012; 2:22.
55. Alcántara Torres M, Rodríguez Merlo R, Repiso Ortega A, et al. DNA aneuploidy in colorectal adenomas. Role in the adenoma-carcinoma sequence. Rev Esp Enferm Dig. 2005;97 (1): 7-15.
56. Lin S-H, Raju G S, Huff C, et al. The somatic mutation landscape of premalignant colorectal adenoma. Gut. 2018; 67 (7): 1299-1305.
57. Wolff R K, Hoffman M D, Wolff E C, et al. Mutation analysis of adenomas and carcinomas of the colon: Early and late drivers. Genes Chromosomes Cancer. 2018; 57 (7): 366-376.
58. Haile S, Corbett R D, Bilobram S, et al. Sources of erroneous sequences and artifact chimeric reads in next generation sequencing of genomic DNA from formalin-fixed paraffin-embedded samples. Nucleic Acids Res. 2019; 47 (2): e12.
59. Cindy Yang S Y, Lien S C, Wang B X, et al. Pan-cancer analysis of longitudinal metastatic tumors reveals genomic alterations and immune landscape dynamics associated with pembrolizumab sensitivity. Nat Commun. 2021; 12 (1): 5137.
60. Postow M A, Goldman D A, Shoushtari A N, et al. A phase I I study to evaluate the need for >two doses of nivolumab+ipilimumab combination (combo) immunotherapy. J Clin Oncol. 2020; 38 (15_suppl): 10003-10003.
61. Chiou V L, Burotto M. Pseudoprogression and immune-related response in solid tumors. J Clin Oncol. 2015;33 (31): 3541-3543.
62. Zhou L, Zhang M, Li R, Xue J, Lu Y. Pseudoprogression and hyperprogression in lung cancer: a comprehensive review of literature. J Cancer Res Clin Oncol. Published online Aug. 28, 2020. doi: 10.1007/s00432-020-03360-1
63. Chowell D, Yoo S-K, Valero C, et al. Improved prediction of immune checkpoint blockade efficacy across multiple cancer types. Nat Biotechnol. Published online Nov. 1, 2021. doi: 10.1038/s41587-021-01070-8
64. Weber S, van der Leest P, Donker H C, et al. Dynamic Changes of Circulating Tumor DNA Predict Clinical Outcome in Patients With Advanced Non-Small-Cell Lung Cancer Treated With Immune Checkpoint Inhibitors. JCO Precision Oncology. 2021; (5): 1540-1553.
65. Zhang Q, Luo J, Wu S, et al. Prognostic and predictive impact of circulating tumor DNA in patients with advanced cancers treated with immune checkpoint blockade. Cancer Discov. Published online Aug. 14, 2020: CD-20-0047.
66. Lee J H, Menzies A M, Carlino M S, et al. Longitudinal Monitoring of ctDNA in Patients with Melanoma and Brain Metastases Treated with Immune Checkpoint Inhibitors. Clin Cancer Res. 2020; 26 (15): 4064-4071.
67. Wolchok J D, Chiarion-Sileni V, Gonzalez R, et al. Overall Survival with Combined Nivolumab and Ipilimumab in Advanced Melanoma. N Engl J Med. 2017;377 (14): 1345-1356.
68. Bai X, Hu J, Betof Warner A, et al. Early Use of High-Dose Glucocorticoid for the Management of irAE Is Associated with Poorer Survival in Patients with Advanced Melanoma Treated with Anti-PD-1 Monotherapy. Clin Cancer Res. 2021; 27 (21): 5993-6000.
69. Poplin R, Chang P-C, Alexander D, et al. A universal SNP and small-indel variant caller using deep neural networks. Nat Biotechnol. 2018; 36 (10): 983-987.
70. Luo R, Sedlazeck F J, Lam T-W, Schatz M C. A multi-task convolutional deep neural network for variant calling in single molecule sequencing. Nat Commun. 2019; 10 (1): 998.
71. Kourou K, Exarchos T P, Exarchos K P, Karamouzis M V, Fotiadis D I. Machine learning applications in cancer prognosis and prediction. Comput Struct Biotechnol J. 2015; 13:8-17.
72. Klein E A, Richards D, Cohn A, et al. Clinical validation of a targeted methylation-based multi-cancer early detection test using an independent validation set. Ann Oncol. 2021; 32 (9): 1167-1177.
73. Chabon J J, Hamilton E G, Kurtz D M, et al. Integrating genomic features for non-invasive early lung cancer detection. Nature. 2020; 580 (7802): 245-251.
74. U S Preventive Services Task Force, Davidson K W, Barry M J, et al. Screening for Colorectal Cancer: U S Preventive Services Task Force Recommendation Statement. JAMA. 2021; 325 (19): 1965-1977.
75. Razavi P, Li B T, Brown D N, et al. High-intensity sequencing reveals the sources of plasma circulating cell-free DNA variants. Nat Med. 2019; 25 (12): 1928-1937.
76. Hu Y, Ulrich B C, Supplee J, et al. False-Positive Plasma Genotyping Due to Clonal Hematopoiesis. Clin Cancer Res. 2018; 24 (18): 4437-4443.
77. Wang B, Huang F, Shen M, et al. Clonal hematopoiesis mutations in plasma cfDNA RAS/BRAF genotyping of metastatic colorectal cancer. Ann Oncol. 2019; 30 (Supplement_5): v237.
78. Martincorena I, Fowler J C, Wabik A, et al. Somatic mutant clones colonize the human esophagus with age. Science. 2018; 362 (6417): 911-917.
79. Yokoyama A, Kakiuchi N, Yoshizato T, et al. Age-related remodelling of oesophageal epithelia by mutated cancer drivers. Nature. 2019; 565 (7739): 312-317.
80. Shain A H, Yeh I, Kovalyshyn I, et al. The Genetic Evolution of Melanoma from Precursor Lesions. N Engl J Med. 2015;373 (20): 1926-1936.
81. Gerstung M, Jolly C, Leshchiner I, et al. The evolutionary history of 2,658 cancers. Nature. 2020; 578 (7793): 122-128.
82. Corces M R, Granja J M, Shams S, et al. The chromatin accessibility landscape of primary human cancers. Science. 2018; 362 (6413). doi: 10.1126/science.aav1898
83. Ernst J, Kellis M. ChromHMM: automating chromatin-state discovery and characterization. Nat Methods. 2012; 9 (3): 215-216.
84. TruSeq DNA PCR-Free Reference Guide. Published online 2017. https://support.illumina.com/content/dam/illumina-support/documents/documentation/chemistry_documentation/samplepreps_truseq/trus eq-dna-pcr-free-workflow/truseq-dna-pcr-free-workflow-reference-1000000039279-00.pdf
85. Reinert T, Schøler L V, Thomsen R, et al. Analysis of circulating tumour DNA to monitor disease burden following colorectal cancer surgery. Gut. 2016; 65 (4): 625-634.
86. Li H, Durbin R. Fast and accurate short read alignment with Burrows-Wheeler transform. Bioinformatics. 2009; 25 (14): 1754-1760.
87. Jiang H, Lei R, Ding S-W, Zhu S. Skewer: a fast and accurate adapter trimmer for next-generation sequencing paired-end reads. BMC Bioinformatics. 2014; 15:182.
88. Bergmann E A, Chen B-J, Arora K, Vacic V, Zody M C. Conpair: concordance and contamination estimator for matched tumor-normal pairs. Bioinformatics. 2016; 32 (20): 3196-3198.
89. Favero F, Joshi T, Marquard A M, et al. Sequenza: allele-specific copy number and mutation profiles from tumor sequencing data. Ann Oncol. 2015;26 (1): 64-70.
90. Arora K, Shah M, Johnson M, et al. Deep whole-genome sequencing of 3 cancer cell lines on 2 sequencing platforms. Sci Rep. 2019; 9 (1): 19123.
91. Maffucci P, Bigio B, Rapaport F, et al. Blacklisting variants common in private cohorts but not in public databases optimizes human exome analysis. Proc Natl Acad Sci USA. 2019; 116 (3): 950-959.
92. Karczewski K J, Francioli L C, Tiao G, et al. The mutational constraint spectrum quantified from variation in 141,456 humans. Nature. 2020; 581 (7809): 434-443.
93. Amemiya H M, Kundaje A, Boyle A P. The ENCODE Blacklist: Identification of Problematic Regions of the Genome. Sci Rep. 2019; 9 (1): 9354.
94. Benjamin D, Sato T, Cibulskis K, Getz G, Stewart C, Lichtenstein L. Calling Somatic SNVs and Indels with Mutect2. bioRxiv. Published online Dec. 2, 2019:861054. doi: 10.1101/861054
95. ENCODE Project Consortium. An integrated encyclopedia of DNA elements in the human genome. Nature. 2012; 489 (7414): 57-74.
96. Rozowsky J, Euskirchen G, Auerbach R K, et al. PeakSeq enables systematic scoring of ChIP-seq experiments relative to controls. Nat Biotechnol. 2009; 27 (1): 66-75.
97. Xiong K, Ma J. Revealing Hi-C subcompartments by imputing inter-chromosomal chromatin interactions. Nat Commun. 2019; 10 (1): 5069.
98. Sabarinathan R, Mularoni L, Deu-Pons J, Gonzalez-Perez A, López-Bigas N. Nucleotide excision repair is impaired by binding of transcription factors to DNA. Nature. 2016; 532 (7598): 264-267.
99. Pich O, Muiños F, Sabarinathan R, Reyes-Salazar I, Gonzalez-Perez A, Lopez-Bigas N. Somatic and germline mutation periodicity follow the orientation of the DNA minor groove around nucleosomes. Cell. 2018; 175 (4): 1074-1087.e18.
100. Feng Z, Clemente J C, Wong B, Schadt E E. Detecting and phasing minor single-nucleotide variants from long-read sequencing data. Nat Commun. 2021; 12 (1): 3032.
101. Vierstra J, Wang H, John S, Sandstrom R, Stamatoyannopoulos J A. Coupling transcription factor occupancy to nucleosome architecture with DNase-FLASH. Nat Methods. 2014; 11 (1): 66-72.
102. Cheng D T, Mitchell T N, Zehir A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn. 2015; 17 (3): 251-264.
103. Shen R, Seshan V E. FACETS: allele-specific copy number and clonal heterogeneity analysis tool for high-throughput DNA sequencing. Nucleic Acids Res. 2016; 44 (16): e131.
104. Davidson-Pilon C. Lifelines, Survival Analysis in Python.; 2021. doi: 10.5281/zenodo.5512044.

### INCORPORATION B Y REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### Appendix 1

| | | | | | | | | somatic | |
|---|---|---|---|---|---|---|---|---|---|
| MEL | | TOTAL_{—} | | | | QC | | pipeline | |
| Tumor | | READS | | Mean | MEDIAN_{—} | metrics | | # of | |
| Patient | Tissue | TOTAL_{—} | PercentTotal- | Coverage | INSERT_{—} | Conpair- | auto- | mutation | |
| ID | Type | READS | Duplication | (X) | SIZE (bp) | Concordance | correlation | detected | Notes |
| MEL-01 | Fresh | 1159000380 | 15.9545 | 48.1903 | 425 | 99.92 | 0.0896 | 411615 | |
| | frozen | | | | | | | | |

| MEL | | | | | | | |
|---|---|---|---|---|---|---|---|
| Normal/ | sequencing | | | | QC | | |
| PBMC | metrics | | Mean | MEDIAN_{—} | metrics | | |
| Patient | TOTAL_{—} | PercentTotal- | Coverage | INSERT_{—} | Conpair- | auto- | |
| ID | READS | Duplication | (X) | SIZE (bp) | Concordance | correlation | Notes |
| MEL-01 | 820566084 | 7.5412 | 36.8332 | 435 | 99.92 | 0.0072 | |

| | pre | | | | | | | | |
|---|---|---|---|---|---|---|---|---|---|
| | sequencing | | | | | | | | |
| MEL | QC | | | | | | | sequencing | |
| Plasma | Blood | | | | | | | metrics | |
| Patient | Collection | Sequencing | Sequencing | extraction | total | library | # of PCR | library | |
| ID | Tube | Platform | Location | kit | mass (ng) | prep kit | cycles | mass (ng) | Notes |
| MEL-01 | Streck | Illumina | NYGC | Omega | 9.875 | Kapa | 6 | 9.8 | |
| | | HiSeq X | | | | Hyper | | | |

| High- | | | | | | | |
|---|---|---|---|---|---|---|---|
| Burden | | | | | | | |
| LUAD | | | | | | | |
| Normal/ | sequencing | | | | | | |
| PBMC | metrics | | Mean | MEDIAN_{—} | QC metrics | | |
| Patient | TOTAL_{—} | PercentTotal- | Coverage | INSERT_{—} | Conpair- | auto- | |
| ID | READS | Duplication | (X) | SIZE (bp) | Concordance | correlation | Notes |
| CM-6 | 9.44E+08 | 10.2506 | 41.3995 | 417 | 96.38% | 0.035 | |
| CM-30 | 8.62E+08 | 9.385 | 38.7107 | 435 | 99.84% | 0.0061 | |

| High- | pre | | | | | | | |
|---|---|---|---|---|---|---|---|---|
| Burden | sequencing | | | | | | | |
| LUAD | QC | | | | | | | sequencing |
| Plasma | Blood | | | | total | | # of | metrics |
| Patient | Collection | Sequencing | Sequencing | extraction | mass | library | PCR | library |
| ID | Tube | Platform | Location | kit | (ng) | prep kit | cycles | mass (ng) |
| CM-6 | Streck | NovaSeq | NYGC | Omega | 17.34 | Kapa | 6 | 25 |
| | | v1.0 | | | | Hyper | | |
| CM-30 | Streck | NovaSeq | NYGC | Omega | 37.8 | Kapa | 6 | 25 |
| | | v1.0 | | | | Hyper | | |

| High- | | | | | | |
|---|---|---|---|---|---|---|
| Burden | | | | QC | | |
| LUAD | | | | metrics | | |
| Plasma | | Percent- | Mean | MEDIAN_{—} | | |
| Patient | TOTAL_{—} | Total- | Coverage | INSERT_{—} | Conpair- | Auto- |
| ID | READS | Duplication | (X) | SIZE (bp) | Concordance | correlation |
| CM-6 | 9.78E+08 | 6.597 | 30.8783 | 179 | 96.38% | 0.036455 |
| CM-30 | 2.29E+09 | 8.5526 | 66.1881 | 169 | 99.84% | 0.044411 |

*library mass capped at 25 ng

| Adaptive | | | | | | |
|---|---|---|---|---|---|---|
| Dosing | | | | | | |
| Melanoma | | sequencing | | | | QC |
| Normal/PBMC | | metrics | Mean | MEDIAN_{—} | | metrics |
| Patient | TOTAL_{—} | PercentTotal- | Coverage | INSERT_{—} | Conpair- | auto- |
| ID | READS | Duplication | (X) | SIZE (bp) | Concordance | correlation |
| AD-05 | 9.33E+08 | 9.2198 | 42.8758 | 445 | NA | 0.0048 |

| Adaptive | | pre | | | | | | | |
|---|---|---|---|---|---|---|---|---|---|
| Dosing | | sequencing | | | | | | | |
| Melanoma | | QC | | | | | | | sequencing |
| Plasma | | Blood | | | | | | # of | metrics |
| Patient | | Collection | Sequencing | Sequencing | extraction | total | library | PCR | library |
| ID | Timepoint | Tube | Platform | Location | kit | mass (ng) | prep kit | cycles | mass (ng) |
| AD-01_A | Pre- | Streck | Illumina | NYGC | Omega | 20.53 | Kapa | 6 | 20.5344 |
| | treatment | | Novaseq v1.0 | | | | Hyper | | |
| AD-01_B | Week 3 | Streck | Illumina | NYGC | Omega | 10.8 | Kapa | 6 | 10.803 |
| | | | Novaseq v1.0 | | | | Hyper | | |
| AD-01_C | Week 6 | Streck | Illumina | NYGC | Omega | 30.96 | Kapa | 6 | 25 |
| | | | Novaseq v1.0 | | | | Hyper | | |
| AD-01_D | Week 9 | Streck | Illumina | NYGC | Omega | 18.2 | Kapa | 6 | 18.205 |
| | | | Novaseq v1.0 | | | | Hyper | | |
| AD-01_E | Week 12 | Streck | Illumina | NYGC | Omega | 25.54 | Kapa | 6 | 25 |
| | | | Novaseq v1.0 | | | | Hyper | | |
| AD-02_A | Pre- | Streck | Illumina | NYGC | Omega | 46.81 | Kapa | 6 | 25 |
| | treatment | | Novaseq v1.0 | | | | Hyper | | |
| AD-02_B | Week 3 | Streck | Illumina | NYGC | Omega | 37.95 | Kapa | 6 | 25 |
| | | | Novaseq v1.0 | | | | Hyper | | |
| AD-02_C | Week 6 | Streck | Illumina | NYGC | Omega | 9.97 | Kapa | 6 | 9.972 |
| | | | Novaseq v1.0 | | | | Hyper | | |
| AD-04_A | Pre- | Streck | Illumina | NYGC | Omega | 15.68 | Kapa | 6 | 15.6798 |
| | treatment | | Novaseq v1.0 | | | | Hyper | | |
| AD-04_B | Week 3 | Streck | Illumina | NYGC | Omega | 10.64 | Kapa | 6 | 10.64 |
| | | | Novaseq v1.0 | | | | Hyper | | |
| AD-04_C | Week 6 | Streck | Illumina | NYGC | Omega | 17.73 | Kapa | 6 | 17.728 |
| | | | Novaseq v1.0 | | | | Hyper | | |
| AD-04_D | Week 9 | Streck | Illumina | NYGC | Omega | 13.26 | Kapa | 6 | 13.2632 |
| | | | Novaseq v1.0 | | | | Hyper | | |
| AD-05_A | Pre- | Streck | Illumina | NYGC | Omega | 13.44 | Kapa | 6 | 13.4368 |
| | treatment | | Novaseq v1.0 | | | | Hyper | | |
| AD-05_B | Week 3 | Streck | Illumina | NYGC | Omega | 6.83 | Kapa | 6 | 6.832 |
| | | | Novaseq v1.0 | | | | Hyper | | |
| AD-05_C | Week 6 | Streck | Illumina | NYGC | Omega | 37.94 | Kapa | 6 | 25 |
| | | | Novaseq v1.0 | | | | Hyper | | |
| AD-05_D | Week 9 | Streck | Illumina | NYGC | Omega | 27.24 | Kapa | 6 | 25 |
| | | | Novaseq v1.0 | | | | Hyper | | |
| AD-11_A | Pre- | Streck | Illumina | NYGC | Omega | 6.18 | Kapa | 6 | 6.1824 |
| | treatment | | Novaseq v1.0 | | | | Hyper | | |
| AD-11_B | Week 3 | Streck | Illumina | NYGC | Omega | 66.6 | Kapa | 6 | 4.125 |
| | | | Novaseq v1.0 | | | | Hyper | | |
| AD-11_C | Week 6 | Streck | Illumina | NYGC | Omega | 12.77 | Kapa | 6 | 12.7699 |
| | | | Novaseq v1.0 | | | | Hyper | | |
| AD-12_A | Pre- | Streck | Illumina | NYGC | Omega | 15.12 | Kapa | 6 | 15.125 |
| | treatment | | Novaseq v1.0 | | | | Hyper | | |
| AD-12_B | Week 3 | Streck | Illumina | NYGC | Omega | 37.47 | Kapa | 6 | 25 |
| | | | Novaseq v1.0 | | | | Hyper | | |
| AD-12_C | Week 6 | Streck | Illumina | NYGC | Omega | 20.59 | Kapa | 6 | 20.5884 |
| | | | Novaseq v1.0 | | | | Hyper | | |
| AD-16_A | Pre- | Streck | Illumina | NYGC | Omega | 7.84 | Kapa | 6 | 7.844 |
| | treatment | | Novaseq v1.0 | | | | Hyper | | |
| AD-16_B | Week 3 | Streck | Illumina | NYGC | Omega | 6.37 | Kapa | 6 | 6.371 |
| | | | Novaseq v1.0 | | | | Hyper | | |
| AD-16_C | Week 6 | Streck | Illumina | NYGC | Omega | 10.27 | Kapa | 6 | 10.2672 |
| | | | Novaseq v1.0 | | | | Hyper | | |
| AD-17_A | Pre- | Streck | Illumina | NYGC | Omega | 39.74 | Kapa | 6 | 25 |
| | treatment | | Novaseq v1.0 | | | | Hyper | | |
| AD-17_B | Week 3 | Streck | Illumina | NYGC | Omega | 8.86 | Kapa | 6 | 8.856 |
| | | | Novaseq v1.0 | | | | Hyper | | |
| AD-17_C | Week 6 | Streck | Illumina | NYGC | Omega | 13.38 | Kapa | 6 | 13.3837 |
| | | | Novaseq v1.0 | | | | Hyper | | |
| AD-18_A | Pre- | Streck | Illumina | NYGC | Omega | 5.45 | Kapa | 6 | 5.4514 |
| | treatment | | Novaseq v1.0 | | | | Hyper | | |
| AD-18_B | Week 3 | Streck | Illumina | NYGC | Omega | 7.62 | Kapa | 6 | 7.622 |
| | | | Novaseq v1.0 | | | | Hyper | | |
| AD-18_C | Week 6 | Streck | Illumina | NYGC | Omega | 6.1 | Kapa | 6 | 6.104 |
| | | | Novaseq v1.0 | | | | Hyper | | |
| AD-20_A | Pre- | Streck | Illumina | NYGC | Omega | 5.09 | Kapa | 6 | 5.0864 |
| | treatment | | Novaseq v1.0 | | | | Hyper | | |
| AD-20_B | Week 3 | Streck | Illumina | NYGC | Omega | 10.89 | Kapa | 6 | 10.89 |
| | | | Novaseq v1.0 | | | | Hyper | | |
| AD-20_C | Week 6 | Streck | Illumina | NYGC | Omega | 19.65 | Kapa | 6 | 4.7644 |
| | | | Novaseq v1.0 | | | | Hyper | | |
| AD-25_A | Pre- | Streck | Illumina | NYGC | Omega | 23.38 | Kapa | 6 | 23.375 |
| | treatment | | Novaseq v1.0 | | | | Hyper | | |
| AD-25_B | Week 3 | Streck | Illumina | NYGC | Omega | 5.5 | Kapa | 6 | 5.5044 |
| | | | Novaseq v1.0 | | | | Hyper | | |
| AD-25_C | Week 6 | Streck | Illumina | NYGC | Omega | 14.95 | Kapa | 6 | 14.9492 |
| | | | Novaseq v1.0 | | | | Hyper | | |
| AD-25_D | Week 9 | Streck | Illumina | NYGC | Omega | 12.48 | Kapa | 6 | 12.4764 |
| | | | Novaseq v1.0 | | | | Hyper | | |
| AD-26_A | Pre- | Streck | Illumina | NYGC | Omega | 33.63 | Kapa | 6 | 25 |
| | treatment | | Novaseq v1.0 | | | | Hyper | | |
| AD-26_B | Week 3 | Streck | Illumina | NYGC | Omega | 11.69 | Kapa | 6 | 11.6896 |
| | | | Novaseq v1.0 | | | | Hyper | | |
| AD-26_C | Week 6 | Streck | Illumina | NYGC | Omega | 31.2 | Kapa | 6 | 4.8198 |
| | | | Novaseq v1.0 | | | | Hyper | | |
| Acral-01_A | Pre- | Streck | Illumina | NYGC | Omega | 63.57 | Kapa | 6 | 25 |
| | treatment | | Novaseq v1.0 | | | | Hyper | | |
| Acral-01_B | Week 3 | Streck | Illumina | NYGC | Omega | 17.5 | Kapa | 6 | 17.4984 |
| | | | Novaseq v1.0 | | | | Hyper | | |
| Acral-01_C | Week 6 | Streck | Illumina | NYGC | Omega | 85.8 | Kapa | 6 | 4.8024 |
| | | | Novaseq v1.0 | | | | Hyper | | |
| AD-32_A | Pre- | Streck | Illumina | NYGC | Omega | 5.94 | Kapa | 6 | 5.94 |
| | treatment | | Novaseq v1.0 | | | | Hyper | | |
| AD-32_B | Week 3 | Streck | Illumina | NYGC | Omega | 7.7 | Kapa | 6 | 7.704 |
| | | | Novaseq v1.0 | | | | Hyper | | |
| AD-32_C | Week 6 | Streck | Illumina | NYGC | Omega | 9.55 | Kapa | 6 | 9.5472 |
| | | | Novaseq v1.0 | | | | Hyper | | |
| AD-34_A | Pre- | Streck | Illumina | NYGC | Omega | 7.08 | Kapa | 6 | 7.0848 |
| | treatment | | Novaseq v1.0 | | | | Hyper | | |
| AD-34_B | Week 3 | Streck | Illumina | NYGC | Omega | 12.91 | Kapa | 6 | 4.56 |
| | | | Novaseq v1.0 | | | | Hyper | | |
| AD-34_C | Week 6 | Streck | Illumina | NYGC | Omega | 9.62 | Kapa | 6 | 9.6248 |
| | | | Novaseq v1.0 | | | | Hyper | | |
| AD-35_A | Pre- | Streck | Illumina | NYGC | Omega | 88.13 | Kapa | 6 | 25 |
| | treatment | | Novaseq v1.0 | | | | Hyper | | |
| AD-35_B | Week 3 | Streck | Illumina | NYGC | Omega | 66.42 | Kapa | 6 | 25 |
| | | | Novaseq v1.0 | | | | Hyper | | |
| AD-36_A | Pre- | Streck | Illumina | NYGC | Omega | 5.09 | Kapa | 6 | 5.092 |
| | treatment | | Novaseq v1.0 | | | | Hyper | | |
| AD-36_B | Week 3 | Streck | Illumina | NYGC | Omega | 11.18 | Kapa | 6 | 11.178 |
| | | | Novaseq v1.0 | | | | Hyper | | |
| AD-36_C | Week 6 | Streck | Illumina | NYGC | Omega | 5.28 | Kapa | 6 | 5.2768 |
| | | | Novaseq v1.0 | | | | Hyper | | |
| AD-38_A | Pre- | Streck | Illumina | NYGC | Omega | 34.5 | Kapa | 6 | 4.266 |
| | treatment | | Novaseq v1.0 | | | | Hyper | | |
| AD-38_B | Week 3 | Streck | Illumina | NYGC | Omega | 8.61 | Kapa | 6 | 8.6093 |
| | | | Novaseq v1.0 | | | | Hyper | | |
| AD-38_C | Week 6 | Streck | Illumina | NYGC | Omega | 10.36 | Kapa | 6 | 10.3584 |
| | | | Novaseq v1.0 | | | | Hyper | | |
| AD-40_A | Pre- | Streck | Illumina | NYGC | Omega | 110.25 | Kapa | 6 | 25 |
| | treatment | | Novaseq v1.0 | | | | Hyper | | |
| AD-40_B | Week 3 | Streck | Illumina | NYGC | Omega | 18.86 | Kapa | 6 | 18.865 |
| | | | Novaseq v1.0 | | | | Hyper | | |
| AD-40_C | Week 6 | Streck | Illumina | NYGC | Omega | 20.98 | Kapa | 6 | 20.976 |
| | | | Novaseq v1.0 | | | | Hyper | | |
| AD-41_A | Pre- | Streck | Illumina | NYGC | Omega | 6.27 | Kapa | 6 | 6.2738 |
| | treatment | | Novaseq v1.0 | | | | Hyper | | |
| AD-41_B | Week 3 | Streck | Illumina | NYGC | Omega | 20 | Kapa | 6 | 19.9985 |
| | | | Novaseq v1.0 | | | | Hyper | | |
| AD-41_C | Week 6 | Streck | Illumina | NYGC | Omega | 10.62 | Kapa | 6 | 10.6172 |
| | | | Novaseq v1.0 | | | | Hyper | | |
| AD-42_A | Pre- | Streck | Illumina | NYGC | Omega | 5.64 | Kapa | 6 | 5.6368 |
| | treatment | | Novaseq v1.0 | | | | Hyper | | |
| AD-42_B | Week 3 | Streck | Illumina | NYGC | Omega | 7.62 | Kapa | 6 | 7.616 |
| | | | Novaseq v1.0 | | | | Hyper | | |
| AD-43_A | Pre- | Streck | Illumina | NYGC | Omega | 86.72 | Kapa | 6 | 25 |
| | treatment | | Novaseq v1.0 | | | | Hyper | | |
| AD-43_C | Week 3 | Streck | Illumina | NYGC | Omega | 18.99 | Kapa | 6 | 18.9856 |
| | | | Novaseq v1.0 | | | | Hyper | | |
| AD-43_D | Week 6 | Streck | Illumina | NYGC | Omega | 18.99 | Kapa | 6 | 18.9886 |
| | | | Novaseq v1.0 | | | | Hyper | | |
| AD-44_A | Pre- | Streck | Illumina | NYGC | Omega | 12.36 | Kapa | 6 | 12.3617 |
| | treatment | | Novaseq v1.0 | | | | Hyper | | |
| AD-44_B | Week 3 | Streck | Illumina | NYGC | Omega | 55.83 | Kapa | 6 | 25 |
| | | | Novaseq v1.0 | | | | Hyper | | |
| AD-44_C | Week 6 | Streck | Illumina | NYGC | Omega | 7.26 | Kapa | 6 | 7.261 |
| | | | Novaseq v1.0 | | | | Hyper | | |
| AD-45_A | Pre- | Streck | Illumina | NYGC | Omega | 73.2 | Kapa | 6 | 4.8416 |
| | treatment | | Novaseq v1.0 | | | | Hyper | | |
| AD-45_B | Week 3 | Streck | Illumina | NYGC | Omega | 48.3 | Kapa | 6 | 4.08 |
| | | | Novaseq v1.0 | | | | Hyper | | |
| AD-45_C | Week 6 | Streck | Illumina | NYGC | Omega | 13.86 | Kapa | 6 | 13.86 |
| | | | Novaseq v1.0 | | | | Hyper | | |
| AD-46_A | Pre- | Streck | Illumina | NYGC | Omega | 5.88 | Kapa | 6 | 5.8752 |
| | treatment | | Novaseq v1.0 | | | | Hyper | | |
| AD-46_B | Week 3 | Streck | Illumina | NYGC | Omega | 6.24 | Kapa | 6 | 6.2408 |
| | | | Novaseq v1.0 | | | | Hyper | | |
| AD-46_C | Week 6 | Streck | Illumina | NYGC | Omega | 40.2 | Kapa | 6 | 3.7122 |
| | | | Novaseq v1.0 | | | | Hyper | | |
| AD-48_A | Pre- | Streck | Illumina | NYGC | Omega | 6.51 | Kapa | 6 | 6.5148 |
| | treatment | | Novaseq v1.0 | | | | Hyper | | |
| AD-48_B | Week 3 | Streck | Illumina | NYGC | Omega | 5.6 | Kapa | 6 | 5.5952 |
| | | | Novaseq v1.0 | | | | Hyper | | |
| AD-48_C | Week 6 | Streck | Illumina | NYGC | Omega | 13.35 | Kapa | 6 | 13.35 |
| | | | Novaseq v1.0 | | | | Hyper | | |
| AD-50_A | Pre- | Streck | Illumina | NYGC | Omega | 5.18 | Kapa | 6 | 5.178880119 |
| | treatment | | Novaseq v1.0 | | | | Hyper | | |
| AD-50_B | Week 3 | Streck | Illumina | NYGC | Omega | 76.8 | Kapa | 6 | 4.2588 |
| | | | Novaseq v1.0 | | | | Hyper | | |
| AD-50_C | Week 6 | Streck | Illumina | NYGC | Omega | 31.5 | Kapa | 6 | 4.090879941 |
| | | | Novaseq v1.0 | | | | Hyper | | |

| Adaptive | | | | | | | | |
|---|---|---|---|---|---|---|---|---|
| Dosing | | | | QC | | | | |
| Melanoma | | | | metrics | | | | |
| Plasma | | Percent- | Mean | MEDIAN_{—} | | | | |
| Patient | TOTAL_{—} | Total- | Coverage | INSERT_{—} | Conpair- | Pileup- | Auto- | |
| ID | READS | Duplication | (X) | SIZE (bp) | Concordance | Size | correlation | Notes |
| AD-01_A | 9.17E+08 | 6.3551 | 32.3796 | 191 | 99.82 | 48411838 | 0.02732999 | |
| AD-01_B | 9.13E+08 | 6.2882 | 28.5835 | 172 | 99.9 | 23096371 | 0.06663636 | |
| AD-01_C | 9.3E+08 | 6.7867 | 30.2968 | 175 | 99.82 | 43044154 | 0.02034583 | |
| AD-01_D | 1.05E+09 | 6.4426 | 32.9091 | 171 | 99.84 | 27051241 | 0.08101972 | |
| AD-01_E | 9.9E+08 | 5.8537 | 33.7634 | 183 | 99.87 | 53988135 | 0.1045253 | |
| AD-02_A | 2.16E+09 | 8.2666 | 80.9947 | 253 | 99.82 | 123864160 | 0.07486098 | |
| AD-02_B | 9.34E+08 | 5.3472 | 35.6483 | 240 | 99.71 | 56805096 | 0.03411255 | |
| AD-02_C | 1.08E+09 | 6.8632 | 33.9924 | 174 | 99.81 | 25272624 | 0.1022875 | |
| AD-04_A | 9.4E+08 | 6.8904 | 30.0937 | 176 | 99.82 | 33805436 | 0.0733372 | |
| AD-04_B | 1E+09 | 6.8631 | 30.3567 | 171 | 99.79 | 29148519 | 0.07138279 | |
| AD-04_C | 1.17E+09 | 5.8453 | 37.2967 | 174 | 99.79 | 30329952 | 0.0647977 | |
| AD-04_D | 9.28E+08 | 6.27 | 29.2434 | 174 | 99.74 | 21746378 | 0.06725574 | |
| AD-05_A | 9.31E+08 | 7.5113 | 28.1456 | 168 | 99.84 | 28804107 | 0.1325735 | |
| AD-05_B | 1.05E+09 | 8.1065 | 32.7634 | 174 | 99.79 | 36985918 | 0.1148754 | |
| AD-05_C | 7.18E+08 | 6.1352 | 24.734 | 182 | 99.74 | 43828156 | 0.1638985 | |
| AD-05_D | 1.4E+09 | 5.7767 | 47.346 | 177 | 99.84 | 52794215 | 0.1831796 | |
| AD-11_A | 9.17E+08 | 7.6261 | 27.7061 | 170 | 99.82 | 25080716 | 0.05837027 | |
| AD-11_B | 9.26E+08 | 9.8669 | 28.1278 | 173 | 99.77 | 25481962 | 0.067741 | |
| AD-11_C | 1.02E+09 | 6.9993 | 33.2307 | 176 | 99.84 | 45277040 | 0.1616924 | |
| AD-12_A | 1.13E+09 | 6.4167 | 34.1752 | 169 | 99.87 | 28172232 | 0.1067267 | |
| AD-12_B | 1.09E+09 | 6.3734 | 35.499 | 173 | 99.92 | 36232756 | 0.05084546 | |
| AD-12_C | 7.92E+08 | 6.9767 | 23.3483 | 169 | 99.82 | 23407836 | 0.07064373 | |
| AD-16_A | 9.42E+08 | 7.8428 | 28.4465 | 172 | 99.84 | 25762692 | 0.04855819 | |
| AD-16_B | 1.11E+09 | 10.445 | 34.048 | 176 | 99.81 | 41493892 | 0.1085483 | |
| AD-16_C | 8.44E+08 | 5.9061 | 26.05 | 172 | 99.92 | 19486080 | 0.08552197 | |
| AD-17_A | 9.28E+08 | 10.014 | 28.5924 | 175 | NA | 34567948 | 0.03712122 | |
| AD-17_B | 7.7E+08 | 6.6904 | 23.5987 | 171 | NA | 24917054 | 0.06514705 | |
| AD-17_C | 1.23E+09 | 9.3856 | 37.8379 | 173 | NA | 43658815 | 0.09798803 | |
| AD-18_A | 8.66E+08 | 7.5935 | 25.9216 | 171 | 99.84 | 24629446 | 0.08058997 | |
| AD-18_B | 9.51E+08 | 6.9484 | 29.4973 | 173 | 99.79 | 27441810 | 0.05446942 | |
| AD-18_C | 1.06E+09 | 9.6593 | 32.7811 | 176 | 99.87 | 35965575 | 0.07813889 | |
| AD-20_A | 8.98E+08 | 7.7167 | 26.1208 | 171 | 99.87 | 26999255 | 0.06326216 | |
| AD-20_B | 1.48E+09 | 8.5044 | 41.1887 | 168 | 99.92 | 38483072 | 0.1003804 | |
| AD-20_C | 1.21E+09 | 8.6028 | 35.5685 | 171 | 99.92 | 27255732 | 0.08018032 | |
| AD-25_A | 9.47E+08 | 7.0303 | 30.0704 | 174 | 99.87 | 34023346 | 0.05600333 | |
| AD-25_B | 1.17E+09 | 11.089 | 35.9737 | 176 | 99.9 | 38260709 | 0.05179411 | |
| AD-25_C | 1.32E+09 | 7.6685 | 41.5992 | 174 | 99.95 | 31358209 | 0.06336935 | |
| AD-25_D | 9.56E+08 | 5.5032 | 30.2597 | 173 | 99.92 | 24850022 | 0.05693587 | |
| AD-26_A | 7.56E+08 | 8.79 | 21.6875 | 170 | 99.92 | 39853843 | 0.052583 | |
| AD-26_B | 1.04E+09 | 7.3057 | 30.9544 | 172 | 99.9 | 27728080 | 0.06667941 | |
| AD-26_C | 1.16E+09 | 7.8336 | 32.7299 | 167 | 99.87 | 23192649 | 0.06836471 | |
| Acral-01_A | 1.21E+09 | 5.3802 | 44.7776 | 242 | 99.82 | 72415242 | 0.02652707 | |
| Acral-01_B | 9.6E+08 | 6.3124 | 34.1728 | 213 | 99.84 | 52723503 | 0.1408548 | |
| Acral-01_C | 1.16E+09 | 9.7101 | 33.5451 | 171 | 99.87 | 23640866 | 0.06991639 | |
| AD-32_A | 7.77E+08 | 6.9667 | 24.633 | 174 | 99.92 | 26842617 | 0.07190328 | |
| AD-32_B | 1.07E+09 | 8.3753 | 32.881 | 173 | 99.89 | 32010005 | 0.08049727 | |
| AD-32_C | 9.94E+08 | 14.849 | 28.7089 | 175 | 99.82 | 28961456 | 0.06753418 | |
| AD-34_A | 8.47E+08 | 7.0275 | 26.0072 | 173 | 99.9 | 46456888 | 0.06278318 | |
| AD-34_B | 1.06E+09 | 12.9124 | 30.4443 | 173 | 35.67 | 26743760 | 0.05089087 | |
| AD-34_C | 1.22E+09 | 16.0686 | 35.81 | 179 | 35.63 | 41915551 | 0.06275248 | |
| AD-35_A | 1.92E+09 | 8.4526 | 55.3344 | 166 | 99.84 | 35177042 | 0.08803991 | |
| AD-35_B | 1.3E+09 | 7.1318 | 37.549 | 166 | 99.97 | 26582859 | 0.09192994 | |
| AD-36_A | 1.15E+09 | 7.9749 | 33.7984 | 171 | 99.79 | 31648903 | 0.03352981 | |
| AD-36_B | 8.57E+08 | 6.1517 | 25.3009 | 168 | 99.79 | 21023479 | 0.037596 | |
| AD-36_C | 1.2E+09 | 8.3868 | 35.0246 | 170 | 99.79 | 33187659 | 0.03905656 | |
| AD-38_A | 1.43E+09 | 10.3973 | 43.9463 | 177 | 99.87 | 39498568 | 0.04689902 | |
| AD-38_B | 9.73E+08 | 8.854 | 28.8532 | 171 | 99.92 | 24248039 | 0.08884703 | |
| AD-38_C | 1.02E+09 | 8.3365 | 30.8011 | 172 | 99.95 | 28126154 | 0.06305716 | |
| AD-40_A | 1.39E+09 | 6.4647 | 45.0657 | 172 | 99.95 | 42801903 | 0.04991221 | |
| AD-40_B | 1.06E+09 | 5.6033 | 36.477 | 181 | 99.87 | 47191297 | 0.07587351 | |
| AD-40_C | 9.46E+08 | 5.3427 | 29.0758 | 169 | 99.84 | 23748030 | 0.0494062 | |
| AD-41_A | 9.65E+08 | 7.9356 | 28.4741 | 169 | 99.89 | 27052612 | 0.08983282 | |
| AD-41_B | 1.04E+09 | 5.6033 | 33.7186 | 176 | 99.97 | 28327425 | 0.06602152 | |
| AD-41_C | 9.11E+08 | 7.3717 | 26.6024 | 169 | 99.97 | 26416009 | 0.06850866 | |
| AD-42_A | 9.19E+08 | 7.2536 | 27.4201 | 169 | NA | 24056984 | 0.05662109 | |
| AD-42_B | 9.39E+08 | 7.1116 | 27.9448 | 170 | NA | 22455617 | 0.06792469 | |
| AD-43_A | 1.2E+09 | 6.6803 | 35.2154 | 168 | 99.87 | 24183715 | 0.05538083 | |
| AD-43_C | 1E+09 | 5.8019 | 29.3449 | 168 | 99.84 | 43393756 | 0.06140052 | |
| AD-43_D | 9.94E+08 | 6.1819 | 29.601 | 169 | 99.84 | 23782735 | 0.08358163 | |
| AD-44_A | 9.78E+08 | 7.3681 | 29.398 | 170 | 99.85 | 26622403 | 0.06047469 | |
| AD-44_B | 7.62E+08 | 5.5751 | 29.4782 | 256 | 99.9 | 51617310 | 0.2571813 | |
| AD-44_C | 9.01E+08 | 7.3803 | 27.2182 | 172 | 99.8 | 26368417 | 0.04068179 | |
| AD-45_A | 1.31E+09 | 10.503 | 38.7327 | 174 | 99.73 | 28223317 | 0.05663921 | |
| AD-45_B | 1.21E+09 | 8.9578 | 35.7629 | 172 | 99.81 | 26097925 | 0.05748843 | |
| AD-45_C | 8.42E+08 | 6.5097 | 25.1803 | 170 | 99.73 | 24660009 | 0.04746766 | |
| AD-46_A | 1.81E+09 | 10.1047 | 53.7093 | 172 | 99.95 | 33823916 | 0.08132688 | |
| AD-46_B | 8.23E+08 | 7.152 | 25.191 | 171 | 99.77 | 25219663 | 0.04645097 | |
| AD-46_C | 1.09E+09 | 9.625 | 32.3229 | 172 | 99.9 | 23553335 | 0.08313506 | |
| AD-48_A | 8.34E+08 | 7.0691 | 26.0489 | 173 | 99.9 | 24246058 | 0.08627503 | |
| AD-48_B | 8.79E+08 | 6.9462 | 26.9391 | 172 | 99.9 | 27017808 | 0.08476272 | |
| AD-48_C | 1.01E+09 | 6.1421 | 31.9111 | 173 | 99.9 | 24741017 | 0.07419537 | |
| AD-50_A | 8.7E+08 | 11.0586 | 26.2523 | 175 | 99.84 | 24470048 | 0.06435319 | |
| AD-50_B | 1.05E+09 | 8.6437 | 33.4058 | 177 | 99.87 | 36881303 | 0.06839073 | |
| AD-50_C | 8.3E+08 | 12.7739 | 23.3036 | 171 | 99.9 | 24329692 | 0.07305205 | |

*library mass capped at 25 ng

| Aarhus | | | | TOTAL_{—} | Percent- | | | QC | | |
|---|---|---|---|---|---|---|---|---|---|---|
| University | | | | READS | Total- | Mean | MEDIAN_{—} | metrics | auto- | # of |
| Patient | Sample | Tissue | Sequencing | TOTAL_{—} | Dupli- | Coverage | INSERT_{—} | Conpair | corre- | mutation |
| ID | ID | Type | platform | READS | cation | (X) | SIZE (bp) | Concordance | lation | detected |
| Aar-01 | MF-3930 | Fresh | Illumina | 2050478570 | 3.6324 | 91.0865 | 354 | 99.95 | 0.0173 | 10604 |
| | | frozen | NovaSeq v1.0 | | | | | | | |
| Aar-02 | MF-5766 | Fresh | Illumina | 2236564818 | 3.7754 | 99.7875 | 371 | 100 | 0.0337 | 6993 |
| | | frozen | NovaSeq v1.0 | | | | | | | |
| Aar-03 | MF-5812 | Fresh | Illumina | 1830157426 | 3.8829 | 83.7483 | 363 | 99.87 | 0.0264 | 7084 |
| | | frozen | NovaSeq v1.0 | | | | | | | |
| Aar-04 | MF-6596 | Fresh | Illumina | 1985553238 | 3.323 | 90.0687 | 357 | 99.77 | 0.0669 | 51501 |
| | | frozen | NovaSeq v1.0 | | | | | | | |
| Aar-05 | MF-5823 | Fresh | Illumina | 1706685500 | 3.6478 | 77.2226 | 351 | 99.83 | 0.0171 | 4511 |
| | | frozen | NovaSeq v1.0 | | | | | | | |
| Aar-06 | MF-6025 | FFPE | Illumina | 1747895888 | 10.2671 | 53.1046 | 189 | 99.61 | 0.0585 | 7455 |
| | | | NovaSeq v1.0 | | | | | | | |
| Aar-07 | MF-4165 | FFPE | Illumina | 1907357132 | 17.236 | 51.0431 | 185 | 99.9 | 0.0906 | 6449 |
| | | | NovaSeq v1.0 | | | | | | | |
| Aar-08 | MF-2900 | FFPE | Illumina | 2002180798 | 13.0587 | 59.0641 | 191 | 99.82 | 0.0611 | 4460 |
| | | | NovaSeq v1.0 | | | | | | | |
| Aar-09 | MF-3511 | FFPE | Illumina | 1774697338 | 13.1748 | 56.0798 | 217 | 99.87 | 0.0641 | 6173 |
| | | | NovaSeq v1.0 | | | | | | | |
| Aar-10 | MF-8594 | FFPE | Illumina | 2013294438 | 10.1478 | 62.4421 | 186 | 99.73 | 0.0603 | 6037 |
| | | | NovaSeq v1.0 | | | | | | | |
| Aar-11 | MF-5427 | FFPE | Illumina | 2052526926 | 15.5625 | 58.7842 | 194 | 99.8 | 0.129 | 4159 |
| | | | NovaSeq v1.0 | | | | | | | |
| Aar-12 | MF-5287 | FFPE | Illumina | 1662638240 | 11.7144 | 47.4771 | 179 | 99.67 | 0.0601 | 6536 |
| | | | NovaSeq v1.0 | | | | | | | |
| Aar-13 | MF-7637 | FFPE | Illumina | 1761538970 | 14.4051 | 47.5133 | 171 | 99.63 | 0.0622 | 9254 |
| | | | NovaSeq v1.0 | | | | | | | |
| Aar-14 | MF-9859 | FFPE | Illumina | 1897375204 | 14.0367 | 57.3105 | 204 | 99.77 | 0.059 | 10477 |
| | | | NovaSeq v1.0 | | | | | | | |
| Aar-15 | MF-9144 | FFPE | Illumina | 2257978172 | 12.7167 | 67.5607 | 193 | 99.88 | 0.0594 | 813 |
| | | | NovaSeq v1.0 | | | | | | | |
| Aar-16 | MF-1255 | FFPE | Illumina | 2084367306 | 12.2214 | 66.1878 | 214 | 99.85 | 0.0578 | 3155 |
| | | | NovaSeq v1.0 | | | | | | | |
| Aar-17 | MF-8145 | FFPE | Illumina | 1816038758 | 11.1747 | 57.8145 | 209 | 99.92 | 0.0605 | 3503 |
| | | | NovaSeq v1.0 | | | | | | | |
| Aar-18 | MF-1566 | FFPE | Illumina | 2175388248 | 14.8898 | 67.4676 | 213 | 99.72 | 0.0624 | 3331 |
| | | | NovaSeq v1.0 | | | | | | | |
| Aar-19 | MF-5738 | FFPE | Illumina | 2662158096 | 13.0354 | 87.0623 | 228 | 99.77 | 0.0574 | 27227 |
| | | | NovaSeq v1.0 | | | | | | | |
| Aar-20 | MF-3793 | FFPE | Illumina | 2375543556 | 12.3055 | 83.8404 | 255 | 99.87 | 0.0662 | 8796 |
| | | | NovaSeq v1.0 | | | | | | | |
| Aar-21 | MF-4629 | FFPE | Illumina | 2076666530 | 10.7683 | 68.0642 | 214 | 99.77 | 0.0576 | 3829 |
| | | | NovaSeq v1.0 | | | | | | | |
| Aar-22 | MF-9004 | FFPE | Illumina | 1780706766 | 10.3788 | 58.0884 | 216 | 99.9 | 0.0623 | 5210 |
| | | | NovaSeq v1.0 | | | | | | | |
| Aar-23 | MF-1203 | FFPE | Illumina | 1772759938 | 10.4535 | 59.6536 | 221 | 99.81 | 0.0603 | 6480 |
| | | | NovaSeq v1.0 | | | | | | | |
| Aar-24 | MF-1208 | FFPE | Illumina | 1853039712 | 15.2734 | 51.8532 | 186 | 99.9 | 0.0592 | 7729 |
| | | | NovaSeq v1.0 | | | | | | | |
| Aar-25 | MF-5642 | FFPE | Illumina | 1716763694 | 9.6176 | 59.1553 | 227 | 99.87 | 0.0531 | 3474 |
| | | | NovaSeq v1.0 | | | | | | | |
| Aar-26 | MF-8291 | FFPE | Illumina | 2242842124 | 12.6412 | 75.1421 | 234 | 99.83 | 0.06 | 4216 |
| | | | NovaSeq v1.0 | | | | | | | |
| Aar-27 | MF-3108 | FFPE | Illumina | 1774697338 | 13.1748 | 56.0798 | 217 | 99.87 | 0.0641 | 6607 |
| | | | NovaSeq v1.0 | | | | | | | |
| Aar-28 | MF-1794 | FFPE | Illumina | 1880256590 | 10.5022 | 61.3861 | 208 | 99.82 | 0.0576 | 5663 |
| | | | NovaSeq v1.0 | | | | | | | |
| Aar-29 | MF-9921 | FFPE | Illumina | 2093780814 | 12.678 | 71.5416 | 241 | 99.87 | 0.0812 | 6380 |
| | | | NovaSeq v1.0 | | | | | | | |
| Aar-30 | MF-0187 | FFPE | Illumina | 2236508988 | 15.2871 | 69.4804 | 213 | 99.9 | 0.0618 | 7937 |
| | | | NovaSeq v1.0 | | | | | | | |
| Aar-31 | MF-1673 | FFPE | Illumina | 1984885618 | 11.3249 | 64.8774 | 219 | 99.77 | 0.0546 | |
| | | | NovaSeq v1.0 | | | | | | | |
| Aar-32 | MF-1137 | FFPE | Illumina | 2028367930 | 12.1942 | 65.083 | 212 | 99.74 | 0.0607 | |
| | | | NovaSeq v1.0 | | | | | | | |
| Aar-33 | MF-1590 | FFPE | Illumina | 2074045548 | 10.9902 | 72.2257 | 247 | 99.82 | 0.0651 | |
| | | | NovaSeq v1.0 | | | | | | | |
| Aar-34 | MF-1103 | FFPE | Illumina | 2302634572 | 14.2417 | 70.402 | 205 | 99.8 | 0.0623 | |
| | | | NovaSeq v1.0 | | | | | | | |
| Aar-35 | MF-1060 | FFPE | Illumina | 2105893000 | 10.7194 | 74.3364 | 243 | 99.8 | 0.0631 | |
| | | | NovaSeq v1.0 | | | | | | | |

| | | | | | | Total | | |
|---|---|---|---|---|---|---|---|---|
| Aarhus | somatic | | | | | Mbp of | | |
| University | pipeline | | | Total | Total | copy- | Tumor | |
| Patient | # of | # of | # of copy- | Mbp of | Mbp of | neutral | purity | |
| ID | amplification | deletion | neutral LOH | amplification | deletion | LOH | (%) | Notes |
| Aar-01 | 18 | 29 | 5 | 369.849 | 325.113 | 178.661 | 55 | |
| Aar-02 | 40 | 41 | 5 | 683.829 | 913.452 | 136.973 | 81 | |
| Aar-03 | 15 | 20 | 2 | 1182.69 | 969.921 | 85.6128 | 46 | |
| Aar-04 | 8 | 9 | 1 | 303.765 | 74.0318 | 88.8514 | 81 | |
| Aar-05 | 15 | 31 | 0 | 611.745 | 1144.89 | 0 | 39 | |
| Aar-06 | 2 | 6 | 0 | 162.594 | 380.277 | 0 | 92 | |
| Aar-07 | 0 | 7 | 0 | 0 | 1008.79 | 0 | 33 | |
| Aar-08 | 3 | 2 | 1 | 292.559 | 78.5209 | 95.268 | 46 | |
| Aar-09 | 5 | 3 | 4 | 13.0583 | 5.16523 | 184.432 | 82 | |
| Aar-10 | 8 | 9 | 0 | 754.453 | 763.995 | 0 | 63 | |
| Aar-11 | 6 | 9 | 1 | 731.377 | 556.267 | 54.9976 | 58 | |
| Aar-12 | 2 | 7 | 0 | 112.41 | 288.848 | 0 | 64 | |
| Aar-13 | 4 | 0 | 1 | 411.283 | 0 | 53.9583 | 82 | |
| Aar-14 | 12 | 15 | 0 | 1153.85 | 1625.01 | 0 | 62 | |
| Aar-15 | 1 | 0 | 0 | 38.2373 | 0 | 0 | 29 | Excluded for low |
| | | | | | | | | tumor purity |
| | | | | | | | | (<30%) precluding |
| | | | | | | | | accurate |
| | | | | | | | | identification of |
| | | | | | | | | somatic mutations |
| | | | | | | | | in FFPE |
| Aar-16 | NA | NA | NA | NA | NA | NA | NA | |
| Aar-17 | 1 | 0 | 1 | 159.314 | 0 | 109.985 | 69 | |
| Aar-18 | NA | NA | NA | NA | NA | NA | NA | |
| Aar-19 | 0 | 0 | 3 | 0 | 0 | 200.987 | 77 | |
| Aar-20 | 6 | 1 | 1 | 366.608 | 26.2578 | 121.742 | 88 | |
| Aar-21 | 1 | 0 | 0 | 98.354 | 0 | 0 | 77 | |
| Aar-22 | NA | NA | NA | NA | NA | NA | NA | |
| Aar-23 | 1 | 1 | 0 | 159.301 | 39.8033 | 0 | 77 | |
| Aar-24 | 3 | 4 | 0 | 195.174 | 165.798 | 0 | 85 | |
| Aar-25 | NA | NA | NA | NA | NA | NA | NA | |
| Aar-26 | 3 | 0 | 0 | 430.755 | 0 | 0 | 100 | |
| Aar-27 | 0 | 0 | 1 | 0 | 0 | 18.2823 | 78 | |
| Aar-28 | 1 | 1 | 0 | 87.2942 | 31.4215 | 0 | 78 | |
| Aar-29 | NA | NA | NA | NA | NA | NA | NA | |
| Aar-30 | 1 | 1 | 1 | 95.8972 | 29.071 | 38.937 | 83 | |
| Aar-31 | | | | | | | | |
| Aar-32 | | | | | | | | |
| Aar-33 | | | | | | | | |
| Aar-34 | | | | | | | | |
| Aar-35 | | | | | | | | |

| | | sequencing | | | MEDIAN_{—} | QC | | |
|---|---|---|---|---|---|---|---|---|
| Aarhus | | metrics | | Mean | INSERT_{—} | metrics | | |
| University | Sample | TOTAL_{—} | PercentTo- | Coverage | SIZE | ConpairCon- | auto- | |
| Patient ID | ID | READS | talDuplication | (X) | (bp) | cordance | correlation | Notes |
| Aar-01 | MF-3930 | 1.19E+09 | 3.9579 | 53.841 | 366 | 99.95 | 0.004 | |
| Aar-02 | MF-5766 | 1.526E+09 | 5.4869 | 68.229 | 395 | 100 | 0.0249 | |
| Aar-03 | MF-5812 | 1.051E+09 | 3.6188 | 46.2425 | 341 | 99.87 | 0.0398 | |
| Aar-04 | MF-6596 | 933674544 | 3.4175 | 42.7083 | 380 | 99.77 | 0.0081 | |
| Aar-05 | MF-5823 | 1.176E+09 | 3.4177 | 54.973 | 402 | 99.83 | 0.0095 | |
| Aar-06 | MF-6025 | 1.077E+09 | 3.9887 | 49.7584 | 386 | 99.61 | 0.0064 | |
| Aar-07 | MF-4165 | 976783182 | 3.9517 | 43.7707 | 356 | 99.9 | 0.0034 | |
| Aar-08 | MF-2900 | 904799322 | 3.4821 | 41.85 | 370 | 99.82 | 0.0019 | |
| Aar-09 | MF-3511 | 1.111E+09 | 3.8317 | 50.5259 | 384 | 99.82 | 0.0069 | |
| Aar-10 | MF-8594 | 1.048E+09 | 3.7767 | 48.2415 | 373 | 99.73 | 0.005 | |
| Aar-11 | MF-5427 | 896125060 | 3.2251 | 42.0095 | 385 | 99.8 | 0.019 | |
| Aar-12 | MF-5287 | 1.117E+09 | 3.6999 | 50.903 | 374 | 99.67 | 0.0157 | |
| Aar-13 | MF-7637 | 834740270 | 3.3532 | 39.04 | 391 | 99.63 | 0.0063 | |
| Aar-14 | MF-9859 | 915830750 | 3.2642 | 42.0432 | 387 | 99.77 | 0.0091 | |
| Aar-15 | MF-9144 | 1.18E+09 | 3.8393 | 54.6768 | 385 | 99.88 | 0.0057 | |
| Aar-16 | MF-1255 | 829066526 | 2.8179 | 37.9473 | 371 | 99.85 | 0.0028 | |
| Aar-17 | MF-8145 | 772678548 | 2.8498 | 35.3737 | 363 | 99.92 | 0.0004 | |
| Aar-18 | MF-1566 | 985590436 | 3.6239 | 45.3488 | 368 | 99.72 | −0.0013 | |
| Aar-19 | MF-5738 | 886782310 | 3.4023 | 40.1064 | 374 | 99.77 | 0.0075 | |
| Aar-20 | MF-3793 | 2.376E+09 | 12.3055 | 83.8404 | 255 | 99.87 | 0.0662 | |

| | | | | | | | |
|---|---|---|---|---|---|---|---|
| 8117 | 11 | 0 | 0 | 1121.99 | 0 | 0 | 100 |
| 2665 | 0 | 0 | 1 | 0 | 0 | 111.781 | 63 |
| 3918 | 7 | 1 | 0 | 774.263 | 80.2511 | 0 | 80 |
| 5129 | 4 | 0 | 0 | 540.511 | 0 | 0 | 58 |
| 6839 | 3 | 0 | 1 | 105.908 | 0 | 107.526 | 81 |

| | | | | | | | |
|---|---|---|---|---|---|---|---|
| Aar-21 | MF-4629 | 1.312E+09 | 3.9827 | 60.3776 | 368 | 99.77 | 0.0068 |
| Aar-22 | MF-9004 | 967276742 | 3.263 | 44.036 | 363 | 99.9 | 0.0021 |
| Aar-23 | MF-1203 | 956448274 | 3.3627 | 44.6295 | 391 | 99.81 | 0.0028 |
| Aar-24 | MF-1208 | 1.579E+09 | 4.3093 | 72.5698 | 391 | 99.9 | 0.005 |
| Aar-25 | MF-5642 | 752409838 | 2.7301 | 34.9063 | 360 | 99.87 | 0.0097 |
| Aar-26 | MF-8291 | 1.26E+09 | 3.2291 | 56.9836 | 358 | 99.83 | 0.0026 |
| Aar-27 | MF-3108 | 972350490 | 2.9436 | 44.2669 | 365 | 99.87 | 0.092 |
| Aar-28 | MF-1794 | 800635292 | 2.8276 | 37.1681 | 363 | 99.82 | 0.0022 |
| Aar-29 | MF-9921 | 913565640 | 2.8361 | 41.3876 | 358 | 99.87 | 0.0014 |
| Aar-30 | MF-0187 | 862281328 | 2.8833 | 40.2671 | 366 | 99.9 | 0.0026 |
| Aar-31 | MF-1673 | 1.188E+09 | 3.7447 | 53.5087 | 345 | 99.77 | 0.0015 |
| Aar-32 | MF-1137 | 877419154 | 3.5843 | 40.7579 | 390 | 99.74 | 0.0081 |
| Aar-33 | MF-1590 | 918076128 | 2.8877 | 41.7373 | 362 | 99.82 | 0.0029 |
| Aar-34 | MF-1103 | 1.004E+09 | 2.831 | 46.5356 | 363 | 99.8 | 0.061 |
| Aar-35 | MF-1060 | 918459462 | 3.0533 | 42.4623 | 365 | 99.8 | 0.073 |

| | | pre | | | | | | | |
|---|---|---|---|---|---|---|---|---|---|
| Aarhus | | sequencing | | | | | | | |
| University | | QC | | | | | | | |
| Plasma | | Blood | | | | total | library | # of | library |
| Patient | Sample | Collection | Sequencing | Sequencing | extraction | mass | prep | PCR | mass |
| ID | ID | Tube | Platform | Location | kit | (ng) | kit | cycles | (ng) |
| Aar-01 | MF-3930 | K2-EDTA | Illumina | Aarhus | QIAamp | 5.8 | Kapa | 7 | 607.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-02 | MF-5766 | K2-EDTA | Illumina | Aarhus | QIAamp | 11.9 | Kapa | 7 | 985.5 |
| | | | Novaseq v1.5 | University | Circulating | | Hyper | | |
| | | | | | Nucleic | | | | |
| | | | | | Acid Kit | | | | |
| | | | | | (qiagen) | | | | |

| sequencing | | | MEDIAN_{—} | | | | | |
|---|---|---|---|---|---|---|---|---|
| metrics | | Mean | INSERT_{—} | QC metrics | | Neutral | SNV | |
| TOTAL_{—} | PercentTo- | Coverage | SIZE | ConpairCon- | Auto- | regions | Pileup | |
| READS | talDuplication | (X) | (bp) | cordance | correlation | ZScore | size | Notes |
| 1.22E+09 | 8.0332 | 31.2 | 169 | 99.93 | 0.0642 | NA | 2.21E+07 | |
| 1.28E+09 | 7.6232 | 32.8 | 168 | 100 | 0.0525 | NA | 2.20E+07 | |

| | | | | | | | | | |
|---|---|---|---|---|---|---|---|---|---|
| Aar-03 | MF-5812 | K2-EDTA | Illumina | Aarhus | QIAamp | 43.6 | Kapa | 7 | 2263.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-04 | MF-6596 | K2-EDTA | Illumina | Aarhus | QIAamp | 4 | Kapa | 7 | 370.35 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-05 | MF-5823 | K2-EDTA | Illumina | Aarhus | QIAamp | 8.4 | Kapa | 7 | 706.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-06 | MF-6025 | K2-EDTA | Illumina | Aarhus | QIAamp | 8.1 | Kapa | 7 | 679.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-07 | MF-4165 | K2-EDTA | Illumina | Aarhus | QIAamp | 5.3 | Kapa | 7 | 394.2 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-08 | MF-2900 | K2-EDTA | Illumina | Aarhus | QIAamp | 7.4 | Kapa | 7 | 607.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-09 | MF-3511 | K2-EDTA | Illumina | Aarhus | QIAamp | 8.7 | Kapa | 7 | 774 |
| | | | Novaseq v1.5 | University | Circulating | | Hyper | | |
| | | | | | Nucleic | | | | |
| | | | | | Acid Kit | | | | |
| | | | | | (qiagen) | | | | |

| | | | | | | | |
|---|---|---|---|---|---|---|---|
| 1.29E+09 | 7.2728 | 33.3 | 167 | 99.87 | 0.1157 | NA | 2.15E+07 |
| 1.27E+09 | 9.0292 | 33.3 | 172 | 99.82 | 0.0907 | NA | 2.19E+07 |
| 1.32E+09 | 8.5075 | 34 | 170 | 99.79 | 0.0312 | NA | 2.75E+07 |
| 1.43E+09 | 9.9473 | 39.2 | 179 | 99.9 | 0.1217 | 0.946269111 | 3.48E+07 |
| 1.39E+09 | 13.2658 | 34.7 | 174 | 99.92 | 0.0743 | 0.595036439 | 3.48E+07 |
| 1.37E+09 | 9.1034 | 36.2 | 174 | 99.92 | 0.065 | 0.417183927 | 2.20E+07 |
| 1.31E+09 | 11.8522 | 31 | 166 | 99.87 | 0.0567 | 1.456727016 | 2.63E+07 |

| | | | | | | | | | |
|---|---|---|---|---|---|---|---|---|---|
| Aar-10 | MF-8594 | K2-EDTA | Illumina | Aarhus | QIAamp | 4 | Kapa | 7 | 468 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-11 | MF-5427 | K2-EDTA | Illumina | Aarhus | QIAamp | 4.6 | Kapa | 7 | 351 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-12 | MF-5287 | K2-EDTA | Illumina | Aarhus | QIAamp | 4 | Kapa | 7 | 282.15 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-13 | MF-7637 | K2-EDTA | Illumina | Aarhus | QIAamp | 4.9 | Kapa | 7 | 454.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-14 | MF-9859 | K2-EDTA | Illumina | Aarhus | QIAamp | 4 | Kapa | 7 | 282.6 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-15 | MF-9144 | K2-EDTA | Illumina | Aarhus | QIAamp | 6.2 | Kapa | 7 | 472.5 |
| | | | Novaseq v1.5 | University | Circulating | | Hyper | | |
| | | | | | Nucleic | | | | |
| | | | | | Acid Kit | | | | |
| | | | | | (qiagen) | | | | |

| | | | | | | | | |
|---|---|---|---|---|---|---|---|---|
| 1.44E+09 | 10.6384 | 36.3 | 170 | 99.89 | 0.0594 | −0.159254977 | 2.45E+07 | Excluded for low |
| 1.28E+09 | 10.585 | 31.8 | 172 | 99.87 | 0.0502 | −0.904918872 | 2.14E+07 | tumor purity |
| 1.14E+09 | 11.0737 | 29.8 | 176 | 99.97 | 0.0465 | 1.273490718 | 2.78E+07 | (<30%) precluding |
| 1.55E+09 | 11.2359 | 40.5 | 175 | 99.9 | 0.0597 | 0.353587268 | 3.20E+07 | accurate |
| 1.46E+09 | 11.0169 | 38.5 | 175 | 99.82 | 0.0729 | NA | 2.99E+07 | identification of |
| 1.57E+09 | 10.0264 | 43.2 | 178 | 99.92 | 0.0656 | −0.449194918 | 3.76E+07 | somatic mutations |
| | | | | | | | | in FFPE |

| | | | | | | | | | |
|---|---|---|---|---|---|---|---|---|---|
| Aar-16 | MF-1255 | K2-EDTA | Illumina | Aarhus | QIAamp | 4.1 | Kapa | 7 | 481.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-17 | MF-8145 | K2-EDTA | Illumina | Aarhus | QIAamp | 4 | Kapa | 7 | 270 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-18 | MF-1566 | K2-EDTA | Illumina | Aarhus | QIAamp | 4 | Kapa | 7 | 296.55 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-19 | MF-5738 | K2-EDTA | Illumina | Aarhus | QIAamp | 6.4 | Kapa | 7 | 387 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-20 | MF-3793 | K2-EDTA | Illumina | Aarhus | QIAamp | 4 | Kapa | 7 | 438.75 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-21 | MF-4629 | K2-EDTA | Illumina | Aarhus | QIAamp | 8.5 | Kapa | 7 | 526.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-22 | MF-9004 | K2-EDTA | Illumina | Aarhus | QIAamp | 4 | Kapa | 7 | 314.1 |
| | | | Novaseq v1.5 | University | Circulating | | Hyper | | |
| | | | | | Nucleic | | | | |
| | | | | | Acid Kit | | | | |
| | | | | | (qiagen) | | | | |

| | | | | | | | |
|---|---|---|---|---|---|---|---|
| 1.27E+09 | 13.1234 | 33.2 | 175 | 99.97 | 0.0699 | NA | 3.20E+07 |
| 1.38E+09 | 11.5324 | 34.7 | 172 | 99.9 | 0.1024 | 0.014050077 | 3.57E+07 |
| 1.16E+09 | 13.4446 | 28.2 | 174 | 99.9 | 0.0384 | NA | 2.36E+07 |
| 1.32E+09 | 10.4271 | 34 | 173 | 99.97 | 0.0768 | 0.481452062 | 2.51E+07 |
| 1.34E+09 | 13.0409 | 34.3 | 175 | 99.95 | 0.0609 | 0.24349956 | 2.67E+07 |
| 1.35E+09 | 10.2093 | 34.6 | 173 | 99.95 | 0.0513 | −0.370999881 | 2.49E+07 |
| 1.36E+09 | 11.2092 | 35.1 | 173 | 99.97 | 0.0989 | NA | 2.90E+07 |

| | | | | | | | | | |
|---|---|---|---|---|---|---|---|---|---|
| Aar-23 | MF-1203 | K2-EDTA | Illumina | Aarhus | QIAamp | 4 | Kapa | 7 | 394.2 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-24 | MF-1208 | K2-EDTA | Illumina | Aarhus | QIAamp | 8.3 | Kapa | 7 | 612 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-25 | MF-5642 | K2-EDTA | Illumina | Aarhus | QIAamp | 6.4 | Kapa | 7 | 448.65 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-26 | MF-8291 | K2-EDTA | Illumina | Aarhus | QIAamp | 4 | Kapa | 7 | 437.85 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-27 | MF-3108 | K2-EDTA | Illumina | Aarhus | QIAamp | 6.6 | Kapa | 7 | 612 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-28 | MF-1794 | K2-EDTA | Illumina | Aarhus | QIAamp | 4 | Kapa | 7 | 481.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-29 | MF-9921 | K2-EDTA | Illumina | Aarhus | QIAamp | 5 | Kapa | 7 | 428.85 |
| | | | Novaseq v1.5 | University | Circulating | | Hyper | | |
| | | | | | Nucleic | | | | |
| | | | | | Acid Kit | | | | |
| | | | | | (qiagen) | | | | |

| | | | | | | | |
|---|---|---|---|---|---|---|---|
| 1.43E+09 | 14.2244 | 35.9 | 174 | 99.82 | 0.144 | −0.149796661 | 3.05E+07 |
| 1.44E+09 | 13.0955 | 38.4 | 179 | 99.84 | 0.0877 | −1.141486378 | 3.48E+07 |
| 1.26E+09 | 10.2676 | 30.9 | 169 | 99.9 | 0.0669 | NA | 2.13E+07 |
| 1.42E+09 | 10.885 | 35.4 | 170 | 99.9 | 0.1102 | −0.31922606 | 2.22E+07 |
| 1.49E+09 | 12.9797 | 37.8 | 172 | 99.92 | 0.092 | 0.688102736 | 2.91E+07 |
| 1.28E+09 | 12.9989 | 31.2 | 170 | 99.92 | 0.0706 | 0.757215799 | 2.31E+07 |
| 1.39E+09 | 10.3259 | 39.8 | 183 | 99.92 | 0.0823 | NA | 4.27E+07 |

| | | | | | | | | | |
|---|---|---|---|---|---|---|---|---|---|
| Aar-30 | MF-0187 | K2-EDTA | Illumina | Aarhus | QIAamp | 6.5 | Kapa | 7 | 535.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-31 | MF-1673 | K2-EDTA | Illumina | Aarhus | QIAamp | 5 | Kapa | 7 | 428.85 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-32 | MF-1137 | K2-EDTA | Illumina | Aarhus | QIAamp | 7.4 | Kapa | 7 | 499.5 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-33 | MF-1590 | K2-EDTA | Illumina | Aarhus | QIAamp | 4 | Kapa | 7 | 307.8 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-34 | MF-1103 | K2-EDTA | Illumina | Aarhus | QIAamp | 4.1 | Kapa | 7 | 237.15 |
| | | | Novaseq v1.5 | University | Circulating | | Hyper | | |
| | | | | | Nucleic | | | | |
| | | | | | Acid Kit | | | | |
| | | | | | (qiagen) | | | | |
| Aar-35 | MF-1060 | K2-EDTA | Illumina | Aarhus | QIAamp | 14.3 | Kapa | 7 | 508.5 |
| | | | Novaseq v1.5 | University | Circulating | | Hyper | | |
| | | | | | Nucleic | | | | |
| | | | | | Acid Kit | | | | |
| | | | | | (qiagen) | | | | |

| Control | pre | | | | | | | |
|---|---|---|---|---|---|---|---|---|
| Cohort | sequencing | | | | | | sequencing | |
| A | QC | | | | | | metrics | |
| 1.41E+09 | 9.2699 | 38.5 | 175 | 99.84 | 0.0764 | 0.767169258 | 3.04E+07 | Excluded for outlier |
| 1.35E+09 | 13.643 | 33 | 172 | 99.84 | 0.0759 | 1.037978948 | 2.53E+07 | Z score in neutral |
| 1.48E+09 | 14.1786 | 39.7 | 181 | 99.81 | 0.0612 | −0.292702964 | 4.96E+07 | regions (>10) |
| 1.31E+09 | 14.162 | 31.2 | 171 | 99.79 | 0.0831 | 0.861394376 | 2.24E+07 | precluding accurate |
| 1.19E+09 | 10.3645 | 32.1 | 177 | 99.97 | 0.0616 | 0.371275604 | 2.69E+07 | assessment of read |
| 1.37E+09 | 11.2962 | 31.5 | 166 | 99.9 | 0.0739 | 21.23478749 | 6.54E+07 | depth skews |

| | Blood | | | | total | | | library | |
|---|---|---|---|---|---|---|---|---|---|
| | collection | Sequencing | Sequencing | extraction | mass | library | # of PCR | mass | TOTAL_{—} |
| Patient ID | tube | Platform | Location | kit | (ng) | prep kit | cycles | (ng) | READS |
| Control01 | Streck | Illumina | NYGC | Omega | 2.67 | Kapa | 5 | 339.2 | 789858466 |
| | | HiSeq X | | | | Hyper | | | |
| Control03 | Streck | Illumina | NYGC | Omega | 8.25 | Kapa | 5 | 148.86 | 836157356 |
| | | HiSeq X | | | | Hyper | | | |
| Control04 | Streck | Illumina | NYGC | Omega | 9.6 | Kapa | 5 | 224.88 | 946275796 |
| | | HiSeq X | | | | Hyper | | | |
| Control05 | Streck | Illumina | NYGC | Omega | 4.86 | Kapa | 5 | 144.9 | 782434050 |
| | | HiSeq X | | | | Hyper | | | |
| Control06 | Streck | Illumina | NYGC | Omega | 17.83 | Kapa | 5 | N/A | 911087416 |
| | | HiSeq X | | | | Hyper | | | |
| Control07 | Streck | Illumina | NYGC | Omega | 22.68 | Kapa | 5 | 137.267 | 733283062 |
| | | HiSeq X | | | | Hyper | | | |
| Control08 | Streck | Illumina | NYGC | Omega | 15.96 | Kapa | 5 | 91.4588 | 751392866 |
| | | HiSeq X | | | | Hyper | | | |
| Control09 | Streck | Illumina | NYGC | Omega | 34.8 | Kapa | 5 | 239.752 | 826103658 |
| | | HiSeq X | | | | Hyper | | | |
| Control10 | Streck | Illumina | NYGC | Omega | 7.5 | NEXTflex | 5 | N/A | 920821992 |
| | | HiSeq X | | | | | | | |
| Control11 | Streck | Illumina | NYGC | Omega | 35.4 | Kapa | 5 | 227.421 | 860581576 |
| | | HiSeq X | | | | Hyper | | | |
| Control12 | Streck | Illumina | NYGC | Omega | 24.06 | Kapa | 5 | 218.108 | 692806584 |
| | | HiSeq X | | | | Hyper | | | |
| Control13 | Streck | Illumina | NYGC | Omega | 33.9 | Kapa | 5 | 181.984 | 853441796 |
| | | HiSeq X | | | | Hyper | | | |
| Control15 | Streck | Illumina | NYGC | Omega | 24.6 | Kapa | 5 | 181.2 | 713152810 |
| | | HiSeq X | | | | Hyper | | | |
| Control16 | Streck | Illumina | NYGC | Omega | 105 | Kapa | 5 | 302.73 | 893704580 |
| | | HiSeq X | | | | Hyper | | | |
| Control17 | Streck | Illumina | NYGC | Omega | 17.28 | Kapa | 5 | 169.202 | 870655114 |
| | | HiSeq X | | | | Hyper | | | |
| Control19 | Streck | Illumina | NYGC | Omega | 46.5 | Kapa | 5 | 263.384 | 822871044 |
| | | HiSeq X | | | | Hyper | | | |
| Control20 | Streck | Illumina | NYGC | Omega | 30.3 | Kapa | 5 | 329.883 | 780113986 |
| | | HiSeq X | | | | Hyper | | | |

| Percent- | Mean | MEDIAN_{—} | | |
|---|---|---|---|---|
| Total- | Coverage | INSERT_{—} | Auto- | |
| Duplication | (X) | SIZE (bp) | correlation | Notes |
| 0.113779 | 23.135787 | 175 | 0.04588902 | |
| 0.123332 | 23.963951 | 175 | 0.06907927 | |
| 0.142511 | 26.336945 | 174 | 0.05296935 | |
| 0.133918 | 23.064915 | 178 | 0.1037549 | |
| 0.1098 | 29.341163 | 174 | 0.07388784 | |
| 0.088995 | 23.25369 | 179 | 0.06084299 | |
| 0.110687 | 21.221917 | 170 | 0.0278342 | |
| 0.100672 | 25.25728 | 174 | 0.04074561 | |
| 0.6477 | 11.809975 | 188 | 0.04099832 | |
| 0.108264 | 26.236777 | 177 | 0.04639367 | |
| 0.112351 | 19.0633 | 176 | 0.05102338 | |
| 0.097603 | 25.394404 | 174 | 0.04790997 | |
| 0.097073 | 21.091066 | 174 | 0.03887713 | |
| 0.090932 | 28.342527 | 176 | 0.03887713 | |
| 0.114987 | 26.124183 | 175 | 0.06488159 | |
| 0.092606 | 24.690559 | 171 | 0.04774423 | |
| 0.097284 | 23.708725 | 175 | 0.04627138 | |

| | | | | | | | | | |
|---|---|---|---|---|---|---|---|---|---|
| Control22 | Streck | Illumina | NYGC | Omega | 16.56 | Kapa | 5 | 181.847 | 873962842 |
| | | HiSeq X | | | | Hyper | | | |
| Control23 | Streck | Illumina | NYGC | Omega | 23.94 | Kapa | 5 | 155.583 | 913465942 |
| | | HiSeq X | | | | Hyper | | | |
| Control24 | Streck | Illumina | NYGC | Omega | 25.29 | Kapa | 5 | 173.809 | 862630112 |
| | | HiSeq X | | | | Hyper | | | |
| Control25 | Streck | Illumina | NYGC | Omega | 42.9 | Kapa | 5 | 286.941 | 872314532 |
| | | HiSeq X | | | | Hyper | | | |
| Control26 | Streck | Illumina | NYGC | Omega | 29.7 | Kapa | 5 | 155.681 | 729628840 |
| | | HiSeq X | | | | Hyper | | | |
| Control27 | Streck | Illumina | NYGC | Omega | 22.86 | Kapa | 5 | 147.944 | 891804778 |
| | | HiSeq X | | | | Hyper | | | |
| Control28 | Streck | Illumina | NYGC | Omega | 18.66 | Kapa | 5 | 136.387 | 667038560 |
| | | HiSeq X | | | | Hyper | | | |
| Control29 | Streck | Illumina | NYGC | Omega | 28.77 | Kapa | 5 | 143.104 | 766733204 |
| | | HiSeq X | | | | Hyper | | | |
| Control30 | Streck | Illumina | NYGC | Omega | 357 | Kapa | 5 | 148.241 | 849408178 |
| | | HiSeq X | | | | Hyper | | | |
| Control31 | Streck | Illumina | NYGC | Omega | 8.73 | NEXTflex | 5 | N/A | 871172416 |
| | | HiSeq X | | | | | | | |
| Control32 | Streck | Illumina | NYGC | Omega | 9.27 | Kapa | 5 | 184.2 | 919023222 |
| | | HiSeq X | | | | Hyper | | | |
| Control33 | Streck | Illumina | NYGC | Omega | 10.1 | NEXTflex | 5 | N/A | 881910872 |
| | | HiSeq X | | | | | | | |
| Control34 | Streck | Illumina | NYGC | Omega | 9.78 | Kapa | 5 | N/A | 775111974 |
| | | HiSeq X | | | | Hyper | | | |
| Control35 | Streck | Illumina | NYGC | Omega | 22.62 | Kapa | 5 | 148.377 | 903019548 |
| | | HiSeq X | | | | Hyper | | | |
| Control36 | Streck | Illumina | NYGC | Omega | 23.7 | Kapa | 5 | 170.25 | 861184834 |
| | | HiSeq X | | | | Hyper | | | |
| Control37 | Streck | Illumina | NYGC | Omega | 75.6 | Kapa | 5 | 347.222 | 876738398 |
| | | HiSeq X | | | | Hyper | | | |
| Control38 | Streck | Illumina | NYGC | Omega | 41.7 | Kapa | 5 | 217.087 | 868327440 |
| | | HiSeq X | | | | Hyper | | | |

* previously reported in Zviran et al. Nature Med 2020

Control Cohort

| | | | |
|---|---|---|---|
| 0.111613 | 26.134683 | 175 | 0.09049992 |
| 0.12389 | 26.212227 | 173 | 0.04783184 |
| 0.121396 | 25.131907 | 174 | 0.04638046 |
| 0.100399 | 25.91248 | 174 | 0.03494828 |
| 0.077642 | 22.645333 | 175 | 0.05137754 |
| 0.120816 | 26.156054 | 173 | 0.07192883 |
| 0.079548 | 20.433893 | 176 | 0.0623225 |
| 0.084346 | 23.392275 | 175 | 0.03957313 |
| 0.09028 | 24.291075 | 171 | 0.05209242 |
| 0.5203 | 14.405062 | 182 | 0.09381432 |
| 10.7905 | 27.79237 | 179 | 0.0468411 |
| 0.408031 | 18.185102 | 183 | 0.04147149 |
| 0.0932 | 22.131727 | 173 | 0.04077665 |
| 0.123213 | 26.312031 | 175 | 0.05235295 |
| 0.113308 | 25.601384 | 173 | 0.05328715 |
| 0.095074 | 26.969984 | 176 | 0.04773302 |
| 0.106999 | 26.786833 | 178 | 0.05639913 |

| | pre sequencing | | | | | | | | sequencing |
|---|---|---|---|---|---|---|---|---|---|
| Control | QC Blood | | | | total | | | library | metrics |
| Plasma | Collection | Sequencing | Sequencing | extraction | mass | library | # of PCR | mass | TOTAL_{—} |
| Patient ID | Tube | Platform | Location | kit | (ng) | prep kit | cycles | (ng) | READS |
| Donor333 | K2-EDTA | Illumina | Aarhus | QIAsymphony | 3.2 | Kapa | 7 | 212.85 | 1055238088 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor334 | K2-EDTA | Illumina | Aarhus | QIAsymphony | 7.6 | Kapa | 7 | 298.8 | 945885816 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor335 | K2-EDTA | Illumina | Aarhus | QIAsymphony | 3.3 | Kapa | 7 | 139.95 | 1025395882 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor336 | K2-EDTA | Illumina | Aarhus | QIAsymphony | 4.7 | Kapa | 7 | 241.2 | 1051344276 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor337 | K2-EDTA | Illumina | Aarhus | QIAsymphony | 6.1 | Kapa | 7 | 341.1 | 938285944 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor338 | K2-EDTA | Illumina | Aarhus | QIAsymphony | 6.7 | Kapa | 7 | 292.5 | 942363812 |
| | | Novaseq | University | DSP | | Hyper | | | |
| | | v1.5 | | Circulating | | | | | |
| | | | | DNA Kit | | | | | |
| | | | | (Qiagen) | | | | | |

| Percent- | Mean | MEDIAN_{—} | SNV | |
|---|---|---|---|---|
| Total- | Coverage | INSERT_{—} | Pileup | |
| Duplication | (X) | SIZE (bp) | size | Notes |
| 10.418 | 26.67034 | 170 | 2.60E+07 | |
| 10.019 | 24.887184 | 172 | 2.47E+07 | |
| 11.157 | 26.834555 | 174 | 2.54E+07 | |
| 10.1577 | 27.838124 | 172 | 2.64E+07 | |
| 10.2302 | 23.806223 | 168 | 2.31E+07 | |
| 9.063 | 26.201163 | 176 | 3.32E+07 | |

| | | | | | | | | | |
|---|---|---|---|---|---|---|---|---|---|
| Donor340 | K2-EDTA | Illumina | Aarhus | QIAsymphony | 9.4 | Kapa | 7 | 463.5 | 1019441576 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor343 | K2-EDTA | Illumina | Aarhus | QIAsymphony | 1.6 | Kapa | 7 | 88.2 | 988200396 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor344 | K2-EDTA | Illumina | Aarhus | QIAsymphony | 9.5 | Kapa | 7 | 396.9 | 1133884122 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor347 | K2-EDTA | Illumina | Aarhus | QIAsymphony | 15.6 | Kapa | 7 | 625.5 | 1056969754 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor349 | K2-EDTA | Illumina | Aarhus | QIAsymphony | 4.7 | Kapa | 7 | 271.8 | 1005145492 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor352 | K2-EDTA | Illumina | Aarhus | QIAsymphony | 14.5 | Kapa | 7 | 679.5 | 1076414482 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor353 | K2-EDTA | Illumina | Aarhus | QIAsymphony | 4.4 | Kapa | 7 | 229.95 | 1134415310 |
| | | Novaseq | University | DSP | | Hyper | | | |
| | | v1.5 | | Circulating | | | | | |
| | | | | DNA Kit | | | | | |
| | | | | (Qiagen) | | | | | |

| | | | |
|---|---|---|---|
| 9.8713 | 27.0313 | 172 | 2.55E+07 |
| 12.7535 | 24.123778 | 172 | 2.49E+07 |
| 10.4546 | 29.364167 | 170 | 2.85E+07 |
| 9.7928 | 29.019624 | 175 | 2.97E+07 |
| 10.1568 | 26.069127 | 171 | 2.49E+07 |
| 9.4759 | 29.876747 | 176 | 3.05E+07 |
| 10.619 | 28.97638 | 170 | 2.83E+07 |

| | | | | | | | | | |
|---|---|---|---|---|---|---|---|---|---|
| Donor356 | K2-EDTA | Illumina | Aarhus | QIAsymphony | 4.1 | Kapa | 7 | 167.85 | 942707130 |
| | | Novaseq | University | DSP | | Hyper | | | |
| | | v1.5 | | Circulating | | | | | |
| | | | | DNA Kit | | | | | |
| | | | | (Qiagen) | | | | | |
| Donor358 | K2-EDTA | Illumina | Aarhus | QIAsymphony | 7 | Kapa | 7 | 318.6 | 1011141704 |
| | | Novaseq | University | DSP | | Hyper | | | |
| | | v1.5 | | Circulating | | | | | |
| | | | | DNA Kit | | | | | |
| | | | | (Qiagen) | | | | | |

| | pre sequencing | | | | | | | sequencing | |
|---|---|---|---|---|---|---|---|---|---|
| Control Cohort | QC Blood | | | | total | | | metrics | |
| C | Collection | Sequencing | Sequencing | extraction | mass | library | # of PCR | library | TOTAL_{—} |
| Patient ID | Tube | Platform | Location | kit | (ng) | prep kit | cycles | mass (ng) | READS |
| C-01 | Streck | Illumina | NYGC | Omega | 4.76 | Kappa | 6 | 4.7642 | 922059462 |
| | | NovaSeq | | | | Hyper | | | |
| | | v1.0 | | | | | | | |
| C-04 | Streck | Illumina | NYGC | Omega | 7.42 | Kappa | 6 | 7.4168 | 1102393506 |
| | | NovaSeq | | | | Hyper | | | |
| | | v1.0 | | | | | | | |
| C-05 | Streck | Illumina | NYGC | Omega | 8.17 | Kappa | 6 | 8.174 | 1209658046 |
| | | NovaSeq | | | | Hyper | | | |
| | | v1.0 | | | | | | | |
| C-06 | Streck | Illumina | NYGC | Omega | 10.99 | Kappa | 6 | 10.989 | 1178536778 |
| | | NovaSeq | | | | Hyper | | | |
| | | v1.0 | | | | | | | |
| C-07 | Streck | Illumina | NYGC | Omega | 12.82 | Kappa | 6 | 12.818 | 1130556838 |
| | | NovaSeq | | | | Hyper | | | |
| | | v1.0 | | | | | | | |
| C-08 | Streck | Illumina | NYGC | Omega | 15.64 | Kappa | 6 | 15.6354 | 1101872290 |
| | | NovaSeq | | | | Hyper | | | |
| | | v1.0 | | | | | | | |

| | | | |
|---|---|---|---|
| 10.2555 | 24.913741 | 173 | 2.38E+07 |
| 9.616 | 27.022832 | 173 | 2.54E+07 |

| Percent- | Mean | MEDIAN_{—} | | |
|---|---|---|---|---|
| Total- | Coverage | INSERT_{—} | Auto- | |
| Duplication | (X) | SIZE (bp) | correlation | Notes |
| 8.6764 | 28.7285 | 177 | 0.1038907 | |
| 8.1829 | 33.6893 | 176 | 0.1139046 | |
| 7.7255 | 38.368 | 178 | 0.0708321 | |
| 7.6222 | 38.4232 | 180 | 0.0680637 | |
| 8.4303 | 37.0486 | 180 | 0.05449893 | |
| 6.3741 | 35.5614 | 178 | 0.07560779 | |

| | | | | | | | | | |
|---|---|---|---|---|---|---|---|---|---|
| C-09 | Streck | Illumina | NYGC | Omega | 13.22 | Kappa | 6 | 13.2158 | 1022316208 |
| | | NovaSeq | | | | Hyper | | | |
| | | v1.0 | | | | | | | |
| C-10 | Streck | Illumina | NYGC | Omega | 16.27 | Kappa | 6 | 16.2708 | 951311180 |
| | | NovaSeq | | | | Hyper | | | |
| | | v1.0 | | | | | | | |
| C-11 | Streck | Illumina | NYGC | Omega | 16.63 | Kappa | 6 | 16.632 | 982378280 |
| | | NovaSeq | | | | Hyper | | | |
| | | v1.0 | | | | | | | |
| C-12 | Streck | Illumina | NYGC | Omega | 14.44 | Kappa | 6 | 14.4356 | 935689726 |
| | | NovaSeq | | | | Hyper | | | |
| | | v1.0 | | | | | | | |
| C-13 | Streck | Illumina | NYGC | Omega | 9.06 | Kappa | 6 | 9.06 | 1229905924 |
| | | NovaSeq | | | | Hyper | | | |
| | | v1.0 | | | | | | | |
| C-14 | Streck | Illumina | NYGC | Omega | 10.38 | Kappa | 6 | 10.38 | 1052951874 |
| | | NovaSeq | | | | Hyper | | | |
| | | v1.0 | | | | | | | |
| C-15 | Streck | Illumina | NYGC | Omega | 12.3 | Kappa | 6 | 12.3004 | 993926260 |
| | | NovaSeq | | | | Hyper | | | |
| | | v1.0 | | | | | | | |
| C-16 | Streck | Illumina | NYGC | Omega | 17.39 | Kappa | 6 | 17.388 | 1074956094 |
| | | NovaSeq | | | | Hyper | | | |
| | | v1.0 | | | | | | | |
| C-17 | Streck | Illumina | NYGC | Omega | 6.43 | Kappa | 6 | 6.4272 | 1218995288 |
| | | NovaSeq | | | | Hyper | | | |
| | | v1.0 | | | | | | | |
| C-19 | Streck | Illumina | NYGC | Omega | 7.62 | Kappa | 6 | 7.623 | 1021658660 |
| | | NovaSeq | | | | Hyper | | | |
| | | v1.0 | | | | | | | |
| C-20 | Streck | Illumina | NYGC | Omega | 16.86 | Kappa | 6 | 16.8636 | 1214994704 |
| | | NovaSeq | | | | Hyper | | | |
| | | v1.0 | | | | | | | |
| C-21 | Streck | Illumina | NYGC | Omega | 13.62 | Kappa | 6 | 13.623 | 946722852 |
| | | NovaSeq | | | | Hyper | | | |
| | | v1.0 | | | | | | | |

| | | | |
|---|---|---|---|
| 6.4173 | 32.6349 | 178 | 0.05851543 |
| 5.9573 | 30.5444 | 177 | 0.05181143 |
| 6.4856 | 29.7336 | 171 | 0.08211027 |
| 5.8288 | 29.3147 | 175 | 0.07551185 |
| 7.9974 | 40.0081 | 184 | 0.06455295 |
| 7.1461 | 31.4391 | 171 | 0.03642748 |
| 6.0119 | 30.6205 | 174 | 0.06478629 |
| 6.3575 | 33.0915 | 174 | 0.04730562 |
| 8.3942 | 37.1319 | 174 | 0.05852952 |
| 7.2874 | 31.9992 | 176 | 0.09202064 |
| 7.3635 | 40.7199 | 180 | 0.06002903 |
| 5.9606 | 29.5034 | 176 | 0.05371299 |

| | | | | | | | | | |
|---|---|---|---|---|---|---|---|---|---|
| C-22 | Streck | Illumina | NYGC | Omega | 4.28 | Kappa | 6 | 4.284 | 938851134 |
| | | NovaSeq | | | | Hyper | | | |
| | | v1.0 | | | | | | | |
| C-23 | Streck | Illumina | NYGC | Omega | 18.01 | Kappa | 6 | 18.0056 | 1058310564 |
| | | NovaSeq | | | | Hyper | | | |
| | | v1.0 | | | | | | | |
| C-24 | Streck | Illumina | NYGC | Omega | 15.28 | Kappa | 6 | 15.2796 | 1073714324 |
| | | NovaSeq | | | | Hyper | | | |
| | | v1.0 | | | | | | | |
| C-25 | Streck | Illumina | NYGC | Omega | 7.7 | Kappa | 6 | 7.6956 | 987554180 |
| | | NovaSeq | | | | Hyper | | | |
| | | v1.0 | | | | | | | |
| C-26 | Streck | Illumina | NYGC | Omega | 7.59 | Kappa | 6 | 7.5922 | 948690090 |
| | | NovaSeq | | | | Hyper | | | |
| | | v1.0 | | | | | | | |
| C-27 | Streck | Illumina | NYGC | Omega | 13.17 | Kappa | 6 | 13.166 | 1099874300 |
| | | NovaSeq | | | | Hyper | | | |
| | | v1.0 | | | | | | | |
| C-28 | Streck | Illumina | NYGC | Omega | 8.01 | Kappa | 6 | 8.0064 | 963780660 |
| | | NovaSeq | | | | Hyper | | | |
| | | v1.0 | | | | | | | |
| C-29 | Streck | Illumina | NYGC | Omega | 21.38 | Kappa | 6 | 21.3756 | 841661356 |
| | | NovaSeq | | | | Hyper | | | |
| | | v1.0 | | | | | | | |
| C-30 | Streck | Illumina | NYGC | Omega | 13.35 | Kappa | 6 | 13.348 | 1016381116 |
| | | NovaSeq | | | | Hyper | | | |
| | | v1.0 | | | | | | | |
| C-31 | Streck | Illumina | NYGC | Omega | 10.96 | Kappa | 6 | 10.962 | 964852616 |
| | | NovaSeq | | | | Hyper | | | |
| | | v1.0 | | | | | | | |
| C-32 | Streck | Illumina | NYGC | Omega | 14.51 | Kappa | 6 | 14.508 | 1033556406 |
| | | NovaSeq | | | | Hyper | | | |
| | | v1.0 | | | | | | | |
| C-33 | Streck | Illumina | NYGC | Omega | 11.25 | Kappa | 6 | 11.2464 | 905660482 |
| | | NovaSeq | | | | Hyper | | | |
| | | v1.0 | | | | | | | |

| | | | |
|---|---|---|---|
| 8.1227 | 28.9967 | 176 | 0.07415747 |
| 6.4141 | 33.9312 | 175 | 0.07792659 |
| 6.5198 | 32.9524 | 174 | 0.05161793 |
| 6.961 | 29.3778 | 170 | 0.06624456 |
| 7.4432 | 29.4938 | 175 | 0.06465402 |
| 10.0881 | 33.5646 | 174 | 0.06645676 |
| 11.3373 | 28.8334 | 174 | 0.05690012 |
| 8.0351 | 25.3575 | 174 | 0.0272583 |
| 12.556 | 29.1199 | 172 | 0.04942987 |
| 11.2795 | 28.2066 | 173 | 0.01852411 |
| 7.5369 | 32.4343 | 174 | 0.0428256 |
| 9.0005 | 28.6933 | 177 | 0.02999187 |

| | | | | | | | | | |
|---|---|---|---|---|---|---|---|---|---|
| C-34 | Streck | Illumina | NYGC | Omega | 8.09 | Kappa | 6 | 8.0934 | 1042291534 |
| | | NovaSeq | | | | Hyper | | | |
| | | v1.0 | | | | | | | |
| C-35 | Streck | Illumina | NYGC | Omega | 7.98 | Kappa | 6 | 7.98 | 1027810848 |
| | | NovaSeq | | | | Hyper | | | |
| | | v1.0 | | | | | | | |
| C-36 | Streck | Illumina | NYGC | Omega | 13.8 | Kappa | 6 | 13.8 | 954941718 |
| | | NovaSeq | | | | Hyper | | | |
| | | v1.0 | | | | | | | |
| C-37 | Streck | Illumina | NYGC | Omega | 32.51 | Kappa | 6 | 25 | 1215284372 |
| | | NovaSeq | | | | Hyper | | | |
| | | v1.0 | | | | | | | |
| C-38 | Streck | Illumina | NYGC | Omega | 13.79 | Kappa | 6 | 13.786 | 1216562382 |
| | | NovaSeq | | | | Hyper | | | |
| | | v1.0 | | | | | | | |

*library mass capped at 25 ng

| | | | | | | | | somatic | |
|---|---|---|---|---|---|---|---|---|---|
| | | TOTAL_{—} | | | MEDIAN_{—} | QC | | pipeline | |
| Early-stage | | READS | Percent- | Mean | INSERT_{—} | metrics | | # of | |
| CRC Tumor | | TOTAL_{—} | Total- | Coverage | SIZE | Conpair- | auto- | mutation | # of |
| Patient ID | Tissue Type | READS | Duplication | (X) | (bp) | Concordance | correlation | detected | amplification |
| CRC 1 | Fresh frozen | 1.066E+09 | 0.072005 | 48.384276 | 450 | 99.67% | 0.00399 | 11613 | 3 |
| CRC 2 | Fresh frozen | 1.098E+09 | 0.078152 | 49.112644 | 453 | 91.05% | 0.0076 | 1936 | 2 |
| CRC 3 | Fresh frozen | 1.085E+09 | 0.076353 | 49.112658 | 429 | 99.91% | 0.00799 | 9939 | 24 |
| CRC 4 | Fresh frozen | 777913144 | 0.068288 | 35.416132 | 458 | 99.85% | 0.1194 | 38706 | 0 |
| CRC 5 | Fresh frozen | 1.25E+09 | 0.076341 | 56.479102 | 457 | 99.95% | 0.08735 | 61250 | 0 |
| CRC 6 | Fresh frozen | 1.067E+09 | 0.070632 | 48.589753 | 451 | 99.81% | 0.01582 | 15057 | 16 |
| CRC 7 | Fresh frozen | 1.563E+09 | 0.101054 | 68.724108 | 454 | 99.85% | 0.03576 | 7709 | 21 |
| CRC 8 | Fresh frozen | 1.023E+09 | 0.073312 | 46.092847 | 455 | 94.88% | 0.02624 | 62453 | 0 |
| CRC 9 | Fresh frozen | 1.113E+09 | 0.078663 | 50.215438 | 447 | 95.32% | 0.01034 | 9162 | 31 |
| CRC 10 | Fresh frozen | 1.553E+09 | 0.077057 | 70.311893 | 452 | 99.67% | 0.00197 | 14491 | 0 |
| CRC 11 | Fresh frozen | 1.004E+09 | 0.064881 | 45.561452 | 462 | 99.85% | 0.02225 | 104739 | 1 |

| | | | |
|---|---|---|---|
| 8.0264 | 34.6231 | 186 | 0.1064437 |
| 7.6709 | 32.7854 | 178 | 0.07839858 |
| 6.5126 | 29.8248 | 175 | 0.09220944 |
| 6.7572 | 38.1131 | 176 | 0.07587976 |
| 8.4654 | 38.523 | 178 | 0.07136225 |

| | | | | Total Mbp of | | |
|---|---|---|---|---|---|---|
| # of | # of copy- | Total Mbp of | Total Mbp of | copy-neutral | Tumor purity | |
| deletion | neutral LOH | amplification | deletion | LOH | (%) | Notes |
| 0 | 0 | 321.711 | 0 | 0 | 22 | |
| 1 | 0 | 101.221 | 18.273 | 0 | 20 | |
| 17 | 1 | 318.029 | 198.038 | 145.25 | 80 | |
| 0 | 2 | 0 | 0 | 77.4937 | 55 | |
| 0 | 2 | 0 | 0 | 64.5936 | 76 | |
| 12 | 2 | 569.357 | 833.09 | 12.0912 | 57 | |
| 20 | 2 | 263.485 | 698.667 | 159.619 | 41 | |
| 0 | 1 | 0 | 0 | 46.5079 | 79 | |
| 21 | 1 | 572.69 | 1141.9 | 10.0013 | 29 | |
| 2 | 2 | 0 | 56.4288 | 175.778 | 29 | |
| 1 | 0 | 96.7008 | 16.7948 | 0 | 49 | |

| | | | | | | | | | |
|---|---|---|---|---|---|---|---|---|---|
| CRC 12 | Fresh frozen | 866293756 | 0.069236 | 39.30034 | 445 | 99.66% | 0.01149 | 11701 | 7 |
| CRC 13 | Fresh frozen | 949509186 | 0.069208 | 43.115049 | 456 | 97.95% | 0.01891 | 68962 | 2 |
| CRC 14 | Fresh frozen | 1.217E+09 | 0.083377 | 54.411571 | 450 | 99.74% | 0.09291 | 12933 | 23 |
| CRC 15 | Fresh frozen | 717233616 | 0.070963 | 32.585856 | 449 | 99.97% | 0.00095 | 11188 | 6 |
| CRC 16 | Fresh frozen | 839430354 | 0.074652 | 38.08649 | 451 | 99.88% | 0.09191 | 8530 | 12 |
| CRC 17 | Fresh frozen | 1.521E+09 | 0.080502 | 67.887396 | 459 | 99.93% | 0.05152 | 6764 | 88 |
| CRC 18 | Fresh frozen | 1.624E+09 | 0.099871 | 72.36846 | 449 | 99.84% | 0.14935 | 56901 | NA |
| CRC 19 | Fresh frozen | 1.247E+09 | 0.073336 | 56.347868 | 456 | 99.58% | 0.01715 | 4610 | 50 |

* previously reported in Zviran et al. Nature Med 2020

| | sequencing | | | | | | |
|---|---|---|---|---|---|---|---|
| | metrics | | | | | | |
| | | | | MEDIAN_{—} | | | |
| Early-stage | | Percent- | Mean | INSERT_{—} | QC metrics | | |
| CRC | TOTAL_{—} | Total- | Coverage | SIZE | Conpair- | auto- | |
| Patient ID | READS | Duplication | (X) | (bp) | Concordance | correlation | Notes |

| | | | | | | |
|---|---|---|---|---|---|---|
| CRC 1 | 842875042 | 0.163474 | 34.485292 | 448 | 99.67% | 0.00014 |
| CRC 2 | 770625096 | 0.164131 | 31.32474 | 453 | 91.05% | −0.00582 |
| CRC 3 | 835980264 | 0.162592 | 34.229731 | 455 | 99.91% | 0.01233 |
| CRC 4 | 989864866 | 0.182899 | 39.461498 | 450 | 99.85% | −0.00227 |
| CRC 5 | 800221540 | 0.159414 | 32.928302 | 455 | 99.95% | −0.0027 |
| CRC 6 | 817148940 | 0.163121 | 33.22916 | 453 | 99.81% | 0.00213 |
| CRC 7 | 1036040912 | 0.179748 | 41.274797 | 455 | 99.85% | 0.02604 |
| CRC 8 | 855196922 | 0.165892 | 34.740464 | 451 | 94.88% | −0.00266 |
| CRC 9 | 888626860 | 0.161885 | 36.365502 | 455 | 95.32% | −0.00166 |
| CRC 10 | 1065354177 | 0.200282 | 41.877122 | 451 | 99.67% | 0.00187 |
| CRC 11 | 865361110 | 0.17091 | 34.788932 | 446 | 99.85% | −0.00217 |
| CRC 12 | 1158513040 | 0.192514 | 45.590242 | 454 | 99.66% | 0.00959 |
| CRC 13 | 889371398 | 0.159293 | 36.401821 | 452 | 97.95% | 0.02527 |
| CRC 14 | 897680692 | 0.160375 | 36.633372 | 454 | 99.74% | −0.00304 |
| CRC 15 | 831930266 | 0.16029 | 34.106774 | 439 | 99.97% | 0.00324 |
| CRC 16 | 976580712 | 0.168063 | 39.681972 | 449 | 99.88% | 0.00235 |
| CRC 17 | 823285970 | 0.177327 | 33.03315 | 443 | 99.93% | −0.00474 |
| CRC 18 | 937909678 | 0.166471 | 38.377594 | 441 | 99.84% | 0.04523 |
| CRC 19 | 874435004 | 0.165825 | 35.40181 | 456 | 99.58% | −0.00183 |

* previously reported in Zviran et al. Nature Med 2020

| | | | | | |
|---|---|---|---|---|---|
| 7 | 3 | 187.477 | 546.445 | 126.691 | 93 |
| 0 | 0 | 30.5964 | 0 | 0 | 34 |
| 16 | 0 | 1306.23 | 1000.78 | 0 | 75 |
| 4 | 0 | 303.186 | 223.622 | 0 | 53 |
| 9 | 4 | 696.833 | 822.868 | 262.007 | 66 |
| 14 | 2 | 879.426 | 477.834 | 115.538 | 29 |
| NA | NA | NA | NA | NA | NA |
| 72 | 1 | 552.374 | 1211.63 | 242.173 | 17 |

| | | pre | | | | | | | |
|---|---|---|---|---|---|---|---|---|---|
| | | sequencing | | | | | | | |
| | | QC | | | | | | | sequencing |
| Early-stage | | Blood | | | | total | | | metrics |
| CRC Plasma | | collection | Sequencing | Sequencing | extraction | mass | library | # of PCR | library mass |
| Patient ID | Timepoint | tube | Platform | Location | kit | (ng) | prep kit | cycles | (ng) |
| CRC 1 | preoperative | Streck | Illumina | NYGC | Omega | 12 | Kapa | 5 | 198.6328181 |
| | | | HiSeq X | | | | Hyper | | |
| CRC 2 | preoperative | Streck | Illumina | NYGC | Omega | 16.38 | Kapa | 5 | 261.0481692 |
| | | | HiSeq X | | | | Hyper | | |
| CRC 3 | preoperative | Streck | Illumina | NYGC | Omega | 11.7 | Kapa | 5 | 431.6457264 |
| | | | HiSeq X | | | | Hyper | | |
| CRC 4 | preoperative | Streck | Illumina | NYGC | Omega | 17.67 | Kapa | 5 | 217.7226894 |
| | | | HiSeq X | | | | Hyper | | |
| CRC 5 | preoperative | Streck | Illumina | NYGC | Omega | 12.57 | Kapa | 5 | 190.3596467 |
| | | | HiSeq X | | | | Hyper | | |
| CRC 6 | preoperative | Streck | Illumina | NYGC | Omega | 9.33 | Kapa | 5 | 236.2813032 |
| | | | HiSeq X | | | | Hyper | | |
| CRC 7 | preoperative | Streck | Illumina | NYGC | Omega | 96.9 | Kapa | 5 | 130.5764539 |
| | | | HiSeq X | | | | Hyper | | |
| CRC 8 | preoperative | Streck | Illumina | NYGC | Omega | 6.57 | Kapa | 5 | 153.5799577 |
| | | | HiSeq X | | | | Hyper | | |
| CRC 9 | preoperative | Streck | Illumina | NYGC | Omega | 9.93 | Kapa | 5 | 179.6609771 |
| | | | HiSeq X | | | | Hyper | | |
| CRC 10 | preoperative | Streck | Illumina | NYGC | Omega | 28.32 | Kapa | 5 | 224.5264433 |
| | | | HiSeq X | | | | Hyper | | |
| CRC 11 | preoperative | Streck | Illumina | NYGC | Omega | 16.83 | Kapa | 5 | 176.0144036 |
| | | | HiSeq X | | | | Hyper | | |
| CRC 12 | preoperative | Streck | Illumina | NYGC | Omega | 8.3 | Kapa | 5 | 162.1668439 |
| | | | HiSeq X | | | | Hyper | | |
| CRC 13 | preoperative | Streck | Illumina | NYGC | Omega | 51 | Kapa | 5 | 104.110562 |
| | | | HiSeq X | | | | Hyper | | |
| CRC 14 | preoperative | Streck | Illumina | NYGC | Omega | 23.43 | Kapa | 5 | 192.7569954 |
| | | | HiSeq X | | | | Hyper | | |

| | | | QC metrics | | | | |
|---|---|---|---|---|---|---|---|
| | | | MEDIAN_{—} | | | | |
| | Percent- | Mean | INSERT_{—} | | | Neutral | |
| TOTAL_{—} | Total- | Coverage | SIZE | Conpair- | Auto- | Regions | |
| READS | Duplication | (X) | (bp) | Concordance | correlation | Z Score | Notes |
| 8.17E+08 | 0.095973 | 24.618508 | 175 | 99.71% | 0.08586144 | −0.038247 | |
| 9.09E+08 | 0.111401 | 25.796083 | 170 | 99.71% | 0.05998804 | −0.55102 | |
| 1.05E+09 | 0.113597 | 30.873489 | 172 | 99.05% | 0.03990826 | 2.938799 | |
| 7.98E+08 | 0.104203 | 23.02172 | 171 | 99.74% | 0.0855362 | 0.404263 | |
| 9.45E+08 | 0.124307 | 26.586612 | 170 | 99.77% | 0.1202944 | −1.197272 | |
| 9.09E+08 | 0.117628 | 24.681857 | 169 | 99.74% | 0.06522213 | 1.318337 | |
| 7.25E+08 | 0.108039 | 20.027975 | 170 | 99.58% | 0.05939493 | −0.741147 | |
| 8.21E+08 | 0.124358 | 22.876972 | 170 | 99.69% | 0.04045408 | −0.383288 | |
| 9.41E+08 | 0.119415 | 25.378658 | 169 | 99.67% | 0.0515381 | 2.2668 | |
| 7.8E+08 | 0.11136 | 21.895186 | 170 | 99.60% | 0.06138345 | −0.446054 | |
| 9.4E+08 | 0.107023 | 26.865914 | 171 | 99.66% | 0.05957135 | −1.97303 | |
| 7.69E+08 | 0.116378 | 20.860396 | 168 | 99.66% | 0.05382121 | 0.228965 | |
| 7.56E+08 | 0.117542 | 21.220254 | 172 | 99.52% | 0.06432957 | 1.409572 | |
| 8.64E+08 | 0.10241 | 24.872 | 171 | 99.66% | 0.05613317 | −1.556646 | |

| | | | | | | | | | |
|---|---|---|---|---|---|---|---|---|---|
| CRC 15 | preoperative | Streck | Illumina | NYGC | Omega | 65.4 | Kapa | 5 | 226.2300386 |
| | | | HiSeq X | | | | Hyper | | |
| CRC 16 | preoperative | Streck | Illumina | NYGC | Omega | 90 | Kapa | 5 | 359.8693037 |
| | | | HiSeq X | | | | Hyper | | |
| CRC 17 | preoperative | Streck | Illumina | NYGC | Omega | 7.38 | Kapa | 5 | 168.1232677 |
| | | | HiSeq X | | | | Hyper | | |
| CRC 18 | preoperative | Streck | Illumina | NYGC | Omega | 28.47 | Kapa | 5 | 290.5251567 |
| | | | HiSeq X | | | | Hyper | | |
| CRC 19 | preoperative | Streck | Illumina | NYGC | Omega | 5.97 | Kapa | 5 | 161.3757592 |
| | | | HiSeq X | | | | Hyper | | |
| CRC 1 | postoperative | Streck | Illumina | NYGC | Omega | 5.34 | Kapa | 5 | 97.17328055 |
| | | | HiSeq X | | | | Hyper | | |
| CRC 2 | postoperative | Streck | Illumina | NYGC | Omega | 18.27 | Kapa | 5 | 272.7902067 |
| | | | HiSeq X | | | | Hyper | | |
| CRC 3 | postoperative | Streck | Illumina | NYGC | Omega | 123 | Kapa | 5 | 524.5115628 |
| | | | HiSeq X | | | | Hyper | | |
| CRC 4 | postoperative | Streck | Illumina | NYGC | Omega | 17.61 | Kapa | 5 | 213.3256252 |
| | | | HiSeq X | | | | Hyper | | |
| CRC 5 | postoperative | Streck | Illumina | NYGC | Omega | 29.7 | Kapa | 5 | 273.4478687 |
| | | | HiSeq X | | | | Hyper | | |
| CRC 6 | postoperative | Streck | Illumina | NYGC | Omega | 5.61 | Kapa | 5 | 87.59937114 |
| | | | HiSeq X | | | | Hyper | | |
| CRC 7 | postoperative | Streck | Illumina | NYGC | Omega | 213.6 | Kapa | 5 | 58.27665438 |
| | | | HiSeq X | | | | Hyper | | |
| CRC 8 | postoperative | Streck | Illumina | NYGC | Omega | 15.54 | Kapa | 5 | 287.161284 |
| | | | HiSeq X | | | | Hyper | | |
| CRC 9 | postoperative | Streck | Illumina | NYGC | Omega | 81.9 | Kapa | 5 | 54.42572534 |
| | | | HiSeq X | | | | Hyper | | |
| CRC 10 | postoperative | Streck | Illumina | NYGC | Omega | 38.4 | Kapa | 5 | 182.0745224 |
| | | | HiSeq X | | | | Hyper | | |
| CRC 11 | postoperative | Streck | Illumina | NYGC | Omega | 13.65 | Kapa | 5 | 270.0374843 |
| | | | HiSeq X | | | | Hyper | | |
| CRC 12 | postoperative | Streck | Illumina | NYGC | Omega | 7.47 | Kapa | 5 | 157.3988837 |
| | | | HiSeq X | | | | Hyper | | |
| CRC 13 | postoperative | Streck | Illumina | NYGC | Omega | 87.6 | Kapa | 5 | 635.0613019 |
| | | | HiSeq X | | | | Hyper | | |

| | | | | | | |
|---|---|---|---|---|---|---|
| 9.01E+08 | 0.106115 | 25.907639 | 171 | 99.76% | 0.07636585 | 1.20888 |
| 8.25E+08 | 0.101486 | 23.896575 | 171 | 99.74% | 0.0721882 | 0.948695 |
| 8.09E+08 | 0.104325 | 23.390544 | 171 | 99.75% | 0.06183019 | −0.709386 |
| 8.54E+08 | 0.108961 | 24.311004 | 172 | 99.66% | 0.0531407 | NA |
| 7.73E+08 | 0.099711 | 23.153375 | 173 | 99.73% | 0.06185105 | 2.249649 |
| 7.71E+08 | 0.121448 | 22.110986 | 176 | 99.63% | 0.06339257 | −0.239148 |
| 8.69E+08 | 0.107608 | 25.729961 | 176 | 99.73% | 0.06011905 | 1.731356 |
| 8.47E+08 | 0.119974 | 26.185462 | 177 | 98.85% | 0.02144101 | 1.374159 |
| 8.41E+08 | 0.115477 | 23.5958 | 172 | 99.71% | 0.04890647 | −1.050264 |
| 9.04E+08 | 0.110798 | 25.567587 | 171 | 99.82% | 0.08457824 | 0.853841 |
| 9.05E+08 | 0.126971 | 26.300299 | 177 | 99.76% | 0.04536958 | 2.791941 |
| 8.93E+08 | 0.13775 | 24.473906 | 175 | 99.69% | 0.05459132 | −1.042194 |
| 9.61E+08 | 0.119754 | 27.670492 | 175 | 99.70% | 0.031962 | −0.776399 |
| 9.64E+08 | 0.135544 | 28.768618 | 183 | 99.71% | 0.06998479 | 0.616264 |
| 7.98E+08 | 0.097382 | 24.449565 | 178 | 99.65% | 0.04236713 | 0.98817 |
| 9.35E+08 | 0.120616 | 28.15287 | 180 | 99.69% | 0.07172533 | −0.245276 |
| 8.12E+08 | 0.118596 | 23.182496 | 175 | 99.74% | 0.05214594 | 2.056857 |
| 9.03E+08 | 0.128262 | 26.605018 | 175 | 99.63% | 0.1020482 | 1.035369 |

| | | | | | | | | | |
|---|---|---|---|---|---|---|---|---|---|
| CRC 14 | postoperative | Streck | Illumina | NYGC | Omega | 11.94 | Kapa | 5 | 234.528478 |
| | | | HiSeq X | | | | Hyper | | |
| CRC 15 | postoperative | Streck | Illumina | NYGC | Omega | 17.88 | Kapa | 5 | 229.9029857 |
| | | | HiSeq X | | | | Hyper | | |
| CRC 16 | postoperative | Streck | Illumina | NYGC | Omega | 34.8 | Kapa | 5 | 368.5589244 |
| | | | HiSeq X | | | | Hyper | | |
| CRC 17 | postoperative | Streck | Illumina | NYGC | Omega | 8.73 | Kapa | 5 | 211.951445 |
| | | | HiSeq X | | | | Hyper | | |
| CRC 18 | postoperative | Streck | Illumina | NYGC | Omega | 9.06 | Kapa | 5 | 162.3934427 |
| | | | HiSeq X | | | | Hyper | | |
| CRC 19 | postoperative | Streck | Illumina | NYGC | Omega | 8.73 | Kapa | 5 | 152.4550859 |
| | | | HiSeq X | | | | Hyper | | |

* previously reported in Zviran et al. Nature Med 2020

| | | TOTAL_READS | | | | | | somatic | |
|---|---|---|---|---|---|---|---|---|---|
| Early-stage | | | | | MEDIAN_{—} | | | pipeline | |
| LUAD | | | Percent- | Mean | INSERT_{—} | QC metrics | | # of | |
| Tumor | Tissue | TOTAL_{—} | Total- | Coverage | SIZE | Conpair- | auto- | mutation | # of |
| Patient ID | Type | READS | Duplication | (X) | (bp) | Concordance | correlation | detected | amplification |
| LUAD01 | Fresh frozen | 760869570 | 0.066582 | 34.967658 | 444 | 99.92% | 0.04673 | 8164 | 7 |
| LUAD02 | Fresh frozen | 776460166 | 0.073862 | 35.225186 | 439 | 99.89% | 0.0458 | 20285 | 21 |
| LUAD03 | Fresh frozen | 771984320 | 0.070421 | 35.500664 | 446 | 99.90% | 0.05551 | 13322 | 3 |
| LUAD04 | Fresh frozen | 1.19E+09 | 0.083747 | 54.1174 | 439 | 99.97 | 0.5211 | 5575 | 7 |
| LUAD05 | Fresh frozen | 795032986 | 0.051938 | 37.189623 | 413 | 99.82% | 0.10649 | 35796 | 11 |
| LUAD06 | Fresh frozen | 799141354 | 0.081228 | 35.6514 | 426 | 99.97 | 0.0051 | 2637 | 64 |
| LUAD07 | Fresh frozen | 907213986 | 0.079167 | 40.898896 | 442 | 99.85% | 0.0266 | 9988 | 2 |
| LUAD08 | Fresh frozen | 873232932 | 0.073975 | 39.670721 | 434 | 99.88% | 0.16469 | 944 | 8 |
| LUAD09 | Fresh frozen | 956426206 | 0.080129 | 43.543531 | 435 | 98.57% | 0.14788 | 39464 | 5 |
| LUAD10 | Fresh frozen | 853430422 | 0.088571 | 37.1756 | 418 | 99.8 | 0.0292 | 1167 | 70 |
| LUAD11 | Fresh frozen | 654141638 | 0.0714 | 29.6 | 439 | 99.9 | 0.00096 | 6305 | 10 |
| LUAD12 | Fresh frozen | 726370760 | 0.125178 | 31.4164 | 415 | 99.92 | 0.1852 | 11026 | 7 |
| LUAD13 | Fresh frozen | 806005466 | 0.070597 | 37.122148 | 436 | 99.69% | 0.06551 | 18517 | 5 |
| LUAD14 | Fresh frozen | 1.115E+09 | 0.160216 | 45.734246 | 417 | 99.92 | 0.01899 | 1174 | 17 |
| LUAD15 | Fresh frozen | 987087460 | 0.104467 | 43.636633 | 441 | 99.95 | 0.09421 | 943 | 7 |
| LUAD16 | Fresh frozen | 943429998 | 0.07911 | 42.899078 | 451 | 99.58% | 0.07802 | 115609 | 15 |

| | | | | | | |
|---|---|---|---|---|---|---|
| 8.46E+08 | 0.109462 | 24.075772 | 172 | 99.74% | 0.05956155 | 0.192033 |
| 8.43E+08 | 0.107199 | 24.422147 | 174 | 99.71% | 0.05033529 | 1.058602 |
| 7.92E+08 | 0.123464 | 22.436281 | 173 | 99.73% | 0.05828118 | −0.226084 |
| 8.94E+08 | 0.115229 | 25.352624 | 173 | 99.74% | 0.05973068 | −1.521489 |
| 8.54E+08 | 0.120926 | 24.586873 | 175 | 99.71% | 0.03834434 | NA |
| 7.35E+08 | 0.098453 | 21.974939 | 176 | 99.74% | 0.05265092 | −0.592839 |

| | Total | Total | | |
|---|---|---|---|---|
| # of | Mbp of | Mbp of | Tumor | |
| deletion | amplification | deletion | purity | Notes |
| 3 | 201 | 68 | 9 | |
| 3 | 1141 | 59 | 36 | |
| 3 | 106 | 186 | 18 | |
| 7 | 306 | 214 | 15 | |
| 20 | 345 | 1222 | 28 | |
| 14 | 217.71017 | 293.332445 | 20 | |
| 21 | 65 | 1143 | 23 | |
| 0 | 182 | 0 | 5 | |
| 24 | 233 | 1055 | 30 | |
| 9 | 284.13059 | 89.5747 | 28 | |
| 12 | 372.432 | 584.709 | 35 | |
| 32 | 259.759 | 213.308 | 37 | |
| 15 | 192 | 838 | 27 | |
| 3 | 970 | 106 | 6 | |
| 7 | 265 | 354 | 7 | |
| 24 | 336 | 854 | 53 | |

| | | | | | | | | | |
|---|---|---|---|---|---|---|---|---|---|
| LUAD17 | Fresh frozen | 1.181E+09 | 0.071027 | 54.375947 | 452 | 99.92% | 0.13713 | 2242 | 5 |
| LUAD18 | Fresh frozen | 1.252E+09 | 0.129608 | 52.1456 | 409 | 99.93 | 0.02805 | 26359 | 34 |
| LUAD19 | Fresh frozen | 681533694 | 0.139502 | 28.4782 | 438 | 99.85 | 0.1652 | 2442 | 27 |
| LUAD20 | Fresh frozen | 943480264 | 0.137554 | 39.4586 | 432 | 99.87 | 0.1377 | 3109 | 86 |
| LUAD21 | Fresh frozen | 526868616 | 0.074974 | 23.546874 | 447 | 99.89% | 0.04011 | 14480 | 8 |
| LUAD22 | Fresh frozen | 1.06E+09 | 0.156166 | 43.609143 | 428 | NA | 0.00792 | 17947 | 14 |
| LUAD23 | Fresh frozen | 1.038E+09 | 0.165588 | 42.816316 | 440 | 99.79 | 0.18071 | 2766 | 17 |
| LUAD24 | Fresh frozen | 788287174 | 0.047937 | 36.537192 | 408 | 99.90% | 0.08614 | 3616 | 11 |
| LUAD25 | Fresh frozen | 1.206E+09 | 0.92499 | 54.2113 | 451 | 99.9 | 0.4251 | 20165 | 9 |
| LUAD26 | Fresh frozen | 1.083E+09 | 0.94637 | 47.9011 | 438 | 99.55 | 0.22138 | 11981 | 4 |
| LUAD27 | Fresh frozen | 1.192E+09 | 0.179503 | 48.537355 | 426 | NA | 0.09441 | 6633 | 17 |
| LUAD28 | Fresh frozen | 995712358 | 0.156688 | 40.516358 | 412 | 99.87 | 0.0197 | 2222 | 11 |
| LUAD29 | Fresh frozen | 818081484 | 0.042499 | 38.532171 | 411 | 99.63% | 0.11442 | 4874 | 10 |
| LUAD30 | Fresh frozen | 761947686 | 0.068724 | 35.093899 | 449 | 99.88% | 0.11461 | 27323 | 7 |
| LUAD31 | Fresh frozen | 805289030 | 0.1366 | 11.32 | 138 | 99.76 | 0.03588 | 2805 | 122 |
| LUAD32 | Fresh frozen | 614279816 | 0.48521 | 28.124 | 444 | 99.92 | 0.01024 | 2341 | 8 |
| LUAD33 | Fresh frozen | 1.104E+09 | 0.07372 | 50.659562 | 446 | 99.92% | 0.11995 | 10858 | 10 |
| LUAD34 | Fresh frozen | 1.259E+09 | 0.093382 | 56.051307 | 435 | 98.68% | 0.0752 | 27973 | 9 |
| LUAD35 | Fresh frozen | 1.03E+09 | 0.154478 | 42.662322 | 419 | 99.85 | 0.01972 | 7034 | 6 |
| LUAD36 | Fresh frozen | 925726302 | 0.169294 | 37.743606 | 429 | 99.95 | 0.108 | 1235 | 6 |
| LUAD37 | Fresh frozen | 778414884 | 0.062471 | 35.6111 | 427 | 99.93 | 0.0047 | 2353 | 3 |
| LUAD38 | Fresh frozen | 721743163 | 0.038584 | 33.6598 | 419 | 99.83 | 0.0295 | 3763 | 19 |
| LUAD39 | Fresh frozen | 655853156 | 0.053199 | 30.203 | 430 | 99.95 | 0.0098 | 33621 | 4 |

* previously reported in Zviran et al. Nature Med 2020

| | sequencing metrics | | | | | | |
|---|---|---|---|---|---|---|---|
| | | | | MEDIAN_{—} | | | |
| Early Stage | | Percent- | Mean | INSERT_{—} | QC metrics | | |
| LUAD | TOTAL_{—} | Total- | Coverage | SIZE | Conpair- | auto- | |
| Patient ID | READS | Duplication | (X) | (bp) | Concordance | correlation | Notes |
| LUAD01 | 778509698 | 0.180845 | 31.378826 | 439 | 99.92% | 0.11705 | |
| LUAD02 | 812927982 | 0.184315 | 32.556192 | 432 | 99.89% | 0.07695 | |
| LUAD03 | 810449440 | 0.177703 | 32.789747 | 433 | 99.90% | 0.00989 | |
| LUAD04 | 907682714 | 0.110458 | 39.1856 | 439 | 99.97 | 0.0098 | |

| | | | |
|---|---|---|---|
| 20 | 162 | 693 | 41 |
| 31 | 308.471 | 635.826 | 45 |
| 20 | 172.72 | 343.141 | 12 |
| 34 | 509.5 | 1045.78 | 24 |
| 27 | 181 | 1193 | 50 |
| 14 | 624 | 838 | 23 |
| 5 | 405 | 91 | 6 |
| 2 | 268 | 35 | 6 |
| 3 | 196 | 80 | 10 |
| 5 | 145 | 223 | 28 |
| 3 | 721 | 106 | 20 |
| 3 | 580 | 162 | 6 |
| 1 | 293 | 21 | 6 |
| 12 | 241 | 433 | 13 |
| 69 | 533.651 | 806.458 | 48 |
| 16 | 100.093 | 688.07 | 23 |
| 15 | 414 | 819 | 50 |
| 22 | 293 | 897 | 64 |
| 8 | 525 | 424 | 17 |
| 0 | 57 | 0 | NA |
| 4 | 365.245 | 219.355 | 23 |
| 12 | 225.859 | 266.849 | 33 |
| 25 | 115.076 | 735.795 | 30 |

| | | | | | | |
|---|---|---|---|---|---|---|
| LUAD05 | 812247988 | 0.086283 | 36.542961 | 439 | 99.82% | 0.07076 |
| LUAD06 | 793157034 | 0.049375 | 35.7462 | 433 | 99.97 | 0.0266 |
| LUAD07 | 847410510 | 0.08163 | 38.430558 | 443 | 99.85% | 0.13742 |
| LUAD08 | 872436794 | 0.083971 | 39.218143 | 423 | 99.88% | 0.09888 |
| LUAD09 | 806969674 | 0.187062 | 32.283227 | 423 | 98.57% | 0.02127 |
| LUAD10 | 1010330464 | 0.111988 | 43.8825 | 453 | 99.8 | 0.0033 |
| LUAD11 | 949438980 | 0.1196 | 40.91 | 442 | 99.9 | −0.00606 |
| LUAD12 | 853452680 | 0.140478 | 36.7143 | 434 | 99.92 | 0.0666 |
| LUAD13 | 858923982 | 0.086671 | 38.740084 | 437 | 99.69% | 0.20822 |
| LUAD14 | 784459779 | 0.048567 | 36.892477 | 429 | 99.92% | 0.1331 |
| LUAD15 | 769812148 | 0.074797 | 35.281596 | 430 | 99.95 | 0.12812 |
| LUAD16 | 820541586 | 0.206589 | 31.98237 | 433 | 99.58% | 0.10793 |
| LUAD17 | 824856416 | 0.208486 | 32.232299 | 437 | 99.92% | 0.09999 |
| LUAD18 | 751763446 | 0.100714 | 32.7085 | 438 | 99.93 | 0.00844 |
| LUAD19 | 817892350 | 0.137449 | 34.7208 | 416 | 99.85 | 0.0222 |
| LUAD20 | 868713128 | 138799 | 37.218 | 427 | 99.87 | 0.0387 |
| LUAD21 | 771036328 | 0.088164 | 34.453434 | 426 | 99.89% | 0.0328 |
| LUAD22 | NA | NA | NA | NA | NA | NA |
| LUAD23 | 795957954 | 0.077541 | 36.267608 | 430 | 99.79 | 0.10183 |
| LUAD24 | 790026456 | 0.178096 | 31.861696 | 436 | 99.90% | 0.14964 |
| LUAD25 | 882722692 | 0.092161 | 39.0421 | 432 | 99.9 | 0.0286 |
| LUAD26 | 1136516610 | 0.103251 | 48.6134 | 446 | 99.55 | 0.0451 |
| LUAD27 | NA | NA | NA | NA | NA | NA |
| LUAD28 | 791769424 | 0.062783 | 36.759126 | 433 | 99.87 | 0.12489 |
| LUAD29 | 846928524 | 0.096858 | 37.416409 | 431 | 99.63% | 0.14934 |
| LUAD30 | 781140114 | 0.183733 | 31.584145 | 435 | 99.88% | 0.15676 |
| LUAD31 | 1495787608 | 0.180472 | 19.376601 | 135 | 99.76% | 0.0129 |
| LUAD32 | 865444064 | 0.62102 | 39.1525 | 443 | 99.92 | −3.88E−05 |
| LUAD33 | 864685442 | 0.095543 | 38.729711 | 442 | 99.92% | 0.15356 |
| LUAD34 | 800481126 | 0.186757 | 32.17393 | 459 | 98.68% | 0.22639 |
| LUAD35 | 756196368 | 0.049932 | 35.544777 | 440 | 99.85 | 0.13267 |
| LUAD36 | 785103206 | 0.047085 | 36.629414 | 432 | 99.95 | 0.05814 |
| LUAD37 | 787510884 | 0.112333 | 34.3825 | 460 | 99.93 | −0.0008 |
| LUAD38 | 825206824 | 0.112714 | 35.9828 | 452 | 99.83 | −0.0016 |
| LUAD39 | 895242462 | 0.114283 | 38.8289 | 453 | 99.95 | −0.0041 |

* previously reported in Zviran et al. Nature Med 2020

| | | pre | | | | | | | sequencing |
|---|---|---|---|---|---|---|---|---|---|
| Early-stage | | sequencing | | | | | | | metrics |
| LUAD | | QC | Blood | | | total | library | # of | library |
| Plasma | | Sequencing | collection | Sequencing | extraction | mass | prep | PCR | mass |
| Patient ID | Timepoint | Platform | tube | Location | kit | (ng) | kit | cycles | (ng) |
| LUAD01 | preoperative | Illumina | Streck | NYGC | Omega | 11.76 | Kapa | 5 | 84.06990377 |
| | | HiSeq X | | | | | Hyper | | |
| LUAD02 | preoperative | Illumina | Streck | NYGC | Omega | 11.55 | Kapa | 5 | 108.4324652 |
| | | HiSeq X | | | | | Hyper | | |
| LUAD03 | preoperative | Illumina | Streck | NYGC | Omega | 6.57 | Kapa | 5 | 202.567422 |
| | | HiSeq X | | | | | Hyper | | |
| LUAD04 | preoperative | Illumina | Streck | NYGC | Omega | 12.68 | NEXTflex | 10 | 13.28 |
| | | HiSeq X | | | | | Cell | | |
| | | | | | | | Free | | |
| | | | | | | | DNA- | | |
| | | | | | | | Seq Kit | | |
| LUAD05 | preoperative | Illumina | Streck | NYGC | Omega | 12.99 | Kapa | 5 | 121.600833 |
| | | HiSeq X | | | | | Hyper | | |
| LUAD06 | preoperative | Illumina | Streck | NYGC | Omega | 8.48 | NEXTflex | 10 | 41.2 |
| | | HiSeq X | | | | | Cell | | |
| | | | | | | | Free | | |
| | | | | | | | DNA- | | |
| | | | | | | | Seq Kit | | |
| LUAD07 | preoperative | Illumina | Streck | NYGC | Omega | 14.04 | Kapa | 5 | 274.9211134 |
| | | HiSeq X | | | | | Hyper | | |
| LUAD08 | preoperative | Illumina | Streck | NYGC | Omega | 19.41 | Kapa | 5 | 365.378476 |
| | | HiSeq X | | | | | Hyper | | |
| LUAD09 | preoperative | Illumina | Streck | NYGC | Omega | 12.15 | Kapa | 5 | 200.4969148 |
| | | HiSeq X | | | | | Hyper | | |
| LUAD10 | preoperative | Illumina | Streck | NYGC | Omega | 6.63 | Kapa | 5 | 99.54 |
| | | HiSeq X | | | | | Hyper | | |
| LUAD11 | preoperative | Illumina | Streck | NYGC | Omega | 1.38 | Kapa | 10 | 42.075 |
| | | HiSeq X | | | | | Hyper | | |

| | | | QC metrics | | |
|---|---|---|---|---|---|
| | | | MEDIAN_{—} | | |
| | Percent- | Mean | INSERT_{—} | | |
| TOTAL_{—} | Total- | Coverage | SIZE | Conpair- | |
| READS | Duplication | (X) | (bp) | Concordance | Notes |
| 7.15E+08 | 0.088438 | 20.814924 | 171 | 99.58% | |
| 6.26E+08 | 0.096298 | 18.433533 | 172 | 99.62% | |
| 9.59E+08 | 0.099901 | 29.059211 | 174 | 99.69% | |
| 9.14E+08 | 0.3738898 | 18.007293 | 171 | 99.65 | |
| 6.54E+08 | 0.077493 | 19.119752 | 169 | 99.62% | |
| 9.28E+08 | 0.4043732 | 16.798926 | 170 | 99.51 | |
| 7.97E+08 | 0.094305 | 23.219926 | 171 | 99.58% | |
| 9.32E+08 | 0.108589 | 26.910696 | 169 | 99.69% | |
| 8.64E+08 | 0.106902 | 25.470112 | 173 | 99.63% | |
| 9.44E+08 | 0.1374476 | 25.719729 | 172 | 99.53 | |
| 7.08E+08 | 0.197248 | 17.509776 | 171 | 99.68 | |

| | | | | | | | | | |
|---|---|---|---|---|---|---|---|---|---|
| LUAD12 | preoperative | Illumina | Streck | NYGC | Omega | 19.17 | Kapa | 5 | 116.8 |
| | | HiSeq X | | | | | Hyper | | |
| LUAD13 | preoperative | Illumina | Streck | NYGC | Omega | 7.2 | Kapa | 5 | 269.1222385 |
| | | HiSeq X | | | | | Hyper | | |
| LUAD14 | preoperative | Illumina | Streck | NYGC | Omega | 7.26 | Kapa | 5 | 137.0074937 |
| | | HiSeq X | | | | | Hyper | | |
| LUAD15 | preoperative | Illumina | Streck | NYGC | Omega | 10.17 | Kapa | 5 | 228.0992763 |
| | | HiSeq X | | | | | Hyper | | |
| LUAD16 | preoperative | Illumina | Streck | NYGC | Omega | 10.17 | Kapa | 5 | 149.9852259 |
| | | HiSeq X | | | | | Hyper | | |
| LUAD17 | preoperative | Illumina | Streck | NYGC | Omega | 276.6 | Kapa | 5 | 155.4464442 |
| | | HiSeq X | | | | | Hyper | | |
| LUAD18 | preoperative | Illumina | Streck | NYGC | Omega | 6.09 | Kapa | 5 | 38.4734658 |
| | | HiSeq X | | | | | Hyper | | |
| LUAD19 | preoperative | Illumina | Streck | NYGC | Omega | 13.14 | Kapa | 5 | 108.4 |
| | | HiSeq X | | | | | Hyper | | |
| LUAD20 | preoperative | Illumina | Streck | NYGC | Omega | 12.45 | Kapa | 5 | 69.6 |
| | | HiSeq X | | | | | Hyper | | |
| LUAD21 | preoperative | Illumina | Streck | NYGC | Omega | 6.33 | Kapa | 5 | 179.2694693 |
| | | HiSeq X | | | | | Hyper | | |
| LUAD22 | preoperative | Illumina | Streck | NYGC | Omega | 22.71 | Kapa | 5 | 136.8302801 |
| | | HiSeq X | | | | | Hyper | | |
| LUAD23 | preoperative | Illumina | Streck | NYGC | Omega | 6.27 | Kapa | 5 | 168.2890049 |
| | | HiSeq X | | | | | Hyper | | |
| LUAD24 | preoperative | Illumina | Streck | NYGC | Omega | 40.8 | Kapa | 5 | 188.9769341 |
| | | HiSeq X | | | | | Hyper | | |
| LUAD25 | preoperative | Illumina | Streck | NYGC | Omega | 4.72 | NEXTflex | 10 | 10.44 |
| | | HiSeq X | | | | | Cell | | |
| | | | | | | | Free | | |
| | | | | | | | DNA- | | |
| | | | | | | | Seq Kit | | |
| LUAD26 | preoperative | Illumina | Streck | NYGC | Omega | 6.57 | NEXTflex | 10 | 19.5 |
| | | HiSeq X | | | | | Cell | | |
| | | | | | | | Free | | |
| | | | | | | | DNA- | | |
| | | | | | | | Seq Kit | | |

| | | | | |
|---|---|---|---|---|
| 8.34E+08 | 0.9483884 | 24.827619 | 174 | 99.73 |
| 8.71E+08 | 0.100403 | 25.336277 | 169 | 99.70% |
| 9.36E+08 | 0.131861 | 25.801508 | 172 | 99.69% |
| 9.22E+08 | 0.115221 | 26.441377 | 173 | 97.77% |
| 9.26E+08 | 0.119661 | 26.976105 | 173 | 99.70% |
| 7.42E+08 | 0.083051 | 23.164643 | 177 | 99.66% |
| 9.12E+08 | 0.1351827 | 24.005264 | 170 | 99.72 |
| 7.83E+08 | 0.0966473 | 23.144885 | 175 | 99.71 |
| 8.34E+08 | 0.0994056 | 24.310334 | 172 | 99.67 |
| 8.37E+08 | 0.103267 | 24.383015 | 172 | 99.65% |
| 8.1E+08 | 0.122324 | 22.078402 | 169 | No PBMC/NA |
| 9.11E+08 | 0.132497 | 24.767481 | 171 | 99.66% |
| 7.54E+08 | 0.095405 | 21.842632 | 171 | 99.74% |
| 9.55E+08 | 0.363102 | 19.050409 | 170 | 99.64 |
| 9.13E+08 | 0.6461836 | 10.908089 | 179 | 100 |

| | | | | | | | | | |
|---|---|---|---|---|---|---|---|---|---|
| LUAD27 | preoperative | Illumina | Streck | NYGC | Omega | 41.7 | Kapa | 5 | 156.0895091 |
| | | HiSeq X | | | | | Hyper | | |
| LUAD28 | preoperative | Illumina | Streck | NYGC | Omega | 9.57 | Kapa | 5 | 303.576138 |
| | | HiSeq X | | | | | Hyper | | |
| LUAD29 | preoperative | Illumina | Streck | NYGC | Omega | 35.7 | Kapa | 5 | 254.736027 |
| | | HiSeq X | | | | | Hyper | | |
| LUAD30 | preoperative | Illumina | Streck | NYGC | Omega | 12.72 | Kapa | 5 | 219.9235248 |
| | | HiSeq X | | | | | Hyper | | |
| LUAD31 | preoperative | Illumina | Streck | NYGC | Omega | NA | Kapa | 5 | NA |
| | | HiSeq X | | | | | Hyper | | |
| LUAD32 | preoperative | Illumina | Streck | NYGC | Omega | 0.536 | Kapa | 13 | 86.75 |
| | | HiSeq X | | | | | Hyper | | |
| LUAD33 | preoperative | Illumina | Streck | NYGC | Omega | 13.95 | Kapa | 5 | 203.4414365 |
| | | HiSeq X | | | | | Hyper | | |
| LUAD34 | preoperative | Illumina | Streck | NYGC | Omega | 49.2 | Kapa | 5 | 295.147233 |
| | | HiSeq X | | | | | Hyper | | |
| LUAD35 | preoperative | Illumina | Streck | NYGC | Omega | 10.53 | Kapa | 5 | 269.9439529 |
| | | HiSeq X | | | | | Hyper | | |
| LUAD36 | preoperative | Illumina | Streck | NYGC | Omega | 16.68 | Kapa | 5 | 156.4233107 |
| | | HiSeq X | | | | | Hyper | | |
| LUAD37 | preoperative | Illumina | Streck | NYGC | Omega | 11.4 | Kapa | 5 | 41.4 |
| | | HiSeq X | | | | | Hyper | | |
| LUAD38 | preoperative | Illumina | Streck | NYGC | Omega | 13.77 | Kapa | 5 | 50.2 |
| | | HiSeq X | | | | | Hyper | | |
| LUAD39 | preoperative | Illumina | Streck | NYGC | Omega | 13.74 | Kapa | 5 | 28.875 |
| | | HiSeq X | | | | | Hyper | | |

| | | | | | |
|---|---|---|---|---|---|
| 7.43E+08 | 0.105852 | 21.978316 | 174 | No PBMC/NA | |
| 7.84E+08 | 0.109773 | 21.802741 | 169 | 99.70% | |
| 7.84E+08 | 0.09741 | 22.98133 | 170 | 99.58% | |
| 7.85E+08 | 0.104654 | 22.794576 | 172 | 99.64% | |
| 1.56E+09 | 0.155711 | 37.775631 | 171 | 99.58 | |
| 1.11E+09 | 0.180631 | 29.150785 | 175 | 99.79 | |
| 8.54E+08 | 0.108509 | 25.118305 | 174 | 99.74% | |
| 7.93E+08 | 0.101862 | 22.694761 | 171 | 99.71% | |
| 8.38E+08 | 0.107597 | 23.971608 | 171 | 99.78% | |
| 7.86E+08 | 0.117514 | 22.823154 | 174 | 99.71% | |
| 9.72E+08 | 0.12151 | 28.176987 | 175 | 41.16 | Excluded for |
| | | | | | low |
| | | | | | concordance |
| | | | | | (<99%) |
| 9.72E+08 | 0.12151 | 28.176987 | 175 | 93.34 | Excluded for |
| | | | | | low |
| | | | | | concordance |
| | | | | | (<99%) |
| 7.44E+08 | 0.1207881 | 21.280884 | 172 | 38.12 | Excluded for |
| | | | | | low |
| | | | | | concordance |
| | | | | | (<99%) |

| | | | | | | | | | |
|---|---|---|---|---|---|---|---|---|---|
| LUAD04 | postoperative | Illumina | Streck | NYGC | Omega | 5.16 | NEXTflex | 10 | 8.36 |
| | | HiSeq X | | | | | Cell | | |
| | | | | | | | Free | | |
| | | | | | | | DNA- | | |
| | | | | | | | Seq Kit | | |
| LUAD06 | postoperative | Illumina | Streck | NYGC | Omega | 3.66 | NEXTflex | 10 | 8.12 |
| | | HiSeq X | | | | | Cell | | |
| | | | | | | | Free | | |
| | | | | | | | DNA- | | |
| | | | | | | | Seq Kit | | |
| LUAD10 | postoperative | Illumina | Streck | NYGC | Omega | 5.82 | Kapa | 7 | 71.16 |
| | | HiSeq X | | | | | Hyper | | |
| LUAD11 | postoperative | Illumina | Streck | NYGC | Omega | 2.061 | Kapa | 10 | 55.525 |
| | | HiSeq X | | | | | Hyper | | |
| LUAD12 | postoperative | Illumina | Streck | NYGC | Omega | 9.81 | Kapa | 5 | 63.6 |
| | | HiSeq X | | | | | Hyper | | |
| LUAD14 | postoperative | Illumina | Streck | NYGC | Omega | 5.82 | Kapa | 5 | 92.66795085 |
| | | HiSeq X | | | | | Hyper | | |
| LUAD15 | postoperative | Illumina | Streck | NYGC | Omega | 8.97 | Kapa | 5 | 212.4828899 |
| | | HiSeq X | | | | | Hyper | | |
| LUAD18 | postoperative | Illumina | Streck | NYGC | Omega | 10.56 | Kapa | 7 | 149.6 |
| | | HiSeq X | | | | | Hyper | | |
| LUAD19 | postoperative | Illumina | Streck | NYGC | Omega | 9.51 | Kapa | 5 | 54.8 |
| | | HiSeq X | | | | | Hyper | | |
| LUAD20 | postoperative | Illumina | Streck | NYGC | Omega | 8.43 | Kapa | 5 | 67.6 |
| | | HiSeq X | | | | | Hyper | | |
| LUAD22 | postoperative | Illumina | Streck | NYGC | Omega | 7.26 | Kapa | 5 | 184.6651628 |
| | | HiSeq X | | | | | Hyper | | |
| LUAD23 | postoperative | Illumina | Streck | NYGC | Omega | 5.01 | Kapa | 5 | 116.6850612 |
| | | HiSeq X | | | | | Hyper | | |
| LUAD25 | postoperative | Illumina | Streck | NYGC | Omega | 7.86 | NEXTflex | 10 | 14.12 |
| | | HiSeq X | | | | | Cell | | |
| | | | | | | | Free | | |
| | | | | | | | DNA- | | |
| | | | | | | | Seq Kit | | |

| | | | | |
|---|---|---|---|---|
| 9.3E+08 | 0.5018184 | 14.79117 | 174 | 99.4 |
| 8.91E+08 | 0.3792072 | 16.987169 | 168 | 99.65 |
| 8.13E+08 | 0.1182231 | 22.597948 | 172 | 99.62 |
| 6.56E+08 | 0.1446523 | 17.65154 | 171 | 99.67 |
| 8.24E+08 | 0.0972736 | 25.652071 | 177 | 99.67 |
| 8.08E+08 | 0.129909 | 21.614913 | 170 | 99.67% |
| 8.81E+08 | 0.110188 | 25.091261 | 172 | 97.83% |
| 1.07E+09 | 0.0912513 | 31.171318 | 171 | 99.76 |
| 8.18E+08 | 0.0123776 | 23.547133 | 173 | 99.69 |
| 8.18E+08 | 0.0910127 | 24.463084 | 174 | 99.75 |
| 9.1E+08 | 0.127703 | 25.867272 | 174 | No PBMC/NA |
| 6.95E+08 | 0.098911 | 19.475952 | 171 | 99.60% |
| 8.95E+08 | 0.3529525 | 18.201522 | 170 | 99.63 |

| | | | | | | | | | |
|---|---|---|---|---|---|---|---|---|---|
| LUAD26 | postoperative | Illumina | Streck | NYGC | Omega | 4.11 | NEXTflex | 10 | 10.035 |
| | | HiSeq X | | | | | Cell | | |
| | | | | | | | Free | | |
| | | | | | | | DNA- | | |
| | | | | | | | Seq Kit | | |
| LUAD27 | postoperative | Illumina | Streck | NYGC | Omega | 9.57 | Kapa | 5 | 225.6456087 |
| | | HiSeq X | | | | | Hyper | | |
| LUAD28 | postoperative | Illumina | Streck | NYGC | Omega | 23.19 | Kapa | 5 | 293.6021538 |
| | | HiSeq X | | | | | Hyper | | |
| LUAD31 | postoperative | Illumina | Streck | NYGC | Omega | NA | Kapa | NA | NA |
| | | HiSeq X | | | | | Hyper | | |
| LUAD32 | postoperative | Illumina | Streck | NYGC | Omega | 0.648 | Kapa | 13 | 35.5 |
| | | HiSeq X | | | | | Hyper | | |
| LUAD35 | postoperative | Illumina | Streck | NYGC | Omega | 16.8 | Kapa | 5 | 243.2009736 |
| | | HiSeq X | | | | | Hyper | | |
| LUAD37 | postoperative | Illumina | Streck | NYGC | Omega | 11.13 | Kapa | 5 | 31.525 |
| | | HiSeq X | | | | | Hyper | | |
| LUAD38 | postoperative | Illumina | Streck | NYGC | Omega | 6.96 | Kapa | 7 | 65.65 |
| | | HiSeq X | | | | | Hyper | | |
| LUAD39 | postoperative | Illumina | Streck | NYGC | Omega | 2.139 | Kapa | 10 | 56.35 |
| | | HiSeq X | | | | | Hyper | | |

* previously reported in Zviran et al. Nature Med 2020

| | | | | | | | | | somatic |
|---|---|---|---|---|---|---|---|---|---|
| | | | TOTAL_{—} | | | MEDIAN_{—} | QC | | pipeline |
| | | | READS | Percent- | Mean | INSERT_{—} | metrics | | # of |
| Neo Tumor | | Tissue | TOTAL_{—} | Total- | Coverage | SIZE | Conpair | auto- | mutation |
| Patient ID | Sample ID | Type | READS | Duplication | (X) | (bp) | Concordance | correlation | detected |
| Neo-01 | NA-18 | Fresh | 851681720 | 10.2859 | 36.8366 | 390 | 99.79 | 0.0972 | 16287 |
| | | frozen | | | | | | | |
| Neo-02 | NA-40 | Fresh | 518582254 | 18.7548 | 20.8158 | 386 | 99.94 | 0.0853 | 43839 |
| | | frozen | | | | | | | |
| Neo-03 | NA-36 | Fresh | 1037874304 | 9.2262 | 47.0561 | 402 | 99.74 | 0.1476 | 31138 |
| | | frozen | | | | | | | |

| | | | | |
|---|---|---|---|---|
| 9E+08 | 0.757713 | 7.575821 | 181 | 100 |
| 7.8E+08 | 0.106957 | 22.324676 | 172 | No PBMC/NA |
| 7.43E+08 | 0.100357 | 20.80418 | 170 | 99.73% |
| 7.92E+08 | 0.1042621 | 19.794253 | 168 | 99.57 |
| 1.15E+09 | 0.2772693 | 27.213808 | 174 | 99.71 |
| 8.64E+08 | 0.103246 | 25.755422 | 174 | 99.73% |
| 8.19E+08 | 0.1043323 | 24.082231 | 172 | 99.8 |
| 8.43E+08 | 0.1299222 | 24.038656 | 173 | 99.65 |
| 7.22E+08 | 0.2050572 | 18.857996 | 172 | 99.7 |

| # of | # of | Total Mbp of | Total Mbp of | | |
|---|---|---|---|---|---|
| amplification | deletion | amplification | deletion | CNLOH | Notes |
| 14 | 7 | 253.2 | 141.4 | 10 | |
| 77 | 76 | 618.215 | 1229.89 | 154.284 | |
| 29 | 22 | 1006 | 851 | 830 | |

| | | sequencing | | | MEDIAN_{—} | QC | | |
|---|---|---|---|---|---|---|---|---|
| Neo Normal/ | | metrics | Percent- | | INSERT_{—} | metrics | | |
| PBMC | | TOTAL_{—} | Total- | Mean | SIZE | Conpair- | auto- | |
| Patient ID | Sample ID | READS | Duplication | Coverage (X) | (bp) | Concordance | correlation | Notes |
| Neo-01 | NA-18 | 748592808 | 10.7047 | 33.1071 | 441 | 99.79 | 0.0122 | |
| Neo-02 | NA-40 | 876047448 | 25.2697 | 32.9481 | 427 | 99.94 | 0.089 | |
| Neo-03 | NA-36 | 536484210 | 11.1043 | 24.0244 | 418 | 99.74 | 0.0981 | |

| | | | pre | | | | | | |
|---|---|---|---|---|---|---|---|---|---|
| | | | sequencing | | | | | | |
| | | | QC | | | | total | library | library |
| Neo Plasma | | | Blood | Sequencing | Sequencing | extraction | mass | mass | prep |
| Patient ID | Sample ID | Timepoint | collection tube | Platform | Location | kit | (ng) | (ng) | kit |
| Neo-01 | NA-18_B | Day 3 | Streck | Illumina | NYGC | Omega | 22.71 | 22.7076 | Kapa |
| | | | | NovaSeq v1.0 | | | | | Hyper |
| | NA-18_C | Week 4 | Streck | Illumina | NYGC | Omega | 5.88 | 5.875 | Kapa |
| | | | | NovaSeq v1.0 | | | | | Hyper |
| | NA-18_D | Week 6 | Streck | Illumina | NYGC | Omega | 4.98 | 4.977 | Kapa |
| | | | | NovaSeq v1.0 | | | | | Hyper |
| | NA-18_E | Postoperative | Streck | Illumina | NYGC | Omega | 5.45 | 5.451 | Kapa |
| | | Month 3 | | NovaSeq v1.0 | | | | | Hyper |
| Neo-02 | NA-40_A | Pretreatment | Streck | Illumina | NYGC | Omega | 50.87 | 25 | Kapa |
| | | | | NovaSeq v1.0 | | | | | Hyper |
| | NA-40_D | Week 6 | Streck | Illumina | NYGC | Omega | 55 | 25 | Kapa |
| | | | | NovaSeq v1.0 | | | | | Hyper |
| | NA-40_E | Postoperative | Streck | Illumina | NYGC | Omega | 45.62 | 25 | Kapa |
| | | Month 3 | | NovaSeq v1.0 | | | | | Hyper |
| Neo-03 | NA-36_A | Pretreatment | Streck | Illumina | NYGC | Omega | 6.78 | 6.776 | Kapa |
| | | | | NovaSeq v1.0 | | | | | Hyper |
| | NA-36_C | Week 4 | Streck | Illumina | NYGC | Omega | 13.82 | 13.824 | Kapa |
| | | | | NovaSeq v1.0 | | | | | Hyper |

| | | | | QC | |
|---|---|---|---|---|---|
| | | | | metrics | |
| sequencing | | | | MEDIAN_{—} | |
| metrics | | Percent- | Mean | INSERT_{—} | |
| # of PCR | TOTAL_{—} | Total- | Coverage | SIZE | |
| cycles | READS | Duplication | (X) | (bp) | Notes |
| 6 | 1.097E+09 | 10.9249 | 33.6319 | 177 | |
| 6 | 1.074E+09 | 7.7541 | 30.9364 | 170 | |
| 6 | 1.052E+09 | 7.5528 | 31.6967 | 172 | |
| 6 | 1.051E+09 | 7.6822 | 32.619 | 175 | |
| 6 | 797901410 | 17.5102 | 22.211 | 174 | |
| 6 | 1.051E+09 | 9.9104 | 33.7036 | 180 | |
| 6 | 1.323E+09 | 8.7483 | 42.7531 | 178 | |
| 6 | 2.255E+09 | 11.5334 | 64.0543 | 170 | |
| 6 | 1.224E+09 | 7.5797 | 36.6608 | 171 | |

| | | | | | | | | |
|---|---|---|---|---|---|---|---|---|
| NA-36_D | Week 6 | Streck | Illumina | NYGC | Omega | 19.87 | 19.872 | Kapa |
| | | | NovaSeq v1.0 | | | | | Hyper |
| NA-36_E | Postoperative | Streck | Illumina | NYGC | Omega | 8.64 | 8.642 | Kapa |
| | Month 3 | | NovaSeq v1.0 | | | | | Hyper |

*library mass capped at 25 ng

| | | | | | | | | sequencing | |
|---|---|---|---|---|---|---|---|---|---|
| Conventional | pre | | | | | | | metrics | |
| Immunotherapy | sequencing | Blood | | | | total | library | # of | library |
| Plasma | QC | Collection | Sequencing | Sequencing | extraction | mass | prep | PCR | mass |
| Patient ID | Timepoint | Tube | Platform | Location | kit | (ng) | kit | cycles | (ng) |
| MSK-32_A | Pretreatment | K2-EDTA | Illumina | NYGC | Omega | 28.5 | Kapa | 6 | 28.5 |
| | | | HiSeq X | | | | Hyper | | |
| MSK-32_C | Week 3 | K2-EDTA | Illumina | NYGC | Omega | 4.475 | Kapa | 6 | 4.475 |
| | | | HiSeq X | | | | Hyper | | |
| MSK-32_D | Week 6 | K2-EDTA | Illumina | NYGC | Omega | 8.975 | Kapa | 6 | 8.975 |
| | | | HiSeq X | | | | Hyper | | |
| MSK-32_G | Week 12 | K2-EDTA | Illumina | NYGC | Omega | 8.6 | Hyper | 6 | 8.6 |
| | | | HiSeq X | | | | Kapa | | |
| MSK-33_A | Pretreatment | K2-EDTA | Illumina | NYGC | Omega | 11.75 | Kapa | 6 | 11.75 |
| | | | HiSeq X | | | | Hyper | | |
| MSK-33_C | Week 3 | K2-EDTA | Illumina | NYGC | Omega | 28.5 | Kapa | 6 | 28.5 |
| | | | HiSeq X | | | | Hyper | | |
| MSK-33_D | Week 6 | K2-EDTA | Illumina | NYGC | Omega | 14.35 | Kapa | 6 | 14.35 |
| | | | HiSeq X | | | | Hyper | | |
| MSK-33_G | Week 12 | K2-EDTA | Illumina | NYGC | Omega | 19.575 | Kapa | 6 | 19.575 |
| | | | HiSeq X | | | | Hyper | | |
| MSK-34_A | Pretreatment | K2-EDTA | Illumina | NYGC | Omega | 8.9 | Kapa | 6 | 8.9 |
| | | | HiSeq X | | | | Hyper | | |
| MSK-34_F | Week 3 | K2-EDTA | Illumina | NYGC | Omega | 27.25 | Kapa | 6 | 25 |
| | | | HiSeq X | | | | Hyper | | |

| | | | | |
|---|---|---|---|---|
| 6 | 1.043E+09 | 6.4044 | 31.8518 | 172 |
| 6 | 2.101E+09 | 13.1887 | 62.7741 | 177 |

| | | QC | | | | | |
|---|---|---|---|---|---|---|---|
| | | metrics | MEDIAN_{—} | | | | |
| | Percent- | Mean | INSERT_{—} | | | | |
| TOTAL_{—} | Total- | Coverage | SIZE | Conpair- | Pileup- | Auto- | |
| READS | Duplication | (X) | (bp) | Concordance | Size | correlation | Notes |
| 1.11E+09 | 10.2876 | 31.3539 | 200.9524 | 99.82 | 34988746 | 0.06691062 | |
| 1.02E+09 | 10.6395 | 26.6636 | 181.5969 | 99.84 | 17304837 | 0.07718508 | |
| 1.06E+09 | 12.1073 | 27.9475 | 185.597 | 99.9 | 19632736 | 0.0811522 | |
| 1.05E+09 | 12.056 | 27.386 | 185.9823 | 99.87 | 19151539 | 0.08528573 | |
| 1.06E+09 | 10.5549 | 28.2538 | 190.3012 | 99.97 | 21016043 | 0.06300264 | |
| 1.17E+09 | 12.4704 | 30.5422 | 192.8816 | 99.92 | 25820866 | 0.06435689 | |
| 1.12E+09 | 11.6224 | 29.1653 | 188.949 | 99.97 | 21436871 | 0.05607227 | |
| 1.26E+09 | 12.0441 | 33.6694 | 197.8723 | 99.95 | 30737824 | 0.06226284 | |
| 1.14E+09 | 11.0208 | 30.6627 | 194.6228 | 99.92 | 30242013 | 0.1225945 | |
| 1.14E+09 | 11.0764 | 30.6585 | 182.9388 | 99.87 | 18492434 | 0.0642385 | |

| | | | | | | | | | |
|---|---|---|---|---|---|---|---|---|---|
| MSK-34_I | Week 6 | K2-EDTA | Illumina | NYGC | Omega | 16.15 | Kapa | 6 | 16.5 |
| | | | HiSeq X | | | | Hyper | | |
| MSK-34_M | Week 12 | K2-EDTA | Illumina | NYGC | Omega | 41.75 | Kapa | 6 | 25 |
| | | | HiSeq X | | | | Hyper | | |
| MSK-37_A | Pretreatment | K2-EDTA | Illumina | NYGC | Omega | 12.4 | Hyper | 6 | 12.4 |
| | | | HiSeq X | | | | Kapa | | |
| MSK-37_C | Week 3 | K2-EDTA | Illumina | NYGC | Omega | 16.325 | Kapa | 6 | 16.325 |
| | | | HiSeq X | | | | Hyper | | |
| MSK-37_D | Week 6 | K2-EDTA | Illumina | NYGC | Omega | 7.3 | Kapa | 6 | 7.3 |
| | | | HiSeq X | | | | Hyper | | |
| MSK-37_G | Week 12 | K2-EDTA | Illumina | NYGC | Omega | 9.175 | Kapa | 6 | 9.175 |
| | | | HiSeq X | | | | Hyper | | |
| MSK-38_A | Pretreatment | K2-EDTA | Illumina | NYGC | Omega | 20.225 | Kapa | 6 | 20.225 |
| | | | HiSeq X | | | | Hyper | | |
| MSK-38_C | Week 3 | K2-EDTA | Illumina | NYGC | Omega | 4.175 | Kapa | 6 | 4.175 |
| | | | HiSeq X | | | | Hyper | | |
| MSK-38_D | Week 6 | K2-EDTA | Illumina | NYGC | Omega | 35.75 | Kapa | 6 | 25 |
| | | | HiSeq X | | | | Hyper | | |
| MSK-38_H | Week 12 | K2-EDTA | Illumina | NYGC | Omega | 10.05 | Kapa | 6 | 10.05 |
| | | | HiSeq X | | | | Hyper | | |
| MSK-40_A | Pretreatment | K2-EDTA | Illumina | NYGC | Omega | 27.75 | Kapa | 6 | 25 |
| | | | HiSeq X | | | | Hyper | | |
| MSK-40_E | Week 3 | K2-EDTA | Illumina | NYGC | Omega | 21.225 | Kapa | 6 | 21.225 |
| | | | HiSeq X | | | | Hyper | | |
| MSK-40_H | Week 6 | K2-EDTA | Illumina | NYGC | Omega | 17.65 | Kapa | 6 | 17.65 |
| | | | HiSeq X | | | | Hyper | | |
| MSK-40_L | Week 12 | K2-EDTA | Illumina | NYGC | Omega | 14.3 | Kapa | 6 | 14.3 |
| | | | HiSeq X | | | | Hyper | | |
| MSK-41_A | Pretreatment | K2-EDTA | Illumina | NYGC | Omega | 10.375 | Kapa | 6 | 10.375 |
| | | | HiSeq X | | | | Hyper | | |
| MSK-41_C | Week 3 | K2-EDTA | Illumina | NYGC | Omega | 10.175 | Kapa | 6 | 10.175 |
| | | | HiSeq X | | | | Hyper | | |
| MSK-41_D | Week 6 | K2-EDTA | Illumina | NYGC | Omega | 16.275 | Kapa | 6 | 16.275 |
| | | | HiSeq X | | | | Hyper | | |
| MSK-42_A | Pretreatment | K2-EDTA | Illumina | NYGC | Omega | 18.85 | Kapa | 6 | 18.85 |
| | | | HiSeq X | | | | Hyper | | |

| | | | | | | |
|---|---|---|---|---|---|---|
| 1.13E+09 | 11.2391 | 31.1346 | 204.0806 | 99.84 | 38515428 | 0.07659964 |
| 1.16E+09 | 10.6782 | 31.2784 | 174.3234 | 99.92 | 19861691 | 0.03718054 |
| 1.1E+09 | 10.2503 | 25.6584 | 181.6116 | 99.89 | 16964417 | 0.06568659 |
| 1.1E+09 | 10.4805 | 28.0164 | 179.6258 | 99.92 | 18106119 | 0.07378796 |
| 1.08E+09 | 11.2785 | 27.4503 | 176.9264 | 99.87 | 16914865 | 0.06536061 |
| 1.04E+09 | 10.3464 | 27.9056 | 178.7398 | 99.87 | 16092231 | 0.06121356 |
| 1.22E+09 | 11.9855 | 32.4425 | 189.6845 | 99.89 | 24275904 | 0.07291756 |
| 1.04E+09 | 11.4981 | 26.2326 | 181.4272 | 99.87 | 18411614 | 0.08679172 |
| 1.25E+09 | 10.5154 | 34.8285 | 204.232 | 99.95 | 39777115 | 0.09545447 |
| 1.06E+09 | 10.9741 | 28.6057 | 183.8128 | 99.87 | 21637391 | 0.06694671 |
| 9.31E+08 | 10.6888 | 23.1302 | 169.1468 | 99.84 | 13834075 | 0.06313198 |
| 1.16E+09 | 9.3953 | 32.2348 | 205.2164 | 99.87 | 35542572 | 0.1660227 |
| 1.24E+09 | 11.0604 | 33.6652 | 190.1638 | 99.79 | 24130751 | 0.09401989 |
| 1.01E+09 | 10.8519 | 26.5983 | 174.7645 | 99.79 | 16030818 | 0.05774208 |
| 1.1E+09 | 10.071 | 30.5074 | 181.1631 | 99.84 | 21407732 | 0.0496435 |
| 9.62E+08 | 10.1683 | 25.9994 | 174.2099 | 99.92 | 15301175 | 0.05690717 |
| 1.11E+09 | 10.0821 | 30.9844 | 183.3404 | 99.84 | 19813206 | 0.05900503 |
| 1.13E+09 | 9.7758 | 32.4535 | 219.4067 | 99.82 | 49370461 | 0.03741848 |

| | | | | | | | | | |
|---|---|---|---|---|---|---|---|---|---|
| MSK-42_F | Week 3 | K2-EDTA | Illumina | NYGC | Omega | 11.55 | Kapa | 6 | 11.5 |
| | | | HiSeq X | | | | Hyper | | |
| MSK-42_I | Week 6 | K2-EDTA | Illumina | NYGC | Omega | 8.325 | Kapa | 6 | 8.325 |
| | | | HiSeq X | | | | Hyper | | |
| MSK-42_M | Week 12 | K2-EDTA | Illumina | NYGC | Omega | 7.55 | Kapa | 6 | 7.55 |
| | | | HiSeq X | | | | Hyper | | |
| MSK-45_A | Pretreatment | K2-EDTA | Illumina | NYGC | Omega | 18.825 | Kapa | 5 | 18.825 |
| | | | HiSeq X | | | | Hyper | | |
| MSK-45_C | Week 3 | K2-EDTA | Illumina | NYGC | Omega | 16.775 | Kapa | 5 | 16.775 |
| | | | HiSeq X | | | | Hyper | | |
| MSK-45_D | Week 6 | K2-EDTA | Illumina | NYGC | Omega | 15.325 | Kapa | 5 | 15.325 |
| | | | HiSeq X | | | | Hyper | | |
| MSK-45_E | Week 12 | K2-EDTA | Illumina | NYGC | Omega | 43.25 | Kapa | 5 | 43.25 |
| | | | HiSeq X | | | | Hyper | | |
| MSK-53_A | Pretreatment | K2-EDTA | Illumina | NYGC | Omega | 7.6 | Hyper | 5 | 7.6 |
| | | | HiSeq X | | | | Kapa | | |
| MSK-53_E | Week 3 | K2-EDTA | Illumina | NYGC | Omega | 221 | Kapa | 5 | 25 |
| | | | HiSeq X | | | | Hyper | | |
| MSK-53_F | Week 6 | K2-EDTA | Illumina | NYGC | Omega | 137.5 | Kapa | 5 | 25 |
| | | | HiSeq X | | | | Hyper | | |
| MSK-54_A | Pretreatment | K2-EDTA | Illumina | NYGC | Omega | 13.025 | Kapa | 5 | 13.025 |
| | | | HiSeq X | | | | Hyper | | |
| MSK-54_D | Week 3 | K2-EDTA | Illumina | NYGC | Omega | 88 | Kapa | 5 | 25 |
| | | | HiSeq X | | | | Hyper | | |
| MSK-54_E | Week 6 | K2-EDTA | Illumina | NYGC | Omega | 234.75 | Kapa | 5 | 25 |
| | | | HiSeq X | | | | Hyper | | |
| MSK-54_G | Week 12 | K2-EDTA | Illumina | NYGC | Omega | 600 | Kapa | 5 | 25 |
| | | | HiSeq X | | | | Hyper | | |
| MSK-55_A | Pretreatment | K2-EDTA | Illumina | NYGC | Omega | 19.9 | Kapa | 5 | 19.9 |
| | | | HiSeq X | | | | Hyper | | |
| MSK-55_D | Week 6 | K2-EDTA | Illumina | NYGC | Omega | 122.75 | Kapa | 5 | 25 |
| | | | HiSeq X | | | | Hyper | | |

| | | | | | | | |
|---|---|---|---|---|---|---|---|
| 9.38E+08 | 9.5579 | 25.8803 | 188.9788 | 99.79 | 18509519 | 0.03185549 | |
| 9.98E+08 | 11.6562 | 26.6778 | 188.0185 | 99.76 | 19893390 | 0.04017157 | |
| 1.03E+09 | 11.0653 | 29.0853 | 197.5049 | 99.87 | 28687958 | 0.0331781 | |
| 9.75E+08 | 9.5287 | 27.3915 | 187.9476 | 99.87 | 22259579 | 0.06532424 | |
| 9.22E+08 | 10.9061 | 22.8648 | 177.0223 | 99.82 | 17867891 | 0.09768872 | |
| 9.01E+08 | 18.9433 | 20.146 | 179.6611 | 99.84 | 16491691 | 0.05889882 | |
| 8.98E+08 | 9.9439 | 24.5899 | 184.4423 | 99.82 | 19097759 | 0.05219029 | |
| 9.24E+08 | 8.5553 | 25.8421 | 185.774 | 99.87 | 19666515 | 0.04391161 | |
| 1.28E+09 | 8.7727 | 34.9515 | 181.6614 | 99.84 | 26912492 | 0.09038024 | |
| 1.04E+09 | 9.9668 | 28.59 | 176.693 | 99.95 | 15966581 | 0.06301841 | |
| 1.07E+09 | 8.2932 | 32.3754 | 250.3176 | 99.85 | 57086107 | 0.02466991 | |
| 1.08E+09 | 7.8658 | 29.9857 | 183.94 | 99.9 | 17683545 | 0.02602525 | |
| 1.07E+09 | 9.2455 | 28.7411 | 170.462 | 99.85 | 14467517 | 0.06385017 | |
| 1.41E+09 | 8.2238 | 37.9175 | 174.4319 | 99.85 | 18572394 | 0.06053011 | |
| 9.3E+08 | 9.5654 | 23.2967 | 174.7593 | 35.15 | 17327169 | 0.07257793 | Excluded for low concordance |
| | | | | | | | (<99%) between pretreatment |
| | | | | | | | timepoint and subsequent |
| | | | | | | | timepoints) |
| 1.01E+09 | 7.5715 | 27.0062 | 185.1081 | 99.79 | 21771202 | 0.04967905 | Excluded for low concordance |
| | | | | | | | (<99%) between pretreatment |
| | | | | | | | timepoint and subsequent |
| | | | | | | | timepoints) |

| | | | | | | | | | |
|---|---|---|---|---|---|---|---|---|---|
| MSK-55_F | Week 12 | K2-EDTA | Illumina | NYGC | Omega | 21.35 | Kapa | 5 | 21.35 |

| | pre | | | | | | | | |
|---|---|---|---|---|---|---|---|---|---|
| | sequencing | | | | | | | | |
| | QC | | | | | | Hyper | sequencing | |
| HiSeq PON | Blood | | HiSeq X | | | library | # of | metrics | |
| Plasma | Collection | Sequencing | Sequencing | extraction | total | prep | PCR | library | TOTAL_{—} |
| Patient ID | Tube | Platform | Location | kit | mass | kit | cycles | mass | READS |
| HiSeq PON-1 | Streck | Illumina | NYGC | Omega | 143.1 | Kapa | 6 | 119.25 | 1.06E+09 |
| | | HiSeq X | | | | Hyper | | | |
| HiSeq PON-2 | Streck | Illumina | NYGC | Omega | 42 | Kapa | 6 | 35 | 9.45E+08 |
| | | HiSeq X | | | | Hyper | | | |
| HiSeq PON-3 | Streck | Illumina | NYGC | Omega | 31.5 | Kapa | 6 | 26.25 | 7.83E+08 |
| | | HiSeq X | | | | Hyper | | | |
| HiSeq PON-4 | Streck | Illumina | NYGC | Omega | 25.62 | Kapa | 6 | 21.35 | 1.32E+01 |
| | | HiSeq X | | | | Hyper | | | |
| HiSeq PON-5 | Streck | Illumina | NYGC | Omega | 9.8 | Kapa | 6 | 9.8 | 7.86E+08 |
| | | HiSeq X | | | | Hyper | | | |
| HiSeq PON-6 | Streck | Illumina | NYGC | Omega | 19.525 | Kapa | 6 | 19.525 | 1.06E+09 |
| | | HiSeq X | | | | Hyper | | | |
| HiSeq PON-7 | Streck | Illumina | NYGC | Omega | 33.5 | Kapa | 6 | 25 | 9.67E+08 |
| | | HiSeq X | | | | Hyper | | | |
| HiSeq PON-8 | Streck | Illumina | NYGC | Omega | 5.475 | Kapa | 6 | 5.5 | 8.62E+08 |
| | | HiSeq X | | | | Hyper | | | |

| | | | | | | |
|---|---|---|---|---|---|---|
| 9.33E+08 | 13.1767 | 183.5927 | 99.66 | 18799584 | 0.0615897 | Excluded for low concordance |
| | | | | | | (<99%) between pretreatment |
| | | | | | | timepoint and subsequent |
| | | | | | | timepoints) |

| | | QC metrics | | | |
|---|---|---|---|---|---|
| Percent- | | MEDIAN_{—} | | | |
| Total- | Mean | INSERT_{—} | Conpair- | Auto- | |
| Duplication | Coverage | SIZE | Concordance | correlation | Notes |
| 9.5367 | 29.7647 | 176.5491 | NA | 0.0521721 | |
| 10.9932 | 25.1701 | 173.7452 | NA | 0.0642385 | |
| 9.3594 | 19.7805 | 171.9292 | NA | 0.07659964 | |
| 13.1767 | 23.1686 | 1.32E+01 | NA | 0.0615897 | |
| 8.0893 | 21.4504 | 173 | NA | 0.0336 | |
| 11.3325 | 29.1876 | 175 | NA | 0.053 | |
| 11.4483 | 28.2226 | 189 | NA | 0.0451 | |
| 11.6955 | 22.9875 | 176 | NA | 0.0514 | |

* these samples were included in the panel of normal samples for Illumina HiSeq X data and not included in any other analysis
*library mass cal [symbol] [symbol]
pped at 25 ng [symbol]
indicates data missing or illegible when filed

### Appendix 2

SNV fragment deep learning model training and validation samples

| | | | | | Post filter | | |
|---|---|---|---|---|---|---|---|
| Cancer | Data Set | Sample | | Samples | fragments | iChorCNA | |
| Type | Type | type | Label | Used | contributed | TF | Label annotation |
| Melanoma | Training | Melanoma | TRUE | AD-05_A ∩ | 270648 | 0.24 and 0.14 | True label in melanoma is the |
| | fragments | | | AD-05_B | | | intersection of SNV fragments called |
| | | | | | | | using Mutect2 from two high burden |
| | | | | | | | plasma samples from the same patient |
| | | | | | | | (AD-05) at the pretreatment (‘A’) and |
| | | | | | | | Week 3 timepoint (‘B’). The intersection |
| | | | | | | | of the 2 plasma samples is performed to |
| | | | | | | | increase specificity for true ctDNA |
| | | | | | | | mutations in the positive label set |
| | | Healthy | FALSE | C-24 | 45108 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-12 | 45108 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-14 | 45108 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-32 | 45108 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-36 | 45108 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Melanoma | FALSE | AD-05_D | 45108 | <0.05 | Randomly selected post-filter cfDNA |
| | | | | | | | SNV pileup fragments from patient AD- |
| | | | | | | | 05 at the Week 9 (‘D’) timepoint following |
| | | | | | | | a major response to immunotherapy. |
| | | | | | | | Included to reduce patient-specific bias |
| | | | | | | | during model training. Germline is |
| | | | | | | | excluded through variant allele frequency |
| | | | | | | | filter (<0.2). Fragment contribution is |
| | | | | | | | designed to match true label fragment |
| | | | | | | | corpus size with equal contribution from |
| | | | | | | | false label samples |
| | Held-out | Melanoma | TRUE | MEL-01 | 180390 | 0.02 (low TF | SNV mutation calling was performed on |
| | validation | | | | | setting) to | matched tumor and PBMC samples |
| | fragments | | | | | 0.06 (low TF | using the NYGC somatic mutation calling |
| | | | | | | setting) | pipeline. Selected fragments were |
| | | | | | | | confined to SNVs in plasma that match |
| | | | | | | | the tumor-informed mutation |
| | | | | | | | compendium |
| | | Healthy | FALSE | C-38 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-10 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-21 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-05 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-16 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-35 | 30065 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| LUAD | Training | LUAD | TRUE | CM-6_0w | 62650 | 0.14 | SNV mutations called via Mutect2 |
| | fragments | | | | | | consensus mutation detection directly in |
| | | | | | | | high burden plasma |
| | | LUAD | TRUE | CM-30_0w | 62650 | 0.12 | SNV mutations called via Mutect2 |
| | | | | | | | consensus mutation detection directly in |
| | | | | | | | high burden plasma |
| | | Healthy | FALSE | C-24 | 25060 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-12 | 25060 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-14 | 25060 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-32 | 25060 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-31 | 25060 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | Held-out | LUAD | TRUE | LUAD-05 | 3706 | 0.04 | SNV mutation calling was performed on |
| | validation | | | preoperative | | | matched tumor and PBMC samples |
| | fragments | | | | | | using the NYGC somatic mutation calling |
| | | | | | | | pipeline. Selected fragments were |
| | | | | | | | confined to SNVs in plasma that match |
| | | | | | | | the tumor-informed mutation |
| | | | | | | | compendium |
| | | LUAD | TRUE | LUAD-34 | 3706 | 0.05 | SNV mutation calling was performed on |
| | | | | preoperative | | | matched tumor and PBMC samples |
| | | | | | | | using the NYGC somatic mutation calling |
| | | | | | | | pipeline. Selected fragments were |
| | | | | | | | confined to SNVs in plasma that match |
| | | | | | | | the tumor-informed mutation |
| | | | | | | | compendium |
| | | Healthy | FALSE | C-17 | 1482 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-26 | 1482 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-35 | 1482 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | C-20 | 1482 | N/A | Randomly subsampled post-filter cfDNA |
| | | control | | | | | SNV from plasma SNV pileup designed |
| | | | | | | | to match true label fragment corpus size |
| | | | | | | | with equal contribution from false label |
| | | | | | | | samples. Germline is excluded through |
| | | | | | | | variant allele frequency filter (<0.2). |
| CRC | Training | CRC | TRUE | MF-5812 | 12790 | 0.23 | SNV mutation calling was performed on |
| | fragments | | | | | | matched tumor and PBMC samples |
| | | | | | | | using the NYGC somatic mutation calling |
| | | | | | | | pipeline. Fragments used in training |
| | | | | | | | were confined to SNVs in plasma that |
| | | | | | | | match the tumor-informed mutation |
| | | | | | | | compendium. ctDNA SNVs were |
| | | | | | | | downsampled to ensure equal fragment |
| | | | | | | | contribution from all true label samples |
| | | CRC | TRUE | MF-3930 | 12790 | 0.12 | SNV mutation calling was performed on |
| | | | | | | | matched tumor and PBMC samples |
| | | | | | | | using the NYGC somatic mutation calling |
| | | | | | | | pipeline. Fragments used in training |
| | | | | | | | were confined to SNVs in plasma that |
| | | | | | | | match the tumor-informed mutation |
| | | | | | | | compendium. ctDNA SNVs were |
| | | | | | | | downsampled to ensure equal fragment |
| | | | | | | | contribution from all true label samples |
| | | CRC | TRUE | MF-6596 | 12790 | 0.09 | SNV mutation calling was performed on |
| | | | | | | | matched tumor and PBMC samples |
| | | | | | | | using the NYGC somatic mutation calling |
| | | | | | | | pipeline. Fragments used in training |
| | | | | | | | were confined to SNVs in plasma that |
| | | | | | | | match the tumor-informed mutation |
| | | | | | | | compendium. ctDNA SNVs were |
| | | | | | | | downsampled to ensure equal fragment |
| | | | | | | | contribution from all true label samples |
| | | CRC | TRUE | MF-5766 | 12790 | 0.1 | SNV mutation calling was performed on |
| | | | | | | | matched tumor and PBMC samples |
| | | | | | | | using the NYGC somatic mutation calling |
| | | | | | | | pipeline. Fragments used in training |
| | | | | | | | were confined to SNVs in plasma that |
| | | | | | | | match the tumor-informed mutation |
| | | | | | | | compendium. ctDNA SNVs were |
| | | | | | | | downsampled to ensure equal fragment |
| | | | | | | | contribution from all true label samples |
| | | Healthy | FALSE | Donor333 | 12790 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | Donor358 | 12790 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | Donor340 | 12790 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | Donor356 | 12790 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |
| | Held-out | CRC | TRUE | MF-5823 | 13079 | 0.11 | SNV mutation calling was performed on |
| | validation | | | | | | matched tumor and PBMC samples |
| | fragments | | | | | | using the NYGC somatic mutation calling |
| | | | | | | | pipeline. Selected fragments were |
| | | | | | | | confined to SNVs in plasma that match |
| | | | | | | | the tumor-informed mutation |
| | | | | | | | compendium |
| | | Healthy | FALSE | Donor337 | 6539 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |
| | | Healthy | FALSE | Donor343 | 6539 | N/A | Randomly subsampled post-filter cfDNA |
| | | control | | (Control | | | SNV from plasma SNV pileup designed |
| | | | | Cohort B) | | | to match true label fragment corpus size |
| | | | | | | | with equal contribution from false label |
| | | | | | | | samples. Germline is excluded through |
| | | | | | | | variant allele frequency filter (<0.2). |

Training and validation performance

| Cancer | Data Set | | Accuracy | |
|---|---|---|---|---|
| Type | Type | F1 (%) | (%) | AUC (%) |
| Melanoma | Training | 90.5 | 90.5 | 96.1 |
| | fragments | | | |
| | Held-out | 88.6 | 88.8 | 95.2 |
| | validation | | | |
| | fragments | | | |
| NSCLC | Training | 79.5 | 79.3 | 87.3 |
| | fragments | | | |
| | Held-out | 78.6 | 78.9 | 86.8 |
| | validation | | | |
| | fragments | | | |
| CRC | Training | 75.7 | 75.8 | 84.3 |
| | fragments | | | |
| | Held-out | 75.6 | 75.2 | 83.6 |
| | validation | | | |
| | fragments | | | |

TF admixtures

| | | | | | | | Pretreatment | Posttreatment | | |
|---|---|---|---|---|---|---|---|---|---|---|
| | | | Low TF/ | Highest | Lowest | Tumor | aneuploidy on | aneuploidyon | | |
| SNV or | Cancer | High TF | control | mix | mix | Aneuploidy | iChorCNA | iChorCNA | | |
| CNV | Type | sample | sample | fraction | fraction | (if CNV) | (if CNV) | (if CNV) | Replicates | Coverage |
| SNV | Melanoma | MEL-01 | C-16 | 10⁻³ | 10⁻⁷ | | | | 20 | 16X |
| CNV | Melanoma | AD-12_A | AD-12_D | 10⁻³ | 10⁻⁶ | 1.6 Gb | Yes | No | 50 | 35X |
| | | (pretreatment) | (posttreatment) | | | | | | | |
| CNV | NSCLC | Neo-03 | Neo-03 | 10⁻³ | 10⁻⁶ | 1.8 Gb | Yes | No | 20 | 40X |
| | | preoperative | postoperative | | | | | | | |

### Appendix 3

| Model Type | Filters applied |
|---|---|
| Melanoma | Mean read base quality ≥ 10 |
| | Read depth ≥ 10 |
| | Variant base quality ≥ 25 |
| | 40 bp ≤ Fragment length ≤ 240 |
| | Variant allele frequency ≤ 0.2 unless iChorCNA est. TF > 0.2 |
| | Variant present on both paired, overlapping reads |

| Feature Used | svROC | Source | ENCODE identifier |
|---|---|---|---|
| Primary Melanocyte H3K27ac | 0.590786109 | ENCODE | ENCFF449ZJA |
| Primary Melanocyte H3K27me3 | 0.561887283 | ENCODE | ENCFF653ZQK |
| Primary Melanocyte H3K36me3 | 0.627993827 | ENCODE | ENCFF374UAV |
| Primary Melanocyte H3K4me1 | 0.640657273 | ENCODE | ENCFF462CRG |
| Astrocyte H3K4me2 | 0.616720151 | ENCODE | ENCFF871OQF |
| PBMC H3K4me3 | 0.510223358 | ENCODE | ENCFF513BFG |
| PBMC H3K9ac | 0.611767884 | ENCODE | ENCFF072IGM |
| CD4 T-cell H3K9me3 | 0.57722118 | ENCODE | ENCFF616YFF |
| Primary Melanocyte H3K9me3 | 0.50317072 | ENCODE | ENCFF613SAA |
| Number of low quality bases (BQ < 20) on Read 1 | 0.504998219 | Alignment file | |
| Melanoma ATAC-seq accessibiilty | 0.616856444 | TCGA¹ | |
| Umbilical Vein CTCF TF Binding | 0.584826073 | ENCODE | ENCFF209BDU |
| Primary Melanocyte DNase Hypersensitivity | 0.64020356 | ENCODE | ENCFF454SUH |
| T-cell Hi-C compartment | 0.595840967 | HI-C SNIPER² | |
| Primary Melanocyte chromHMM annotations | 0.623419556 | RegulomeDB | TSTFF372537 |
| Variant on coding strand | 0.511677296 | Haradhvala et al.³ | |
| Variant on template strand | 0.513512836 | Haradvalla et al.³ | |
| Transcription direction (from 3′ end) | 0.529763322 | Haradvalla et al.³ | |
| Transcription direction (from 5′ end) | 0.528316189 | Haradvalla et al.³ | |
| Mean RNA Expression | 0.62315935 | Haradvalla et al.³ | |
| Primary Melanocyte PCAWG SNV mutation density | 0.693508217 | PCAWG⁴ | |
| Plasma WGS sequencing error density | 0.526171606 | Internal data | |
| Replication timing | 0.593513764 | Haradhvala et al.³ | |
| Melanocyte RNA Expression | 0.639533614 | ENCODE | ENCFF864WZG |

| LUAD | Filters Applied |
|---|---|
| | Mean read base quality ≥ 10 |
| | Read depth ≥ 10 |
| | Variant base quality ≥ 25 |
| | 40bp ≤ Fragment length ≤ 240 |
| | Variant allele frequency ≤ 0.2 unless iChorCNA est. TF > 0.2 |

| Feature Used | svROC | Source | ENCODE identifier |
|---|---|---|---|
| Trophoblast H3K27ac | 0.628474803 | ENCODE | ENCFF543JKQ |
| Breast Epithelium H3K36me3 | 0.516340788 | ENCODE | ENCFF046ZVO |
| Bronchial Epithelial Cell H3K36me3 | 0.621938584 | ENCODE | ENCFF743JIC |
| Keratinocyte H3K4me1 | 0.637172172 | ENCODE | ENCFF040MAX |
| Keratinocyte H3K4me2 | 0.636439095 | ENCODE | ENCFF049LTK |
| Foreskin Fibroblast H3K4me3 | 0.639578601 | ENCODE | ENCFF955FBX |
| Neuron H9 H3K9me3 | 0.581359003 | ENCODE | ENCFF 169TUP |
| Suprapubic Skin H3K9me3 | 0.570072264 | ENCODE | ENCFF993GFH |
| Number of low quality bases (BQ < 20) on R1 | 0.706454757 | Alignment file | |
| Lung ATAC seq | 0.6741188 | TCGA¹ | |
| Lung fibroblast CTCF TF Binding | 0.586665976 | ENCODE | ENCFF892QTE |
| Lung DNase Hypersensitivity | 0.665075637 | ENCODE | ENCFF690UKD |
| T-cell Hi-C compartment | 0.665813755 | HI-C SNIPER² | |
| Lung chromHMM Regions | 0.590246257 | RegulomeDB | TSTFF258425 |
| Variant on coding strand | 0.510782811 | Haradhvala et al.³ | |
| Variant on template strand | 0.513419619 | Haradvalla et al.³ | |
| Transcription direction(3′ end) | 0.537006131 | Haradvalla et al.³ | |
| Transcription direction(5′ end) | 0.54262886 | Haradvalla et al.³ | |
| Lung PCAWG mutation density | 0.668310164 | PCAWG⁴ | |
| Mean RNA Expression | 0.675245633 | Haradhvala et al.³ | |
| Plasma WGS sequencing error density | 0.599543332 | Internal data | |
| Replication timing | 0.594134236 | Haradhvala et al.³ | |
| Lung RNA Expression | 0.628747397 | ENCODE | ENCFF967XNR |

| CRC | Filters Applied |
|---|---|
| | Mean read base quality ≥ 10 |
| | Read depth ≥ 10 |
| | Variant base quality ≥ 25 |
| | 40bp ≤ Fragment length ≤ 240 |
| | Variant allele frequency ≤ 0.2 unless iChorCNA est. TF > 0.2 |
| | Variant present on both paired, overlapping reads |

| Feature Used | svROC | Source | ENCODE identifier |
|---|---|---|---|
| Primary Melanocyte H3K27ac | 0.570391093 | ENCODE | ENCFF449ZJA |
| Trophoblast H3K27ac | 0.593424904 | ENCODE | ENCFF543JKQ |
| Primary Melanocyte H3K27me3 | 0.500972745 | ENCODE | ENCFF653ZQK |
| Breast Epithelium H3K36me3 | 0.517263137 | ENCODE | ENCFF046ZVO |
| Bronchial Epithelial Cell H3K36me3 | 0.585737349 | ENCODE | ENCFF743JIC |
| Primary Melanocyte H3K36me3 | 0.587465624 | ENCODE | ENCFF374UAV |
| Keratinocyte H3K4me1 | 0.596658674 | ENCODE | ENCFF040MAX |
| Primary Melanocyte H3K4me1 | 0.587655368 | ENCODE | ENCFF462CRG |
| Keratinocyte H3K4me2 | 0.596953088 | ENCODE | ENCFF049LTK |
| Neural cell H3K4me2 | 0.524441562 | ENCODE | ENCFF454FGI |
| PBMC H3K4me3 | 0.515903162 | ENCODE | ENCFF513BFG |
| Foreskin Fibroblast H3K4me3 | 0.600498698 | ENCODE | ENCFF955FBX |
| Primary Melanocyte H3K9me3 | 0.50330674 | ENCODE | ENCFF613SAA |
| Neuron H9 H3K9me3 | 0.554437937 | ENCODE | ENCFF 169TUP |
| Suprapubic Skin H3K9me3 | 0.55298138 | ENCODE | ENCFF993GFH |
| R1 # of low quality bases (BQ < 20) | 0.543485896 | Alignment file | |
| R2 # of low quality bases (BQ < 20) | 0.520062999 | Alignment file | |
| Bound TF Distance | 0.613768048 | Sabarinathan et al.⁵ | |
| Lung fibroblast CTCF TF Binding | 0.55747897 | ENCODE | ENCFF892QTE |
| Umbilical Vein CTCF TF Binding | 0.572361297 | ENCODE | ENCFF209BDU |
| Primary Melanocyte DNase Hypersensitivity | 0.611848658 | ENCODE | ENCFF454SUH |
| Dyad Distance | 0.511009235 | Pech et al.⁶ | |
| gm12878 cell line Hi-C | 0.627272363 | HI-C SNIPER² | |
| HSPC cell line Hi-C compartment | 0.624452143 | HI-C SNIPER² | |
| HUVEC cell line compartment Hi-C | 0.628210019 | HI-C SNIPER² | |
| T-cell Hi-C compartment | 0.630217819 | HI-C SNIPER² | |
| Lung chromHMM Regions | 0.557292415 | RegulomeDB | |
| Primary Melanocyte chromHMM Regions | 0.570227014 | RegulomeDB | |
| Variant on coding strand | 0.511374714 | Haradhvala et al.³ | |
| Variant on template strand | 0.510769231 | Haradhvala et al.³ | |
| Transcription direction(from 3′) | 0.526717441 | Haradhvala et al.³ | |
| Transcription direction(from 5′) | 0.528808073 | Haradhvala et al.³ | |
| Colon PCAWG mutational density | 0.60067494 | PCAWG⁴ | |
| Plasma WGS sequencing error density | 0.588046715 | Internal data | |
| Replication timing | 0.558471803 | Haradhvala et al.³ | |
| Colon RNA Expression | 0.603296139 | ENCODE | ENCFF329ENM |
| Prostate Epithelial CTCF TF Binding | 0.54623062 | ENCODE | ENCFF608KCO |
| h1 Trophoblast H3K9ac | 0.567232551 | ENCODE | ENCFF313IDN |
| Small intestine H3K36me3 | 0.598058548 | ENCODE | ENCFF674FLQ |
| PBMC H3K4me1 | 0.598242764 | ENCODE | ENCFF581RRW |
| Thyroid Gland H3K36me3 | 0.515248176 | ENCODE | ENCFF527VVQ |
| Human vcap H3K27ac | 0.577340819 | ENCODE | ENCFF458HWQ |
| Mononuclear H3K9me3 | 0.571663824 | ENCODE | ENCFF027UIW |

¹Corces MR, Granja JM, Shams S, et al. The chromatin accessibility landscape of primary human cancers. Science. 2018; 362(6413). doi: 10.1126/science.aav1898
²Xiong K, Ma J. Revealing Hi-C subcompartments by imputing inter-chromosomal chromatin interactions. Nat Commu 2019; 10(1): 5069
³Haradhvala NJ, Polak P, Stojanov P, et al. Mutational strand asymmetries in cancer genomes reveal mechanisms of and repair. Cell. 2016; 164(3): 538-549.
⁴Gerstung M, Jolly C, Leshchiner I, et al. The evolutionary history of 2,658 cancers. Nature. 2020; 578(7793): 122-128.
⁵Sabarinathan R, Mularoni L, Deu-Pons J, Gonzalez-Perez A, López-Bigas N. Nucleotide excision repair is impaired b transcription factors to DNA. Nature. 2016; 532(7598): 264-267.
⁶Pich O, Mui\ddot{n}os F, Sabarinathan R, Reyes-Salazar I, Gonzalez-Perez A, Lopez-Bigas N. Somatic and germline mutat follow the orientation of the DNA minor groove around nucleosomes. Cell. 2018; 175(4): 1074-1087.e18.

| Panel of | | |
|---|---|---|
| normal | | |
| samples | | |
| (PON) | | Illumina |
| patient IDs | Illumina HiSeq X | NovaSeq |
| | Control-05 | donor333 |
| | Control-06 | donor336 |
| | Control-08 | donor340 |
| | Control-10 | donor352 |
| | Control-11 | donor358 |
| | Control-13 | Aar-16 |
| | Control-14 | Aar-18 |
| | Control-15 | Aar-21 |
| | Control-16 | Aar-22 |
| | Control-17 | Aar-25 |
| | Control-18 | C-01 |
| | Control-20 | C-04 |
| | Control-22 | C-05 |
| | Control-27 | C-06 |
| | Control-28 | C-07 |
| | Control-29 | C-08 |
| | Control-30 | C-09 |
| | Control-31 | C-10 |
| | Control-32 | C-11 |
| | Control-33 | C-12 |
| | Control-34 | C-13 |
| | Control-35 | C-14 |
| | Control-36 | C-15 |
| | Control-37 | C-16 |
| | HiSeq PON 1 | C-17 |
| | HiSeq PON 2 | C-19 |
| | HiSeq PON 3 | C-20 |
| | HiSeq PON 4 | C-21 |
| | HiSeq PON 5 | C-22 |
| | HiSeq PON 6 | C-23 |
| | HiSeq PON 7 | C-24 |
| | HiSeq PON 8 | C-25 |
| | MSK-32_C | C-26 |
| | MSK-37_D | C-27 |
| | MSK-37_G | C-28 |
| | MSK-38_C | C-29 |
| | MSK-38_D | C-30 |
| | MSK-38_H | C-31 |
| | MSK-40_E | C-32 |
| | MSK-40_H | C-33 |
| | MSK-40_L | C-34 |
| | MSK-41_D | C-35 |
| | MSK-42_M | C-36 |
| | MSK-45_E | C-37 |
| | MSK-53_F | C-38 |
| | MSK-54_D | |
| | MSK-54_G | |
| | MSK-55_F | |

Interval size

| | |
|---|---|
| 100 | Kb |
| 100 | Kb |
| 100 | Kb |
| 100 | Kb |
| 100 | Kb |
| 100 | Kb |
| 100 | Kb |
| 100 | Kb |
| 100 | Kb |
| 500 | Kb |
| 200 | Kb |
| 100 | Kb |
| 1 | Mb |
| 500 | Kb |
| 1 | Mb |
| 500 | Kb |
| 1 | Mb |
| 1 | Mb |
| 500 | Kb |
| 500 | Kb |
| 1 | Mb |
| 1 | Mb |
| 1 | Mb |
| 100 | Kb |
| 1 | Mb |
| 100 | Kb |
| 500 | Kb |
| 1 | Mb |
| 100 | Kb |
| 500 | Kb |
| 100 | Kb |
| 1 | Mb |
| 500 | Kb |
| 100 | Kb |
| 1 | Mb |
| 100 | Kb |
| 500 | Kb |
| 500 | Kb |
| 1 | Mb |
| 100 | Kb |
| 200 | Kb |
| 1 | Mb |
| 500 | Kb |
| 500 | Kb |
| 500 | Kb |
| 500 | Kb |
| 500 | Kb |
| 1 | Mb |
| 500 | Kb |

n. DNA damage
y binding of ion periodicity

### Appendix 4

| | | | | | | | | | | | pre- |
|---|---|---|---|---|---|---|---|---|---|---|---|
| | | | | | | | | | | | operative |
| | | | | | | CANCER | TUMOR | Adj | | RFS | plasma |
| Cohort | ID | Histology | AGE | Gender | SMOKER | STAGE | SIZE (CM) | treatment | Recurrance | [months] | sample |
| Early- | LUAD01 | Adenocarcinoma | 72 | M | Former | IA | 1.8 | | Not | Not | + |
| stage | | | | | | | | | applicable | applicable | |
| LUAD | | | | | | | | | | | |
| Early- | LUAD02 | Squamous | 67 | M | Former | IA | 2.4 | | Not | Not | + |
| stage | | | | | | | | | applicable | applicable | |
| LUAD | | | | | | | | | | | |
| Early- | LUAD03 | Adenocarcinoma | 62 | F | Former | IA | 2.1 | | Not | Not | + |
| stage | | | | | | | | | applicable | applicable | |
| LUAD | | | | | | | | | | | |
| Early- | LUAD04 | LUAD | 72 | F | Former | IA | 2.6 | | No | 12 | + |
| stage | | | | | | | | | | | |
| LUAD | | | | | | | | | | | |
| Early- | LUAD05 | Squamous | 84 | F | Former | IA | 2.8 | | Not | Not | + |
| stage | | | | | | | | | applicable | applicable | |
| LUAD | | | | | | | | | | | |
| Early- | LUAD06 | LUAD | 73 | F | Former | IA | 3 | | No | 45 | + |
| stage | | | | | | | | | | | |
| LUAD | | | | | | | | | | | |
| Early- | LUAD07 | Squamous | 73 | M | Former | IA | 2.8 | | Not | Not | + |
| stage | | | | | | | | | applicable | applicable | |
| LUAD | | | | | | | | | | | |
| Early- | LUAD08 | Squamous | 79 | M | Former | IA | 2.6 | | Not | Not | + |
| stage | | | | | | | | | applicable | applicable | |
| LUAD | | | | | | | | | | | |
| Early- | LUAD09 | Adenocarcinoma | 78 | F | Current | IA | 1.3 | | Not | Not | + |
| stage | | | | | | | | | applicable | applicable | |
| LUAD | | | | | | | | | | | |
| Early- | LUAD10 | LUAD | 76 | M | Former | IA | 2.6 | | No | 47 | + |
| stage | | | | | | | | | | | |
| LUAD | | | | | | | | | | | |
| Early- | LUAD11 | LUAD | 56 | M | Current | IA | 1.4 | | No | 36 | + |
| stage | | | | | | | | | | | |
| LUAD | | | | | | | | | | | |
| Early- | LUAD12 | LUAD | 75 | F | Former | IA | 0.8 | | No | 35 | + |
| stage | | | | | | | | | | | |
| LUAD | | | | | | | | | | | |
| Early- | LUAD13 | squamous | 77 | F | Former | IA | 2.5 | | Not | Not | + |
| stage | | cell | | | | | | | applicable | applicable | |
| LUAD | | carcinoma | | | | | | | | | |
| Early- | LUAD14 | Adenocarcinoma | 67 | F | Former | IA | 2 | | No | 34 | + |
| stage | | | | | | | | | | | |
| LUAD | | | | | | | | | | | |
| Early- | LUAD15 | Adenocarcinoma | 78 | F | Never | IA | 2.3 | | No | 18 | + |
| stage | | | | | | | | | | | |
| LUAD | | | | | | | | | | | |
| Early- | LUAD16 | Pleomorphic | 75 | M | Former | IB | 3.3 | | Not | Not | + |
| stage | | carcinoma | | | | | | | applicable | applicable | |
| LUAD | | | | | | | | | | | |
| Early- | LUAD17 | Adenocarcinoma | 75 | F | Former | IB | 4.7 | | Not | Not | + |
| stage | | | | | | | | | applicable | applicable | |
| LUAD | | | | | | | | | | | |
| Early- | LUAD18 | LUAD | 77 | M | Former | IB | 2.8 | | Yes | 6 | + |
| stage | | | | | | | | | | | |
| LUAD | | | | | | | | | | | |
| Early- | LUAD19 | LUAD | 72 | M | Former | IB | 3.7 | | No | 40 | + |
| stage | | | | | | | | | | | |
| LUAD | | | | | | | | | | | |
| Early- | LUAD20 | LUAD | 76 | M | Former | IB | 2.6 | | No | 42 | + |
| stage | | | | | | | | | | | |
| LUAD | | | | | | | | | | | |
| Early- | LUAD21 | Large cell | 69 | F | Former | IB | 2.5 | | Not | Not | + |
| stage | | neuroendocrine | | | | | | | applicable | applicable | |
| LUAD | | carcinoma | | | | | | | | | |
| Early- | LUAD22 | Adenocarcinoma | 65 | M | Former | IB | 3.3 | | No | 37 | + |
| stage | | | | | | | | | | | |
| LUAD | | | | | | | | | | | |
| Early- | LUAD23 | Adenocarcinoma | 65 | M | Current | IB | 2.8 | | No | 31 | + |
| stage | | | | | | | | | | | |
| LUAD | | | | | | | | | | | |
| Early- | LUAD24 | Squamous | 84 | M | Former | IIA | 5.5 | | Not | Not | + |
| stage | | | | | | | | | applicable | applicable | |
| LUAD | | | | | | | | | | | |
| Early- | LUAD25 | LUAD | 79 | M | Former | IIA | 2.3 | | No | 26 | + |
| stage | | | | | | | | | | | |
| LUAD | | | | | | | | | | | |
| Early- | LUAD26 | LUAD | 69 | F | Former | IIA | 2.1 | | Yes | 6 | + |
| stage | | | | | | | | | | | |
| LUAD | | | | | | | | | | | |
| Early- | LUAD27 | Adenocarcinoma | 63 | F | Never | IIA | 2.4 | Yes | Yes | 7 | + |
| stage | | | | | | | | | | | |
| LUAD | | | | | | | | | | | |
| Early- | LUAD28 | Adenocarcinoma | 66 | M | Current | IIA | 1.4 | | No | 35 | + |
| stage | | | | | | | | | | | |
| LUAD | | | | | | | | | | | |
| Early- | LUAD29 | Squamous | 66 | M | Former | IIB | 2.5 | | Not | Not | + |
| stage | | | | | | | | | applicable | applicable | |
| LUAD | | | | | | | | | | | |
| Early- | LUAD30 | Adenocarcinoma | 67 | F | Former | IIB | LLL- | | Not | Not | + |
| stage | | | | | | | 1.40 cm; | | applicable | applicable | |
| LUAD | | | | | | | LLL- | | | | |
| | | | | | | | 1.00 cm; | | | | |
| | | | | | | | LLL- | | | | |
| | | | | | | | 0.90 cm | | | | |
| Early- | LUAD31 | LUAD | 87 | F | Former | IIIA | 4.4 | | Yes | 6 | + |
| stage | | | | | | | | | | | |
| LUAD | | | | | | | | | | | |
| Early- | LUAD32 | LUAD | 65 | F | Current | IIIA | 2 | Yes | No | 33 | + |
| stage | | | | | | | | | | | |
| LUAD | | | | | | | | | | | |
| Early- | LUAD33 | Carcinosarcoma | 75 | F | Current | IIIA | 4.5 | | Not | Not | + |
| stage | | | | | | | | | applicable | applicable | |
| LUAD | | | | | | | | | | | |
| Early- | LUAD34 | squamous | 72 | F | Current | IIIA | 6.5 | | Not | Not | + |
| stage | | cell | | | | | | | applicable | applicable | |
| LUAD | | carcinoma | | | | | | | | | |
| Early- | LUAD35 | Adenocarcinoma | 81 | F | Former | IIIA | 1.7 | Yes | No | 34 | + |
| stage | | | | | | | | | | | |
| LUAD | | | | | | | | | | | |
| Early- | LUAD36 | Adenocarcinoma | 67 | M | Former | IV | No | | Not | Not | + |
| stage | | | | | | | residual | | applicable | applicable | |
| LUAD | | | | | | | viable | | | | |
| | | | | | | | carcinoma | | | | |
| Early- | LUAD37 | LUAD | 77 | F | Former | IA | 2.2 | | No | 39 | − |
| stage | | | | | | | | | | | |
| LUAD | | | | | | | | | | | |
| Early- | LUAD38 | LUAD | 60 | F | Former | IA | 1.9 | | No | 54 | − |
| stage | | | | | | | | | | | |
| LUAD | | | | | | | | | | | |
| Early- | LUAD39 | LUAD | 80 | M | Former | IA | 1.2 | | No | 42 | − |
| stage | | | | | | | | | Only | Only | |
| LUAD | | | | | | | | | included if | included if | |
| | | | | | | | | | post- | post- | |
| | | | | | | | | | operative | operative | |
| | | | | | | | | | plasma | plasma | |
| | | | | | | | | | sample | sample | |
| | | | | | | | | | collected | collected | |

| | | | | | | | | | | pre- | post- | | |
|---|---|---|---|---|---|---|---|---|---|---|---|---|---|
| | | | | | | | | | | operative | operative | | |
| | | | | | | CANCER | Adj | Recur- | RFS | plasma | plasma | post-operative | |
| Cohort | ID | Histology | AGE | Gender | MSI | STAGE | treatment | rance | [months] | sample | sample | plasma sample | |
| CRC | CRC 1 | Adenocarcinoma | 82 | F | | IIA | NA | | 24 | + | + | − | + |
| CRC | CRC 2 | Adenocarcinoma | 81 | M | | IIA | NA | | 44 | + | + | − | − |
| CRC | CRC 3 | Sigmoid Colon | 72 | M | | IIA | NA | | 17 | + | + | − | + |
| CRC | CRC 4 | NA | 72 | M | Yes | IIA | NA | | 50 | + | + | + | + |
| CRC | CRC 5 | Adenocarcinoma | 60 | M | Yes | IIA | NA | | 28 | + | + | − | − |
| CRC | CRC 6 | Adenocarcinoma | 35 | F | | IIA | Yes | | 37 | + | + | + | + |
| CRC | CRC 7 | Adenocarcinoma | 35 | M | | IIA | Yes | | 37 | + | + | − | + |
| CRC | CRC 8 | NA | 68 | M | Yes | IIB | NA | | 52 | + | + | − | + |
| CRC | CRC 9 | NA | 65 | M | | IIB | Yes | | 43 | + | + | − | + |
| CRC | CRC 10 | NA | 82 | F | | IIB | Yes | | 38 | + | + | + | − |
| CRC | CRC 11 | NA | 43 | M | Yes | IIB | NA | | 37 | + | + | + | − |
| CRC | CRC 12 | NA | 52 | M | | III | Yes | | 50 | + | + | + | + |
| CRC | CRC 13 | NA | 52 | F | Yes | III | Yes | | 19 | + | + | − | + |
| CRC | CRC 14 | Adenocarcinoma | 62 | M | | III | NA | Yes | 20 | + | + | + | − |
| CRC | CRC 15 | Adenocarcinoma | 50 | F | | III | Yes | | 51 | + | + | + | − |
| CRC | CRC 16 | NA | 47 | F | | III | Yes | Yes | 17 | + | + | − | + |
| CRC | CRC 17 | NA | 58 | M | | III | Yes | | 38 | + | + | − | − |
| CRC | CRC 18 | NA | 77 | M | Yes | III | Yes | Yes | 15 | + | + | + | + |
| CRC | CRC 19 | NA | 46 | M | | IV | Yes | Yes | 6 | + | + | + | + |
| | | | | | | | | | | | | | + |

| Cohort | ID | Histology | AGE | Gender | SMOKER |
|---|---|---|---|---|---|
| Control Cohort A | Control01 | Healthy/Benign | 74 | F | Former |
| Control Cohort A | Control02 | Healthy/Benign | 70 | F | Former |
| Control Cohort A | Control03 | Healthy/Benign | 76 | M | Former |
| Control Cohort A | Control04 | Healthy/Benign | 90 | F | Former |
| Control Cohort A | Control05 | Healthy/Benign | 80 | F | Former |
| Control Cohort A | Control06 | Healthy/Benign | 64 | F | Never |
| Control Cohort A | Control07 | Healthy/Benign | 55 | M | Current |
| Control Cohort A | Control08 | Healthy/Benign | 86 | M | current |
| Control Cohort A | Control09 | Healthy/Benign | 84 | M | Former |
| Control Cohort A | Control10 | Healthy/Benign | 75 | M | Current |
| Control Cohort A | Control11 | Healthy/Benign | 58 | M | Former |
| Control Cohort A | Control12 | Healthy/Benign | 63 | M | Former |
| Control Cohort A | Control13 | Healthy/Benign | 67 | M | Former |
| Control Cohort A | Control14 | Healthy/Benign | 69 | F | Former |
| Control Cohort A | Control15 | Healthy/Benign | 55 | M | Former |
| Control Cohort A | Control16 | Healthy/Benign | 67 | F | Former |
| Control Cohort A | Control17 | Healthy/Benign | 49 | M | Former |
| Control Cohort A | Control18 | Healthy/Benign | 69 | M | Former |
| Control Cohort A | Control19 | Healthy/Benign | 41 | F | Current |
| Control Cohort A | Control20 | Healthy/Benign | 69 | M | Current |
| Control Cohort A | Control21 | Healthy/Benign | 73 | M | Former |
| Control Cohort A | Control22 | Healthy/Benign | 56 | F | Former |
| Control Cohort A | Control23 | Healthy/Benign | 59 | F | Former |
| Control Cohort A | Control24 | Healthy/Benign | 76 | M | Current |
| Control Cohort A | Control25 | Healthy/Benign | 59 | F | Former |
| Control Cohort A | Control26 | Healthy/Benign | 60 | F | Former |
| Control Cohort A | Control27 | Healthy/Benign | 68 | M | Former |
| Control Cohort A | Control28 | Healthy/Benign | 52 | M | Current |
| Control Cohort A | Control29 | Healthy/Benign | 48 | M | Former |
| Control Cohort A | Control30 | Healthy/Benign | 76 | M | Former |
| Control Cohort A | Control31 | Healthy/Benign | 64 | M | Former |
| Control Cohort A | Control32 | Healthy/Benign | 71 | F | Former |
| Control Cohort A | Control33 | Healthy/Benign | 70 | F | Former |
| Control Cohort A | Control34 | Healthy/Benign | 68 | M | Current |
| Control Cohort A | Control35 | Healthy/Benign | 61 | F | Current |
| Control Cohort A | Control36 | Healthy/Benign | 65 | F | Former |
| Control Cohort A | Control37 | Healthy/Benign | 58 | M | Current |
| Control Cohort A | Control38 | Healthy/Benign | 64 | M | Former |

| Cohort | ID | Registry_ID | Cancer Stage | Age | Gender |
|---|---|---|---|---|---|
| Aarhus University | Aar- 1 | MF-3930 | Stage IV | 67 | F |
| Aarhus University | Aar- 2 | MF-5766 | Stage IV | 71 | F |
| Aarhus University | Aar- 3 | MF-5812 | Stage IV | 79 | M |
| Aarhus University | Aar- 4 | MF-6596 | Stage IV | 85 | F |
| Aarhus University | Aar- 5 | MF-5823 | Stage IV | 80 | M |
| Aarhus University | Aar- 6 | MF-6025 | pT1 | 74 | M |
| Aarhus University | Aar- 7 | MF-4165 | pT1 | 63 | M |
| Aarhus University | Aar- 8 | MF-2900 | pT1 | 67 | M |
| Aarhus University | Aar- 9 | MF-3511 | pT1 | 70 | F |
| Aarhus University | Aar- 10 | MF-8594 | pT1 | 61 | M |
| Aarhus University | Aar- 11 | MF-5427 | pT1 | 67 | M |
| Aarhus University | Aar- 12 | MF-5287 | pT1 | 53 | F |
| Aarhus University | Aar- 13 | MF-7637 | pT1 | 56 | M |
| Aarhus University | Aar- 14 | MF-9859 | pT1 | 73 | F |
| Aarhus University | Aar- 15 | MF-9144 | pT1 | 70 | M |
| Aarhus University | Aar- 16 | MF-1255 | Adenoma | 74 | F |
| Aarhus University | Aar- 17 | MF-8145 | Adenoma | 50 | F |
| Aarhus University | Aar- 18 | MF-1566 | Adenoma | 75 | M |
| Aarhus University | Aar- 19 | MF-5738 | Adenoma MSI | 50 | F |
| Aarhus University | Aar- 20 | MF-3793 | Adenoma | 75 | F |
| Aarhus University | Aar- 21 | MF-4629 | Adenoma | 50 | M |
| Aarhus University | Aar- 22 | MF-9004 | Adenoma | 55 | F |
| Aarhus University | Aar- 23 | MF-1203 | Adenoma | 65 | M |
| Aarhus University | Aar- 24 | MF-1208 | Adenoma | 66 | M |
| Aarhus University | Aar- 25 | MF-5642 | Adenoma | 58 | M |
| Aarhus University | Aar- 26 | MF-8291 | Adenoma | 65 | F |
| Aarhus University | Aar- 27 | MF-3108 | Adenoma | 65 | F |
| Aarhus University | Aar- 28 | MF-1794 | Adenoma | 60 | M |
| Aarhus University | Aar- 29 | MF-9921 | Adenoma | 66 | F |
| Aarhus University | Aar- 30 | MF-0187 | Adenoma | 55 | M |
| Aarhus University | Aar- 31 | MF-1673 | Adenoma | 60 | M |
| Aarhus University | Aar- 32 | MF-1137 | Adenoma | 73 | M |
| Aarhus University | Aar- 33 | MF-1590 | Adenoma | 62 | F |
| Aarhus University | Aar- 34 | MF-1103 | Adenoma | 68 | M |
| Aarhus University | Aar- 35 | MF-1060 | Adenoma | 67 | M |

| Cohort | ID | Histology | Age | Gender |
|---|---|---|---|---|
| Control Cohort B | Donor333 | Healthy/Benign | 51 | F |
| Control Cohort B | Donor334 | Healthy/Benign | 58 | M |
| Control Cohort B | Donor335 | Healthy/Benign | 53 | F |
| Control Cohort B | Donor336 | Healthy/Benign | 46 | F |
| Control Cohort B | Donor337 | Healthy/Benign | 62 | M |
| Control Cohort B | Donor338 | Healthy/Benign | 58 | M |
| Control Cohort B | Donor340 | Healthy/Benign | 61 | M |
| Control Cohort B | Donor343 | Healthy/Benign | 59 | M |
| Control Cohort B | Donor344 | Healthy/Benign | 61 | M |
| Control Cohort B | Donor347 | Healthy/Benign | 57 | M |
| Control Cohort B | Donor349 | Healthy/Benign | 58 | F |
| Control Cohort B | Donor352 | Healthy/Benign | 62 | M |
| Control Cohort B | Donor353 | Healthy/Benign | 58 | M |
| Control Cohort B | Donor356 | Healthy/Benign | 63 | M |
| Control Cohort B | Donor358 | Healthy/Benign | 50 | F |

| | | | | | | | Early | |
|---|---|---|---|---|---|---|---|---|
| | | | | | | | steroids | |
| | | | | | | Week 6 | (<8 | PFS |
| Cohort | ID | Histology | Age | Gender | Stage | RECIST | weeks) | Time |
| Adaptive | AD- 1 | Cutaneous | M | 50 | IVB | PD | | 1.1 |
| dosing | | | | | | | | |
| melanoma | | | | | | | | |
| Adaptive | AD- 2 | Cutaneous | M | 43 | IVB | PR | Yes | 36.3 |
| dosing | | | | | | | | |
| melanoma | | | | | | | | |
| Adaptive | AD- 4 | Cutaneous | M | 78 | IVC | PR | Yes | 11.0 |
| dosing | | | | | | | | |
| melanoma | | | | | | | | |
| Adaptive | AD- 5 | Cutaneous | M | 65 | IVC | PR | | 35.9 |
| dosing | | | | | | | | |
| melanoma | | | | | | | | |
| Adaptive | AD- 11 | Cutaneous | M | 71 | IVC | PR | | 35.8 |
| dosing | | | | | | | | |
| melanoma | | | | | | | | |
| Adaptive | AD- 12 | Cutaneous | M | 67 | IVC | SD | | 35.9 |
| dosing | | | | | | | | |
| melanoma | | | | | | | | |
| Adaptive | AD- 16 | Cutaneous | F | 43 | IVC | SD | | 36.1 |
| dosing | | | | | | | | |
| melanoma | | | | | | | | |
| Adaptive | AD- 17 | Cutaneous | M | 45 | IVC | PD | Yes | 1.2 |
| dosing | | | | | | | | |
| melanoma | | | | | | | | |
| Adaptive | AD- 18 | Cutaneous | M | 78 | IVC | PR | | 35.9 |
| dosing | | | | | | | | |
| melanoma | | | | | | | | |
| Adaptive | AD- 20 | Cutaneous | F | 19 | IVB | PD | | 1.3 |
| dosing | | | | | | | | |
| melanoma | | | | | | | | |
| Adaptive | AD- 25 | Cutaneous | M | 57 | IVC | PD | Yes | 1.3 |
| dosing | | | | | | | | |
| melanoma | | | | | | | | |
| Adaptive | AD- 26 | Cutaneous | F | 58 | IVD | SD | Yes | 2.8 |
| dosing | | | | | | | | |
| melanoma | | | | | | | | |
| Adaptive | AD- 32 | Cutaneous | M | 64 | IVD | SD | | 31.3 |
| dosing | | | | | | | | |
| melanoma | | | | | | | | |
| Adaptive | AD- 34 | Cutaneous | M | 35 | III | SD | | 9.4 |
| dosing | | | | | | | | |
| melanoma | | | | | | | | |
| Adaptive | AD- 35 | Cutaneous | M | 77 | IVC | SD | Yes | 5.6 |
| dosing | | | | | | | | |
| melanoma | | | | | | | | |
| Adaptive | AD- 36 | Cutaneous | M | 63 | III | PR | Yes | 2.6 |
| dosing | | | | | | | | |
| melanoma | | | | | | | | |
| Adaptive | AD- 38 | Cutaneous | M | 53 | IVB | SD | | 26.6 |
| dosing | | | | | | | | |
| melanoma | | | | | | | | |
| Adaptive | AD- 40 | Cutaneous | M | 80 | IVB | PD | Yes | 1.2 |
| dosing | | | | | | | | |
| melanoma | | | | | | | | |
| Adaptive | AD- 41 | Cutaneous | M | 65 | III | PR | | 25.0 |
| dosing | | | | | | | | |
| melanoma | | | | | | | | |
| Adaptive | AD- 42 | Cutaneous | M | 55 | IVD | SD | Yes | 3.0 |
| dosing | | | | | | | | |
| melanoma | | | | | | | | |
| Adaptive | AD- 43 | Cutaneous | F | 79 | IVD | PR | Yes | 5.3 |
| dosing | | | | | | | | |
| melanoma | | | | | | | | |
| Adaptive | AD- 44 | Cutaneous | M | 41 | IVC | PD | | 1.1 |
| dosing | | | | | | | | |
| melanoma | | | | | | | | |
| Adaptive | AD- 45 | Cutaneous | F | 61 | IVA | PR | Yes | 5.8 |
| dosing | | | | | | | | |
| melanoma | | | | | | | | |
| Adaptive | AD- 46 | Cutaneous | M | 71 | IVC | PR | | 21.0 |
| dosing | | | | | | | | |
| melanoma | | | | | | | | |
| Adaptive | AD- 48 | Cutaneous | M | 49 | IVD | PR | | 20.2 |
| dosing | | | | | | | | |
| melanoma | | | | | | | | |
| Adaptive | AD- 50 | Cutaneous | M | 57 | IVD | SD | | 22.1 |
| dosing | | | | | | | | |
| melanoma | | | | | | | | |
| Adaptive | Acral-01 | Acral | F | 40 | IVC | SD | | 32.0 |
| dosing | | | | | | | | |
| melanoma | | | | | | | | |

| | | | | # of sites | | | | | |
|---|---|---|---|---|---|---|---|---|---|
| | | | | evaluated | | | | | |
| | | | | in tumor- | | | | | |
| | PFS | OS | OS | informed | | Week | Week | Week | Week |
| Cohort | Event | Time | Event | panel | Pretreatment | 3 | 6 | 9 | 12 |
| Adaptive | Yes | 18.3 | Yes | N/A | + | + | + | + | + |
| dosing | | | | | | | | | |
| melanoma | | | | | | | | | |
| Adaptive | | 36.3 | | N/A | + | + | + | − | − |
| dosing | | | | | | | | | |
| melanoma | | | | | | | | | |
| Adaptive | Yes | 29.1 | | 29 | + | + | + | + | − |
| dosing | | | | | | | | | |
| melanoma | | | | | | | | | |
| Adaptive | | 35.9 | | 7 | + | + | + | + | − |
| dosing | | | | | | | | | |
| melanoma | | | | | | | | | |
| Adaptive | | 35.8 | | 14 | + | + | + | − | − |
| dosing | | | | | | | | | |
| melanoma | | | | | | | | | |
| Adaptive | | 35.9 | | N/A | + | + | + | − | − |
| dosing | | | | | | | | | |
| melanoma | | | | | | | | | |
| Adaptive | | 36.1 | | 17 | + | + | + | − | − |
| dosing | | | | | | | | | |
| melanoma | | | | | | | | | |
| Adaptive | Yes | 16.4 | | 7 | + | + | + | − | − |
| dosing | | | | | | | | | |
| melanoma | | | | | | | | | |
| Adaptive | | 35.9 | | 16 | + | + | + | − | − |
| dosing | | | | | | | | | |
| melanoma | | | | | | | | | |
| Adaptive | Yes | 26.4 | Yes | 2 | + | + | + | − | − |
| dosing | | | | | | | | | |
| melanoma | | | | | | | | | |
| Adaptive | Yes | 20.3 | | N/A | + | + | + | + | − |
| dosing | | | | | | | | | |
| melanoma | | | | | | | | | |
| Adaptive | Yes | 12.8 | Yes | N/A | + | + | + | − | − |
| dosing | | | | | | | | | |
| melanoma | | | | | | | | | |
| Adaptive | | 31.3 | | N/A | + | + | + | − | − |
| dosing | | | | | | | | | |
| melanoma | | | | | | | | | |
| Adaptive | | 21.2 | | N/A | + | + | + | − | − |
| dosing | | | | | | | | | |
| melanoma | | | | | | | | | |
| Adaptive | Yes | 5.6 | Yes | N/A | + | + | − | − | − |
| dosing | | | | | | | | | |
| melanoma | | | | | | | | | |
| Adaptive | Yes | 12.4 | | 8 | + | + | + | − | − |
| dosing | | | | | | | | | |
| melanoma | | | | | | | | | |
| Adaptive | | 26.6 | | 8 | + | + | + | − | − |
| dosing | | | | | | | | | |
| melanoma | | | | | | | | | |
| Adaptive | Yes | 17.9 | | 6 | + | + | + | − | − |
| dosing | | | | | | | | | |
| melanoma | | | | | | | | | |
| Adaptive | | 25.0 | | N/A | + | + | + | − | − |
| dosing | | | | | | | | | |
| melanoma | | | | | | | | | |
| Adaptive | Yes | 3.6 | Yes | N/A | + | + | − | − | − |
| dosing | | | | | | | | | |
| melanoma | | | | | | | | | |
| Adaptive | Yes | 7.9 | Yes | 5 | + | + | + | − | − |
| dosing | | | | | | | | | |
| melanoma | | | | | | | | | |
| Adaptive | Yes | 17.7 | | 2 | + | + | + | − | − |
| dosing | | | | | | | | | |
| melanoma | | | | | | | | | |
| Adaptive | Yes | 21.2 | | 3 | + | + | + | − | − |
| dosing | | | | | | | | | |
| melanoma | | | | | | | | | |
| Adaptive | | 21.0 | | N/A | + | + | + | − | − |
| dosing | | | | | | | | | |
| melanoma | | | | | | | | | |
| Adaptive | | 20.2 | | N/A | + | + | + | − | − |
| dosing | | | | | | | | | |
| melanoma | | | | | | | | | |
| Adaptive | | 22.1 | | 7 | + | + | + | − | − |
| dosing | | | | | | | | | |
| melanoma | | | | | | | | | |
| Adaptive | | 32.0 | | N/A | + | + | + | − | − |
| dosing | | | | | | | | | |
| melanoma | | | | | | | | | |

| | | | | | | | 12 week | PFS |
|---|---|---|---|---|---|---|---|---|
| Cohort | ID | Histology | Age | Gender | Stage | Treatment | Recist | Time |
| Conventional | MSK- 32 | Cutaneous | 38 | M | IV | NIVO | SD | 12 |
| immunotherapy | | | | | | | | |
| melanoma | | | | | | | | |
| Conventional | MSK- 33 | Cutaneous | 60 | M | IV | NIVO | PD | 1 |
| immunotherapy | | | | | | | | |
| melanoma | | | | | | | | |
| Conventional | MSK- 34 | Cutaneous | 60 | M | IV | IPI/ | CR | 72 |
| immunotherapy | | | | | | NIVO | | |
| melanoma | | | | | | | | |
| Conventional | MSK- 37 | Cutaneous | 48 | M | IV | NIVO | SD | 61 |
| immunotherapy | | | | | | | | |
| melanoma | | | | | | | | |
| Conventional | MSK- 38 | Cutaneous | 73 | M | IV | NIVO | PR | 72 |
| immunotherapy | | | | | | | | |
| melanoma | | | | | | | | |
| Conventional | MSK- 40 | Cutaneous | 70 | F | IV | IPI/ | CR | 72 |
| immunotherapy | | | | | | NIVO | | |
| melanoma | | | | | | | | |
| Conventional | MSK- 41 | Cutaneous | 69 | F | IV | NIVO | PR | 53 |
| immunotherapy | | | | | | | | |
| melanoma | | | | | | | | |
| Conventional | MSK- 42 | Cutaneous | 58 | M | IV | IPI/ | PR | 28 |
| immunotherapy | | | | | | NIVO | | |
| melanoma | | | | | | | | |
| Conventional | MSK- 45 | Cutaneous | 65 | F | IV | NIVO | SD | 28 |
| immunotherapy | | | | | | | | |
| melanoma | | | | | | | | |
| Conventional | MSK- 53 | Cutaneous | 53 | M | IV | IPI/ | PR | 72 |
| immunotherapy | | | | | | NIVO | | |
| melanoma | | | | | | | | |
| Conventional | MSK- 54 | Cutaneous | 59 | F | IV | IPI/ | SD | 72 |
| immunotherapy | | | | | | NIVO | | |
| melanoma | | | | | | | | |

| | | | | | Plasma | | | | |
|---|---|---|---|---|---|---|---|---|---|
| | PFS | OS | OS | Early | timepoints | Week | Week | Week | Week |
| Cohort | Event | Time | Event | Steroids | Pretreatment | 3 | 6 | 9 | 12 |
| Conventional | Yes | 21 | Yes | | + | + | + | − | + |
| immunotherapy | | | | | | | | | |
| melanoma | | | | | | | | | |
| Conventional | Yes | 33 | Yes | | + | + | + | − | + |
| immunotherapy | | | | | | | | | |
| melanoma | | | | | | | | | |
| Conventional | | 72 | | | + | + | + | − | + |
| immunotherapy | | | | | | | | | |
| melanoma | | | | | | | | | |
| Conventional | Yes | 68 | Yes | | + | + | + | − | + |
| immunotherapy | | | | | | | | | |
| melanoma | | | | | | | | | |
| Conventional | | 72 | | | + | + | + | − | + |
| immunotherapy | | | | | | | | | |
| melanoma | | | | | | | | | |
| Conventional | | 72 | | | + | + | + | − | + |
| immunotherapy | | | | | | | | | |
| melanoma | | | | | | | | | |
| Conventional | Yes | 72 | | | + | + | + | − | − |
| immunotherapy | | | | | | | | | |
| melanoma | | | | | | | | | |
| Conventional | Yes | 56 | Yes | | + | + | + | − | + |
| immunotherapy | | | | | | | | | |
| melanoma | | | | | | | | | |
| Conventional | Yes | 75 | Yes | | + | + | + | − | + |
| immunotherapy | | | | | | | | | |
| melanoma | | | | | | | | | |
| Conventional | | 72 | | | + | + | + | − | − |
| immunotherapy | | | | | | | | | |
| melanoma | | | | | | | | | |
| Conventional | | 72 | | | + | + | + | − | + |
| immunotherapy | | | | | | | | | |
| melanoma | | | | | | | | | |

| | | | | | Cancer | |
|---|---|---|---|---|---|---|
| Cohort | ID | Histology | Age | Gender | Stage | Treatment |
| Tumor | MEL-01 | Cutaneous | 71 | M | IV | Pembrolizumab |
| confirmed | | | | | | |
| melanoma | | | | | | |

| Cohort | ID | Sample ID | Age | Gender | Smoking | Histology | pTNM | Stage |
|---|---|---|---|---|---|---|---|---|
| Neoadjuvant | Neo- 1 | NA-18 | 83 | F | Former | Adenocarcinoma | 2 | IIB and |
| immunotherapy | | | | | | | primaries- | IA |
| NSCLC | | | | | | | ypT2bN1M0 & | |
| | | | | | | | ypT1bN0M0 | |
| Neoadjuvant | Neo- 2 | NA-40 | 63 | M | Current | NOS | ypT2aN0M0 | IB |
| immunotherapy | | | | | | | | |
| NSCLC | | | | | | | | |
| Neoadjuvant | Neo- 3 | NA-36 | 72 | M | Former | Squamous | ypT3N1M0 | IIIA |
| immunotherapy | | | | | | | | |
| NSCLC | | | | | | | | |

| | | | | | | Current |
|---|---|---|---|---|---|---|
| Cohort | ID | Sample ID | Histology | Age | Gender | Smoker |
| Control | C- 1 | CB-001 | Healthy/ | 53 | M | Yes |
| Cohort C | | | Benign | | | |
| Control | C- 3 | CB-003 | Healthy/ | 75 | M | No |
| Cohort C | | | Benign | | | |
| Control | C- 4 | CB-004 | Healthy/ | 56 | M | Yes |
| Cohort C | | | Benign | | | |
| Control | C- 5 | CB-005 | Healthy/ | 78 | M | No |
| Cohort C | | | Benign | | | |
| Control | C- 6 | CB-006 | Healthy/ | 49 | M | No |
| Cohort C | | | Benign | | | |
| Control | C- 7 | CB-007 | Healthy/ | 65 | F | Yes |
| Cohort C | | | Benign | | | |
| Control | C- 8 | CB-008 | Healthy/ | 75 | F | Yes |
| Cohort C | | | Benign | | | |
| Control | C- 9 | CB-009 | Healthy/ | 66 | F | Yes |
| Cohort C | | | Benign | | | |
| Control | C- 10 | CB-010 | Healthy/ | 56 | M | Yes |
| Cohort C | | | Benign | | | |
| Control | C- 11 | CB-011 | Healthy/ | 82 | F | No |
| Cohort C | | | Benign | | | |
| Control | C- 12 | CB-012 | Healthy/ | 78 | F | Yes |
| Cohort C | | | Benign | | | |
| Control | C- 13 | CB-013 | Healthy/ | 53 | M | Yes |
| Cohort C | | | Benign | | | |
| Control | C- 14 | CB-014 | Healthy/ | 77 | F | No |
| Cohort C | | | Benign | | | |
| Control | C- 15 | CB-015 | Healthy/ | 66 | F | No |
| Cohort C | | | Benign | | | |
| Control | C- 16 | CB-016 | Healthy/ | 57 | M | Yes |
| Cohort C | | | Benign | | | |
| Control | C- 20 | CB-020 | Healthy/ | 33 | M | Yes |
| Cohort C | | | Benign | | | |
| Control | C- 21 | CB-021 | Healthy/ | 83 | M | No |
| Cohort C | | | Benign | | | |
| Control | C- 22 | CB-022 | Healthy/ | 76 | F | Yes |
| Cohort C | | | Benign | | | |
| Control | C- 23 | CB-023 | Healthy/ | 64 | M | No |
| Cohort C | | | Benign | | | |
| Control | C- 24 | CB-024 | Healthy/ | 73 | M | No |
| Cohort C | | | Benign | | | |
| Control | C- 25 | CB-025 | Healthy/ | 76 | F | No |
| Cohort C | | | Benign | | | |
| Control | C- 26 | CB-026 | Healthy/ | 87 | M | No |
| Cohort C | | | Benign | | | |
| Control | C- 27 | CB-027 | Healthy/ | 41 | M | Yes |
| Cohort C | | | Benign | | | |
| Control | C- 28 | CB-028 | Healthy/ | 62 | M | No |
| Cohort C | | | Benign | | | |
| Control | C- 29 | CB-029 | Healthy/ | 58 | M | No |
| Cohort C | | | Benign | | | |
| Control | C- 30 | CB-030 | Healthy/ | 64 | F | No |
| Cohort C | | | Benign | | | |
| Control | C- 31 | CB-031 | Healthy/ | 80 | M | Yes |
| Cohort C | | | Benign | | | |
| Control | C- 32 | CB-032 | Healthy/ | 67 | M | Yes |
| Cohort C | | | Benign | | | |
| Control | C- 33 | CB-033 | Healthy/ | 75 | M | Yes |
| Cohort C | | | Benign | | | |
| Control | C- 34 | CB-034 | Healthy/ | 49 | M | No |
| Cohort C | | | Benign | | | |
| Control | C- 35 | CB-035 | Healthy/ | 44 | F | N/A |
| Cohort C | | | Benign | | | |
| Control | C- 36 | CB-036 | Healthy/ | 78 | M | No |
| Cohort C | | | Benign | | | |
| Control | C- 37 | CB-037 | Healthy/ | 28 | F | Yes |
| Cohort C | | | Benign | | | |
| Control | C- 38 | CB-038 | Healthy/ | 62 | F | Yes |
| Cohort C | | | Benign | | | |
| Control | C- 39 | CB-039 | Healthy/ | 75 | M | Yes |
| Cohort C | | | Benign | | | |

| Cohort | ID | Histology | Age | Gender | Smoker |
|---|---|---|---|---|---|
| High | CM6 | Adenocarcinoma | 73 | F | Former |
| burden | | | | | |
| LUAD | | | | | |
| High | CM30 | Adenocarcinoma | 79 | F | Former |
| burden | | | | | |
| LUAD | | | | | |

Pathological
Response
None
None
None

### Appendix 5

| | | MRD-EDGE CNV Z | | |
|---|---|---|---|---|
| Patient ID | MRD-EDGE SNV Z Score | Score | Cancer Type | Timepoint |
| CRC 01 | 2.488216 | 1.560116 | CRC | Preoperative |
| CRC 02 | 10 | −0.012955 | CRC | Preoperative |
| CRC 03 | 5.135133 | 5.880186 | CRC | Preoperative |
| CRC 04 | 10 | 1.369827 | CRC | Preoperative |
| CRC 05 | 5.449564 | 1.46225 | CRC | Preoperative |
| CRC 06 | 1.47369 | 0.641322 | CRC | Preoperative |
| CRC 07 | 1.546357 | 0.44481 | CRC | Preoperative |
| CRC 08 | 10 | −0.176742 | CRC | Preoperative |
| CRC 09 | 10 | 5.003901 | CRC | Preoperative |
| CRC 10 | 10 | −1.113519 | CRC | Preoperative |
| CRC 11 | 10 | 7.569149 | CRC | Preoperative |
| CRC 12 | 2.801802 | 1.11748 | CRC | Preoperative |
| CRC 13 | 6.973304 | 1.512824 | CRC | Preoperative |
| CRC 14 | 10 | 9.256662 | CRC | Preoperative |
| CRC 15 | 10 | 2.60636 | CRC | Preoperative |
| CRC 16 | 10 | 10 | CRC | Preoperative |
| CRC 17 | 3.149166 | 0.619038 | CRC | Preoperative |
| CRC 18 | 10 | I A | CRC | Preoperative |
| CRC 19 | 10 | 10 | CRC | Preoperative |
| CRC 01 | 0.630042 | 1.168191 | CRC | Postoperative |
| CRC 02 | 0.693595 | −0.402922 | CRC | Postoperative |
| CRC 03 | 1.639242 | 4.464172 | CRC | Postoperative |
| CRC 04 | 0.97997 | 0.536941 | CRC | Postoperative |
| CRC 05 | 3.695736 | 0.492404 | CRC | Postoperative |
| CRC 06 | −0.231804 | −0.358382 | CRC | Postoperative |
| CRC 07 | 1.197527 | −1.024675 | CRC | Postoperative |
| CRC 08 | 0.859193 | 1.156656 | CRC | Postoperative |
| CRC 09 | −0.798823 | −2.833297 | CRC | Postoperative |
| CRC 10 | 10 | 0.878089 | CRC | Postoperative |
| CRC 11 | 3.859677 | 1.004868 | CRC | Postoperative |
| CRC 12 | 0.185213 | 0.689773 | CRC | Postoperative |
| CRC 13 | 0.159579 | 0.270605 | CRC | Postoperative |
| CRC 14 | 1.590017 | −0.025673 | CRC | Postoperative |
| CRC 15 | 0.870831 | 0.807216 | CRC | Postoperative |
| CRC 16 | 10 | 10 | CRC | Postoperative |
| CRC 17 | −0.419806 | 0.228275 | CRC | Postoperative |
| CRC 18 | 10 | 0 | CRC | Postoperative |
| CRC 19 | 10 | 0.549238 | CRC | Postoperative |
| LUAD 01 | −0.255643 | N/A | LUAD | Preoperative |
| LUAD 02 | 7.30911 | N/A | LUAD | Preoperative |
| LUAD 03 | 0.455582 | N/A | LUAD | Preoperative |
| LUAD 04 | −0.262541 | N/A | LUAD | Preoperative |
| LUAD 05 | 10 | N/A | LUAD | Preoperative |
| LUAD 06 | 0.033565 | N/A | LUAD | Preoperative |
| LUAD 07 | 1.335293 | N/A | LUAD | Preoperative |
| LUAD 08 | 2.75062 | N/A | LUAD | Preoperative |
| LUAD 09 | 1.281743 | N/A | LUAD | Preoperative |
| LUAD 10 | −0.266546 | N/A | LUAD | Preoperative |
| LUAD 11 | −0.349363 | N/A | LUAD | Preoperative |
| LUAD 12 | 0.638424 | N/A | LUAD | Preoperative |
| LUAD 13 | 7.723141 | N/A | LUAD | Preoperative |
| LUAD 14 | 4.276323 | N/A | LUAD | Preoperative |
| LUAD 15 | 10 | N/A | LUAD | Preoperative |
| LUAD 16 | 1.000242 | N/A | LUAD | Preoperative |
| LUAD 17 | 1.375513 | N/A | LUAD | Preoperative |
| LUAD 18 | 1.228149 | N/A | LUAD | Preoperative |
| LUAD 19 | 0.062446 | N/A | LUAD | Preoperative |
| LUAD 20 | 0.133733 | N/A | LUAD | Preoperative |
| LUAD 21 | 10 | N/A | LUAD | Preoperative |
| LUAD 22 | 10 | N/A | LUAD | Preoperative |
| LUAD 23 | 0.738552 | N/A | LUAD | Preoperative |
| LUAD 24 | 10 | N/A | LUAD | Preoperative |
| LUAD 25 | 0.187295 | N/A | LUAD | Preoperative |
| LUAD 26 | 7.042274 | N/A | LUAD | Preoperative |
| LUAD 27 | 10 | N/A | LUAD | Preoperative |
| LUAD 28 | 1.340718 | N/A | LUAD | Preoperative |
| LUAD 29 | 4.138961 | N/A | LUAD | Preoperative |
| LUAD 30 | 0.00012 | N/A | LUAD | Preoperative |
| LUAD 31 | 10 | N/A | LUAD | Preoperative |
| LUAD 32 | 2.731145 | N/A | LUAD | Preoperative |
| LUAD 33 | 10 | N/A | LUAD | Preoperative |
| LUAD 34 | 10 | N/A | LUAD | Preoperative |
| LUAD 35 | 9.536151 | N/A | LUAD | Preoperative |
| LUAD 36 | −0.244976 | N/A | LUAD | Preoperative |
| LUAD-04 | −0.518077 | N/A | LUAD | Postoperative |
| LUAD-06 | −0.209113 | N/A | LUAD | Postoperative |
| LUAD-10 | 2.399194 | N/A | LUAD | Postoperative |
| LUAD-11 | 3.694809 | N/A | LUAD | Postoperative |
| LUAD-12 | 0.353618 | N/A | LUAD | Postoperative |
| LUAD-14 | 5.288524 | N/A | LUAD | Postoperative |
| LUAD-15 | 10 | N/A | LUAD | Postoperative |
| LUAD-18 | 1.867181 | N/A | LUAD | Postoperative |
| LUAD-19 | −0.724229 | N/A | LUAD | Postoperative |
| LUAD-20 | −0.09484 | N/A | LUAD | Postoperative |
| LUAD-22 | 10 | N/A | LUAD | Postoperative |
| LUAD-23 | −0.469271 | N/A | LUAD | Postoperative |
| LUAD-25 | −0.153122 | N/A | LUAD | Postoperative |
| LUAD-26 | 4.030056 | N/A | LUAD | Postoperative |
| LUAD-27 | 10 | N/A | LUAD | Postoperative |
| LUAD-28 | 1.415818 | N/A | LUAD | Postoperative |
| LUAD-31 | 10 | N/A | LUAD | Postoperative |
| LUAD-32 | 2.586671 | N/A | LUAD | Postoperative |
| LUAD-35 | 5.938387 | N/A | LUAD | Postoperative |
| LUAD-37 | −0.304597 | N/A | LUAD | Postoperative |
| LUAD-38 | 0.123247 | N/A | LUAD | Postoperative |
| LUAD-39 | 0.545432 | N/A | LUAD | Postoperative |

*SNV detection threshold for early-stage CRC is Z = 1.33 as per 90% specificity in preoperative samples. CNV detection threshold for early-stage CRC is Z = 1.29 as per 90% specificity in preoperative samples. SNV detection threshold for early-stage LUAD is Z = 0.66602 as per 90% specificity in preoperative samples

| | MRD-EDGE SNV Z score | | | |
|---|---|---|---|---|
| Patient ID | Pretreatment/Day 3 | Week 4 | Week 6 | Postoperative 3 months |
| Neo-01 | 2.60 | 0.83 | 0.93 | 1.69 |
| Neo-02 | 10.00 | N/A | 10.00 | −0.30 |
| Neo-03 | 10.00 | 10.00 | 10.00 | 1.61 |

*SNV detection threshold is Z = 0.66602 as prespecified in early-stage LUAD cohort
*MRD-EDGE SNV and CNV detection metrics for CRC, LUAD and Neo cohorts. Z-scores are calculated using a patient plasma sample compared to a panel of control samples (SNV n = 38 for CRC and LUAD , n = 30 for Neo, CNV n = 10 for CRC). Positive Z Scores are capped at 10
*I A—Insufficient aneuploidy
*N/A MRD-EDGE CNV was not applied to LUAD cohorts due to low matched tumor purity precluding accurate assignment of tumor ploidy and allelic imbalance

| | | MRD-EDGE SNV | |
|---|---|---|---|
| Patient ID | MRD-EDGE SNV (Control) | (Cross-patient) | MRD-EDGE CNV |
| Aar-01 | 10 | 10 | 10 |
| Aar-02 | 10 | 10 | 10 |
| Aar-03 | 10 | 10 | 10 |
| Aar-04 | 10 | 10 | 10 |
| Aar-05 | 10 | 10 | 10 |
| Aar-06 | −0.71622 | −1.028786 | 0.244501 |
| Aar-07 | 10 | 3.251604 | 0.928617 |
| Aar-08 | 1.573872 | 1.507096 | 0.886507 |
| Aar-09 | 2.104262 | 0.800592 | I A |
| Aar-10 | 1.735528 | 0.622099 | −1.013388 |
| Aar-11 | 1.885074 | 1.34876 | 2.137901 |
| Aar-12 | 0.998798 | 0.823164 | 2.733419 |
| Aar-13 | 0.486212 | −0.716582 | 1.991542 |
| Aar-14 | 7.091317 | 3.039195 | 1.200778 |
| Aar-16 | 0.12914 | 0.250203 | IA |
| Aar-17 | 0.869827 | 0.026787 | 0.075071 |
| Aar-18 | −0.449267 | −1.053682 | I A |
| Aar-19 | 5.597337 | 2.092075 | I A |
| Aar-20 | 0.779709 | −0.247385 | −0.919387 |
| Aar-21 | 0.879096 | 0.010756 | 1.627593 |
| Aar-22 | −0.05965 | −0.064083 | I A |
| Aar-23 | 1.696394 | 0.132869 | −1.232762 |
| Aar-24 | 5.870369 | 2.401828 | 0.408942 |
| Aar-25 | 2.980587 | 2.623546 | I A |
| Aar-26 | 0.334468 | 0.710707 | −0.086056 |
| Aar-27 | 0.737131 | −0.068043 | I A |
| Aar-28 | 4.689075 | 2.068011 | 1.122248 |
| Aar-29 | −0.583157 | −0.983494 | I A |
| Aar-30 | 1.170881 | −0.194124 | 0.655308 |
| Aar-31 | 1.587957 | 0.183132 | 1.439046 |
| Aar-32 | 0.420801 | 0.498876 | I A |
| Aar-33 | −0.418518 | −1.097035 | 0.85185 |
| Aar-34 | 0.106266 | −0.144614 | 1.636906 |

*SNV detection threshold is Z = 1.33 as prespecified in early-stage CRC cohort. CNV detection threshold is Z = 1.29 as prespecified in early-stage CRC cohort
*MRD-EDGE SNV and CNV detection metrics for Aarhus University cohort of stage IV and pT1 colorectal carcinomas and colorectal adenomas. Z-scores are calculated using a patient plasma sample compared to a panel of control samples (SNV n = 11, CNV n = 10). Positive Z Scores are capped at 10
*I A—Insufficient aneuploidy

### Appendix 6

MRD-EDGE SNV de novo de

[symbol]

| Patient ID | Pretreatment | Week 3 | Week 6 | Week 9 | Week 12 |
|---|---|---|---|---|---|
| AD-01 | 1.74E−04 | 2.73E−04 | 2.50E−04 | 2.86E−04 | 2.22E−04 |
| AD-02 | 4.15E−05 | 5.35E−05 | 7.18E−05 | N/A | N/A |
| AD-04 | 1.49E−03 | 8.40E−05 | 7.14E−05 | 5.88E−05 | N/A |
| AD-05 | 4.71E−03 | 2.15E−03 | 1.80E−04 | 7.31E−05 | N/A |
| AD-11 | 2.61E−04 | 7.58E−05 | 6.17E−05 | N/A | N/A |
| AD-12 | 4.87E−04 | 3.93E−04 | 1.20E−04 | N/A | N/A |
| AD-16 | 5.44E−04 | 6.69E−05 | 7.59E−05 | N/A | N/A |
| AD-17 | 4.33E−04 | 1.66E−04 | 1.93E−04 | N/A | N/A |
| AD-18 | 1.23E−03 | 2.14E−04 | 7.77E−05 | N/A | N/A |
| AD-20 | 7.57E−05 | 8.74E−05 | 8.03E−05 | N/A | N/A |
| AD-25 | 6.83E−04 | 1.90E−04 | 7.13E−05 | 6.72E−05 | N/A |
| AD-26 | 8.85E−05 | 8.99E−05 | 9.80E−05 | N/A | N/A |
| AD-32 | 5.28E−04 | 5.24E−04 | 2.26E−04 | N/A | N/A |
| AD-34 | 6.08E−05 | 8.26E−05 | 6.97E−05 | N/A | N/A |
| AD-35 | 3.64E−04 | 1.47E−04 | N/A | N/A | N/A |
| AD-36 | 8.60E−05 | 7.80E−05 | 9.59E−05 | N/A | N/A |
| AD-38 | 7.24E−05 | 8.88E−05 | 8.35E−05 | N/A | N/A |
| AD-40 | 4.67E−04 | 2.27E−04 | 1.15E−04 | N/A | N/A |
| AD-41 | 1.84E−04 | 6.12E−05 | 9.22E−05 | N/A | N/A |
| AD-42 | 1.99E−03 | 1.04E−03 | N/A | N/A | N/A |
| AD-43 | 1.15E−04 | 7.98E−05 | 7.99E−05 | N/A | N/A |
| AD-44 | 5.10E−04 | 9.79E−05 | 1.69E−04 | N/A | N/A |
| AD-45 | 4.18E−04 | 7.78E−05 | 7.92E−05 | N/A | N/A |
| AD-46 | 5.63E−04 | 2.71E−04 | 7.11E−05 | N/A | N/A |
| AD-48 | 5.48E−04 | 8.00E−05 | 7.34E−05 | N/A | N/A |
| AD-50 | 1.68E−04 | 1.05E−04 | 9.37E−05 | N/A | N/A |
| MSK-32 | 8.28E−05 | 9.56E−05 | 7.93E−05 | N/A | 8.54E−05 |
| MSK-33 | 8.67E−05 | 9.81E−05 | 1.05E−04 | N/A | 9.25E−05 |
| MSK-34 | 8.75E−05 | 8.40E−05 | 7.19E−05 | N/A | 1.04E−04 |
| MSK-37 | 1.34E−03 | 3.88E−04 | 1.19E−04 | N/A | 8.49E−05 |
| MSK-38 | 6.59E−04 | 9.52E−05 | 7.38E−05 | N/A | 9.49E−05 |
| MSK-40 | 8.37E−04 | 1.26E−04 | 8.43E−05 | N/A | 7.89E−05 |
| MSK-41 | 9.15E−05 | 8.23E−05 | 7.82E−05 | N/A | N/A |
| MSK-42 | 4.33E−04 | 2.23E−04 | 2.89E−04 | N/A | 1.40E−04 |
| MSK-45 | 8.50E−05 | 8.92E−05 | 9.65E−05 | N/A | 8.75E−05 |
| MSK-49 | 6.90E−05 | 6.57E−05 | 8.75E−05 | N/A | N/A |
| MSK-53 | 1.36E−04 | 8.90E−05 | 7.62E−05 | N/A | N/A |
| MSK-54 | 4.70E−05 | 8.59E−05 | 9.64E−05 | N/A | 7.64E−05 |
| Acral-01 | 5.83E−05 | 4.99E−05 | 6.93E−05 | N/A | N/A |

*SNV detection rate threshold for sample-level detection of cutaneou [symbol] [symbol]
indicates data missing or illegible when filed

[symbol]

tection rate
Notes

Excluded from melanoma clinical survival analyses
due to undetectable pretreatment timepoint
Excluded from melanoma clinical survival analyses
due to undetectable pretreatment timepoint
Excluded from melanoma clinical survival analyses
due to undetectable pretreatment timepoint
Negative control acral melanoma not expected to
harbor UV mutagenesis signal [symbol]
s melanoma against healthy controls is 7.237e−05 [symbol]
indicates data missing or illegible when filed

1. A method comprising:
reading a plurality of reference sequences;
reading a plurality of sequence fragments obtained from a biological sample of a patient;
selecting a first read and a second read from the plurality of sequence fragments, wherein
the first read comprises a first portion of a corresponding reference sequence in the plurality of reference sequences and a first position, and wherein
the second read comprises a second portion of the corresponding reference sequence and a second position, wherein at least one of the first read and the second read comprises an alt position;
receiving, from a first trained classifier, a regional probability based on a plurality of regional features of the patient;
generating a tensor comprising the corresponding reference sequence, the first read, the second read, the first position, the second position, and the alt position;
providing the tensor to a second trained classifier comprising a convolutional neural network, and receiving therefrom a local probability based on the tensor; and
determining a label associated with a tumor marker when the regional probability is above a first predetermined threshold and the local probability is above a second predetermined threshold.

2. The method of claim 1, wherein the first read and the second read are paired-reads.

3. The method of claim 1, wherein the label comprises a ctDNA label.

4. The method of claim 1, wherein the label comprises likelihood of cancer mutagenesis.

5. The method of claim 1, wherein the first trained classifier comprises a multilayer perceptron.

6. The method of claim 5, wherein the plurality of regional features comprises one or more of: a local tumor-type specific ATAC density, a local primary cell DNAse hypersensitivity, a local histone chip-seq density, a local cancer type specific mutational density, a local chromatin state, a Hi-C compartmentalization, a replication timing, a transcription direction, an indication of whether transcription is forwards or backwards, a distance to bound transcription factors, an RNA accessibility, and one or more low-quality bases.

7. The method of claim 1, wherein the plurality of regional features are determined around the alt position.

8. The method of claim 5, wherein the multilayer perceptron is configured to output a probability that the input fragment is ctDNA.

9. The method of claim 1, wherein the tensor has a dimension of 18×400, 19×400, or 18×240.

10-11. (canceled)

12. The method of claim 1, wherein rows of the tensor represent the corresponding reference sequence, the first read, the second read, the first position, the second position, and the alt position.

13. The method of claim 12, wherein five consecutive rows represent nucleotides of the reference sequence, nucleotides of the first read, or nucleotides of the second read.

14-15. (canceled)

16. The method of claim 12, wherein a first length of the first read and a second length of the second read are each tracked by a single row of the tensor.

17. The method of claim 16, wherein the first length is measured from a first nucleotide of the first read to a last nucleotide of the first read and the second length is measured from a first nucleotide of the second read to a last nucleotide of the second read.

18-19. (canceled)

20. The method of claim 1, wherein the tensor further includes a corresponding lymphocyte track.

21. The method of claim 1, wherein the tensor is configured to account for all possible CIGAR (Concise Idiosyncratic Gapped Alignment Report) outputs, wherein the possible CIGAR outputs comprise insertions, deletions, mismatches, clips, and soft masks.

22. (canceled)

23. The method of claim 1, wherein columns of the tensor represent nucleotides along a fragment sequence.

24. The method of claim 1, further comprising filtering the plurality of sequence fragments, wherein the plurality of sequence fragments are filtered based on a quality metric that comprises at least one of: an artificial backlist, discordant reads, variant base quality, depth, mapping quality, number of low quality bases, fragment length, and variant allele fraction.

25-26. (canceled)

27. The method of claim 1, wherein the plurality of sequence fragments each have about 40 base pairs to about 240 base pairs, or have a mean of about 170 base pairs.

28. (canceled)

29. The method of claim 1, wherein the first trained classifier operates sequentially before or in parallel with the second trained classifier.

30-31. (canceled)

32. The method of claim 1, wherein the first predetermined threshold is 0.99 and the second predetermined threshold is 0.99.

33. (canceled)

34. A system comprising:
a reference sequence database; a sequence fragment database; a regional feature database;
a computing node comprising a computer readable storage medium having program instructions embodied therewith, the program instructions executable by a processor of the computing node to cause the processor to perform the method of claim 1.

35-66. (canceled)

67. A computer program product comprising a computer readable storage medium having program instructions embodied therewith, the program instructions executable by a processor to cause the processor to perform the method of claim 1.

68-105. (canceled)
